(19) United States
(12) Patent Application Publication
(10) Pub. No.: US 2016/0159805 A1
(43) Pub. Date: Jun. 9, 2016

(54) Heterocyclic sulfonylamino(thio)carbonyl-derivatives with nematicidal properties

(71) Applicant: BAYER CROPSCIENCE AG, Monheim (DE)

(72) Inventors: Jörg GREUL, Leverkusen (DE); Markus HEIL, Leichlingen (DE); Peter JESCHKE, Bergisch Gladbach (DE); Klaus-Helmut MÜLLER, Düsseldorf (DE); Kerstin ILG, Köln (DE); Olga MALSAM, Rösrath (DE); Daniela PORTZ, Vettweiß (DE)

(21) Appl. No.: 14/904,710

(22) PCT Filed: Jul. 14, 2014
(86) PCT No.: PCT/EP2014/065002

§ 371 Date: Jan. 13, 2016

(30) Foreign Application Priority Data
Jul. 17, 2013 (EP) 13176768..

(51) Int. Cl. C07D 487/04; C07D 231/16; C07D 231/12; C07D 233/61; C07D 471/04; A01N 43/90

(52) U.S. Cl. CPC C07D 487/04; C07D 471/04; A01N 43/90; C07D 231/12; C07D 233/61; C07D 231/16

(57) ABSTRACT

Disclosed are compounds of formula (I) which possess nematicidal properties

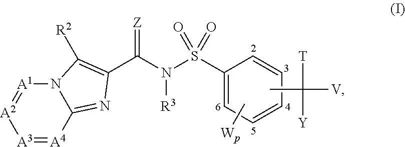

wherein the residues have the meaning as indicated in the description.

This invention relates to certain sulfonamides, their N-oxides, salts and compositions suitable for agronomic and nonagronomic uses, and methods of their use for controlling parasitic nematodes in both agronomic and nonagronomic environments.

The control of plant-parasitic nematodes is extremely important in achieving high crop efficiency. Nematode-induced root damage can cause significant reduction in crop yields and quality and thereby result in increased costs to the consumer. Due to widespread development of resistance to anthelmintic agents in nematode parasites, nematodes continue to cause problems in livestock despite the available chemical therapeutic agents. The need continues for new compounds which are more effective, less costly, less toxic, environmentally safer or have different modes of action.

European Patent Application Publication No. 0 244 166 A2 (referred to as P1) discloses compounds of Formula (i) as herbicides

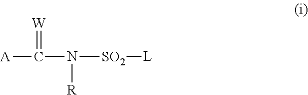

wherein, inter alia, R is H or an organic substituent, W is O or S, L is an aryl or heteroaryl moiety, and A is selected from a list of bi-, tri- and quadricyclic heterocyclic groups.

PCT Patent Application Publication WO 2010/129500 (P2) discloses compounds of Formula (ii) (including all stereoisomers), N-oxides, and salts thereof, and compositions containing them and their use for controlling a parasitic nematode:

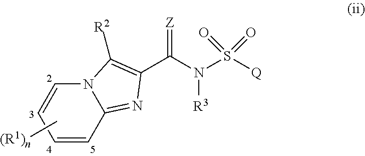

wherein, interalia,
Z is O or S and
Q is phenyl, naphthalenyl, a 5- or 6-membered heteroaromatic ring or an 8-to 10-membered heteroaromatic bicyclic ring system, each optionally substituted with 1 to 5 substituents.

Anonymously disclosed publication IP com Journal 10, 26 (2010) (P3) describes 10 explicitly listed compounds of general formula (ii) in mixtures with various insecticides in several mixture ratios.

PCT Patent Application Publication WO 2012/054233 (P4) discloses compounds of Formula (iii) (including all stereoisomers), N-oxides, and salts thereof, and compositions containing them and their use for controlling a parasitic nematode:

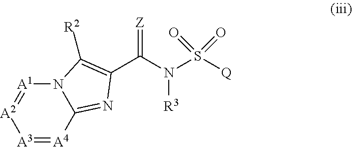

wherein, interalia,
Z is O or S,
Q is phenyl, naphthalenyl, a 5- or 6-membered heteroaromatic ring or an 8-to 10-membered heteroaromatic bicyclic ring system, each optionally substituted with 1 to 5 substituents and
A¹, A², A³ and A⁴ are independently N or CR¹, provided that only one of A¹, A², A³ and A⁴ is N.

PCT Patent Application Publication WO 2013/055584 (P5) discloses solid forms of a certain nematocidal imidazopyridine sulfonamide of formula (ii).

The compounds of the present invention are not disclosed in these publications.

It is an object of the present invention to provide compounds which can be used as nematicides with a satisfactory or improved nematicidal activity, particularly at relatively low application rates, with a high selectivity and high compatibility in crop-plant cultures.

### SUMMARY OF THE INVENTION

This invention is directed to compounds of formula (I) (including all stereoisomers), N-oxides, and salts thereof, and compositions containing them and their use for controlling a parasitic nematode:

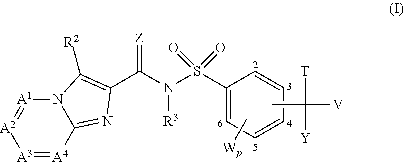

wherein
Z is O or S,
A¹, A², A³ and A⁴ are independently N or C—R¹;
each W is independently hydrogen, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹²; OC(O)R⁷, OC(O)OR⁸, OC(O)N¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R¹ is independently H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)N¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R² is H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R⁷, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, OR^{4a}, and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or C₁-C₆-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)ORS, C(O)NR¹¹R¹², S(O)ₘR⁹, S(O)₂NR¹¹R¹², OC(O)R^{7a} and N(R¹⁰)C(O)R^{7a};
each R⁴ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{4a} is independently H, C₁-C₆-alkyl or C₁-C₆-haloalkyl;
each R⁵ is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{5a} is independently H or C₁-C₆-alkyl;
each R⁶ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{6a} is independently H, C₁-C₆-alkyl, C(O)R¹³ or C(O)OR¹³;
each R⁷ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{7a} is independently C₁-C₆-alkyl or C₁-C₆-haloalkyl;
each R⁸ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{8a} is independently C₁-C₆-alkyl or C₁-C₆-haloalkyl;
each R⁹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl; or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{9a} is independently C₁-C₆-alkyl or C₁-C₆-haloalkyl;
each R¹⁰ is independently H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R^{7a}, C(O)OR⁸a, C(O)NR¹¹R¹², S(O)ₘR^{9a} or S(O)₂NR¹¹R¹²;
each R¹¹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR^{11a}R¹², S(O)ₘR^{9a} and S(O)₂NR^{11a}R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR^{11a}R¹², OR^{4a}, C₂-C₆-alkoxyalkyl, S(O)ₘR^{9a}, S(O)₂NR^{11a}R¹², NR^{5a}R^{6a}, OC(O)R^{7a} and N(R¹⁰)C(O)R^{7a};
each R^{11a} is independently H, C₁-C₆-alkyl, C₂-C₆-alkenyl or C₂-C₆-alkynyl;
each R¹² is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R¹³ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R¹⁴ is independently C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
X is O or S;
T is hydrogen, halogen, C₁-C₄-alkyl or C₁-C₄-haloalkyl;
Y is hydrogen, halogen, C₁-C₄-alkyl or C₁-C₄-haloalkyl;
V is a saturated, partially unsaturated or fully unsaturated via nitrogen bonded heterocyclic group containing 3 to 7 ring atoms;
each m is independently 0, 1 or 2;
p is 0, 1, 2, 3 or 4.

Compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions) and to the mixtures of all the possible stereoisomers, in all proportions. The diastereoisomers and/or the optical isomers can be separated according to the methods which are known per se by the man ordinary skilled in the art.

Compounds of the present invention can also exist in one or more geometric isomer forms depending on the number of double bonds in the compound, especially all syn/anti (or cis/trans) isomers and to all possible syn/anti (or cis/trans) mixtures. The invention thus relates equally to all geometric isomers and to all possible mixtures, in all proportions. The geometric isomers can be separated according to general methods, which are known per se by the man ordinary skilled in the art.

Compounds of formula (I) may be found in its tautomeric form resulting from the shift of the proton of a hydroxy, sulfanyl or amino group. Such tautomeric forms of such compounds are also part of the present invention. More generally speaking, all tautomeric forms of compounds of formula (I), as well as the tautomeric forms of the compounds which can optionally be used as intermediates in the preparation processes and which will be defined in the description of these processes, are also part of the present invention.

Further, this invention is also directed to N-oxides of the compounds of formula (I) (including all stereoisomers), and salts of the compounds of formula (I) (including all stereoisomers).

Further, this invention is directed to compositions comprising compounds of the invention and their use for controlling a parasitic nematode as described above. This invention also provides a composition comprising a compound of formula (I), an N-oxide, or a salt thereof, and at least one additional component selected from the group consisting of surfactants, solid diluents and liquid diluents. In one embodiment, this invention also provides a composition for controlling a parasitic nematode comprising a compound of formula (I), an N-oxide, or a salt thereof, and at least one additional component selected from the group consisting of surfactants, solid diluents and liquid diluents, said composition optionally further comprising at least one additional biologically active compound or agent, preferably a mixing partner as described below.

This invention provides a method for controlling a parasitic nematode comprising contacting the parasitic nematode or its environment with a biologically effective amount of a compound of formula (I), an N-oxide, or a salt thereof, (e.g., as a composition described herein). This invention also relates to such method wherein the parasitic nematode or its environment is contacted with a composition comprising a biologically effective amount of a compound of Formula (I), an N-oxide or a salt thereof, and at least one additional component selected from the group consisting of surfactants, solid diluents and liquid diluents, said composition optionally further comprising a biologically effective amount of at least one additional biologically active compound or agent, preferably a mixing partner as described below.

This invention also provides a method for protecting a seed from a parasitic nematode comprising contacting the seed with a biologically effective amount of a compound of formula (I), an N-oxide, or a salt thereof, (e.g., as a composition described herein). This invention also relates to the treated seed.

### DETAILS OF THE INVENTION

As used herein, the terms “comprises”, “comprising”, “includes”, “including”, “has”, “having”, “contains”, “containing”, “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components or elements, in addition to those literally disclosed, provided that these additional materials, steps, features, components or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used to in the present disclosure and claims, the term “nematode” refers to a living organism of the Phylum Nematoda. As generally defined, a “parasite” lives or grows inside or feeds on another living organism (such as a plant, animal or human) described as the “host”. As referred to in the present disclosure and claims a “parasitic nematode” is particularly a nematode that injures or damages tissue or causes other forms of disease in plants, animals (particularly vertebrates) or humans.

A parasite “infestation” refers to the presence of parasites in numbers that pose a risk to plants, humans or animals. The presence can be in the environment, e.g., in a human or animal house, or surrounding property or structures, on an agricultural crop or other type of plant, in animal bedding, on the skin or fur of an animal, etc. When the infestation that is referred to is within an animal, e.g., in the blood or other internal tissues, the term infestation is also intended to be synonymous with the term, “infection,” as that term is generally understood in the art, unless otherwise stated.

As referred to in the present disclosure and claims, the terms “parasiticidal” and “parasiticidally” refers to observable effects on a parasitic nematode to provide protection of a plant, animal or human from the nematode. Parasiticidal effects typically relate to diminishing the occurrence or activity of the target parasitic nematode. Such effects on the nematode include necrosis, death, retarded growth, diminished mobility or lessened ability to remain on or in the host plant, animal or human, reduced feeding and inhibition of reproduction. These effects on parasitic nematodes provide control (including prevention, reduction or elimination) of parasitic infestation or infection of the plant, animal or human Therefore “control” of a parasitic nematode means achieving a parasiticidal effect on the nematode. The expressions “parasiticidally effective amount” and “biologically effective amount” in the context of applying a chemical compound to control a parasitic nematode refer an amount of the compound that is sufficient to control the parasitic nematode.

The term “agronomic” refers to the production of field crops such as for food and fiber and includes the growth of soybeans and other legumes, cereal (e.g., wheat, oats, barley, rye, rice, maize/com), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from a parasitic nematode by administering a parasiticidally effective (i.e. biologically effective) amount of a compound of the invention, typically in the form of a composition formulated for veterinary use, to the animal to be protected.

In the above recitations, the term “alkyl”, used either alone or in compound words such as “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. “Cycloalkenyl” includes groups such as cyclopentenyl and cyclohexenyl as well as groups with more than 10 one double bond such as 1,3- and 1,4-cyclohexadienyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkenyl”, “haloalkynyl”, and the like, are defined analogously to the term “haloalkyl”. Examples of “haloalkoxy” include CF₃O, CCl₃CH₂O, HCF₂CH₂CH₂O and CF₃CH₂O. Examples of “haloalkenyl” include (Cl)₂C═CHCH₂ and CF₃CH₂CH═CHCH₂. Examples of “haloalkynyl” include HC≡CCHCl, CF₃CC, CCl₃CC and FCH₂C≡CCH₂.

The chemical abbreviation C(O) as used herein represents a carbonyl moiety. For example, C(O)CH₃ represents an acetyl group. The chemical abbreviations CO₂ and C(O)O as used herein represent an ester moiety. For example, CO₂Me and C(O)OMe represent a methyl ester.

“OCN” means —O—C≡N, and “SCN” means —S—C≡N.

The total number of carbon atoms in a substituent group is indicated by the “Cj-Cj” prefix where i and j are numbers from 1 to 14. C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents, e.g. p=0, 1, 2, 3 or 4. When a group contains a substituent which can be hydrogen, for example W, R² or R³, then when this substituent is taken as hydrogen, it is recognized that this is equivalent to said group being unsubstituted. Therefore, hydrogen substituents are not covered by the structural elements, e.g. the structural element p, which indicate how often a specific substituent can be present. Such structural elements therefore refer to substituents other than hydrogen unless otherwise indicated.

Unless otherwise indicated, a “ring” or “ring system” as a component of formula (I) is carbocyclic or heterocyclic. The term “ring system” denotes two or more fused rings. The term “heterocyclic ring” denotes a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. The term “heterocyclic ring system” denotes a ring system in which at least one ring of the ring system is a heterocyclic ring. Unless otherwise indicated, heterocyclic rings and ring systems can be attached through any available carbon or nitrogen by replacement of a hydrogen on said carbon or nitrogen.

As used herein, the following definitions shall apply unless otherwise indicated. The term “optionally substituted” is used interchangeably with the phrase “substituted or unsubstituted” or with the term “(un)substituted”. The expression “optionally substituted with 1 to 4 substituents” means that no substituent is present (i.e. unsubstituted) or that 1, 2, 3 or 4 substituents are present (limited by the number of available bonding positions). Unless otherwise indicated, an optionally substituted group may have a substituent at each substitutable position of the group, and each substitution is independent of the other.

Preferred heterocyclic groups V include the rings V-1 through V-22 illustrated below wherein R^{v} is any substituent as defined in the Summary of the Invention for R¹, R², R³ and r is an integer from 0 to 4, limited by the number of available positions on each V group.

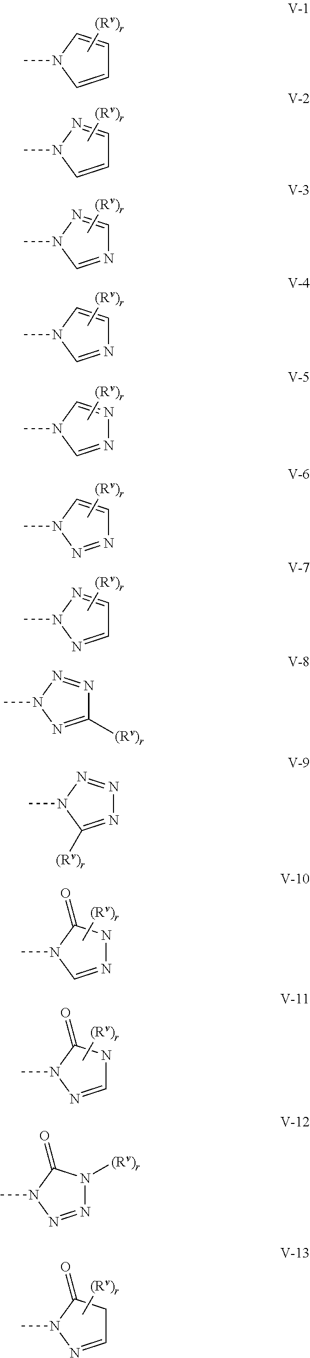

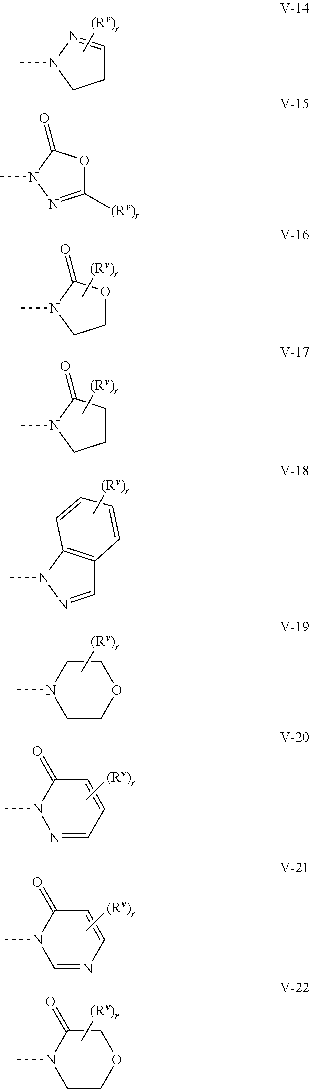

As V-8, V-9, V-12 and V-15 have only one available position, for these V groups r is limited to the integers 0 or 1, and r being 0 means that the V group is unsubstituted and a hydrogen is present at the position indicated by (R^{v})ᵣ

Each W preferably is independently
hydrogen, halogen, cyano, nitro, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of C(X)R⁷, C(O)OR⁸, C(O)NR¹¹R¹², S(O)ₘR⁹ and S(O)₂NR¹¹R¹²;

or phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹² and N(R¹⁰)C(O)R^{7a}.

More preferably, each W is independently
hydrogen, halogen, cyano, nitro, OR⁴, NR⁵R⁶, C₁-C₆-alkyl, C₁-C₆-haloalkyl, C(X)R¹, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹² or C₃-C₇-cycloalkyl, optionally substituted with 1 to 4 substituents independently selected from the group consisting of halogen, cyano, C₁-C₄-alkyl, C₁-C₄-haloalkyl;
or C₁-C₆-alkyl, substituted with 1 to 4 substituents independently selected from the group consisting of S(O)ₘR⁹ and S(O)₂NR¹¹R¹².

Especially preferably, each W is independently
hydrogen, fluorine, chlorine, bromine, methoxy, trifluormethoxy, methyl or ethyl.

Each R¹ preferably is independently
H, halogen, cyano, nitro, OR⁴, NR⁵R⁶, C₁-C₆-alkyl, C₁-C₆-haloalkyl, C₂-C₆-alkenyl, C₂-C₆-haloalkenyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹ or N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, OR⁴, NR⁵R⁶, C(X)R⁷, S(O)ₘR⁹ and S(O)₂NR¹¹R¹².

More preferably, each R¹ is independently
H, halogen, cyano, nitro, OR⁴, NR⁵R⁶, C₁-C₄-alkyl, C₁-C₄-haloalkyl, C(X)R⁷, C(O)OR⁸, C(O)NR¹¹R¹², S(O)ₘR⁹ or S(O)₂NR¹¹R¹²;
or C₃-C₇-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, S(O)ₘR⁹ and S(O)₂NR¹¹R¹².

Especially preferably, each R¹ is independently
H, fluorine, chlorine, bromine, iodine, ethoxy or trifluoromethyl.

R² preferably is
H, halogen, cyano, nitro, OR⁴, NR⁵R⁶, C₁-C₆-alkyl, C₁-C₄-haloalkyl, C₂-C₄-alkenyl, C₂-C₄-haloalkenyl, C₃-C₇-cycloalkyl, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, NR⁵R⁶, S(O)ₘR⁹ and S(O)₂NR¹¹R¹².

More preferably, R² is
H, halogen, cyano, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₄-alkyl substituted with 1 to 2 substituents independently selected from the group consisting of cyano, OR⁴ and S(O)ₘR⁹.

Especially preferably, R² is hydrogen.

R³ preferably is
H, C₁-C₆-alkyl, C₁-C₆-haloalkyl, C₂-C₄-alkenyl, C₂-C₄-haloalkenyl, C₂-C₄-alkynyl, C₂-C₄-haloalkynyl, C(X)R², C(O)OR⁸ or C(O)NR¹¹R¹²;
or C₃-C₇-cycloalkyl or C₄-C₈-cycloalkylalkyl, each optionally substituted with 1 to 4 substituents independently selected from the group consisting of halogen, cyano, OR^{4a} and S(O)ₘR⁹⁸;
or C₁-C₄-alkyl substituted with 1 to 4 substituents independently selected from the group consisting of cyano, OR⁴, C(X)R⁷ and C(O)OR⁸;
or C₁-C₄-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, C₁-C₄-haloalkyl, C₂-C₆-alkoxyalkyl, C(X)R⁷, C(O)OR⁸, S(O)ₘR⁹ and S(O)₂NR¹¹R¹².

More preferably, R³ is
H, C₁-C₄-alkyl, C₂-C₃-alkenyl, C(X)R⁷ or C(O)OR⁸;
or C₁-C₂-alkyl substituted with OR⁴;
or C₁-C₂-alkyl substituted with phenyl.

Especially preferably, R³ is hydrogen.

Each R⁴ preferably is independently
H, C₁-C₄-alkyl or C₁-C₄-haloalkyl;
or C₃-C₇-cycloalkyl or C₄-C₈-cycloalkylalkyl, each optionally substituted with 1 to 4 substituents independently selected from the group consisting of halogen.

More preferably, each R⁴ is independently
H, C₁-C₄-alkyl or C₁-C₄-haloalkyl.

Especially preferably, each R⁴ is methyl, ethyl or trifluoromethyl.

Each R^{4a} preferably is independently
H, C₁-C₆-alkyl or C₁-C₆-haloalkyl.

More preferably, each R^{4a} is independently
C₁-C₄-alkyl or C₁-C₄-haloalkyl.

Especially preferably, each R^{4a} is independently
methyl or ethyl.

Each R⁵ preferably is independently
H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹²;
or C₃-C₇-cycloalkyl or C₄-C₈-cycloalkylalkyl, each optionally substituted with 1 to 4 substituents independently selected from the group consisting of halogen, cyano, C₁-C₄-alkyl; C₁-C₄-haloalkyl, OR^{4a} and S(O)ₘR^{9a}.
   More preferably, each R⁵ is independently

H, C₁-C₄-alkyl, C₁-C₄-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹².

Especially preferably, each R⁵ is independently
H, methyl or ethyl.

Each R^{5a} preferably is independently
H or C₁-C₄-alkyl.

More preferably, each R^{5a} is independently
H or C₁-C₂-alkyl.

Especially preferably, each R^{5a} is independently
methyl or ethyl.

Each R⁶ preferably is independently
H, C₁-C₄-alkyl, C₁-C₄-haloalkyl, C₂-C₄-alkenyl, C₂-C₄-haloalkenyl, C₂-C₄-alkynyl or C₂-C₄-haloalkynyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR^{4a} and S(O)ₘR^{9a}.

More preferably, each R⁶ is independently
H, C₁-C₄-alkyl, C₁-C₄-haloalkyl, C₂-C₄-alkenyl or C₂-C₄-haloalkenyl;
or C₃-C₇-cycloalkyl optionally substituted with 1 to 4 substituents independently selected from the group consisting of halogen, cyano, C₁-C₄-alkyl and C₁-C₄-haloalkyl.

Especially preferably, each R⁶ is independently
H, methyl or ethyl.

Each R^{6a} preferably is independently
H, C₁-C₄-alkyl, C(O)R¹³ or C(O)OR¹³.

More preferably, each R^{6a} is independently
H or C₁-C₄-alkyl.

Especially preferably, each R^{6a} is independently
H, methyl or ethyl.

Each R⁷ preferably is independently
H, C₁-C₆-alkyl, C₁-C₆-haloalkyl, C₂-C₆-alkenyl or C₂-C₆-haloalkenyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR⁴a and S(O)ₘR^{9a}.

More preferably, each R⁷ is independently
H, C₁-C₄-alkyl or C₁-C₄-haloalkyl;
or C₃-C₇-cycloalkyl optionally substituted with 1 to 4 substituents independently selected from the group consisting of halogen, cyano, C₁-C₂-alkyl and C₁-C₂-haloalkyl.

Especially preferably, each R⁷ is independently
H, methyl, ethyl or trifluoromethyl.

Each R^{7a} preferably is independently
C₁-C₄-alkyl or C₁-C₄-haloalkyl.

More preferably, each R^{7a} is independently
C₁-C₄-alkyl or C₁-C₂-haloalkyl.

Especially preferably, each R^{7a} is independently
methyl or ethyl.

Each R⁸ preferably is independently
H, C₁-C₄-alkyl or C₁-C₄-haloalkyl.

More preferably, each R⁸ is independently
C₁-C₂-alkyl or C₁-C₂-haloalkyl.

Especially preferably, each R⁸ is independently
methyl or ethyl.

Each R^{8a} preferably is independently
C₁-C₄-alkyl or C₁-C₄-haloalkyl.

More preferably, each R^{8a} is independently
C₁-C₄-alkyl.

Especially preferably, each R^{8a} is independently
methyl or ethyl.

Each R⁹ preferably is independently
C₁-C₄-alkyl or C₁-C₄-haloalkyl.

More preferably, each R⁹ is independently
C₁-C₂-alkyl, C₁-C₂-haloalkyl.

Especially preferably, each R⁹ is independently
methyl, ethyl or trifluoromethyl.

Each R^{9a} preferably is independently
C₁-C₄-alkyl or C₁-C₄-haloalkyl.

More preferably, each R^{9a} is independently
C₁-C₄-alkyl or C₁-C₂-haloalkyl.

Especially preferably, each R^{9a} is independently
methyl, ethyl or trifluoromethyl.

Each R¹⁰ preferably is independently
H, C₁-C₄-alkyl, C₁-C₄-haloalkyl, C₂-C₃-alkenyl, C₂-C₃-haloalkenyl, C₂-C₃-alkynyl, C₂-C₃-haloalkynyl or C₃-C₇-cycloalkyl.

More preferably, each R¹⁰ is independently
H, C₁-C₄-alkyl, C₂-C₃-alkenyl or C₃-C₇-cycloalkyl.

Especially preferably, each R¹⁰ is independently
H or methyl.

Each R¹¹ preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl or C₃-C₇-cycloalkyl.

More preferably, each R¹¹ is independently
H, C₁-C₄-alkyl, C₁-C₄-haloalkyl.

Especially preferably, each R¹¹ is independently
H, methyl or ethyl.

Each R¹¹ preferably is independently
H, C₁-C₄-alkyl, C₂-C₃-alkenyl or C₂-C₃-alkynyl.

More preferably, each R¹¹ is independently
H, C₁-C₄-alkyl, allyl or propargyl.

Especially preferably, each R^{11a} is independently
H, methyl or ethyl.

Each R¹² preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹² is independently
H, C₁-C₄-alkyl or C₁-C₄-haloalkyl.

Especially preferably, each R¹² is independently
H, methyl or ethyl.

Each R^{H} preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹³ is independently
H, C₁-C₄-alkyl or C₁-C₄-haloalkyl.

Especially preferably, each R¹³ is independently
methyl.

Each R¹⁴ preferably is independently
C₁-C₄-alkyl or C₁-C₄-haloalkyl;
or phenyl optionally substituted with 1 to 5 substituents independently selected from the group consisting of halogen, cyano, OR⁴, NR⁵R⁶, C₁-C₄-alkyl, C(X)R⁷, C(O)OR⁸, C(O)NR¹¹R¹², S(O)ₘR⁹ and S(O)₂NR¹¹R¹².

More preferably, each R¹⁴ is independently
C₁-C₄-alkyl or C₁-C₄-haloalkyl.

Especially preferably, each R¹⁴ is independently
methyl.

X preferably is O or S. Especially preferably X is O.

p preferably is 0, 1, 2, 3 or 4.

More preferably, p is 0, 1, 2 or 3.

Especially preferably, p is 0, 1 or 2.

In all embodiments mentioned above with V is defined to be one of V-1 to V-22.

each R^{v} preferably is independently
H, halogen, cyano, nitro, C₁-C₄-alkyl, C₁-C₄-haloalkyl, O(C₁-C₄-alkyl), O(C₁-C₄-haloalkyl), S(C₁-C₄-alkyl), S(C₁-C₄-haloalkyl), S(O)(C₁-C₄-haloalkyl), S(O)₂(C₁-C₄-haloalkyl), C(O)O(C₁-C₄-alkyl), phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a}.

More preferably, each R^{v} is independently
H, halogen, cyano, nitro, C₁-C₄-alkyl, C₁-C₄-haloalkyl, O(C₁-C₄-alkyl), O(C₁-C₄-haloalkyl), C(O)O(C₁-C₂-alkyl), phenyl optionally substituted with 1 to 3 substituents independently selected from the group consisting of halogen, cyano, nitro, O(C₁-C₄-alkyl), C₁-C₄-alkyl.

Especially preferably, each R^{v} is independently
H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl.

In all embodiments mentioned above with V is defined to be one of V-1 to V-22, r preferably is 0, 1, 2 or 3. More preferably r is 0, 1 or 2. Especially preferably, r is 0, 1 or 2.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers (R- or S-enantiomers) or as an optically active form.

Compounds selected from formula (I), (including all stereoisomers, N-oxides, and salts thereof), typically exist in more than one form, and formula (I) thus includes all crystalline and non-crystalline forms of the compounds that formula (I) represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by formula (I) can exhibit beneficial effects (e.g., suitability for preparation of useful formulations, improved biological performance) relative to another polymorph or a mixture of polymorphs of the same compound represented by formula (I). Preparation and isolation of a particular polymorph of a compound represented by formula (I) can be achieved by methods known to those skilled in the art including, for example, crystallization using selected solvents and temperatures.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of formula (I) are useful for control of parasitic nematodes. The salts of the compounds of formula (I) include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of formula (I) contains an acidic moiety such as a carboxylic acid, phenol or sulfonylamide (i.e. when R³ is H), salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from formula (I), N-oxides and salts thereof.

Embodiments of the present invention as described in the Summary of the Invention include those described below. In the following Embodiments, formula (I) includes stereoisomers, N-oxides, and salts thereof, and reference to “a compound of formula (I)” includes the definitions of substituents specified in the Summary of the Invention unless further defined in the embodiments.

### EMBODIMENTS

The compounds of the invention are defined in general terms by the formula (I). Preferred substituents (′embodiments 1″) and ranges of the radicals given in the formulae referred to above and below are defined,

wherein
Z is O or S,
A¹, A², A³ and A⁴ are C—R¹, or

one of A¹, A², A³ and A⁴ is N and the remaining are C—R¹;

each W is independently hydrogen, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹²; OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₂-C₄-alkenyl, C₂-C₄-alkynyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R¹ is independently H, halogen, cyano, nitro, SF₅, OCN, SCN, Si(R¹⁴)₃, OR⁴, NR⁵R⁶, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R² is H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R⁷, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁹)C(O)NR¹¹R¹², N(R¹⁰)S(O)₂R⁹ or N(R¹⁹)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, OR^{4a}, and S(O)ₘR^{9a};
or C₁-C₆-alkyl substituted with 1 to 4 substituents independently selected from the group consisting of cyano, nitro, OR⁴, NR⁵R⁶, C(X)R¹, C(O)OR⁸, C(O)NR¹¹R¹², S(O)ₘR⁹ and S(O)₂NR¹¹R¹²;
or C₁-C₆-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R⁴ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{4a} is independently H, C₁-C₆-alkyl or C₁-C₆-haloalkyl;
each R⁵ is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{5a} is independently H or C₁-C₆-alkyl;
each R⁶ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{5a} is independently H, C₁-C₆-alkyl, C(O)R¹³ or C(O)OR¹³;
each R⁷ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{7a} is independently C₁-C₆-alkyl or C₁-C₆-haloalkyl;
each R⁸ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{8a} is independently C₁-C₆-alkyl or C₁-C₆-haloalkyl;
each R⁹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl; or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{9a} is independently C₁-C₆-alkyl or C₁-C₆-haloalkyl;
each R¹⁰ is independently H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R^{7a}, C(O)OR⁸a, C(O)NR¹¹R¹², S(O)ₘR^{9a} or S(O)₂NR¹¹R¹²;
each R¹¹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR^{11a}R¹², S(O)ₘR^{9a} and S(O)₂NR^{11a}R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR^{11a}R¹², OR^{4a}, C₂-C₆-alkoxyalkyl, S(O)ₘR^{9a}, S(O)₂NR^{11a}R¹², NR^{5a}R^{6a}, OC(O)R^{7a} and N(R¹⁰)C(O)R^{7a};
each R^{11a} is independently H, C₁-C₆-alkyl, C₂-C₆-alkenyl or C₂-C₆-alkynyl;
each R¹² is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each Rⁿ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl,
each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{N} is independently C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
X is O or S;
T is hydrogen, C₁-C₄-alkyl or C₁-C₄-haloalkyl, particularly hydrogen;
Y is hydrogen, C₁-C₄-alkyl or C₁-C₄-haloalkyl, particularly hydrogen;
V is a saturated, partially unsaturated or fully unsaturated via nitrogen bonded heterocyclic group containing 5 to 6 ring atoms;
each m is independently 0, 1 or 2;
p is 0, 1, 2, 3 or 4.

In an individual type of this embodiment, V is selected from the group consisting of V-1, V-2, V-3, V-4, V-5, V-6, V-7, V-8, V-9, V-10, V-11, V-12, V-13, V-14, V-15, V-16, V-17, V-18, V-19, V-20, V-21 and V-22 as defined above. In another individual type of this embodiment, V is selected from the group consisting of V-2, V-3, V-4, V-6, V-7, V-8, V-9, V-11, V-13, V-14, V-18, V-19 and V-20 as defined above. In another individual type of this embodiment, V is V-1. In another individual type of this embodiment, V is V-2. In another individual type of this embodiment, V is V-3. In another individual type of this embodiment, V is V-4. In another individual type of this embodiment, V is V-5. In another individual type of this embodiment, V is V-6. In another individual type of this embodiment, V is V-7. In another individual type of this embodiment, V is V-8. In another individual type of this embodiment, V is V-9. In another individual type of this embodiment, V is V-10. In another individual type of this embodiment, V is V-11. In another individual type of this embodiment, V is V-12. In another individual type of this embodiment, V is V-13. In another individual type of this embodiment, V is V-14. In another individual type of this embodiment, V is V-15. In another individual type of this embodiment, V is V-16. In another individual type of this embodiment, V is V-17. In another individual type of this embodiment, V is V-18. In another individual type of this embodiment, V is V-19. In another individual type of this embodiment, V is V-20. In another individual type of this embodiment, V is V-21. In another individual type of this embodiment, V is V-22.

In all embodiments (“embodiments 1”) mentioned above,

each W preferably is independently
hydrogen, halogen, cyano, nitro, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of C(X)R⁷, C(O)OR⁸, C(O)NR¹¹R¹², S(O)ₘR⁹ and S(O)₂NR¹¹R¹²;

or phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹² and N(R¹⁰)C(O)R^{7a}.

More preferably, each W is independently
hydrogen, halogen, cyano, nitro, OR⁴, NR⁵R⁶, C₁-C₆-alkyl, C₁-C₆-haloalkyl, C(X)R¹, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹² or C₃-C₇-cycloalkyl, optionally substituted with 1 to 4 substituents independently selected from the group consisting of halogen, cyano, C₁-C₄-haloalkyl;
or C₁-C₆-alkyl, substituted with 1 to 4 substituents independently selected from the group consisting of S(O)ₘR⁹ and S(O)₂NR¹¹R¹².

Especially preferably, each W is independently
hydrogen, fluorine, chlorine, bromine, methoxy, trifluormethoxy, methyl or ethyl.

In all embodiments (“embodiments 1”) mentioned above,

each R¹ preferably is independently
H, halogen, cyano, nitro, OR⁴, NR⁵R⁶, C₁-C₆-haloalkyl, C₂-C₆-alkenyl, C₂-C₆-haloalkenyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, OR⁴, NR⁵R⁶, C(X)R⁷, S(O)ₘR⁹ and S(O)₂NR¹¹R¹².

More preferably, each R¹ is independently
H, halogen, cyano, nitro, OR⁴, NR⁵R⁶, C(X)R¹, C(O)OR⁸, C(O)NR¹¹R¹², S(O)ₘR⁹ or S(O)₂NR¹¹R¹²;
or C₃-C₇-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, S(O)ₘR⁹ and S(O)₂NR¹¹R¹².

Especially preferably, each R¹ is independently
H, fluorine, chlorine, bromine, iodine, ethoxy or trifluoromethyl.

In all embodiments (“embodiments 1”) mentioned above,
R² preferably is H, halogen, cyano, nitro, OR⁴, NR⁵R⁶, C₁-C₄-haloalkyl, C₂-C₄-alkenyl, C₂-C₄-haloalkenyl, C₃-C₇-cycloalkyl, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, NR⁵R⁶, S(O)ₘR⁹ and S(O)₂NR¹¹R¹².

More preferably, R² is
H, halogen, cyano, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₄-alkyl substituted with 1 to 2 substituents independently selected from the group consisting of cyano, OR⁴ and S(O)ₘR⁹.

Especially preferably, R² is hydrogen.

In all embodiments (“embodiments 1”) mentioned above,

R³ preferably is
H, C₁-C₆-alkyl, C₁-C₆-haloalkyl, C₂-C₄-alkenyl, C₂-C₄-haloalkenyl, C₂-C₄-alkynyl, C₂-C₄-haloalkynyl, C(X)R⁷, C(O)OR⁸ or C(O)NR¹¹R¹²;
or C₃-C₇-cycloalkyl or C₄-C₈-cycloalkylalkyl, each optionally substituted with 1 to 4 substituents independently selected from the group consisting of halogen, cyano, OR^{4a} and S(O)ₘR^{9a};
or C₁-C₄-alkyl substituted with 1 to 4 substituents independently selected from the group consisting of cyano, OR⁴, C(X)R⁷ and C(O)OR⁸;
or C₁-C₄-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, C₁-C₄-haloalkyl, C₂-C₆-alkoxyalkyl, C(X)R⁷, C(O)OR⁸, S(O)ₘR⁹ and S(O)₂NR¹¹R¹².

More preferably, R³ is
H, C₁-C₄-alkyl, C₂-C₃-alkenyl, C(X)R⁷ or C(O)OR⁸;
or C₁-C₂-alkyl substituted with OR⁴;
or C₁-C₂-alkyl substituted with phenyl.

Especially preferably, R³ is hydrogen.

In all embodiments (“embodiments 1”) mentioned above, each R⁴ preferably is independently
H, C₁-C₄-alkyl or C₁-C₄-haloalkyl;
or C₃-C₇-cycloalkyl or C₄-C₈-cycloalkylalkyl, each optionally substituted with 1 to 4 substituents independently selected from the group consisting of halogen.

More preferably, each R⁴ is independently
H, C₁-C₄-alkyl or C₁-C₄-haloalkyl.

Especially preferably, each R⁴ is methyl, ethyl or trifluoromethyl.

In all embodiments (“embodiments 1”) mentioned above, each R^{4a} preferably is independently
H, C₁-C₆-alkyl or C₁-C₆-haloalkyl.

More preferably, each R^{4a} is independently
C₁-C₄-alkyl or C₁-C₄-haloalkyl.

Especially preferably, each R^{4a} is independently
methyl or ethyl.

In all embodiments (“embodiments 1”) mentioned above,

each R⁵ preferably is independently
H, C₁-C₆-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹²;
or C₃-C₇-cycloalkyl or C₄-C₈-cycloalkylalkyl, each optionally substituted with 1 to 4 substituents independently selected from the group consisting of halogen, cyano, C₁-C₄-alkyl; C₁-C₄-haloalkyl, OR⁴a and S(O)ₘR^{9a}.

More preferably, each R⁵ is independently
H, C₁-C₄-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹².

Especially preferably, each R⁵ is independently
H, methyl or ethyl.

In all embodiments (“embodiments 1”) mentioned above,

each R^{5a} preferably is independently
H or C₁-C₄-alkyl.

More preferably, each R^{5a} is independently
H or C₁-C₂-alkyl.

Especially preferably, each R^{5a} is independently
methyl or ethyl.

In all embodiments (“embodiments 1”) mentioned above,

each R⁶ preferably is independently
H, C₁-C₄-alkyl, C₁-C₄-haloalkyl, C₂-C₄-alkenyl, C₂-C₄-haloalkenyl, C₂-C₄-alkynyl or C₂-C₄-haloalkynyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR⁴a and S(O)ₘR^{9a}.

More preferably, each R⁶ is independently
H, C₁-C₄-alkyl, C₁-C₄-haloalkyl, C₂-C₄-alkenyl or C₂-C₄-haloalkenyl;
or C₃-C₇-cycloalkyl optionally substituted with 1 to 4 substituents independently selected from the group consisting of halogen, cyano, C₁-C₄-alkyl and C₁-C₄-haloalkyl.

Especially preferably, each R⁶ is independently
H, methyl or ethyl.

In all embodiments (“embodiments 1”) mentioned above,

each R^{6a} preferably is independently
H, C₁-C₄-alkyl, C(O)R¹³ or C(O)OR¹³.

More preferably, each R^{6a} is independently
H or C₁-C₄-alkyl.

Especially preferably, each R^{6a} is independently
H, methyl or ethyl.

In all embodiments (“embodiments 1”) mentioned above, each R⁷ preferably is independently
H, C₁-C₆-alkyl, C₁-C₆-haloalkyl, C₂-C₆-alkenyl or C₂-C₆-haloalkenyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR⁴a and S(O)ₘR^{9a}.

More preferably, each R⁷ is independently
H, C₁-C₄-alkyl or C₁-C₄-haloalkyl;
or C₃-C₇-cycloalkyl optionally substituted with 1 to 4 substituents independently selected from the group consisting of halogen, cyano, C₁-C₂-alkyl and C₁-C₂-haloalkyl.

Especially preferably, each R⁷ is independently
H, methyl, ethyl or trifluoromethyl.

In all embodiments (“embodiments 1”) mentioned above,

each R^{7a} preferably is independently
C₁-C₄-alkyl or C₁-C₄-haloalkyl.

More preferably, each R^{7a} is independently
C₁-C₄-alkyl or C₁-C₂-haloalkyl.

Especially preferably, each R^{7a} is independently methyl or ethyl.

In all embodiments (“embodiments 1”) mentioned above,

each R⁸ preferably is independently
H, C₁-C₄-alkyl or C₁-C₄-haloalkyl.

More preferably, each R⁸ is independently
C₁-C₂-alkyl or C₁-C₂-haloalkyl.

Especially preferably, each R⁸ is independently
methyl or ethyl.

In all embodiments (“embodiments 1”) mentioned above,

each R^{8a} preferably is independently
C₁-C₄-alkyl or C₁-C₄-haloalkyl.

More preferably, each R^{8a} is independently
C₁-C₄-alkyl.

Especially preferably, each R^{8a} is independently
methyl or ethyl.

In all embodiments (“embodiments 1”) mentioned above,

each R⁹ preferably is independently
C₁-C₄-alkyl or C₁-C₄-haloalkyl.

More preferably, each R⁹ is independently
C₁-C₂-alkyl, C₁-C₂-haloalkyl.

Especially preferably, each R⁹ is independently
methyl, ethyl or trifluoromethyl.

In all embodiments (“embodiments 1”) mentioned above,

each R^{9a} preferably is independently
C₁-C₄-alkyl or C₁-C₄-haloalkyl.

More preferably, each R^{9a} is independently
C₁-C₄-alkyl or C₁-C₂-haloalkyl.

Especially preferably, each R^{9a} is independently
methyl, ethyl or trifluoromethyl.

In all embodiments (“embodiments 1”) mentioned above,

each R¹⁰ preferably is independently
H, C₁-C₄-alkyl, C₁-C₄-haloalkyl, C₂-C₃-alkenyl, C₂-C₃-haloalkenyl, C₂-C₃-alkynyl, C₂-C₃-haloalkynyl or C₃-C₇-cycloalkyl.

More preferably, each R¹⁰ is independently
H, C₁-C₄-alkyl, C₂-C₃-alkenyl or C₃-C₇-cycloalkyl.

Especially preferably, each R¹⁰ is independently
H or methyl.

In all embodiments (“embodiments 1”) mentioned above,

each R¹¹ preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl or C₃-C₇-cycloalkyl.

More preferably, each R¹¹ is independently
H, C₁-C₄-alkyl, C₁-C₄-haloalkyl.

Especially preferably, each R¹¹ is independently
H, methyl or ethyl.

In all embodiments (“embodiments 1”) mentioned above,

each R^{11a} preferably is independently
H, C₁-C₄-alkyl, C₂-C₃-alkenyl or C₂-C₃-alkynyl.

More preferably, each R^{11a} is independently
H, C₁-C₄-alkyl, allyl or propargyl.

Especially preferably, each R^{11a} is independently
H, methyl or ethyl.

In all embodiments (“embodiments 1”) mentioned above,

each R¹² preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹² is independently
H, C₁-C₄-alkyl or C₁-C₄-haloalkyl.

Especially preferably, each R¹² is independently
H, methyl or ethyl.

In all embodiments (“embodiments 1”) mentioned above,

each R¹³ preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹³ is independently
H, C₁-C₄-alkyl or C₁-C₄-haloalkyl.

Especially preferably, each R¹³ is independently
methyl.

In all embodiments (“embodiments 1”) mentioned above,

each R^{N} preferably is independently
C₁-C₄-alkyl or C₁-C₄-haloalkyl;
or phenyl optionally substituted with 1 to 5 substituents independently selected from the group consisting of halogen, cyano, OR⁴, NR⁵R⁶, C₁-C₄-alkyl, C(X)R⁷, C(O)OR⁸, C(O)NR¹¹R¹², S(O)ₘR⁹ and S(O)₂NR¹¹R¹².

More preferably, each R¹⁴ is independently
C₁-C₄-alkyl or C₁-C₄-haloalkyl.

Especially preferably, each R¹⁴ is independently
methyl.

In all embodiments (“embodiments 1”) mentioned above, X preferably is O or S. Especially preferably X is O.

In all embodiments (“embodiments 1”) mentioned above, p preferably is 0, 1, 2, 3 or 4.

More preferably, p is 0, 1, 2 or 3.

Especially preferably, p is 0, 1 or 2.

In all embodiments (“embodiments 1”) mentioned above with V is defined to be one of V-1 to V-22,

each R^{v} preferably is independently
H, halogen, cyano, nitro, C₁-C₄-alkyl, C₁-C₄-haloalkyl, O(C₁-C₄-alkyl), O(C₁-C₄-haloalkyl), S(C₁-C₄-alkyl), S(C₁-C₄-haloalkyl), S(O)(C₁-C₄-haloalkyl), S(O)₂(C₁-C₄-haloalkyl), C(O)O(C₁-C₄-alkyl), phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a}.

More preferably, each R^{v} is independently
H, halogen, cyano, nitro, C₁-C₄-alkyl, C₁-C₄-haloalkyl, O(C₁-C₄-alkyl), O(C₁-C₄-haloalkyl), C(O)O(C₁-C₂-alkyl), phenyl optionally substituted with 1 to 3 substituents independently selected from the group consisting of halogen, cyano, nitro, O(C₁-C₄-alkyl), C₁-C₄-alkyl.

Especially preferably, each R^{v} is independently
H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl.

In all embodiments mentioned above with V is defined to be one of V-1 to V-22, r preferably is 0, 1, 2 or 3. More preferably r is 0, 1 or 2. Especially preferably, r is 0, 1 or 2.

The compounds of the invention are defined in general terms by the formula (I). Very preferred (“embodiments 2”) substituents and ranges of the radicals given in the formulae referred to above and below are defined,

wherein
Z is O or S;
A¹, A², A³ and A⁴ are C—R¹, or
A¹ is N and A², A³ and A⁴ are C—R¹, or
A³ is N and A¹, A² and A⁴ are C—R¹;
each W is independently hydrogen, halogen, cyano, nitro, SF₅, OCN, SCN, Si(R¹⁴)₃, OR⁴, NR⁵R⁶, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹²; OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of NR⁵R⁶, C(X)R⁷, C(O)OR⁸, C(O)NR¹¹R¹², S(O)ₘR⁹ and S(O)₂NR¹¹R¹²;

or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
R¹ is independently H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R² is H, halogen, cyano, nitro, SF₅, OCN, SCN, Si(R¹⁴)₃, OR⁴, NR⁵R⁶, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R⁷, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, OR^{4a}, and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or C₁-C₆-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R⁴ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R¹¹ is independently H, C₁-C₆-alkyl or C₁-C₆-haloalkyl;
each R⁵ is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{5a} is independently H or C₁-C₆-alkyl;
each R⁶ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{6a} is independently H, C₁-C₆-alkyl, C(O)R¹³ or C(O)OR¹³;
each R⁷ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{7a} is independently C₁-C₆-alkyl or C₁-C₆-haloalkyl;
each R⁸ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};

or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{8a} is independently C₁-C₆-alkyl or C₁-C₆-haloalkyl;
each R⁹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl; or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{9a} is independently C₁-C₆-alkyl or C₁-C₆-haloalkyl;
each R¹⁰ is independently H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R^{7a}, C(O)OR^{8a}, C(O)NR¹¹R¹², S(O)ₘR^{9a} or S(O)₂NR¹¹R¹²;
each R¹¹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR^{11a}R¹², S(O)ₘR^{9a} and S(O)₂NR^{11a}R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR^{11a}R¹², OR^{4a}, C₂-C₆-alkoxyalkyl, S(O)ₘR^{9a}, S(O)₂NR^{11a}R¹², NR^{5a}R^{6a}, OC(O)R^{7a} and N(R¹⁰)C(O)R^{7a};
each R^{11a} is independently H, C₁-C₆-alkyl, C₂-C₆-alkenyl or C₂-C₆-alkynyl;
each R¹² is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each Rⁿ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R¹⁴ is independently C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 5 substituents independently selected from the group consisting of halogen, cyano, nitro, OR⁴, NR⁵R⁶, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
X is O or S;

T is hydrogen or methyl;
Y is hydrogen or methyl;
V is a fully unsaturated via nitrogen bonded heterocyclic group containing 5 ring atoms;
each m is independently 0, 1 or 2.
p is 0, 1, 2, 3 or 4.

In an individual type of this embodiment, V is selected from the group consisting of V-1, V-2, V-3, V-4, V-5, V-6, V-7, V-8, V-9, V-10, V-11, V-12, V-13, V-14, V-15, V-16, V-17, V-18, V-19, V-20, V-21 and V-22 as defined above. In another individual type of this embodiment, V is selected from the group consisting of V-2, V-3, V-4, V-6, V-7, V-8, V-9, V-11, V-13, V-14, V-18, V-19 and V-20 as defined above. In another individual type of this embodiment, V is V-1. In another individual type of this embodiment, V is V-2. In another individual type of this embodiment, V is V-3. In another individual type of this embodiment, V is V-4. In another individual type of this embodiment, V is V-5. In another individual type of this embodiment, V is V-6. In another individual type of this embodiment, V is V-7. In another individual type of this embodiment, V is V-8. In another individual type of this embodiment, V is V-9. In another individual type of this embodiment, V is V-10. In another individual type of this embodiment, V is V-11. In another individual type of this embodiment, V is V-12. In another individual type of this embodiment, V is V-13. In another individual type of this embodiment, V is V-14. In another individual type of this embodiment, V is V-15. In another individual type of this embodiment, V is V-16. In another individual type of this embodiment, V is V-17. In another individual type of this embodiment, V is V-18. In another individual type of this embodiment, V is V-19. In another individual type of this embodiment, V is V-20. In another individual type of this embodiment, V is V-21. In another individual type of this embodiment, V is V-20. In another individual type of this embodiment, V is V-22.

In all embodiments (“embodiments 2”) mentioned above,

each W preferably is independently
hydrogen, halogen, cyano, nitro, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of C(X)R⁷, C(O)OR⁸, C(O)NR¹¹R¹², S(O)ₘR⁹ and S(O)₂NR¹¹R¹²;

or phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹² and N(R¹⁰)C(O)R^{7a}.

More preferably, each W is independently
hydrogen, halogen, cyano, nitro, OR⁴, NR⁵R⁶, C₁-C₆-alkyl, C₁-C₆-haloalkyl, C(X)R⁷, C(O)OR⁸, C(O)NR¹¹R¹², S(O)ₘR⁹, S(O)₂NR¹¹R¹², N(R¹⁰) C(O)R⁷, N(R¹⁰)C(O)NR¹¹R¹², N(R¹⁰)S(O)₂R⁹, N(R¹⁰)S(O)₂NR¹¹R¹² or C₃-C₇-cycloalkyl, optionally substituted with 1 to 4 substituents independently selected from the group consisting of halogen, cyano, C₁-C₄-alkyl, C₁-C₄-haloalkyl;
or C₁-C₆-alkyl, substituted with 1 to 4 substituents independently selected from the group consisting of S(O)ₘR⁹ and S(O)₂NR¹¹R¹².

Especially preferably, each W is independently
hydrogen, fluorine, chlorine, bromine, methoxy, trifluormethoxy, methyl or ethyl.

In all embodiments (“embodiments 2”) mentioned above,

each R¹⁰ preferably is independently
H, halogen, cyano, nitro, OR⁴, NR⁵R⁶, C₁-C₆-alkyl, C₁-C₆-haloalkyl, C₂-C₆-alkenyl, C₂-C₆-haloalkenyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, OR⁴, NR⁵R⁶, C(X)R⁷, S(O)ₘR⁹ and S(O)₂NR¹¹R¹².

More preferably, each R¹ is independently
H, halogen, cyano, nitro, OR⁴, NR⁵R⁶, C₁-C₄-alkyl, C₁-C₄-haloalkyl, C(X)R⁷, C(O)OR⁸, C(O)NR¹¹R¹², S(O)ₘR⁹ or S(O)₂NR¹¹R¹²;
or C₃-C₇-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, S(O)ₘR⁹ and S(O)₂NR¹¹R¹².

Especially preferably, each R¹ is independently
H, fluorine, chlorine, bromine, iodine, ethoxy or trifluoromethyl.

In all embodiments (“embodiments 2”) mentioned above,
R² preferably is H, halogen, cyano, nitro, OR⁴, NR⁵R⁶, C₁-C₆-alkyl, C₁-C₄-haloalkyl, C₂-C₄-alkenyl, C₂-C₄-haloalkenyl, C₃-C₇-cycloalkyl, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, NR⁵R⁶, S(O)ₘR⁹ and S(O)₂NR¹¹R¹².

More preferably, R² is
H, halogen, cyano, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₄-alkyl substituted with 1 to 2 substituents independently selected from the group consisting of cyano, OR⁴ and S(O)ₘR⁹.

Especially preferably, R² is hydrogen.

In all embodiments (“embodiments 2”) mentioned above,

R³ preferably is
H, C₁-C₆-alkyl, C₁-C₆-haloalkyl, C₂-C₄-alkenyl, C₂-C₄-haloalkenyl, C₂-C₄-alkynyl, C₂-C₄-haloalkynyl, C(X)R⁷, C(O)OR⁸ or C(O)NR¹¹R¹²;
or C₃-C₇-cycloalkyl or C₄-C₈-cycloalkylalkyl, each optionally substituted with 1 to 4 substituents independently selected from the group consisting of halogen, cyano, OR^{4a} and S(O)ₘR^{9a};
or C₁-C₄-alkyl substituted with 1 to 4 substituents independently selected from the group consisting of cyano, OR⁴, C(X)R⁷ and C(O)OR⁸;
or C₁-C₄-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, C₁-C₄-haloalkyl, C₂-C₆-alkoxyalkyl, C(X)R⁷, C(O)OR⁸, S(O)ₘR⁹ and S(O)₂NR¹¹R¹².

More preferably, R³ is
H, C₁-C₄-alkyl, C₂-C₃-alkenyl, C(X)R⁷ or C(O)OR⁸;
or C₁-C₂-alkyl substituted with OR⁴;
or C₁-C₂-alkyl substituted with phenyl.

Especially preferably, R³ is hydrogen.

In all embodiments (“embodiments 2”) mentioned above, each R⁴ preferably is independently
H, C₁-C₄-alkyl or C₁-C₄-haloalkyl;
or C₃-C₇-cycloalkyl or C₄-C₈-cycloalkylalkyl, each optionally substituted with 1 to 4 substituents independently selected from the group consisting of halogen.

More preferably, each R⁴ is independently
H, C₁-C₄-alkyl or C₁-C₄-haloalkyl.

Especially preferably, each R⁴ is methyl, ethyl or trifluoromethyl.

In all embodiments (“embodiments 2”) mentioned above, each R¹¹ preferably is independently
H, C₁-C₆-alkyl or C₁-C₆-haloalkyl.

More preferably, each R^{4a} is independently
C₁-C₄-alkyl or C₁-C₄-haloalkyl.

Especially preferably, each R¹¹ is independently
methyl or ethyl.

In all embodiments (“embodiments 2”) mentioned above,

each R⁵ preferably is independently
H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹²;
or C₃-C₇-cycloalkyl or C₄-C₈-cycloalkylalkyl, each optionally substituted with 1 to 4 substituents independently selected from the group consisting of halogen, cyano, C₁-C₄-alkyl; C₁-C₄-haloalkyl, OR⁴a and S(O)ₘR^{9a}.

More preferably, each R⁵ is independently
H, C₁-C₄-alkyl, C₁-C₄-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹².

Especially preferably, each R⁵ is independently
H, methyl or ethyl.

In all embodiments (“embodiments 2”) mentioned above,

each R^{5a} preferably is independently
H or C₁-C₄-alkyl.

More preferably, each R^{5a} is independently
H or C₁-C₂-alkyl.

Especially preferably, each R^{5a} is independently
methyl or ethyl.

In all embodiments (“embodiments 2”) mentioned above,

each R⁶ preferably is independently
H, C₁-C₄-alkyl, C₁-C₄-haloalkyl, C₂-C₄-alkenyl, C₂-C₄-haloalkenyl, C₂-C₄-alkynyl or C₂-C₄-haloalkynyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR^{4a} and S(O)ₘR^{9a}.

More preferably, each R⁶ is independently
H, C₁-C₄-alkyl, C₁-C₄-haloalkyl, C₂-C₄-alkenyl or C₂-C₄-haloalkenyl;
or C₃-C₇-cycloalkyl optionally substituted with 1 to 4 substituents independently selected from the group consisting of halogen, cyano, C₁-C₄-alkyl and C₁-C₄-haloalkyl.

Especially preferably, each R⁶ is independently
H, methyl or ethyl.

In all embodiments (“embodiments 2”) mentioned above,

each R^{6a} preferably is independently
H, C₁-C₄-alkyl, C(O)R¹³ or C(O)OR¹³.

More preferably, each R^{6a} is independently
H or C₁-C₄-alkyl.

Especially preferably, each R^{6a} is independently
H, methyl or ethyl.

In all embodiments (“embodiments 2”) mentioned above, each R⁷ preferably is independently
H, C₁-C₆-alkyl, C₁-C₆-haloalkyl, C₂-C₆-alkenyl or C₂-C₆-haloalkenyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR⁴a and S(O)ₘR^{9a}.

More preferably, each R⁷ is independently
H, C₁-C₄-alkyl or C₁-C₄-haloalkyl;
or C₃-C₇-cycloalkyl optionally substituted with 1 to 4 substituents independently selected from the group consisting of halogen, cyano, C₁-C₂-alkyl and C₁-C₂-haloalkyl.

Especially preferably, each R⁷ is independently
H, methyl, ethyl or trifluoromethyl.

In all embodiments (“embodiments 2”) mentioned above,

each R^{7a} preferably is independently
C₁-C₄-alkyl or C₁-C₄-haloalkyl.

More preferably, each R^{7a} is independently
C₁-C₄-alkyl or C₁-C₂-haloalkyl.

Especially preferably, each R^{7a} is independently
methyl or ethyl.

In all embodiments (“embodiments 2”) mentioned above,

each R⁸ preferably is independently
H, C₁-C₄-alkyl or C₁-C₄-haloalkyl.

More preferably, each R⁸ is independently
C₁-C₂-alkyl or C₁-C₂-haloalkyl.

Especially preferably, each R⁸ is independently
methyl or ethyl.

In all embodiments (“embodiments 2”) mentioned above,

each R^{8a} preferably is independently
C₁-C₄-alkyl or C₁-C₄-haloalkyl.

More preferably, each R^{8a} is independently
C₁-C₄-alkyl.

Especially preferably, each R^{8a} is independently
methyl or ethyl.

In all embodiments (“embodiments 2”) mentioned above,

each R⁹ preferably is independently
C₁-C₄-alkyl or C₁-C₄-haloalkyl.

More preferably, each R⁹ is independently
C₁-C₂-alkyl, C₁-C₂-haloalkyl.

Especially preferably, each R⁹ is independently
methyl, ethyl or trifluoromethyl.

In all embodiments (“embodiments 2”) mentioned above,

each R^{9a} preferably is independently
C₁-C₄-alkyl or C₁-C₄-haloalkyl.

More preferably, each R^{9a} is independently
C₁-C₄-alkyl or C₁-C₂-haloalkyl.

Especially preferably, each R^{9a} is independently
methyl, ethyl or trifluoromethyl.

In all embodiments (“embodiments 2”) mentioned above,

each R¹⁰ preferably is independently
H, C₁-C₄-alkyl, C₁-C₄-haloalkyl, C₂-C₃-alkenyl, C₂-C₃-haloalkenyl, C₂-C₃-alkynyl, C₂-C₃-haloalkynyl or C₃-C₇-cycloalkyl.

More preferably, each R¹⁰ is independently
H, C₁-C₄-alkyl, C₂-C₃-alkenyl or C₃-C₇-cycloalkyl.

Especially preferably, each R¹⁰ is independently
H or methyl.

In all embodiments (“embodiments 2”) mentioned above,

each R¹¹ preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl or C₃-C₇-cycloalkyl.

More preferably, each R¹¹ is independently
H, C₁-C₄-alkyl, C₁-C₄-haloalkyl.

Especially preferably, each R¹¹ is independently
H, methyl or ethyl.

In all embodiments (“embodiments 2”) mentioned above,

each R^{11a} preferably is independently
H, C₁-C₄-alkyl, C₂-C₃-alkenyl or C₂-C₃-alkynyl.

More preferably, each R^{11a} is independently
H, C₁-C₄-alkyl, allyl or propargyl.

Especially preferably, each R^{11a} is independently
H, methyl or ethyl.

In all embodiments (“embodiments 2”) mentioned above,

each R¹² preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹² is independently
H, C₁-C₄-alkyl or C₁-C₄-haloalkyl.

Especially preferably, each R¹² is independently
H, methyl or ethyl.

In all embodiments (“embodiments 2”) mentioned above,

each R¹³ preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹³ is independently
H, C₁-C₄-alkyl or C₁-C₄-haloalkyl.

Especially preferably, each R¹³ is independently
methyl.

In all embodiments (“embodiments 2”) mentioned above,

each R¹⁴ preferably is independently
C₁-C₄-alkyl or C₁-C₄-haloalkyl;
or phenyl optionally substituted with 1 to 5 substituents independently selected from the group consisting of halogen, cyano, OR⁴, NR⁵R⁶, C₁-C₄-alkyl, C(X)R⁷, C(O)OR⁸, C(O)NR¹¹R¹², S(O)ₘR⁹ and S(O)₂NR¹¹R¹².

More preferably, each R¹⁴ is independently
C₁-C₄-alkyl or C₁-C₄-haloalkyl.

Especially preferably, each R¹⁴ is independently
methyl.

In all embodiments (“embodiments 2”) mentioned above, X preferably is O or S. Especially preferably X is O.

In all embodiments (“embodiments 2”) mentioned above, p preferably is 0, 1, 2, 3 or 4.

More preferably, p is 0, 1, 2 or 3.

Especially preferably, p is 0, 1 or 2.

In all embodiments (“embodiments 2”) mentioned above with V is defined to be one of V-1 to V-22,

each R^{v} preferably is independently
H, halogen, cyano, nitro, C₁-C₄-alkyl, C₁-C₄-haloalkyl, O(C₁-C₄-alkyl), O(C₁-C₄-haloalkyl), S(C₁-C₄-alkyl), S(C₁-C₄-haloalkyl), S(O)(C₁-C₄-haloalkyl), S(O)₂(C₁-C₄-haloalkyl), C(O)O(C₁-C₄-alkyl), phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a}.

More preferably, each R^{v} is independently
H, halogen, cyano, nitro, C₁-C₄-alkyl, C₁-C₄-haloalkyl, O(C₁-C₄-alkyl), O(C₁-C₄-haloalkyl), C(O)O(C₁-C₂-alkyl), phenyl optionally substituted with 1 to 3 substituents independently selected from the group consisting of halogen, cyano, nitro, O(C₁-C₄-alkyl), C₁-C₄-alkyl.

Especially preferably, each R^{v} is independently
H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl.

In all embodiments mentioned above with V is defined to be one of V-1 to V-22, r preferably is 0, 1, 2 or 3. More preferably r is 0, 1 or 2. Especially preferably, r is 0, 1 or 2.

The compounds of the invention are defined in general terms by the formula (I). Especially preferred (“embodiments 3”) substituents and ranges of the radicals given in the formulae referred to above and below are defined,

wherein
Z is O;
A¹, A², A³ and A⁴ are C—R¹;
each W is independently hydrogen, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹²; OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰ S(O)₂R⁹, N(R¹⁰ S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R¹ is independently H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R² is H, halogen, cyano, nitro, SF₅, OCN, SCN, Si(R¹⁴)₃, OR⁴, NR⁵R⁶, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R⁷, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or)N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R³ is H, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, OR^{4a}, and S(O)ₘR^{9a};
or C₁-C₆-alkyl substituted with 1 to 4 substituents independently selected from the group consisting of cyano, nitro, OR⁴, NR⁵R⁶, C(X)R¹, C(O)OR⁸, C(O)NR¹¹R¹², S(O)ₘR⁹ and S(O)₂NR¹¹R¹²;
or C₁-C₆-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R⁴ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{4a} is independently H, C₁-C₆-alkyl or C₁-C₆-haloalkyl;

each R⁵ is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{5a} is independently H or C₁-C₆-alkyl;
each R⁶ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{6a} is independently H, C₁-C₆-alkyl, C(O)R¹³ or C(O)OR¹³;
each R⁷ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{7a} is independently C₁-C₆-alkyl or C₁-C₆-haloalkyl;
each R⁸ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{8a} is independently C₁-C₆-alkyl or C₁-C₆-haloalkyl;
each R⁹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl; or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{9a} is independently C₁-C₆-alkyl or C₁-C₆-haloalkyl;
each R¹⁰ is independently H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R^{7a}, C(O)OR⁸a, C(O)NR¹¹R¹², S(O)ₘR^{9a} or S(O)₂NR¹¹R¹²;
each R¹¹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR^{11a}R¹², S(O)ₘR^{9a} and S(O)₂NR^{11a}R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR^{11a}R¹², OR^{4a}, C₂-C₆-alkoxyalkyl, S(O)ₘR^{9a}, S(O)₂NR^{11a}R¹², NR^{5a}R^{6a}, OC(O)R^{7a} and N(R¹⁰)C(O)R^{7a};
each R^{11a} is independently H, C₂-C₆-alkenyl or C₂-C₆-alkynyl;
each R¹² is independently H, NR^{5a}R^{6a}, C₁-C₆-haloalkyl, C₂-C₆-alkenyl, C₂-C₆-haloalkenyl, C₂-C₆-alkynyl, C₂-C₆-haloalkynyl, C(X)R¹, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R¹³ is independently H, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{N} is independently C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 5 substituents independently selected from the group consisting of halogen, cyano, nitro, OR⁴, NR⁵R⁶, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR8, C(O)NR¹¹R¹², S(O)ₘR⁹, S(O)₂NR¹¹R¹², OC(O)R^{7a} and N(R¹⁰)C(O)R^{7a};
X is O or S;
T is hydrogen or methyl;
Y is hydrogen;
V is selected from the group consisting of V-2, V-3, V-4, V-6, V-7, V-8, V-9, V-11, V-13, V-14, V-18, V-19, V-20
each m is independently 0, 1 or 2.
p is 0, 1, 2, 3 or 4.

In all embodiments (“embodiments 3”) mentioned above,

each W preferably is independently
hydrogen, halogen, cyano, nitro, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of C(X)R⁷, C(O)OR⁸, C(O)NR¹¹R¹², S(O)ₘR⁹ and S(o)₂NR¹¹R¹²;
or phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹² and N(R¹⁰)C(O)R^{7a}.

More preferably, each W is independently
hydrogen, halogen, cyano, nitro, OR⁴, NR⁵R⁶, C₁-C₆-alkyl, C₁-C₆-haloalkyl, C(X)R¹, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹² or C₃-C₇-cycloalkyl, optionally substituted with 1 to 4 substituents independently selected from the group consisting of halogen, cyano, C₁-C₄-alkyl, C₁-C₄-haloalkyl;
or C₁-C₆-alkyl, substituted with 1 to 4 substituents independently selected from the group consisting of S(O)ₘR⁹ and S(O)₂NR¹¹R¹².

Especially preferably, each W is independently
hydrogen, fluorine, chlorine, bromine, methoxy, trifluormethoxy, methyl or ethyl.

In all embodiments (“embodiments 3”) mentioned above,

each R¹ preferably is independently
H, halogen, cyano, nitro, OR⁴, NR⁵R⁶, C₁-C₆-alkyl, C₁-C₆-haloalkyl, C₂-C₆-alkenyl, C₂-C₆-haloalkenyl, C(X)R⁷, C(O)OR⁸, C(O)NR¹¹R¹², S(O)ₘR⁹, S(O)₂NR¹¹R¹², N(R¹⁰)C(O)R⁷, N(R¹⁰)C(O)NR¹¹R¹², N(R¹⁰)S(O)₂R⁹ or N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, OR⁴, NR⁵R⁶, C(X)R⁷, S(O)ₘR⁹ and S(O)₂NR¹¹R¹².

More preferably, each R¹ is independently
H, halogen, cyano, nitro, OR⁴, NR⁵R⁶, C₁-C₄-alkyl, C₁-C₄-haloalkyl, C(X)R⁷, C(O)OR⁸, C(O)NR¹¹R¹², S(O)ₘR⁹ or S(O)₂NR¹¹R¹²;
or C₃-C₇-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, S(O)ₘR⁹ and S(O)₂NR¹¹R¹².

Especially preferably, each R¹ is independently
H, fluorine, chlorine, bromine, iodine, ethoxy or trifluoromethyl.

In all embodiments (“embodiments 3”) mentioned above,
R² preferably is H, halogen, cyano, nitro, OR⁴, NR⁵R⁶, C₁-C₆-alkyl, C₁-C₄-haloalkyl, C₂-C₄-alkenyl, C₂-C₄-haloalkenyl, C₃-C₇-cycloalkyl, S(O)ₘR⁹ or S(O)₂NR¹¹R¹²;
or C₃-C₆-cycloalkyl, optionally substituted with 1 to 4 substituents independently selected from the group consisting of halogen, cyano, C₁-C₄-alkyl and C₁-C₄-haloalkyl;
or C₁-C₆-alkyl substituted with 1 to 4 substituents independently selected from the group consisting of cyano, OR⁴, NR⁵R⁶, S(O)ₘR⁹ and S(O)₂NR¹¹R¹².

More preferably, R² is
H, halogen, cyano, S(O)ₘR⁹ or S(O)₂NR¹¹R¹²;

or C₃-C₆-cycloalkyl, optionally substituted with 1 to 4 substituents independently selected from the group consisting of halogen, cyano, C₁-C₄-alkyl and C₁-C₄-haloalkyl;

or C₁-C₄-alkyl substituted with 1 to 2 substituents independently selected from the group consisting of cyano, OR⁴ and S(O)ₘR⁹.

Especially preferably, R² is hydrogen.

In all embodiments (“embodiments 3”) mentioned above,

R³ preferably is

H, C₁-C₆-alkyl, C₁-C₆-haloalkyl, C₂-C₄-alkenyl, C₂-C₄-haloalkenyl, C₂-C₄-alkynyl, C₂-C₄-haloalkynyl, C(X)R⁷, C(O)OR⁸ or C(O)NR¹¹R¹²;

or C₃-C₇-cycloalkyl or C₄-C₈-cycloalkylalkyl, each optionally substituted with 1 to 4 substituents independently selected from the group consisting of halogen, cyano, OR^{4a} and S(O)ₘR^{9a};

or C₁-C₄-alkyl substituted with 1 to 4 substituents independently selected from the group consisting of cyano, OR⁴, C(X)R⁷ and C(O)OR⁸;

or C₁-C₄-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, C₁-C₄-haloalkyl, C₂-C₆-alkoxyalkyl, C(X)R⁷, C(O)OR⁸, S(O)ₘR⁹ and S(O)₂NR¹¹R¹².

More preferably, R³ is
H, C₁-C₄-alkyl, C₂-C₃-alkenyl, C(X)R⁷ or C(O)OR⁸;
or C₁-C₂-alkyl substituted with OR⁴;
or C₁-C₂-alkyl substituted with phenyl.

Especially preferably, R³ is hydrogen.

In all embodiments (“embodiments 3”) mentioned above, each R⁴ preferably is independently
H, C₁-C₄-alkyl or C₁-C₄-haloalkyl;
or C₃-C₇-cycloalkyl or C₄-C₈-cycloalkylalkyl, each optionally substituted with 1 to 4 substituents independently selected from the group consisting of halogen.

More preferably, each R⁴ is independently
H, C₁-C₄-alkyl or C₁-C₄-haloalkyl.

Especially preferably, each R⁴ is methyl, ethyl or trifluoromethyl.

In all embodiments (“embodiments 3”) mentioned above, each R¹¹ preferably is independently
H, C₁-C₆-alkyl or C₁-C₆-haloalkyl.

More preferably, each R¹¹ is independently
C₁-C₄-alkyl or C₁-C₄-haloalkyl.

Especially preferably, each R¹¹ is independently
methyl or ethyl.

In all embodiments (“embodiments 3”) mentioned above,

each R⁵ preferably is independently
H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹²;
or C₃-C₇-cycloalkyl or C₄-C₈-cycloalkylalkyl, each optionally substituted with 1 to 4 substituents independently selected from the group consisting of halogen, cyano, C₁-C₄-alkyl; C₁-C₄-haloalkyl, OR⁴a and S(O)ₘR^{9a}.

More preferably, each R⁵ is independently
H, C₁-C₄-alkyl, C₁-C₄-haloalkyl, C₂-C₆-alkenyl, C₂-C₆-haloalkenyl, C₂-C₆-alkynyl, C₂-C₆-haloalkynyl, S(O)ₘR⁹ or S(O)₂NR¹¹R¹².

Especially preferably, each R⁵ is independently
H, methyl or ethyl.

In all embodiments (“embodiments 3”) mentioned above,

each R^{5a} preferably is independently
H or C₁-C₄-alkyl.

More preferably, each R^{5a} is independently
H or C₁-C₂-alkyl.

Especially preferably, each R^{5a} is independently
methyl or ethyl.

In all embodiments (“embodiments 3”) mentioned above,

each R⁶ preferably is independently
H, C₁-C₄-alkyl, C₁-C₄-haloalkyl, C₂-C₄-alkenyl, C₂-C₄-haloalkenyl, C₂-C₄-alkynyl or C₂-C₄-haloalkynyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR^{4a} and S(O)ₘR^{9a}.

More preferably, each R⁶ is independently
H, C₁-C₄-alkyl, C₁-C₄-haloalkyl, C₂-C₄-alkenyl or C₂-C₄-haloalkenyl;
or C₃-C₇-cycloalkyl optionally substituted with 1 to 4 substituents independently selected from the group consisting of halogen, cyano, C₁-C₄-alkyl and C₁-C₄-haloalkyl.

Especially preferably, each R⁶ is independently
H, methyl or ethyl.

In all embodiments (“embodiments 3”) mentioned above,

each R^{6a} preferably is independently
H, C₁-C₄-alkyl, C(O)R¹³ or C(O)OR¹³.

More preferably, each R^{6a} is independently
H or C₁-C₄-alkyl.

Especially preferably, each R^{6a} is independently
H, methyl or ethyl.

In all embodiments (“embodiments 3”) mentioned above, each R⁷ preferably is independently
H, C₁-C₆-alkyl, C₁-C₆-haloalkyl, C₂-C₆-alkenyl or C₂-C₆-haloalkenyl;
or C₃-C₇-cycloalkyl or C₄-C₈-cycloalkylalkyl, each optionally substituted with 1 to 4 substituents independently selected from the group consisting of halogen, cyano, C₁-C₄-alkyl, C₁-C₄-haloalkyl, OR⁴a and S(O)ₘR^{9a}.

More preferably, each R⁷ is independently
H, C₁-C₄-alkyl or C₁-C₄-haloalkyl;
or C₃-C₇-cycloalkyl optionally substituted with 1 to 4 substituents independently selected from the group consisting of halogen, cyano, C₁-C₂-alkyl and C₁-C₂-haloalkyl.

Especially preferably, each R⁷ is independently
H, methyl, ethyl or trifluoromethyl.

In all embodiments (“embodiments 3”) mentioned above,

each R^{7a} preferably is independently
C₁-C₄-alkyl or C₁-C₄-haloalkyl.

More preferably, each R^{7a} is independently
C₁-C₄-alkyl or C₁-C₂-haloalkyl.

Especially preferably, each R^{7a} is independently
methyl or ethyl.

In all embodiments (“embodiments 3”) mentioned above,

each R⁸ preferably is independently
H, C₁-C₄-alkyl or C₁-C₄-haloalkyl.

More preferably, each R⁸ is independently
C₁-C₂-alkyl or C₁-C₂-haloalkyl.

Especially preferably, each R⁸ is independently
methyl or ethyl.

In all embodiments (“embodiments 3”) mentioned above,

each R^{8a} preferably is independently
C₁-C₄-alkyl or C₁-C₄-haloalkyl.

More preferably, each R^{8a} is independently
C₁-C₄-alkyl.

Especially preferably, each R^{8a} is independently
methyl or ethyl.

In all embodiments (“embodiments 3”) mentioned above,

each R⁹ preferably is independently
C₁-C₄-alkyl or C₁-C₄-haloalkyl.

More preferably, each R⁹ is independently
C₁-C₂-alkyl, C₁-C₂-haloalkyl.

Especially preferably, each R⁹ is independently
methyl, ethyl or trifluoromethyl.

In all embodiments (“embodiments 3”) mentioned above,

each R^{9a} preferably is independently
C₁-C₄-alkyl or C₁-C₄-haloalkyl.

More preferably, each R^{9a} is independently
C₁-C₄-alkyl or C₁-C₂-haloalkyl.

Especially preferably, each R^{9a} is independently
methyl, ethyl or trifluoromethyl.

In all embodiments (“embodiments 3”) mentioned above,

each R¹⁰ preferably is independently
H, C₁-C₄-alkyl, C₁-C₄-haloalkyl, C₂-C₃-alkenyl, C₂-C₃-haloalkenyl, C₂-C₃-alkynyl, C₂-C₃-haloalkynyl or C₃-C₇-cycloalkyl.

More preferably, each R¹⁰ is independently
H, C₁-C₄-alkyl, C₂-C₃-alkenyl or C₃-C₇-cycloalkyl.

Especially preferably, each R¹⁰ is independently
H or methyl.

In all embodiments (“embodiments 3”) mentioned above,

each R¹¹ preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl or C₃-C₇-cycloalkyl.

More preferably, each R¹¹ is independently
H, C₁-C₄-alkyl, C₁-C₄-haloalkyl.

Especially preferably, each R¹¹ is independently
H, methyl or ethyl.

In all embodiments (“embodiments 3”) mentioned above, each R^{11a} preferably is independently
H, C₁-C₄-alkyl, C₂-C₃-alkenyl or C₂-C₃-alkynyl.

More preferably, each R^{11a} is independently
H, C₁-C₄-alkyl, allyl or propargyl.

Especially preferably, each R^{11a} is independently
H, methyl or ethyl.

In all embodiments (“embodiments 3”) mentioned above,

each R¹² preferably is independently
H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹² is independently
H, C₁-C₄-alkyl or C₁-C₄-haloalkyl.

Especially preferably, each R¹² is independently
H, methyl or ethyl.

In all embodiments (“embodiments 3”) mentioned above,

each R¹³ preferably is independently

H, C₁-C₄-alkyl, C₁-C₄-haloalkyl, C₂-C₃-alkenyl, C₂-C₃-haloalkenyl, C₂-C₃-alkynyl or C₂-C₃-haloalkynyl.

More preferably, each R¹³ is independently
H, C₁-C₄-alkyl or C₁-C₄-haloalkyl.

Especially preferably, each Rⁿ is independently
methyl.

In all embodiments (“embodiments 3”) mentioned above, each R¹⁴ preferably is independently

C₁-C₄-alkyl or C₁-C₄-haloalkyl;

or phenyl optionally substituted with 1 to 5 substituents independently selected from the group consisting of halogen, cyano, OR⁴, NR⁵R⁶, C₁-C₄-alkyl, C(X)R⁷, C(O)OR⁸, C(O)NR¹¹R¹², S(O)ₘR⁹ and S(O)₂NR¹¹R¹².

More preferably, each R¹⁴ is independently
C₁-C₄-alkyl or C₁-C₄-haloalkyl.

Especially preferably, each R¹⁴ is independently
methyl.

In all embodiments (“embodiments 3”) mentioned above, X preferably is O or S. Especially preferably X is O.

In all embodiments (“embodiments 3”) mentioned above, p preferably is 0, 1, 2, 3 or 4.

More preferably, p is 0, 1, 2 or 3.

Especially preferably, p is 0, 1 or 2.

In all embodiments (“embodiments 3”) mentioned above,

each R^{v} preferably is independently
H, halogen, cyano, nitro, C₁-C₄-alkyl, C₁-C₄-haloalkyl, O(C₁-C₄-alkyl), O(C₁-C₄-haloalkyl), S(C₁-C₄-alkyl), S(C₁-C₄-haloalkyl), S(O)(C₁-C₄-haloalkyl), S(O)₂(C₁-C₄-haloalkyl), C(O)O(C₁-C₄-alkyl), phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a}.

More preferably, each R^{v} is independently
H, halogen, cyano, nitro, C₁-C₄-alkyl, C₁-C₄-haloalkyl, O(C₁-C₄-alkyl), O(C₁-C₄-haloalkyl),

C(O)O(C₁-C₂-alkyl), phenyl optionally substituted with 1 to 3 substituents independently selected from the group consisting of halogen, cyano, nitro, O(C₁-C₄-alkyl), C₁-C₄-alkyl.

Especially preferably, each R^{v} is independently
H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl.

In all embodiments mentioned above with V is defined to be one of V-1 to V-22, r preferably is 0, 1, 2 or 3. More preferably r is 0, 1 or 2. Especially preferably, r is 0, 1 or 2.

The definitions of radicals, and explanations, that are given above in general or in ranges of preference may be combined arbitrarily with one another, thus including combinations between the respective ranges and ranges of preference. The definitions and explanations apply to the end products and also to the precursors and intermediates accordingly.

Preferred in accordance with the invention are the compounds of the formula (I) in which each structural element is defined as given above as being preferred (“preferably”). For those structural elements lacking the preferred definition, the definition in the summary of the invention shall apply.

Likewise preferred in accordance with the invention are the compounds of the formula (I) in which V is selected from the group consisting of V-1 to V-22 and in which each further structural element is defined as given above as being preferred (“preferably”). For those structural elements lacking the preferred definition, the definition in the summary of the invention shall apply.

More preferred in accordance with the invention are the compounds of the formula (I) in which each structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Likewise more preferred in accordance with the invention are the compounds of the formula (I) in which V is selected from the group consisting of V-1 to V-22 and in which each further structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Especially preferred in accordance with the invention are the compounds of the formula (I) in which each structural element is defined as given above as being especially preferred (especially preferably). For those structural elements lacking the especially preferred definition, the definition in the summary of the invention shall apply.

Likewise especially preferred in accordance with the invention are the compounds of the formula (I) in which V is selected from the group consisting of V-1 to V-22 and in which each further structural element is defined as given above as being especially preferred (especially preferably). For those structural elements lacking the especially preferred definition, the definition in the summary of the invention shall apply.

With regard to embodiment 1 as defined above, the following applies:

Preferred in accordance with the invention are the compounds of the formula (I) in which each structural element is defined as given above as being preferred (“preferably”). For those structural elements lacking the preferred definition, the definition in the summary of the invention shall apply.

Likewise preferred in accordance with the invention are the compounds of the formula (I) in which V is selected from the group consisting of V-1 to V-22 and in which each further structural element is defined as given above as being preferred (“preferably”). For those structural elements lacking the preferred definition, the definition in the summary of the invention shall apply.

More preferred in accordance with the invention are the compounds of the formula (I) in which each structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Likewise more preferred in accordance with the invention are the compounds of the formula (I) in which V is selected from the group consisting of V-1 to V-22 and in which each further structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Especially preferred in accordance with the invention are the compounds of the formula (I) in which each structural element is defined as given above as being especially preferred (especially preferably). For those structural elements lacking the especially preferred definition, the definition in the summary of the invention shall apply.

Likewise especially preferred in accordance with the invention are the compounds of the formula (I) in which V is selected from the group consisting of V-1 to V-22 and in which each further structural element is defined as given above as being especially preferred (especially preferably). For those structural elements lacking the especially preferred definition, the definition in the summary of the invention shall apply.

With regard to embodiment 2 as defined above, the following applies:

Preferred in accordance with the invention are the compounds of the formula (I) in which each structural element is defined as given above as being preferred (“preferably”). For those structural elements lacking the preferred definition, the definition in the summary of the invention shall apply.

Likewise preferred in accordance with the invention are the compounds of the formula (I) in which V is selected from the group consisting of V-1 to V-22 and in which each further structural element is defined as given above as being preferred (“preferably”). For those structural elements lacking the preferred definition, the definition in the summary of the invention shall apply.

More preferred in accordance with the invention are the compounds of the formula (I) in which each structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Likewise more preferred in accordance with the invention are the compounds of the formula (I) in which V is selected from the group consisting of V-1 to V-22 and in which each further structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Especially preferred in accordance with the invention are the compounds of the formula (I) in which each structural element is defined as given above as being especially preferred (especially preferably). For those structural elements lacking the especially preferred definition, the definition in the summary of the invention shall apply.

Likewise especially preferred in accordance with the invention are the compounds of the formula (I) in which V is selected from the group consisting of V-1 to V-22 and in which each further structural element is defined as given above as being especially preferred (especially preferably). For those structural elements lacking the especially preferred definition, the definition in the summary of the invention shall apply.

With regard to embodiment 3 as defined above, the following applies:

Preferred in accordance with the invention are the compounds of the formula (I) in which each structural element is defined as given above as being preferred (“preferably”). For those structural elements lacking the preferred definition, the definition in the summary of the invention shall apply.

Likewise preferred in accordance with the invention are the compounds of the formula (I) in which V is selected from the group consisting of V-1 to V-22 and in which each further structural element is defined as given above as being preferred (“preferably”). For those structural elements lacking the preferred definition, the definition in the summary of the invention shall apply.

More preferred in accordance with the invention are the compounds of the formula (I) in which each structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Likewise more preferred in accordance with the invention are the compounds of the formula (I) in which V is selected from the group consisting of V-1 to V-22 and in which each further structural element is defined as given above as being more preferred (“more preferably”). For those structural elements lacking the more preferred definition, the definition given above as being especially preferred (“especially preferably”) shall apply with the proviso that for those structural elements lacking the especially preferred definition as well, the definition in the summary of the invention shall apply.

Especially preferred in accordance with the invention are the compounds of the formula (I) in which each structural element is defined as given above as being especially preferred (especially preferably). For those structural elements lacking the especially preferred definition, the definition in the summary of the invention shall apply.

Likewise especially preferred in accordance with the invention are the compounds of the formula (I) in which V is selected from the group consisting of V-1 to V-22 and in which each further structural element is defined as given above as being especially preferred (especially preferably). For those structural elements lacking the especially preferred definition, the definition in the summary of the invention shall apply.

Saturated or unsaturated hydrocarbon radicals such as alkyl, alkanediyl or alkenyl may in each case, both alone and in conjunction with heteroatoms, as in alkoxy, for example, be—where possible—either straight-chain or branched.

Any substituted radicals may, unless indicated otherwise, be substituted one or more times, and the substituents in the case of multiple substitutions may be alike or different.

In the definitions of radicals that are stated as being preferred, halogen (halo) is fluoro, chloro, bromo and iodo, very preferably fluoro, chloro and bromo, and especially preferably fluoro and chloro.

Further specific embodiments of the invention are described hereafter:

A specific embodiment of the invention are the compounds of the formula (I) in which

Z is O;

A¹, A², A³ and A⁴ are independently N or C—R¹;

X is O;

T is hydrogen, halogen, C₁-C₄-alkyl or C₁-C₄-haloalkyl;

Y is hydrogen, halogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as preferred (“preferably”) in embodiments 1, 2 and 3.

Another specific embodiment of the invention are the compounds of the formula (I) in which

Z is O;

A¹, A², A³ and A⁴ are independently N or C—R¹;

X is O;

T is hydrogen, halogen, C₁-C₄-alkyl or C₁-C₄-haloalkyl;

Y is hydrogen, halogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as preferred (“preferably”) in embodiments 1, 2 and 3.

Another specific embodiment of the invention are the compounds of the formula (I) in which

Z is O;

A¹, A², A³ and A⁴ are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

A¹, A², A³ and A⁴ are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R¹¹, R⁵, R^{5a}, R⁶, R^{6a}, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

A¹, A², A³ and A⁴ are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;
p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹, A², A³ and A⁴ are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹, A², A³ and A⁴ are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹, A², A³ and A⁴ are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

one of A¹, A², A³ and A⁴ is N and the remaining are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

one of A¹, A², A³ and A⁴ is N and the remaining are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

one of A¹, A², A³ and A⁴ is N and the remaining are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

one of A¹, A², A³ and A⁴ is N and the remaining are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;
one of A¹, A², A³ and A⁴ is N and the remaining are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

one of A¹, A², A³ and A⁴ is N and the remaining are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹ is N and A², A³ and A⁴ are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;
p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

A¹ is N and A², A³ and A⁴ are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

A¹ is N and A², A³ and A⁴ are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹ is N and A², A³ and A⁴ are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹ is N and A², A³ and A⁴ are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A¹ is N and A², A³ and A⁴ are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A³ is N and A¹, A² and A⁴ are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;
p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

A³ is N and A¹, A² and A⁴ are C—R¹;

X is O;

T is hydrogen, C₁-C₄-alkyl or C₁-C₄-haloalkyl;

Y is hydrogen, C₁-C₄-alkyl or C₁-C₄-haloalkyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R⁴, R^{4a}, R⁵, R^{5a}, R⁶, R^{6a}, R⁷, R^{7a}, R⁸, R^{8a}, R⁹, R^{9a}, R¹⁰, R¹¹, R^{11a}, R¹², R¹³, R¹⁴, R^{v} and r are as defined as more preferred (“more preferably”) in embodiments 1, 2 and 3.

Another specific embodiment of the invention are the compounds of the formula (I) in which

Z is O;

A³ is N and A¹, A² and A⁴ are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A³ is N and A¹, A² and A⁴ are C—R¹;

X is O;

T is hydrogen or methyl;

Y is hydrogen or methyl;

V is selected from the group consisting of V-1 to V-22, wherein the group —C(T)(Y)(V) is positioned at 5-C as indicated in formula (I);

each m is independently 0 or 2;

p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A³ is N and A¹, A² and A⁴ are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;
p is 0, 1, 2 or 3, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (I) in which

Z is O;

A³ is N and A¹, A² and A⁴ are C—R¹;

X is O;

T is hydrogen;

Y is hydrogen;

V is selected from the group consisting of V-1 to V-22, wherein the group —C(T)(Y)(V) is positioned at 5-C as indicated in formula (I);

each m is independently 0 or 2;
p is 0, 1 or 2, wherein if p is 1 then W is positioned at 2-C or 4-C as indicated in formula (I), and if p is 2 then one W is positioned at 2-C and one W is positioned at 3-C or 4-C as indicated in formula (I);

W, R¹, R², R³, R^{v} and r are as defined as especially preferred (“especially preferably”) in embodiments 1, 2 and 3.

Another specific embodiment of the invention are the compounds of the formula (Ia)

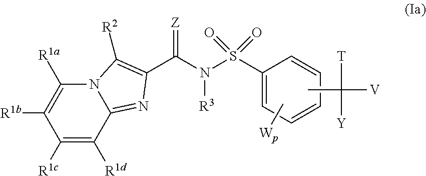

in which

Z is O;

R^{1a}, R^{1b}, R^{1c} and R^{1d} are independently R¹ as defined as especially preferred (“especially preferably”) in embodiments 1, 2 and 3;

T is hydrogen or methyl;

Y is hydrogen;

V is selected from the group consisting of V-1 to V-22;

p is 0, 1 or 2;

W, R², R³, R^{v} and r are as defined as especially preferred (“especially preferably”) in embodiments 1, 2 and 3.

Another specific embodiment of the invention are the compounds of the formula (Ib)

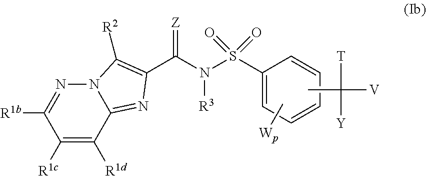

in which

Z is O;

R^{1b}, R^{1c} and R^{1d} are independently R¹ as defined as especially preferred (“especially preferably”) in embodiments 1, 2 and 3;

T is hydrogen or methyl;

Y is hydrogen;

V is selected from the group consisting of V-1 to V-22;
p is 0, 1 or 2;

W, R², R³, R^{v} and r are as defined as especially preferred (“especially preferably”) in embodiments 1, 2 and 3.

Another specific embodiment of the invention are the compounds of the formula (Ic)

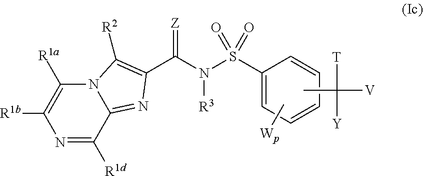

in which

Z is O;

R^{1a}, R^{1b} and R^{1d} are independently R¹ as defined as especially preferred (“especially preferably”) in embodiments 1, 2 and 3;

T is hydrogen or methyl;

Y is hydrogen;

V is selected from the group consisting of V-1 to V-22;
p is 0, 1 or 2;

W, R², R³, R^{v} and r are as defined as especially preferred (“especially preferably”) in embodiments 1, 2 and 3.

Of note is that compounds of this invention are characterized by favorable metabolic and/or soil residual patterns and exhibit activity controlling a spectrum of agronomic and nonagronomic parasitic nematodes.

Of particular note, for reasons of parasitic nematode control spectrum and economic importance, protection of agronomic crops from damage or injury caused by parasitic nematodes by controlling parasitic nematodes are embodiments of the invention. Compounds of this invention because of their favorable translocation properties or systemicity in plants also protect foliar or other plant parts which are not directly contacted with a compound of formula (I) or a composition comprising the compound.

Also noteworthy as embodiments of the present invention are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present invention are compositions for controlling a parasitic nematode comprising a compound of any of the preceding embodiments, as well as any other embodiments described herein, and any combinations thereof, and at least one additional component selected from the group consisting of a surfactant, a solid diluent and a liquid diluent, said compositions optionally further comprising at least one additional biologically active compound or agent. Embodiments of the invention further include methods for controlling a parasitic nematode comprising contacting the parasitic nematode or its environment with a biologically effective amount of a compound of any of the preceding Embodiments (e.g., as a composition described herein).

Embodiments of the invention also include a composition comprising a compound of any of the preceding Embodiments, in the form of a soil drench liquid formulation. Embodiments of the invention further include methods for controlling a parasitic nematode comprising contacting the soil with a liquid composition as a soil drench comprising a biologically effective amount of a compound of any of the preceding Embodiments.

Embodiments of the invention also include a spray composition for controlling a parasitic nematode comprising a biologically effective amount of a compound of any of the preceding Embodiments and a propellant. Embodiments of the invention further include a bait composition for controlling a parasitic nematode comprising a biologically effective amount of a compound of any of the preceding Embodiments, one or more food materials, optionally an attractant, and optionally a humectant.

Embodiments of the invention also include methods for protecting a seed from a parasitic nematode comprising contacting the seed with a biologically effective amount of a compound of any of the preceding Embodiments.

Embodiments of the invention also include methods for controlling a parasitic nematode comprising contacting the parasitic nematode or its environment with a biologically effective amount of a compound of formula (I), an N-oxide, or a salt thereof, (e.g., as a composition described herein), provided that the methods are not methods of medical treatment of a human or animal body by therapy.

This invention also relates to such methods wherein the parasitic nematode or its environment is contacted with a composition comprising a biologically effective amount of a compound of formula (I), an N-oxide, or a salt thereof, and at least one additional component selected from the group consisting of surfactants, solid diluents and liquid diluents, said composition optionally further comprising a biologically effective amount of at least one additional biologically active compound or agent, provided that the methods are not methods of medical treatment of a human or animal body by therapy.

### Procedures and Methods

One or more of the following methods and variations as described in Schemes 1-8 can be used to prepare the compounds of formula (I). The definitions of A¹, A², A³, A⁴, T, V, W, X, Y, Z, R¹, R², R³ and p in the compounds of formulae (Ia), (Ib) and (Ic) and (II) to (X) below are as defined above in the Summary of the Invention unless otherwise noted. Formulae (Ia-Ic) are various subsets of formula (I), and all substituents for formulae (Ia-Ic) are as defined above for formula (I) unless otherwise noted. Room temperature is between about 20 and 25° C.

Compounds of formula (IA) (i.e. formula (I) wherein Z is oxygen and R³ is H) can be prepared by the reaction of carboxylic acids of formula (II) with sulfonamides of formula (IIIA) as shown in Scheme 1. Typically, an amide coupling reagent and a catalyst such as N,N-dimethylaminopyridine (DMAP) are used in the method of Scheme 1. Amide coupling reagents include 1-ethyl-3-(3-dimethylaminopropyl)-carbodiimide hydrochloride (EDC), N,N′-dicyclohexylcarbodiimide (DCC) and 1,1′-carbonyldiimidazole (CDI). The reaction can be carried out at temperatures ranging from room temperature to the reflux temperature of the solvent. Typical solvents include alcohols, ethers, esters, amides and halogenated hydrocarbons.

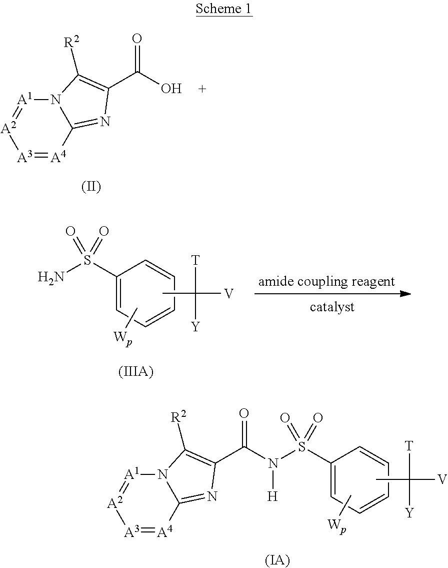

Compounds of formula (IA) can also be prepared by the reaction of carboxylic acid chlorides of formula (IV) with sulfonamides of formula (IIIA) as shown in Scheme 2. The reaction typically involves use of a base such as triethylamine or pyridine and optionally a catalyst such as DMAP in the presence of a solvent. The reaction can be carried out at temperatures ranging from room temperature to the reflux temperature of the solvent. Typical solvents include ethers, esters and halogenated hydrocarbons.

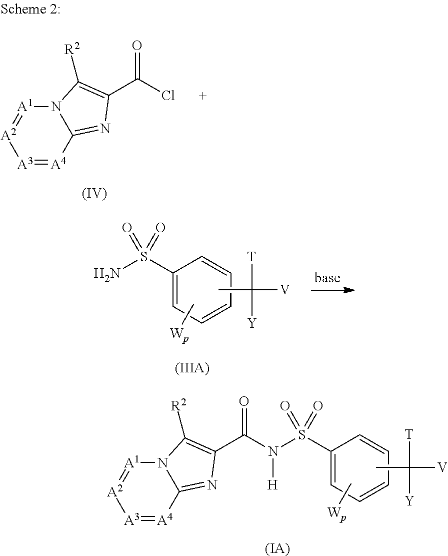

Compounds of formula (IB) wherein R³ is optionally substituted alkyl, alkenyl, alkynyl or cycloalkylalkyl can be prepared by the reaction of compounds of formula (IA) with appropriately substituted alkyl, alkenyl, alkynyl or cycloalkylalkyl halides and base as shown in Scheme 3. Typical reaction conditions comprise potassium carbonate as the base and DMF as the solvent.

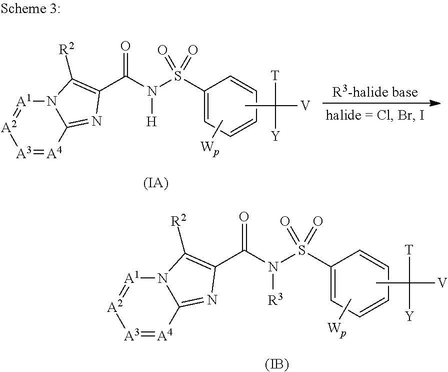

Compounds of formula (IB), wherein R³ is C(X)R⁷, C(O)OR⁸, C(O)NR¹¹R¹², S(O)₂R⁹ or S(O)₂NR¹¹R¹² can be prepared by the reaction of compounds of formula (IA) with acyl or sulfonyl halides (e.g., Cl—C(X)R⁷, Cl—C(O)OR⁸, Cl—C(O)NR¹¹R¹², Cl—S(O)₂R⁹ or Cl—S(O)₂NR¹¹R¹²) by acylation or sulfonylation methods well known in the art.

Compounds of formula (IB) wherein R³ is optionally substituted alkyl, alkenyl, alkynyl, cycloalkyl or phenyl can be prepared by the reaction of acid chlorides of formula (IV) with sulfonamides of formula (MB) as shown in Scheme 4. Alternatively, compounds of formula (IB) wherein R³ is optionally substituted alkyl, alkenyl, alkynyl, cycloalkyl or phenyl can be prepared by the reaction of carboxylic acids of formula (II) with sulfonamides of formula (MB) by the method of Scheme 1.

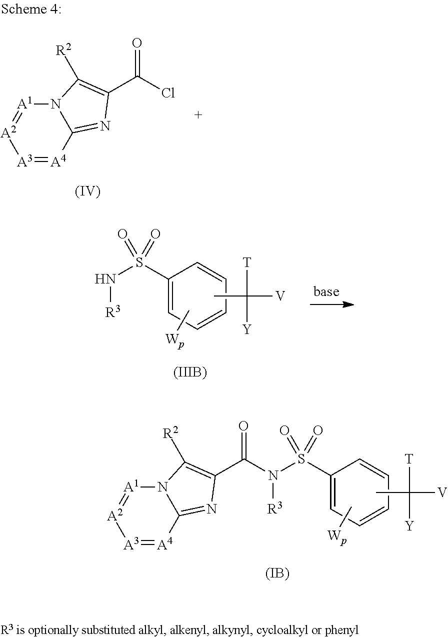

Thioamides of formula (IC) (i.e. formula (I) wherein X is sulfur) can be prepared by the reaction of compounds of formula (IA) or (IB) (i.e. formula (I) wherein X is oxygen) with thiation reagents such as phosphorus pentasulfide or Lawesson's reagent as depicted in Scheme 5.

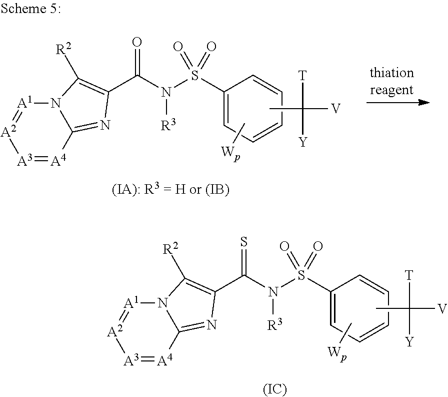

Compounds of formula (IIB) (i.e. formula (II) wherein R² is bromine) can be prepared by the reaction of compounds of formula (IIA) (i.e. formula (II) wherein R² is H) with bromine in acetic acid in the presence of sodium acetate as described in Heterocycles 2002, 57(1), pp. 21-38.

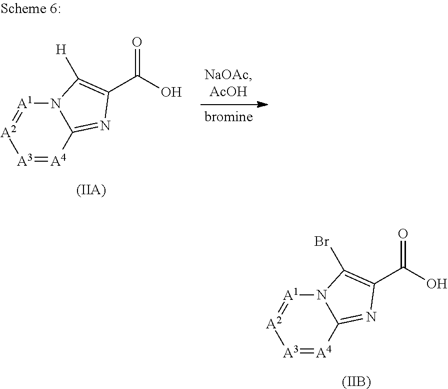

Compounds of formula (II) wherein R² is cyano can be prepared by reaction of compounds of formula (IIB) with CuCN by methods known in the art. Compounds of formula (II) wherein R² is nitro can be prepared by reaction of compounds of formula (IIA) with nitric acid/sulfuric acid as described in Bioorganic Med. Chem. Lett. 2005, 15(11), pp. 2790-2794. Compounds of formula (II) wherein R² is OR⁴, NR⁵R⁶ or SR⁹ can be prepared from compounds of formula (II) wherein R² is F by standard displacement reactions well known in the art. Compounds of formula (II) wherein R² is F can be prepared as described in Russian Chem. Bull. 2005, 54(2), pp. 470-471.

Compounds of formula (I) can also be prepared by the reaction of compounds of formula (V) with heterocycles of formula (H—V) as shown in Scheme 7. If Q represents a leaving group like halogen, OSO₂-alkyl or OSO₂-haloalkyl then the reaction is carried out in the presence of a basic reaction auxiliary like an aliphatic or aromatic amine, alkalihydride or alkali alcoholate in the presence of an inert solvent like tetrahydrofurane, dimethylformamide or dimethylsulfoxide. If Q represents hydroxyl the formation of (I) is accomplished in the presence of an ester of azodicarboxylate and triphenylphosphine in an inert solvent like tetrahydrofurane (Mitsunobu-reaction).

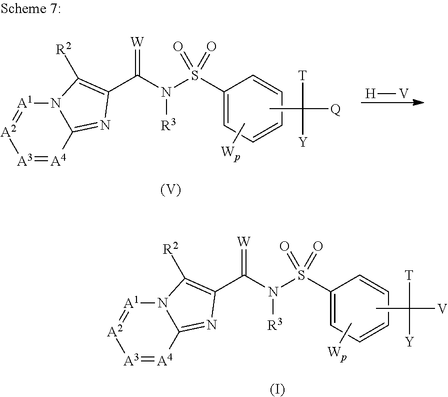

Carboxylic acids of formula (II) and acid chlorides of Formula (IV) can be prepared by the reactions shown in Scheme 8. Reaction of a suitably substituted 2-aminoazine of formula (VI) with a 2-bromopyruvate of formula (VII) wherein R² is H, optionally substituted alkyl, alkenyl, alkynyl, C(O)R⁷, C(O)OR⁸ or C(O)NR¹¹R¹², or an optionally substituted phenyl, naphthalenyl or a 5- or 6-membered heteroaromatic ring at temperatures ranging from room temperature to the boiling temperature of the solvent affords the carboxylic ester of formula (VIII) along with variable amounts of the alcohol of formula (IX) (see, for example, Europ. J. Med. Chem. 1988.23(4), pp. 385-389). Heating the reaction mixture to boiling in a solvent such as 1,2-dimethoxyethane results in complete conversion of alcohol (IX) to ester (VIII). Treatment of a mixture of esters (VIII) and (IX) with an aqueous hydroxide base such as sodium hydroxide in a water-miscible solvent such as ethanol results in ester hydrolysis to form the carboxylic acid of formula (II) after acidification with a strong acid such as hydrochloric acid. Furthermore, ester cleavage occurs in the presence of strong Lewis acids like BBr₃ in CH₂Cl₂, favorable in case of decarboxylation-sensitive carboxylic groups. The carboxylic acid of formula (II) can be converted to the acid chloride of formula (IV) by well-known conventional means such as treatment with thionyl chloride or oxalyl chloride with a catalytic amount of N,N-dimethylformamide (DMF) in moderately polar, aprotic solvents including dichloromethane, dichloroethane, toluene and ethyl acetate. Intermediates of formula (VII) can be prepared by a variety of well-known synthetic methods, including the bromination of optionally substituted pyruvates or lactates (alpha-hydroxy esters). Typical reaction conditions include direct bromination with bromine (see, for example, JACS 1944, 66, pp. 1656-1659) or CuBr₂ in ethyl acetate/chloroform (see, for example, JOC 2002, 67(4), pp. 1102-1108), or reaction of a lactate with N-bromosuccinimide in CCl₄ (see, for example, JACS 1954, 76, pp. 5796-5797).

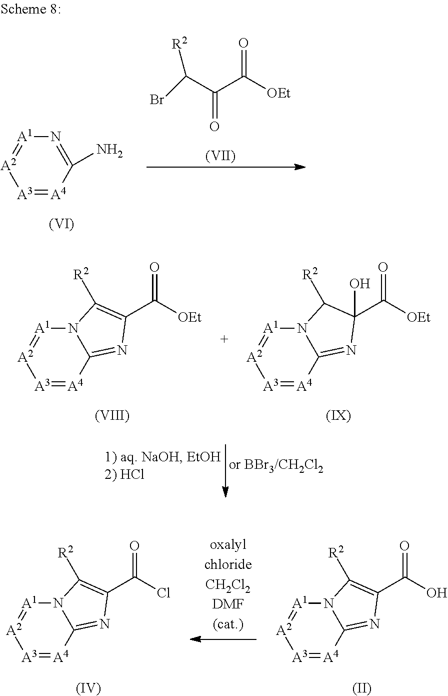

Examples for intermediates of formula (II) or (IV) are in most instances commercially available (com. ay. or can be prepared according to well-known procedures, for example as indicated in table 1.

TABLE 1

| A¹ | A² | A³ | A⁴ | R² | remarks |
|---|---|---|---|---|---|
| C—H | C—H | C—H | C—H | H | com. av. |
| C—H | C—H | C—H | C—F | H | com. av. |
| C—H | C—H | C—H | C—Cl | H | com. av. |
| C—H | C—H | C—H | C—Br | H | com. av. |
| C—H | C—CF₃ | C—H | C—H | H | com. av. |
| C—H | C—CF₃ | C—H | C—F | H | P2 |
| C—H | C—CF₃ | C—H | C—Cl | H | com. av. |
| C—H | C—CF₃ | C—H | C—Br | H | P2 |
| C—H | C—F | C—H | C—H | H | com. av. |
| C—H | C—F | C—H | C—F | H | com. av. (Et- |
| | | | | | ester) |
| C—H | C—F | C—H | C—Cl | H | com. av. |
| C—H | C—F | C—H | C—Br | H | com. av. |
| C—H | C—Cl | C—H | C—H | H | com. av. |
| C—H | C—Cl | C—H | C—F | H | com. av. |
| C—H | C—Cl | C—H | C—Cl | H | com. av. |
| C—H | C—Cl | C—H | C—Br | H | com. av. |
| C—H | C—Br | C—H | C—H | H | com. av. |
| C—H | C—Br | C—H | C—F | H | com. av. |
| C—H | C—Br | C—H | C—Cl | H | |
| C—H | C—Br | C—H | C—Br | H | com. av. |
| N | C—H | C—H | C—H | H | com. av. |
| N | C—H | C—H | C—F | H | P4 |
| N | C—H | C—H | C—Cl | H | P4 |
| N | C—H | C—H | C—Br | H | P4 |
| N | C—CF₃ | C—H | C—H | H | P4 |
| N | C—CF₃ | C—H | C—F | H | P4 |
| N | C—CF₃ | C—H | C—Cl | H | P4 |
| N | C—CF₃ | C—H | C—Br | H | P4 |
| N | C—Cl | C—H | C—H | H | P4 |
| N | C—Cl | C—H | C—F | H | P4 |
| N | C—Cl | C—H | C—Cl | H | P4 |
| N | C—Cl | C—H | C—Br | H | P4 |
| N | C—Br | C—H | C—H | H | P4 |
| N | C—Br | C—H | C—F | H | P4 |
| N | C—Br | C—H | C—Cl | H | P4 |
| N | C—Br | C—H | C—Br | H | P4 |
| C—H | N | C—H | C—H | H | com. av. |
| C—H | N | C—F | C—H | H | P4 |
| C—H | N | C—Cl | C—H | H | com. av. |
| C—H | N | C—Br | C—H | H | P4 |
| C—H | C—H | N | C—H | H | com. av. |
| C—H | C—H | N | C—F | H | P4 |
| C—H | C—H | N | C—Cl | H | com. av. |
| C—H | C—H | N | C—Br | H | P4 |
| C—H | C—CF₃ | N | C—H | H | com. av. |
| C—H | C—CF₃ | N | C—F | H | P4 |
| C—H | C—CF₃ | N | C—Cl | H | P4 |
| C—H | C—CF₃ | N | C—Br | H | P4 |
| C—H | C—Cl | N | C—H | H | com. av. |
| C—H | C—Cl | N | C—F | H | P4 |
| C—H | C—Cl | N | C—Cl | H | P4 |
| C—H | C—Cl | N | C—Br | H | P4 |
| C—H | C—Br | N | C—H | H | com. av. |
| C—H | C—Br | N | C—F | H | P4 |
| C—H | C—Br | N | C—Cl | H | P4 |
| C—H | C—Br | N | C—Br | H | P4 |
| C—H | C—H | C—H | N | H | com. av. |
| C—H | C—CF₃ | C—H | N | H | com. av. |
| C—H | C—F | C—H | N | H | com. av. |
| C—H | C—Cl | C—H | N | H | com. av. |
| C—H | C—Br | C—H | N | H | com. av. |

### Synthesis of Intermediates of Formula (IIIA):

General Synthesis Route of Intermediates of Formula (IIIA), where T=Y═H, Scheme 9:

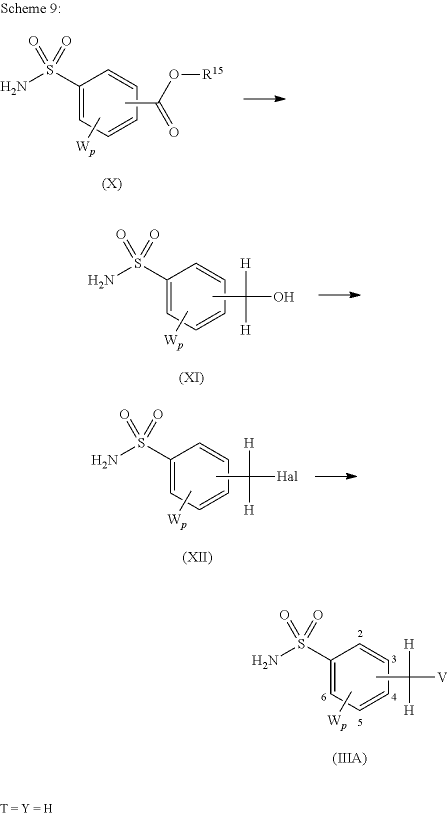

Transformation of carboxylic acids of formula (X) (R¹⁵═H) to alcohols of formula (XI) is well known in the literature, e.g. WO 2003/091204 and WO 2011/071565 (BH₃-tetrahydrofurane complex) as well as reduction of esters of formula (X) (R¹⁵=methyl or ethyl) with various alkali borohydrides or lithium aluminiumhydride in tetrahydrofurane or tetrahydrofurane—ethanol mixtures (WO 2009/118292, WO 2005/082890, US 2010/0009970). Benzylhalogenides of formula (XII) can be prepared from alcohols of formula (XI) with halogenating agents like phosphorus tribromide (PBr₃) or thionylchloride (SOCl₂), (see, for example, WO 2011/071565).

Reaction of benzylic halogenides (e.g. compounds of formula (XII)) with various nitrogen nucleophiles is a process well known in the art.

The compound according to the present invention can be prepared according to the processes described above. It will nevertheless be understood that, on the basis of his general knowledge and of available publications, the skilled worker will be able to adapt this method according to the specifics of each of the compounds, which it is desired to synthesize.

The compounds of the invention can be used as nematicides and/or as insecticides.

A “nematicide” as used herein means that the compound is capable of controlling nematodes.

“Controlling nematodes” according to the invention shall mean to kill nematodes or to prevent their development or growth. The efficacy of the compositions or combinations according to the invention is assessed by comparing the mortality of nematodes, the development of galls, the formation of cysts, the concentration of nematodes per volume of soil, of cysts, the concentration of nematodes per root, the number of nematode eggs per volume of soil, the motility of the nematodes between a plant, a plant part or the soil treated with a composition or combination according to the invention and the untreated plant, plant part or soil (100%). Preferred is a reduction by 25-50% in comparison with the untreated plant, plant part or soil, very preferred a reduction by 51-79%, and particularly preferred the complete killing and the complete prevention of the development or growth by a reduction from 80% to 100% in comparison with the untreated plant, plant part or soil.

“Controlling nematodes” according to the invention shall mean the control of the reproduction of the nematodes (e.g. development of cysts or eggs). The compositions according to the invention can be used for keeping the plants healthy and can be used curatively, preventively or systemically for controlling nematodes.

The skilled person knows methods for determining the mortality of nematodes, the development of galls, the formation of cysts, the concentration of nematodes per volume of soil, of cysts, the concentration of nematodes per root, the number of nematode eggs per volume of soil, the motility of the nematodes between a plant, a plant part or the soil. The treatment according to the invention reduces the damages caused by nematodes to the plant and leads to an increase in yield.

“Nematodes” as used herein encompass all species of the phylum Nematoda and in particular species that are parasitic or cause health problems to plant or to fungi (for example species of the orders Aphelenchida, Meloidogyne, Tylenchida and others) or to humans and animals (for example species of the orders Trichinellida, Tylenchida, Rhabditina, and Spirurida) as well as other parasitic helminths.

“Nematodes” as used herein, refer to plant nematodes meaning all nematodes that cause damage to plants. Plant nematodes encompass plant parasitic nematodes and nematodes living in the soil. Plant parasitic nematodes include, but are not limited to, ectoparasites such as Xiphinema spp., Longidorus spp., and Trichodorus spp.; semiparasites such as Tylenchulus spp.; migratory endoparasites such as Pratylenchus spp., Radopholus spp., and Scutellonerna spp.; sedentary parasites such as Heterodera spp., Globodera spp., and Meloidogyne spp., and stem and leaf endoparasites such as Ditylenchus spp., Aphelenchoides spp., and Hirshmaniella spp. Especially harmful root parasitic soil nematodes are such as cystforming nematodes of the genera Heterodera or Globodera, and/or root knot nematodes of the genus Meloidogyne. Harmful species of these genera are for example Meloidogyne incognita, Heterodera glycines (soybean cyst nematode), Globodera pallida and Globodera rostochiensis (potato cyst nematode), which species are effectively controlled with the compounds described herein. However, the use of the compounds described herein is in no way restricted to these genera or species, but also extends in the same manner to other nematodes.

Plant nematodes include but are not limited to e.g. Aglenchus agricola, Anguina tritici, Aphelenchoides arachidis, Aphelenchoides fragaria and the stem and leaf endoparasites Aphelenchoides spp. in general, Belonolaimus gracilis, Belonolaimus longicaudatus, Belonolaimus nortoni, Bursaphelenchus cocophilus, Bursaphelenchus eremus, Bursaphelenchus xylophilus and Bursaphelenchus spp. in general, Cacopaurus pestis, Criconemella curvata, Criconemella onoensis, Criconemella ornata, Criconemella rusium, Criconemella xenoplax (=Mesocriconema xenoplax) and Criconemella spp. in general, Criconemoides ferniae, Criconemoides onoense, Criconemoides ornatum and Criconemoides spp. in general, Ditylenchus destructor, Ditylenchus dipsaci, Ditylenchus myceliophagus and the stem and leaf endoparasites Ditylenchus spp. in general, Dolichodorus heterocephalus, Globodera pallida (=Heterodera pallida), Globodera rostochiensis (potato cyst nematode), Globodera solanacearum, Globodera tabacum, Globodera virginia and the sedentary, cyst forming parasites Globodera spp. in general, Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp. in general, Hemicriconemoides, Hemicycliophora arenaria, Hemicycliophora nudata, Hemicycliophora parvana, Heterodera avenae, Heterodera cruciferae, Heterodera glycines (soybean cyst nematode), Heterodera oryzae, Heterodera schachtii, Heterodera zeae and the sedentary, cyst forming parasites Heterodera spp. in general, Hirschmaniella gracilis, Hirschmaniella oryzae Hirschmaniella spinicaudata and the stem and leaf endoparasites Hirschmaniella spp. in general, Hoplolaimus aegyptii, Hoplolaimus californicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp. in general, Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the sedentary parasites Meloidogyne spp. in general, Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp. in general, Paratylenchus hamatus, Paratylenchus minutus, Paratylenchus projectus and Paratylenchus spp. in general, Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp. in general, Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp. in general, Rotylenchulus borealis, Rotylenchulus parvus, Rotylenchulus reniformis and Rotylenchulus spp. in general, Rotylenchus laurentinus, Rotylenchus macrodoratus, Rotylenchus robustus, Rotylenchus uniformis and Rotylenchus spp. in general, Scutellonema brachyurum, Scutellonema bradys, Scutellonema clathricaudatum and the migratory endoparasites Scutellonema spp. in general, Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp. in general, Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp. in general, Tylenchulus semipenetrans and the semiparasites Tylenchulus spp. in general, Xiphinema americanum, Xiphinema brevicolle, Xiphinema dimorphicaudatum, Xiphinema index and the ectoparasites Xiphinema spp. in general.

Examples of nematodes to which a nematicide of the present invention is applicable include, but are not limited to, nematodes of the genus Meloidogyne such as the southern root-knot nematode (Meloidogyne incognita), Javanese root-knot nematode (Meloidogyne javanica), northern root-knot nematode (Meloidogyne hapla), and peanut root-knot nematode (Meloidogyne arenaria); nematodes of the genus Ditylenchus such as the potato rot nematode (Ditylenchus destructor) and bulb and stem nematode (Ditylenchus dipsaci); nematodes of the genus Pratylenchus such as the cob root-lesion nematode (Pratylenchus penetrans), chrysanthemum root-lesion nematode (Pratylenchus fallax), coffee root-lesion nematode (Pratylenchus coffeae), tea root-lesion nematode (Pratylenchus loosi), and walnut root-lesion nematode (Pratylenchus vulnus); nematodes of the genus Globodera such as the golden nematode (Globodera rostochiensis) and potato cyst nematode (Globodera pallida); nematodes of the genus Heterodera such as the soybean cyst nematode (Heterodera glycines) and sugar beet cyst nematode (Heterodera schachtii); nematodes of the genus Aphelenchoides such as the rice white-tip nematode (Aphelenchoides besseyi), chrysanthemum foliar nematode (Aphelenchoides ritzemabosi), and strawberry nematode (Aphelenchoides fragariae); nematodes of the genus Aphelenchus such as the mycophagous nematode (Aphelenchus avenae); nematodes of the genus Radopholus such as the burrowing nematode (Radopholus similis); nematodes of the genus Tylenchulus such as the citrus nematode (Tylenchulus semipenetrans); nematodes of the genus Rotylenchulus such as the reniform nematode (Rotylenchulus reniformis); nematodes that occur in trees, such as the pine wood nematode (Bursaphelenchus xylophilus), and the like.

Plants for which a nematicide of the present invention can be used are not particularly limited; for example, plants such as cereals (for example, rice, barley, wheat, rye, oat, corn, and the like), beans (soybeans, azuki beans, broad beans, peas, peanuts and the like), fruit trees/fruits (apples, citrus species, pears, grapes, peaches, Japanese apricots, cherries, walnuts, almonds, bananas, strawberries and the like), vegetables (cabbage, tomato, spinach, broccoli, lettuce, onion, Welsh onion, pepper and the like), root crops (carrot, potato, sweet potato, radish, lotus root, turnip and the like), industrial crops (cotton, hemp, paper mulberry, mitsumata, rape, beet, hop, sugarcane, sugar beet, olive, rubber, palms, coffee, tobacco, tea and the like), pepos (pumpkin, cucumber, watermelon, melon and the like), pasture plants (orchard grass, sorghum, thimosy, clover, alfalfa and the like), lawn grasses (mascarene grass, bent grass and the like), crops for flavorings etc. (lavender, rosemary, thyme, parsley, pepper, ginger and the like), and flower plants (chrysanthemum, rose, orchids and the like) can be mentioned.

The compound(s) and compositions comprising the compound(s) of the present invention is/are particularly useful in controlling nematodes in coffee belonging to at least one species selected from the group of the phytoparasitic nematodes consisting of Pratylenchus brachyurus, Pratylenchus coffeae, Meloidogyne exigua, Meloidogyne incognita, Meloidogyne coffeicola, Helicotylenchus spp. and also consisting of Meloidogyne paranaensis, Rotylenchus spp., Xiphinema spp., Tylenchorhynchus spp., Scutellonema spp.

Compound(s) and compositions comprising compound(s) of the present invention is/are particularly useful in controlling nematodes in potato belonging to at least one species selected from the group of the phytoparasitic nematodes consisting of Pratylenchus brachyurus, Pratylenchus pratensis, Pratylenchus scribneri, Pratylenchus penetrans, Pratylenchus coffeae, Ditylenchus dipsaci and also consisting of Pratylenchus alleni, Pratylenchus andinus, Pratylenchus cerealis, Pratylenchus crenatus, Pratylenchus hexincisus, Pratylenchus loosi, Pratylenchus neglectus, Pratylenchus teres, Pratylenchus thornei, Pratylenchus vulnus, Belonolaimus longicaudatus, Trichodorus cylindricus, Trichodorus primitivus, Trichodorus proximus, Trichodorus similis, Trichodorus sparsus, Paratrichodorus minor, Paratrichodorus allius, Paratrichodorus nanus, Paratrichodorus teres, Meloidogyne arenaria, Meloidogyne fallax, Meloidogyne hapla, Meloidogyne thamesi, Meloidogyne incognita, Meloidogyne chitwoodi, Meloidogyne javanica, Nacobbus aberrans, Globodera rostochiensis, Globodera pallida, Ditylenchus destructor, Radopholus similis, Rotylenchulus reniformis, Neotylenchus vigissi, Paraphelenchus pseudoparietinus, Aphelenchoides fragariae, Meloinema spp.

Compound(s) and compositions comprising the compound(s) of the present invention is/are particularly useful in controlling nematodes in tomato belonging to at least one species selected from the group of the phytoparasitic nematodes consisting of Meloidogyne arenaria, Meloidogyne hapla, Meloidogyne javanica, Meloidogyne incognita, Pratylenchus penetrans and also consisting of Pratylenchus brachyurus, Pratylenchus coffeae, Pratylenchus scribneri, Pratylenchus vulnus, Paratrichodorus minor, Meloidogyne exigua, Nacobbus aberrans, Globodera solanacearum, Dolichodorus heterocephalus, Rotylenchulus reniformis.

The compound(s) and compositions comprising the compound(s) of the present invention is/are particularly useful in controlling nematodes in cucurbits belonging to at least one species selected from the group of the phytoparasitic nematodes consisting of Meloidogyne arenaria, Meloidogyne hapla, Meloidogyne javanica, Meloidogyne incognita, Rotylenchulus reniformis and also consisting of Pratylenchus thornei.

The compound(s) and compositions comprising the compound(s) of the present invention is/are particularly useful in controlling nematodes in cotton belonging to at least one species selected from the group of the phytoparasitic nematodes consisting of Belonolaimus longicaudatus, Meloidogyne incognita, Hoplolaimus columbus, Hoplolaimus galeatus, Rotylenchulus reniformis.

The compound(s) and compositions comprising the compound(s) of the present invention is/are particularly useful in controlling nematodes in corn belonging to at least one species selected from the group of the phytoparasitic nematodes, especially consisting of Belonolaimus longicaudatus, Paratrichodorus minor and also consisting of Pratylenchus brachyurus, Pratylenchus delattrei, Pratylenchus hexincisus, Pratylenchus penetrans, Pratylenchus zeae, (Belonolaimus gracilis), Belonolaimus nortoni, Longidorus breviannulatus, Meloidogyne arenaria, Meloidogyne arenaria thamesi, Meloidogyne graminis, Meloidogyne incognita, Meloidogyne incognita acrita, Meloidogyne javanica, Meloidogyne naasi, Heterodera avenae, Heterodera oryzae, Heterodera zeae, Punctodera chalcoensis, Ditylenchus dipsaci, Hoplolaimus aegyptii, Hoplolaimus magnistylus, Hoplolaimus galeatus, Hoplolaimus indicus, Helicotylenchus digonicus, Helicotylenchus dihystera, Helicotylenchus pseudorobustus, Xiphinema americanum, Dolichodorus heterocephalus, Criconemella ornata, Criconemella onoensis, Radopholus similis, Rotylenchulus borealis, Rotylenchulus parvus, Tylenchorhynchus agri, Tylenchorhynchus clarus, Tylenchorhynchus claytoni, Tylenchorhynchus maximus, Tylenchorhynchus nudus, Tylenchorhynchus vulgaris, Quinisulcius acutus, Paratylenchus minutus, Hemicycliophora parvana, Aglenchus agricola, Anguina tritici, Aphelenchoides arachidis, Scutellonema brachyurum, Subanguina radiciola.

The compound(s) and compositions comprising the compound(s) of the present invention is/are particularly useful in controlling nematodes in soybean belonging to at least one species selected from the group of the phytoparasitic nematodes, especially consisting of Pratylenchus brachyurus, Pratylenchus pratensis, Pratylenchus penetrans, Pratylenchus scribneri, Belonolaimus longicaudatus, Heterodera glycines, Hoplolaimus columbus and also consisting of Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, (Belonolaimus gracilis), Meloidogyne arenaria, Meloidogyne incognita, Meloidogyne javanica, Meloidogyne hapla, Hoplolaimus columbus, Hoplolaimus galeatus, Rotylenchulus reniformis.

The compound(s) and compositions comprising the compound(s) of the present invention is/are particularly useful in controlling nematodes in tobacco belonging to at least one species selected from the group of the phytoparasitic nematodes, especially consisting of Meloidogyne incognita, Meloidogyne javanica and also consisting of Pratylenchus brachyurus, Pratylenchus pratensis, Pratylenchus hexincisus, Pratylenchus penetrans, Pratylenchus neglectus, Pratylenchus crenatus, Pratylenchus thomei, Pratylenchus vulnus, Pratylenchus zeae, Longidorus elongatu, Paratrichodorus lobatus, Trichodorus spp., Meloidogyne arenaria, Meloidogyne hapla, Globodera tabacum, Globodera solanacearum, Globodera virginiae, Ditylenchus dipsaci, Rotylenchus spp., Helicotylenchus spp., Xiphinema americanum, Criconemella spp., Rotylenchulus reniformis, Tylenchorhynchus claytoni, Paratylenchus spp., Tetylenchus nicotianae.

The compound(s) and compositions comprising the compound(s) of the present invention is/are particularly useful in controlling nematodes in citrus belonging to at least one species selected from the group of the phytoparasitic nematodes, especially consisting of Pratylenchus coffeae and also consisting of Pratylenchus brachyurus, Pratylenchus vulnus, Belonolaimus longicaudatus, Paratrichodorus minor, Paratrichodorus porosus, Trichodorus, Meloidogyne incognita, Meloidogyne incognita acrita, Meloidogyne javanica, Rotylenchus macrodoratus, Xiphinema americanum, Xiphinema brevicolle, Xiphinema index, Criconemella spp., Hemicriconemoides, Radopholus similis respectively Radopholus citrophilus, Hemicycliophora arenaria, Hemicycliophora nudata, Tylenchulus semipenetrans.

The compound(s) and compositions comprising the compound(s) of the present invention is/are particularly useful in controlling nematodes in banana belonging to at least one species selected from the group of the phytoparasitic nematodes, especially consisting of Pratylenchus coffeae, Radopholus similis and also consisting of Pratylenchus giibbicaudatus, Pratylenchus loosi, Meloidogyne spp., Helicotylenchus multicinctus, Helicotylenchus dihystera, Rotylenchulus spp.

The compound(s) and compositions comprising the compound(s) of the present invention is/are particularly useful in controlling nematodes in pine apple belonging to at least one species selected from the group of the phytoparasitic nematodes, especially consisting of Pratylenchus zeae, Pratylenchus pratensis, Pratylenchus brachyurus, Pratylenchus goodeyi., Meloidogyne spp., Rotylenchulus reniformis and also consisting of Longidorus elongatus, Longidorus laevicapitatus, Trichodorus primitivus, Trichodorus minor, Heterodera spp., Ditylenchus myceliophagus, Hoplolaimus californicus, Hoplolaimus pararobustus, Hoplolaimus indicus, Helicotylenchus dihystera, Helicotylenchus nannus, Helicotylenchus multicinctus, Helicotylenchus erythrine, Xiphinema dimorphicaudatum, Radopholus similis, Tylenchorhynchus digitatus, Tylenchorhynchus ebriensis, Paratylenchus minutus, Scutellonema clathricaudatum, Scutellonema bradys, Psilenchus tumidus, Psilenchus magnidens, Pseudohalenchus minutus, Criconemoides ferniae, Criconemoides onoense, Criconemoides ornatum.

The compound(s) and compositions comprising the compound(s) of the present invention is/are particularly useful in controlling nematodes in grapes belonging to at least one species selected from the group of the phytoparasitic nematodes, especially consisting of Pratylenchus vulnus, Meloidogyne arenaria, Meloidogyne incognita, Meloidogyne javanica, Xiphinema americanum, Xiphinema index and also consisting of Pratylenchus pratensis, Pratylenchus scribneri, Pratylenchus neglectus, Pratylenchus brachyurus, Pratylenchus thomei, Tylenchulus semipenetrans.

The compound(s) and compositions comprising the compound(s) of the present invention is/are particularly useful in controlling nematodes in tree crops—pome fruits, belonging to at least one species selected from the group of the phytoparasitic nematodes, especially consisting of Pratylenchus penetrans and also consisting of Pratylenchus vulnus, Longidorus elongatus, Meloidogyne incognita, Meloidogyne hapla.

The compound(s) and compositions comprising the compound(s) of the present invention is/are particularly useful in controlling nematodes in tree crops—stone fruits, belonging to at least one species selected from the group of the phytoparasitic nematodes, especially consisting of Pratylenchus penetrans, Pratylenchus vulnus, Meloidogyne arenaria, Meloidogyne hapla, Meloidogyne javanica, Meloidogyne incognita, Criconemella xenoplax and also consisting of Pratylenchus brachyurus, Pratylenchus coffeae, Pratylenchus scribneri, Pratylenchus zeae, Belonolaimus longicaudatus, Helicotylenchus dihystera, Xiphinema americanum, Criconemella curvata, Tylenchorhynchus claytoni, Paratylenchus hamatus, Paratylenchus projectus, Scutellonema brachyurum, Hoplolaimus galeatus.

The compound(s) and compositions comprising the compound(s) of the present invention is/are particularly useful in controlling nematodes in tree crops—nuts, belonging to at least one species selected from the group of the phytoparasitic nematodes, especially consisting of Trichodorus spp., Criconemella rusium and also consisting of Pratylenchus vulnus, Paratrichodorus spp., Meloidogyne incognita, Helicotylenchus spp., Tylenchorhynchus spp., Cacopaurus pestis.

In a like manner, “nematodes” as used herein, refer to nematodes which cause damage to humans or animals

Specific nematode species harmful to humans or animals are:

Trichinellida for example Trichuris spp., Capillaria spp., Trichomosoides spp., Trichinella spp. From the order of the Tylenchida for example Micronema spp., Strongyloides spp.

From the order of the Rhabditina for example Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp.

Many known nematicides are equally active against other parasitic helminths and are therefore used to control human and animal parasitic worms, which do not necessarily belong to the group of nematoda. Therefore, it is envisaged by the present invention that the compounds described herein may also be used as anthelmintic drugs in a more general meaning. Pathogenic endoparasitic helminths include platyhelmintha (e.g. monogenea, cestodes and trematodes), acanthocephala, and pentastoma. The following helminths may be mentioned by way of example and by way of preference—but without any limitation:

Monogenea: e.g.: Gyrodactylus spp., Dactylogyrus spp., Polystoma spp.

Cestodes: From the order of the Pseudophyllidea for example: Diphyllobothrium spp., Spirometra spp., Schistocephalus spp., Ligula spp., Bothridium spp., Diplogonoporus spp.

From the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: From the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.

Acantocephala: From the order of the Oligacanthorhynchida z.B: Macracanthorhynchus spp., Prosthenorchis spp.; from the order of the Polymorphida for example: Filicollis spp.; from the order of the Moniliformida for example: Moniliformis spp.,

From the order of the Echinorhynchida for example Acanthocephalus spp., Echinorhynchus spp., Leptorhynchoides spp.

Pentastoma: From the order of the Porocephalida for example Linguatula spp.

In the veterinary field and in animal keeping, the administration of the active compounds according to the invention is carried out in the known manner directly or enterally, parenterally, dermally or nasally in the form of suitable preparations. Administration can be carried out prophylactically or therapeutically.

A further aspect of the invention are nematicidal compositions, comprising an effective amount of at least one compound as defined herein and at least one of the following: surfactant, solid or liquid diluent, characterized in that the surfactant or the diluent is normally used in nematicidal compositions. In an embodiment, said composition comprises at least two compounds as defined herein.

A related aspect of the invention is a method for preparing a nematicidal composition as described herein, comprising the step of mixing at least one compound as described herein with a surfactant or diluent normally used in nematicidal compositions. In an embodiment, said method comprises mixing least two compounds as defined herein with a surfactant or diluent normally used in nematicidal compositions.

In particular, the present invention relates to nematicidal composition developed to be used in agriculture or horticulture. These nematicidal compositions may be prepared in a manner known per se.

In the animal health field, i.e. in the field of veterinary medicine, the active compounds according to the present invention are active against animal parasites, in particular ectoparasites or endoparasites. The term endoparasite includes in particular helminths and protozoae, such as coccidia. Ectoparasites are typically and preferably arthropods, in particular insects and acarids. The compounds of formula (I) are preferably active against helminths.

In the field of veterinary medicine the compounds according to the invention are suitable, with favourable warm blood toxicity, for controlling parasites which occur in animal breeding and animal husbandry in livestock, breeding, zoo, laboratory, experimental and domestic animals They are active against all or specific stages of development of the parasites.

Agricultural livestock include, for example mammals, such as, sheep, goats, horses, donkeys, camels, buffaloes, rabbits, reindeers, fallow deers, and in particular cattle and pigs; or poultry such as turkeys, ducks, geese, and in particular chickens; or fish or crustaceans e.g. in aquaculture; or as the case may be insects such as bees.

Domestic animals include, for example mammals, such as hamsters, guinea pigs, rats, mice, chinchillas, ferrets or in particular dogs, cats; cage birds; reptiles; amphibians or aquarium fish.

According to a preferred embodiment, the compounds according to the invention are administered to mammals

According to another preferred embodiment, the compounds according to the invention are administered to birds, namely cage birds or in particular poultry.

By using the active compounds according to the invention to control animal parasites, it is intended to reduce or prevent illness, cases of deaths and performance reductions (in the case of meat, milk, wool, hides, eggs, honey and the like), so that more economical and simpler animal keeping is made possible and better animal well-being is achievable.

The term “control” or “controlling” as used herein with regard to the animal health field, means that the active compounds are effective in reducing the incidence of the respective parasite in an animal infected with such parasites to innocuous levels. More specifically, “controlling”, as used herein, means that the active compound is effective in killing the respective parasite, inhibiting its growth, or inhibiting its proliferation.

Exemplary arthropods include, without any limitation:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order of the Diptera and the suborders Nematocerina and Brachy [symbol] cerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order of the Siphonapterida, for example Pulexspp., Ctenocephalides spp., Tunga spp., Xenopsylla spp., Ceratophyllus spp.; from the order of the Heteropterida, for example Cimex spp., Triatoma spp., Rhodnius spp., Panstrongylus spp.; as well as nuisance and hygiene pests from the order of the Blattarida.

Further, among the arthropods, the following acari may be mentioned by way of example, without any limitation:

from the subclass of the Acari (Acarina) and the order of the Metastigmata, for example from the family of argasidae like Argas spp., Ornithodorus spp., Otobius spp., from the family of Ixodidae like Ixodes spp., Amblyomma spp., Rhipicephalus (Boophilus) spp Dermacentor spp., Haemophysalis spp., Hyalomma spp., Rhipicephalus spp. (the original genus of multi host ticks); from the order of mesostigmata like Dermanyssus spp., Ornithonyssus spp., Pneumonyssus spp Raillietia spp., Pneumonyssus spp., Sternostoma spp., Varroa spp., Acarapis spp.; from the order of the Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp, Laminosioptes spp.

Exemplary parasitic protozoa include —, without any limitation:

Mastigophora (Flagellata), such as, for example, Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example, Giardia lamblia, G. canis.

Sarcomastigophora (Rhizopoda), such as Entamoebidae, for example, Entamoeba histolytica, Hartmanellidae, for example, Acanthamoeba sp., Harmanella sp.

Apicomplexa (Sporozoa), such as Eimeridae, for example, Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example, Toxoplasma gondii, Hammondia heydornii, Neospora caninum, Besnoitia besnoitii; such as Sarcocystidae, for example, Sarcocystis bovicanis, S. bovihominis, S. ovicanis, S. ovifelis, S. neurona, S. spec., S. suihominis, such as Leucozoidae, for example, Leucozytozoon simondi, such as Plasmodiidae, for example, Plasmodium berghei, P. falciparum, P. malariae, P. ovale, P. vivax, P. spec., such as Piroplasmea, for example, Babesia argentina, B. bovis, B. canis, B. spec., Theileria parva, Theileria spec., such as Adeleina, for example, Hepatozoon canis, H. spec.

Exemplary pathogenic endoparasites, which are helminths, include platyhelmintha (e.g. monogenea, cestodes and trematodes), nematodes, acanthocephala, and pentastoma. Additional exemplary helminths include —, without any limitation:

Monogenea: e.g.: Gyrodactylus spp., Dactylogyrus spp., Polystoma spp.

Cestodes: From the order of the Pseudophyllidea for example: Diphyllobothrium spp., Spirometra spp., Schistocephalus spp., Ligula spp., Bothridium spp., Diplogonoporus spp.

From the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: From the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

Nematodes: Trichinellida for example: Trichuris spp., Capillaria spp., Trichomosoides spp., Trichinella spp.

From the order of the Tylenchida for example: Micronema spp., Strongyloides spp.

From the order of the Rhabditina for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp.

Acantocephala: From the order of the Oligacanthorhynchida z.B: Macracanthorhynchus spp., Prosthenorchis spp.; from the order of the Polymorphida for example: Filicollis spp.; from the order of the Moniliformida for example: Moniliformis spp.,

From the order of the Echinorhynchida for example Acanthocephalus spp., Echinorhynchus spp., Leptorhynchoides spp.

Pentastoma: From the order of the Porocephalida for example Linguatula spp.

In the veterinary field and in animal keeping, the administration of the active compounds according to the invention is carried out by methods generally known in the art, such as enterally, parenterally, dermally or nasally in the form of suitable preparations. Administration can be carried out prophylactically or therapeutically.

Thus, one embodiment of the present invention refers to compounds according to the invention for use as a medicament.

Another aspect refers to compounds according to the invention for use as an antiendoparasitical agent, in particular a helmithicidal agent or antiprotozoaic agent. For example, compounds according to the invention for use as an antiendoparasitical agent, in particular an helmithicidal agent or antiprotozoaic agent, e.g., in animal husbandry, in animal breeding, in animal housing, in the hygiene sector.

Yet another aspect refers to compounds according to the invention for use as an antiectoparasitical agent, in particular an arthropodicidal agent such as an insecticidal agent or acaricidal agent. For example, compounds according to the invention for use as an antiectoparasitical agent, in particular an arthropodicidal agent such as an insecticidal agent or acaricidal agent, e.g., in animal husbandry, in animal breeding, in animal housing, in the hygiene sector.

The present invention further provides formulations, and application forms prepared from them, as crop protection agents and/or pesticidal agents, such as drench, drip and spray liquors, comprising at least one of the active compounds of the invention. The application forms may comprise further crop protection agents and/or pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example.

Examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible types of formulation are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations may comprise active agrochemical compounds other than one or more active compounds of the invention.

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants. The formulations are prepared in suitable plants or else before or during the application.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used.

Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51, 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

The formulations preferably comprise between 0.00000001% and 98% by weight of active compound or, with particular preference, between 0.01% and 95% by weight of active compound, more preferably between 0.5% and 90% by weight of active compound, based on the weight of the formulation.

The active compound content of the application forms (crop protection products) prepared from the formulations may vary within wide ranges. The active compound concentration of the application forms may be situated typically between 0.00000001% and 95% by weight of active compound, preferably between 0.00001% and 1% by weight, based on the weight of the application form. Application takes place in a customary manner adapted to the application forms.

Preferred plants are those from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term useful plants as used in the present context refers to crop plants which are employed as plants for obtaining foodstuffs, feedstuffs, fuels or for industrial purposes.

The useful plants which can be improved by applying the compounds of formula (I) include for example the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees. This enumeration is no limitation.

The following plants are considered to be particularly suitable target crops: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potato and apple.

Examples of trees which can be improved in accordance with the method according to the invention are: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved in accordance with the method according to the invention are: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiata, P. ponderosa, P. contorta, P. sylvestre, P. elliottii, P. montecola, P. albicaulis, P. resinosa, P. palustris, P. taeda, P. flexilis, P. jeffregi, P. baksiana, P. strobus; from the tree species Eucalyptus: E. grandis, E. globulus, E. camadentis, E. nitens, E. obliqua, E. regnans, E. pilularus.

Especially preferred trees which can be improved in accordance with the method according to the invention are: from the tree species Pinus: P. radiata, P. ponderosa, P. contorta, P. sylvestre, P. strobus; from the tree species Eucalyptus: E. grandis, E. globulus, E. camadentis.

Very particularly preferred trees which can be improved in accordance with the method according to the invention are: horse chestnut, Platanaceae, linden tree, maple tree.

The present invention can also be applied to any turf grasses, including cool-season turf grasses and warm-season turf grasses. Examples of cold-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tennis Sibth.), velvet bentgrass (Agrostis canina L.), South German mixed bentgrass (Agrostis spp. including Agrostis tennis Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turf grasses are beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turf grasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpetgrass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H.B.K.) Lag. ex Griffiths), seashore paspalum (Paspalum vaginatum Swartz) and sideoats grama (Bouteloua curtipendula (Michx. Torr.). Cool-season turf grasses are generally preferred for the use according to the invention. Especially preferred are bluegrass, benchgrass and redtop, fescues and ryegrasses. Bentgrass is especially preferred.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

The control of animal pests by treating the seed of plants has been known for a long time and is a subject of continual improvements. Nevertheless, the treatment of seed entails a series of problems which cannot always be solved in a satisfactory manner Thus, it is desirable to develop methods for protecting the seed and the germinating plant that remove the need for, or at least significantly reduce, the additional delivery of crop protection compositions in the course of storage, after sowing or after the emergence of the plants. It is desirable, furthermore, to optimize the amount of active ingredient employed in such a way as to provide the best-possible protection to the seed and the germinating plant from attack by animal pests, but without causing damage to the plant itself by the active ingredient employed. In particular, methods for treating seed ought also to take into consideration the intrinsic insecticidal and/or nematicidal properties of pest-resistant or pest-tolerant transgenic plants, in order to achieve optimum protection of the seed and of the germinating plant with a minimal use of crop protection compositions.

The present invention therefore also relates in particular to a method for protecting seed and germinating plants from attack by pests, by treating the seed with a compound of formula (I).

The invention likewise relates to the use of the compound of formula (I) for treating seed for the purpose of protecting the seed and the resultant plant against animal pests.

The invention relates, furthermore, to seed which for protection against animal pests has been treated with a compound of formula (I).

Furthermore, the invention relates to seed which, following treatment with a compound of formula (I) of the invention, is subjected to a film-coating process in order to prevent dust abrasion of the seed.

One of the advantages of the present invention is that, owing to the particular systemic properties of the compositions of the invention, the treatment of the seed with these compositions provides protection from animal pests not only to the seed itself but also to the plants originating from the seed, after they have emerged. In this way, it may not be necessary to treat the crop directly at the time of sowing or shortly thereafter.

A further advantage is to be seen in the fact that, through the treatment of the seed with a compound of formula (I) of the invention, germination and emergence of the treated seed may be promoted.

It is likewise considered to be advantageous that compound of formula (I) may also be used, in particular, on transgenic seed.

It is also stated that a compound of formula (I) may be used in combination with agents of the signalling technology, as a result of which, for example, colonization with symbionts is improved, such as rhizobia, mycorrhiza and/or endophytic bacteria, for example, is enhanced, and/or nitrogen fixation is optimized.

The compositions of the invention are suitable for protecting seed of any variety of plant which is used in agriculture, in greenhouses, in forestry or in horticulture. More particularly, the seed in question is that of cereals (e.g. wheat, barley, rye, oats and millet), maize, cotton, soybeans, rice, potatoes, sunflower, coffee, tobacco, canola, oilseed rape, beets (e.g. sugar beet and fodder beet), peanuts, vegetables (e.g. tomato, cucumber, bean, brassicas, onions and lettuce), fruit plants, lawns and ornamentals. Particularly important is the treatment of the seed of cereals (such as wheat, barley, rye and oats) maize, soybeans, cotton, canola, oilseed rape and rice.

As already mentioned above, the treatment of transgenic seed with a compound of formula (I) is particularly important. The seed in question here is that of plants which generally contain at least one heterologous gene that controls the expression of a polypeptide having, in particular, insecticidal and/or nematicidal properties. These heterologous genes in transgenic seed may come from microorganisms such as Bacillus, Rhizobium, Pseudomonas, Serratia, Trichoderma, Clavibacter, Glomus or Gliocladium. The present invention is particularly suitable for the treatment of transgenic seed which contains at least one heterologous gene from Bacillus sp. With particular preference, the heterologous gene in question comes from Bacillus thuringiensis.

For the purposes of the present invention, the compound of formula (I) of the invention is applied alone or in a suitable formulation to the seed. The seed is preferably treated in a condition in which its stability is such that no damage occurs in the course of the treatment. Generally speaking, the seed may be treated at any point in time between harvesting and sowing. Typically, seed is used which has been separated from the plant and has had cobs, hulls, stems, husks, hair or pulp removed. Thus, for example, seed may be used that has been harvested, cleaned and dried to a moisture content of less than 15% by weight. Alternatively, seed can also be used that after drying has been treated with water, for example, and then dried again.

When treating seed it is necessary, generally speaking, to ensure that the amount of the composition of the invention, and/or of other additives, that is applied to the seed is selected such that the germination of the seed is not adversely affected, and/or that the plant which emerges from the seed is not damaged. This is the case in particular with active ingredients which may exhibit phytotoxic effects at certain application rates.

The compositions of the invention can be applied directly, in other words without comprising further components and without having been diluted. As a general rule, it is preferable to apply the compositions in the form of a suitable formulation to the seed. Suitable formulations and methods for seed treatment are known to the skilled person and are described in, for example, the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The compound of formula (I) which can be used in accordance with the invention may be converted into the customary seed-dressing formulations, such as solutions, emulsions, suspensions, powders, foams, slurries or other coating compositions for seed, and also ULV formulations.

These formulations are prepared in a known manner, by mixing the compound of formula (I) with customary adjuvants, such as, for example, customary extenders and also solvents or diluents, colorants, wetters, dispersants, emulsifiers, antifoams, preservatives, secondary thickeners, stickers, gibberellins, and also water.

Colorants which may be present in the seed-dressing formulations which can be used in accordance with the invention include all colorants which are customary for such purposes. In this context it is possible to use not only pigments, which are of low solubility in water, but also water-soluble dyes. Examples include the colorants known under the designations Rhodamin B, C.I. Pigment Red 112 and C.I. Solvent Red 1.

Wetters which may be present in the seed-dressing formulations which can be used in accordance with the invention include all of the substances which promote wetting and which are customary in the formulation of active agrochemical ingredients. Use may be made preferably of alkylnaphthalene-sulphonates, such as diisopropyl- or diisobutyl-naphthalenesulphonates.

Dispersants and/or emulsifiers which may be present in the seed-dressing formulations which can be used in accordance with the invention include all of the nonionic, anionic and cationic dispersants that are customary in the formulation of active agrochemical ingredients. Use may be made preferably of nonionic or anionic dispersants or of mixtures of nonionic or anionic dispersants. Suitable nonionic dispersants are, in particular, ethylene oxide-propylene oxide block polymers, alkylphenol polyglycol ethers and also tristryrylphenol polyglycol ethers, and the phosphated or sulphated derivatives of these. Suitable anionic dispersants are, in particular, lignosulphonates, salts of polyacrylic acid, and arylsulphonate-formaldehyde condensates.

Antifoams which may be present in the seed-dressing formulations which can be used in accordance with the invention include all of the foam inhibitors that are customary in the formulation of active agrochemical ingredients. Use may be made preferably of silicone antifoams and magnesium stearate.

Preservatives which may be present in the seed-dressing formulations which can be used in accordance with the invention include all of the substances which can be employed for such purposes in agrochemical compositions. Examples include dichlorophen and benzyl alcohol hemiformal.

Secondary thickeners which may be present in the seed-dressing formulations which can be used in accordance with the invention include all substances which can be used for such purposes in agrochemical compositions. Those contemplated with preference include cellulose derivatives, acrylic acid derivatives, xanthan, modified clays and highly disperse silica.

Stickers which may be present in the seed-dressing formulations which can be used in accordance with the invention include all customary binders which can be used in seed-dressing products. Preferred mention may be made of polyvinylpyrrolidone, polyvinyl acetate, polyvinyl alcohol and tylose.

Gibberellins which may be present in the seed-dressing formulations which can be used in accordance with the invention include preferably the gibberellins A1, A3 (=gibberellic acid), A4 and A7, with gibberellic acid being used with particular preference. The gibberellins are known (cf. R. Wegler, “Chemie der Pflanzenschutz- and Schadlingsbekämpfungsmittel”, Volume 2, Springer Verlag, 1970, pp. 401-412).

The seed-dressing formulations which can be used in accordance with the invention may be used, either directly or after prior dilution with water, to treat seed of any of a wide variety of types. Accordingly, the concentrates or the preparations obtainable from them by dilution with water may be employed to dress the seed of cereals, such as wheat, barley, rye, oats and triticale, and also the seed of maize, rice, oilseed rape, peas, beans, cotton, sunflowers and beets, or else the seed of any of a very wide variety of vegetables. The seed-dressing formulations which can be used in accordance with the invention, or their diluted preparations, may also be used to dress seed of transgenic plants. In that case, additional synergistic effects may occur in interaction with the substances formed through expression.

For the treatment of seed with the seed-dressing formulations which can be used in accordance with the invention, or with the preparations produced from them by addition of water, suitable mixing equipment includes all such equipment which can typically be employed for seed dressing. More particularly, the procedure when carrying out seed dressing is to place the seed in a mixer, to add the particular desired amount of seed-dressing formulations, either as such or following dilution with water beforehand, and to carry out mixing until the distribution of the formulation on the seed is uniform. This may be followed by a drying operation.

The application rate of the seed-dressing formulations which can be used in accordance with the invention may be varied within a relatively wide range. It is guided by the particular amount of the compound of formula (I) in the formulations, and by the seed. The application rates with regard to the compound of formula (I) are situated generally at between 0.001 and 50 g per kilogram of seed, preferably between 0.01 and 15 g per kilogram of seed.

As already mentioned above, it is possible to treat all plants and their parts in accordance with the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering, if appropriate in combination with conventional methods (Genetically Modified Organisms), and parts thereof are treated. The terms “parts” or “parts of plants” or “plant parts” have been explained above.

More preferably, plants of the plant cultivars which are each commercially available or in use are treated in accordance with the invention. Plant cultivars are understood to mean plants having new properties (“traits”) and which have been obtained by conventional breeding, by mutagenesis or by recombinant DNA techniques. They may be cultivars, biotypes and genotypes.

Depending on the plant species or plant cultivars, and the location and growth conditions (soils, climate, vegetation period, diet) thereof, the inventive treatment may also result in over additive (“synergistic”) effects. For example, possibilities include reduced application rates and/or broadening of the activity spectrum and/or an increase in the activity of the compounds and compositions usable in accordance with the invention, better plant growth, increased tolerance to high or low temperatures, increased tolerance to drought or to levels of water or soil salinity, enhanced flowering performance, easier harvesting, accelerated ripening, higher yields, higher quality and/or higher nutritional value of the harvested products, increased storage life and/or processibility of the harvested products, which exceed the effects normally to be expected.

The transgenic plants or plant cultivars (those obtained by genetic engineering) which are to be treated with preference in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher yields, higher quality and/or a higher nutritional value of the harvested products, better storage life and/or processibility of the harvested products. Further and particularly emphasized examples of such properties are an improved defence of the plants against animal and microbial pests, such as against insects, mites, phytopathogenic fungi, bacteria and/or viruses, and also increased tolerance of the plants to certain herbicidally active ingredients. Examples of transgenic plants include the important crop plants, such as cereals (wheat, rice), maize, soya, potatoes, sugar beet, tomatoes, peas and other vegetable types, cotton, tobacco, oilseed rape, and also fruit plants (with the fruits of apples, pears, citrus fruits and grapes), particular emphasis being given to maize, soya, potatoes, cotton, tobacco and oilseed rape. Traits that are particularly emphasized are improved defence of the plants against insects, arachnids, nematodes, slugs and snails by toxins formed in the plants, especially those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF, and also combinations thereof) (referred to hereinafter as “Bt plants”). Traits that are also particularly emphasized are the improved defence of plants against fungi, bacteria and viruses by systemic acquired resistance (SAR), systemin, phytoalexins, elicitors and also resistance genes and correspondingly expressed proteins and toxins. Traits that are additionally particularly emphasized are the increased tolerance of the plants to certain active herbicidal ingredients, for example imidazolinones, sulphonylureas, glyphosate or phosphinothricin (for example the “PAT” gene). The genes which impart the desired traits in question may also be present in combinations with one another in the transgenic plants. Examples of “Bt plants” include maize varieties, cotton varieties, soya varieties and potato varieties which are sold under the trade names YIELD GARD® (for example maize, cotton, soya), KnockOut® (for example maize), StarLink® (for example maize), Bollgard® (cotton), Nucotn® (cotton) and NewLeaf® (potato). Examples of herbicide-tolerant plants include maize varieties, cotton varieties and soya varieties which are sold under the trade names Roundup Ready® (tolerance to glyphosate, for example maize, cotton, soya), Liberty Link® (tolerance to phosphinothricin, for example oilseed rape), IMI® (tolerance to imidazolinones) and STS® (tolerance to sulphonylureas, for example maize) Herbicide-resistant plants (plants bred in a conventional manner for herbicide tolerance) which may be mentioned include the varieties sold under the name Clearfield® (for example maize) Of course, these statements also apply to plant cultivars which have these genetic traits or genetic traits which are still to be developed and will be developed and/or marketed in the future.

The plants listed can be treated in accordance with the invention in a particularly advantageous manner with the compounds of the general formula (I) and/or the active ingredient mixtures according to the invention. The preferred ranges stated above for the active ingredients or mixtures also apply to the treatment of these plants. Particular emphasis is given to the treatment of plants with the compounds or mixtures specifically mentioned in the present text.

The treatment of the plants and plant parts with the compounds of formula (I) is carried out directly or by acting on the environment, habitat or storage space using customary treatment methods, for example by dipping, spraying, atomizing, misting, evaporating, dusting, fogging, scattering, foaming, painting on, spreading, injecting, drenching, trickle irrigation and, in the case of propagation material, in particular in the case of seed, furthermore by the dry seed treatment method, the wet seed treatment method, the slurry treatment method, by encrusting, by coating with one or more coats and the like. It is furthermore possible to apply the active substances by the ultra-low volume method or to inject the active substance preparation or the active substance itself into the soil.

A preferred direct treatment of the plants is the leaf application treatment, i.e. compounds of formula (I) or compositions containing them are applied to the foliage, it being possible for the treatment frequency and the application rate to be matched to the infection pressure.

In the case of systemically active compounds, compounds of formula (I) or compositions according to the invention reach the plants via the root system. In this case, the treatment of the plants is effected by allowing the compounds of formula (I) or compositions according to the invention to act on the environment of the plant. This can be done for example by drenching, incorporating in the soil or into the nutrient solution, i.e. the location of the plant (for example the soil or hydroponic systems) is impregnated with a liquid form of compounds of formula (I) or compositions according to the invention, or by soil application, i.e. the compounds of formula (I) or compositions according to the invention are incorporated into the location of the plants in solid form (for example in the form of granules). In the case of paddy rice cultures, this may also be done by metering the compounds of formula (I) or compositions according to the invention into a flooded paddy field in a solid use form (for example in the form of granules).

The inventive active ingredient may be present in its commercially available formulations and in the use forms, prepared from these formulations, as a mixture with other active ingredients, such as insecticides, attractants, sterilants, bactericides, acaricides, nematicides, fungicides, growth regulators, herbicides, safeners, fertilizers or semiochemicals. The mixtures thus obtained have a broadened spectrum of activity.

Mixtures with fungicides are particularly advantageous. Examples of suitable fungicide mixing partners can be selected from the list consisting of

1) Inhibitors of the ergosterol biosynthesis, for example (1.1) aldimorph, (1.2) azaconazole, (1.3) bitertanol, (1.4) bromuconazole, (1.5) cyproconazole, (1.6) diclobutrazole, (1.7) difenoconazole, (1.8) diniconazole, (1.9) diniconazole-M, (1.10) dodemorph, (1.11) dodemorph acetate, (1.12) epoxiconazole, (1.13) etaconazole, (1.14) fenarimol, (1.15) fenbuconazole, (1.16) fenhexamid, (1.17) fenpropidin, (1.18) fenpropimorph, (1.19) fluquinconazole, (1.20) flurprimidol, (1.21) flusilazole, (1.22) flutriafol, (1.23) furconazole, (1.24) furconazole-cis, (1.25) hexaconazole, (1.26) imazalil, (1.27) imazalil sulfate, (1.28) imibenconazole, (1.29) ipconazole, (1.30) metconazole, (1.31) myclobutanil, (1.32) naftifine, (1.33) nuarimol, (1.34) oxpoconazole, (1.35) paclobutrazol, (1.36) pefurazoate, (1.37) penconazole, (1.38) piperalin, (1.39) prochloraz, (1.40) propiconazole, (1.41) prothioconazole, (1.42) pyributicarb, (1.43) pyrifenox, (1.44) quinconazole, (1.45) simeconazole, (1.46) spiroxamine, (1.47) tebuconazole, (1.48) terbinafine, (1.49) tetraconazole, (1.50) triadimefon, (1.51) triadimenol, (1.52) tridemorph, (1.53) triflumizole, (1.54) triforine, (1.55) triticonazole, (1.56) uniconazole, (1.57) uniconazole-p, (1.58) viniconazole, (1.59) voriconazole, (1.60) 1-(4-chlorophenyl)-2-(1H-1,2,4-triazol-1-yl)cycloheptanol, (1.61) methyl 1-(2,2-dimethyl-2,3-dihydro-1H-inden-1-yl)-1H-imidazole-5-carboxy late, (1.62) N′-{5-(difluoromethyl)-2-methyl-4-[3-(trimethylsilyl)propoxy]phenyl}-N-ethyl-N-methylimidoformamide, (1.63)N-ethyl-N-methyl-N′-{2-methyl-5-(trifluoromethyl)-4-[3-(trimethylsilyppropoxy]phenyl}imidoformamide, (1.64) O-[1-(4-methoxyphenoxy)-3,3-dimethylbutan-2-yl]1H-imidazole-1-carbothioate, (1.65) Pyrisoxazole.

2) Inhibitors of the respiratory chain at complex I or II, for example (2.1) bixafen, (2.2) boscalid, (2.3) carboxin, (2.4) diflumetorim, (2.5) fenfuram, (2.6) fluopyram, (2.7) flutolanil, (2.8) fluxapyroxad, (2.9) furametpyr, (2.10) furmecyclox, (2.11) isopyrazam (mixture of syn-epimeric racemate 1RS,4SR,9RS and anti-epimeric racemate 1RS,4SR,9SR), (2.12) isopyrazam (anti-epimeric racemate 1RS,4SR,9SR), (2.13) isopyrazam (anti-epimeric enantiomer 1R,4S,9S), (2.14) isopyrazam (anti-epimeric enantiomer 1S,4R,9R), (2.15) isopyrazam (syn epimeric racemate 1RS,4SR,9RS), (2.16) isopyrazam (syn-epimeric enantiomer 1R,4S,9R), (2.17) isopyrazam (syn-epimeric enantiomer 1S,4R,9S), (2.18) mepronil, (2.19) oxycarboxin, (2.20) penflufen, (2.21) penthiopyrad, (2.22) sedaxane, (2.23) thifluzamide, (2.24) 1-methyl-N-[2-(1,1,2,2-tetrafluoroethoxy)phenyl]-3-(trifluoromethyl)-1H-pyrazole-4-carboxamide, (2.25) 3-(difluoromethyl)-1-methyl-N-[2-(1,1,2,2-tetrafluoroethoxy)phenyl]-1H-pyrazole-4-carboxamide, (2.26) 3-(difluoromethy 1)-N-[4-fluoro-2-(1,1,2,3,3,3-hexafluoropropoxy)phenyl]-1-methyl-1H-pyrazole-4-carboxamide, (2.27)N-[1-(2,4-dichlorophenyl)-1-methoxypropan-2-yl]-3-(difluoromethyl)-1-methyl-1H-pyrazole-4-carboxamide, (2.28) 5,8-difluoro-N-[2-(2-fluoro-4-{[4-(trifluoromethyl)pyridin-2-yl]oxy}phenyl)ethyl]quinazolin-4-amine, (2.29) benzovindiflupyr, (2.30)N-[(1S,4R)-9-(dichloromethylene)-1,2,3,4-tetrahydro-1,4-methanonaphthalen-5-yl]-3-(difluoromethyl)-1-methy 1-1H-pyrazole-4-carboxamide, (2.31) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.32) 3-(difluoromethyl)-1-methyl-N-(1,1,3-trimethyl-2,3-dihydro-1H-inden-4-yl)-1H-pyrazole-4-carboxamide, (2.33) 1,3,5-trimethyl-N-(1,1,3-trimethyl-2,3-dihydro-1H-inden-4-yl)-1H-pyrazole-4-carboxamide, (2.34) 1-methyl-3-(trifluoromethyl)-N-(1,1,3-trimethyl-2,3-dihydro-1H-inden-4-yl)-1H-pyrazole-4-carboxamide, (2.35) 1-methyl-3-(trifluoromethyl)-N-[(3R)-1,1,3-trimethyl-2,3-dihydro-1H-inden-4-yl]-1H-pyrazole-4-carboxamide, (2.36) 1-methyl-3-(trifluoromethyl)-N-[(3S)-1,1,3-trimethyl-2,3-dihydro-1H-inden-4-yl]-1H-pyrazole-4-carboxamide, (2.37) 3-(difluoromethyl)-1-methyl-N-[(3S)-1,1,3-trimethyl-2,3-dihydro-1H-inden-4-yl]-1H-pyrazole-4-carboxamide, (2.38) 3-(difluoromethyl)-1-methyl-N-[(3R)-1,1,3-trimethyl-2,3-dihydro-1H-inden-4-yl]-1H-pyrazole-4-carboxamide, (2.39) 1,3,5-trimethyl-N-[(3R)-1,1,3-trimethyl-2,3-dihydro-1H-inden-4-yl]-1H-pyrazole-4-carboxamide, (2.40) 1,3,5-trimethyl-N-[(3S)-1,1,3-trimethyl-2,3-dihydro-1H-inden-4-yl]-1H-pyrazole-4-carboxamide, (2.41) benodanil, (2.42) 2-chloro-N-(1,1,3-trimethyl-2,3-dihydro-1H-inden-4-yl)pyridine-3-carboxamide, (2.43) Isofetamid.

3) Inhibitors of the respiratory chain at complex III, for example (3.1) ametoctradin, (3.2) amisulbrom, (3.3) azoxystrobin, (3.4) cyazofamid, (3.5) coumethoxystrobin, (3.6) coumoxystrobin, (3.7) dimoxystrobin, (3.8) enoxastrobin, (3.9) famoxadone, (3.10) fenamidone, (3.11) flufenoxystrobin, (3.12) fluoxastrobin, (3.13) kresoxim-methyl, (3.14) metominostrobin, (3.15) orysastrobin, (3.16) picoxystrobin, (3.17) pyraclostrobin, (3.18) pyrametostrobin, (3.19) pyraoxystrobin, (3.20) pyribencarb, (3.21) triclopyricarb, (3.22) trifloxystrobin, (3.23) (2E)-2-(2-{[6-(3-chloro-2-methylphenoxy)-5-fluoropyrimidin-4-yl]oxy}phenyl)-2-(methoxyimino)-N-methylacetamide, (3.24) (2E)-2-(methoxyimino)-N-methyl-2-(2-{[({(1E)-1-[3-(trifluoromethyl)phenyl]ethylidene}amino)oxy]methyl}phenyl)acetamide, (3.25) (2E)-2-(methoxyimino)-N-methyl-2-{2-[(E)-({1-[3-(trifluoromethyl)phenyl]ethoxy}imino)methyl]phenyl}acetamide, (3.26) (2E)-2-{2-[({[(1E)-1-(3-{[(E)-1-fluoro-2-phenylvinyl]oxy}phenyl)ethylidene]amino}oxy)methyl]phenyl}-2-(methoxy imino)-N-methylacetamide, (3.27) Fenaminostrobin, (3.28) 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, (3.29) methyl (2E)-2-{2[({cyclopropyl[(4-methoxyphenyl)imino]methyl}sulfanyl)methyl]phenyl}-3-methoxyacrylate, (3.30) N-(3-ethyl-3,5,5-trimethylcyclohexyl)-3-formamido-2-hydroxybenzamide, (3.31) 2-{2-[(2,5-dimethylphenoxy)methyl]phenyl}-2-methoxy-N-methylacetamide, (3.32) 2-{2-[(2,5-dimethylphenoxy)methyl]phenyl}-2-methoxy-N-methylacetamide.

4) Inhibitors of the mitosis and cell division, for example (4.1) benomyl, (4.2) carbendazim, (4.3) chlorfenazole, (4.4) diethofencarb, (4.5) ethaboxam, (4.6) fluopicolide, (4.7) fuberidazole, (4.8) pencycuron, (4.9) thiabendazole, (4.10) thiophanate-methyl, (4.11) thiophanate, (4.12) zoxamide, (4.13) 5-chloro-7-(4-methylpiperidin-1-yl)-6-(2,4,6-trifluorophenyl)[1,2,4]triazolo[1,5-a]pyrimidine, (4.14) 3-chloro-5-(6-chloropyridin-3-yl)-6-methyl-4-(2,4,6-trifluorophenyl)pyridazine.

5) Compounds capable to have a multisite action, for example (5.1) bordeaux mixture, (5.2) captafol, (5.3) captan, (5.4) chlorothalonil, (5.5) copper hydroxide, (5.6) copper naphthenate, (5.7) copper oxide, (5.8) copper oxychloride, (5.9) copper(2+) sulfate, (5.10) dichlofluanid, (5.11) dithianon, (5.12) dodine, (5.13) dodine free base, (5.14) ferbam, (5.15) fluorofolpet, (5.16) folpet, (5.17) guazatine, (5.18) guazatine acetate, (5.19) iminoctadine, (5.20) iminoctadine albesilate, (5.21) iminoctadine triacetate, (5.22) mancopper, (5.23) mancozeb, (5.24) maneb, (5.25) metiram, (5.26) metiram zinc, (5.27) oxine-copper, (5.28) propamidine, (5.29) propineb, (5.30) sulfur and sulfur preparations including calcium polysulfide, (5.31) thiram, (5.32) tolylfluanid, (5.33) zineb, (5.34) ziram, (5.35) anilazine.

6) Compounds capable to induce a host defence, for example (6.1) acibenzolar-S-methyl, (6.2) isotianil, (6.3) probenazole, (6.4) tiadinil, (6.5) laminarin 7) Inhibitors of the amino acid and/or protein biosynthesis, for example (7.1) andoprim, (7.2) blasticidin-S, (7.3) cyprodinil, (7.4) kasugamycin, (7.5) kasugamycin hydrochloride hydrate, (7.6) mepanipyrim, (7.7) pyrimethanil, (7.8) 3-(5-fluoro-3,3,4,4-tetramethyl-3,4-dihydroisoquinolin-1-yl)quinoline, (7.9) oxytetracycline, (7.10) streptomycin.

8) Inhibitors of the ATP production, for example (8.1) fentin acetate, (8.2) fentin chloride, (8.3) fentin hydroxide, (8.4) silthiofam.

9) Inhibitors of the cell wall synthesis, for example (9.1) benthiavalicarb, (9.2) dimethomorph, (9.3) flumorph, (9.4) iprovalicarb, (9.5) mandipropamid, (9.6) polyoxins, (9.7) polyoxorim, (9.8) validamycin A, (9.9) valifenalate, (9.10) polyoxin B.

10) Inhibitors of the lipid and membrane synthesis, for example (10.1) biphenyl, (10.2) chloroneb, (10.3) dicloran, (10.4) edifenphos, (10.5) etridiazole, (10.6) iodocarb, (10.7) iprobenfos, (10.8) isoprothiolane, (10.9) propamocarb, (10.10) propamocarb hydrochloride, (10.11) prothiocarb, (10.12) pyrazophos, (10.13) quintozene, (10.14) tecnazene, (10.15) tolclofos-methyl.

11) Inhibitors of the melanin biosynthesis, for example (11.1) carpropamid, (11.2) diclocymet, (11.3) fenoxanil, (11.4) phthalide, (11.5) pyroquilon, (11.6) tricyclazole, (11.7) 2,2,2-trifluoroethyl {3-methyl-1-[(4-methylbenzoyl)amino]butan-2-yl}carbamate.

12) Inhibitors of the nucleic acid synthesis, for example (12.1) benalaxyl, (12.2) benalaxyl-M (kiralaxyl), (12.3) bupirimate, (12.4) clozylacon, (12.5) dimethirimol, (12.6) ethirimol, (12.7) furalaxyl, (12.8) hymexazol, (12.9) metalaxyl, (12.10) metalaxyl-M (mefenoxam), (12.11) ofurace, (12.12) oxadixyl, (12.13) oxolinic acid, (12.14) octhilinone.

13) Inhibitors of the signal transduction, for example (13.1) chlozolinate, (13.2) fenpiclonil, (13.3) fludioxonil, (13.4) iprodione, (13.5) procymidone, (13.6) quinoxyfen, (13.7) vinclozolin, (13.8) proquinazid.

14) Compounds capable to act as an uncoupler, for example (14.1) binapacryl, (14.2) dinocap, (14.3) ferimzone, (14.4) fluazinam, (14.5) meptyldinocap.

15) Further compounds, for example (15.1) benthiazole, (15.2) bethoxazin, (15.3) capsimycin, (15.4) carvone, (15.5) chinomethionat, (15.6) pyriofenone (chlazafenone), (15.7) cufraneb, (15.8) cyflufenamid, (15.9) cymoxanil, (15.10) cyprosulfamide, (15.11) dazomet, (15.12) debacarb, (15.13) dichlorophen, (15.14) diclomezine, (15.15) difenzoquat, (15.16) difenzoquat metilsulfate, (15.17) diphenylamine, (15.18) ecomate, (15.19) fenpyrazamine, (15.20) flumetover, (15.21) fluoroimide, (15.22) flusulfamide, (15.23) flutianil, (15.24) fosetyl-aluminium, (15.25) fosetyl-calcium, (15.26) fosetyl-sodium, (15.27) hexachlorobenzene, (15.28) irumamycin, (15.29) methasulfocarb, (15.30) methyl isothiocyanate, (15.31) metrafenone, (15.32) mildiomycin, (15.33) natamycin, (15.34) nickel dimethyldithiocarbamate, (15.35) nitrothal-isopropyl, (15.37) oxamocarb, (15.38) oxyfenthiin, (15.39) pentachlorophenol and salts, (15.40) phenothrin, (15.41) phosphorous acid and its salts, (15.42) propamocarb-fosetylate, (15.43) propanosine-sodium, (15.44) pyrimorph, (15.45) (2E)-3-(4-tert-butylphenyl)-3-(2-chloropyridin-4-yl)-1-(morpholin-4-yl)prop-2-en-1-one, (15.46) (2Z)-3-(4-tert-butylphenyl)-3-(2-chloropyridin-4-yl)-1-(morpholin-4-yl)prop-2-en-1-one, (15.47) pyrrolnitrin, (15.48) tebufloquin, (15.49) tecloftalam, (15.50) tolnifanide, (15.51) triazoxide, (15.52) trichlamide, (15.53) zarilamid, (15.54) (3S,6S,7R,8R)-8-benzyl-3-[({3-[(isobutyryloxy)methoxy}-4-methoxypyridin-2-yl}carbonyl)amino]-6-methyl-4,9-dioxo-1,5-dioxonan-7-yl 2-methylpropanoate, (15.55) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.56) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.57) 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.58) 1-(4-methoxyphenoxy)-3,3-dimethylbutan-2-yl 1H-imidazole-1-carboxylate, (15.59) 2,3,5,6-tetrachloro-4-(methylsulfonyl)pyridine, (15.60) 2,3-dibutyl-6-chlorothieno[2,3-d]pyrimidin-4(3H)-one, (15.61) 2,6-dimethyl-1H,5H-[1,4]dithiino[2,3-c:5,6-c′]dipyrrole-1,3,5,7(2H,6H)-tetrone, (15.62) 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl) ethanone, (15.63) 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl) ethanone, (15.64) 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, (15.65) 2-butoxy-6-iodo-3-propyl-4H-chromen-4-one, (15.66) 2-chloro-5-[2-chloro-1-(2,6-difluoro-4-methoxyphenyl)-4-methyl-1H-imidazol-5-yl]pyridine, (15.67) 2-phenylphenol and salts, (15.68) 3-(4,4,5-trifluoro-3,3-dimethyl-3,4-dihydroisoquinolin-1-yl)quinoline, (15.69) 3,4,5-trichloropyridine-2,6-dicarbonitrile, (15.70) 3-chloro-5-(4-chlorophenyl)-4-(2,6-difluorophenyl)-6-methylpyridazine, (15.71) 4-(4-chlorophenyl)-5-(2,6-difluorophenyl)-3,6-dimethylpyridazine, (15.72) 5-amino-1,3,4-thiadiazole-2-thiol, (15.73) 5-chloro-N′-phenyl-N′-(prop-2-yn-1-yl)thiophene-2-sulfonohydrazide, (15.74) 5-fluoro-2-[(4-fluorobenzyl)oxy]pyrimidin-4-amine, (15.75) 5-fluoro-2-[(4-methylbenzyl)oxy]pyrimidin-4-amine, (15.76) 5-methyl-6-octyl[1,2,4]triazolo[1,5-a]pyrimidin-7-amine, (15.77) ethyl(2Z)-3-amino-2-cyano-3-phenylacrylate, (15.78) N′-(4-{[3-(4-chlorobenzyl)-1,2,4-thiadiazol-5-yl]oxy}-2,5-dimethylphenyl)-N-ethyl-N-methylimidoformamide, (15.79)N-(4-chlorobenzyl)-3-[3-methoxy-4-(prop-2-yn-1-yloxy)phenyl]propanamide, (15.80) N-[(4-chlorophenyl) (cyano)methyl]-3-[3-methoxy-4-(prop-2-yn-1-yloxy)phenyl]propanamide, (15.81)N-[(5-bromo-3-chloropyridin-2-yl)methyl]-2,4-dichloronicotinamide, (15.82)N-[1-(5-bromo-3-chloropyridin-2-yl)ethyl]-2,4-dichloronicotinamide, (15.83) N-[1-(5-bromo-3-chloropyridin-2-yl)ethyl]-2-fluoro-4-iodonicotinamide, (15.84)N-{(E)-[(cyclopropylmethoxy)imino][6-(difluoromethoxy)-2,3-difluorophenyl]methyl}-2-phenylacetamide, (15.85) N-{(Z)-[(cyclopropylmethoxy)imino][6-(difluoromethoxy)-2,3-difluorophenyl]methyl}-2-phenylacetamide, (15.86) N′-{4-[(3-tert-butyl-4-cyano-1,2-thiazol-5-yl)oxy]-2-chloro-5-methylphenyl}-N-ethyl-N-methylimidoformamide, (15.87)N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, (15.88) N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, (15.89) N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1,3-thiazole-4-carboxamide, (15.90) pentyl {6-[({[(1-methyl-1H-tetrazol-5-yl)(phenyl)methylene]amino}oxy)methyl]pyridin-2-yl}carbamate, (15.91) phenazine-1-carboxylic acid, (15.92) quinolin-8-ol, (15.93) quinolin-8-ol sulfate (2:1), (15.94) tert-butyl {6-[({[(1-methy 1-1H-tetrazol-5-yl)(phenyl)methylene]amino}oxy)methyl]pyridin-2-yl}carbamate, (15.95) 1-methyl-3-(trifluoromethyl)-N-[2′-(trifluoromethyl)biphenyl-2-yl]-1H-pyrazole-4-carboxamide, (15.96)N-(4′-chlorobiphenyl-2-yl)-3-(difluoromethyl)-1-methyl-1H-pyrazole-4-carboxamide, (15.97)N-(2′,4′-dichlorobiphenyl-2-yl)-3-(difluoromethyl)-1-methyl-1H-pyrazole-4-carboxamide, (15.98) 3-(difluoromethyl)-1-methyl-N-[4′-(trifluoromethyl)biphenyl-2-yl]-1H-pyrazole-4-carboxamide, (15.99) N-(2′,5′-difluorobiphenyl-2-yl)-1-methyl-3-(trifluoromethyl)-1H-pyrazole-4-carboxamide, (15.100) 3-(difluoromethyl)-1-methyl-N-[4′-(prop-1-yn-1-yl)biphenyl-2-yl]-1H-pyrazole-4-carboxamide, (15.101) 5-fluoro-1,3-dimethyl-N-[4′-(prop-1-yn-1-yl)biphenyl-2-yl]-1H-pyrazole-4-carboxamide, (15.102) 2-chloro-N-[4′-(prop-1-yn-1-yl)biphenyl-2-yl]nicotinamide, (15.103) 3-(difluoromethyl)-N-[4′-(3,3-dimethylbut-1-yn-1-yl)biphenyl-2-yl]-1-methyl-1H-pyrazole-4-carboxamide, (15.104)N-[4′-(3,3-dimethylbut-1-yn-1-yl)biphenyl-2-yl]-5-fluoro-1,3-dimethyl-1H-pyrazole-4-carboxamide, (15.105) 3-(difluoromethyl)-N-(4′-ethynylbiphenyl-2-yl)-1-methyl-1H-pyrazole-4-carboxamide, (15.106)N-(4′-ethynylbiphenyl-2-yl)-5-fluoro-1,3-dimethyl-1H-pyrazole-4-carboxamide, (15.107) 2-chloro-N-(4′-ethynylbiphenyl-2-yl)nicotinamide, (15.108) 2-chloro-N-[4′-(3,3-dimethylbut-1-yn-1-yl)biphenyl-2-yl]nicotinamide, (15.109) 4-(difluoromethyl)-2-methyl-N-[4′-(trifluoromethyl)biphenyl-2-yl]-1,3-thiazole-5-carboxamide, (15.110) 5-fluoro-N-[4′-(3-hydroxy-3-methylbut-1-yn-1-yl)biphenyl-2-yl]-1,3-dimethyl-1H-pyrazole-4-carboxamide, (15.111) 2-chloro-N-[4′-(3-hydroxy-3-methylbut-1-yn-1-yl)biphenyl-2-yl]nicotinamide, (15.112) 3-(difluoromethyl)-N-[4′-(3-methoxy-3-methylbut-1-yn-1-yl)biphenyl-2-yl]-1-methyl-1H-pyrazole-4-carboxamide, (15.113) 5-fluoro-N-[4′-(3-methoxy-3-methylbut-1-yn-1-yl)biphenyl-2-yl]-1,3-dimethyl-1H-pyrazole-4-carboxamide, (15.114) 2-chloro-N-[4′-(3-methoxy-3-methylbut-1-yn-1-yl)biphenyl-2-yl]nicotinamide, (15.115) (5-bromo-2-methoxy-4-methylpyridin-3-yl)(2,3,4-trimethoxy-6-methylphenyl)methanone, (15.116) N-[2-(4-{[3-(4-chlorophenyl)prop-2-yn-1-yl]oxy}-3-methoxyphenyl)ethyl]-N2-(methylsulfonyl)valinamide, (15.117) 4-oxo-4-[(2-phenylethyl)amino]butanoic acid, (15.118) but-3-yn-1-yl {6-[({[(Z)-(1-methyl-1H-tetrazol-5-yl)(phenyl)methylene]amino}oxy)methyl]pyridin-2-yl}carbamate, (15.119) 4-amino-5-fluoropyrimidin-2-ol (tautomeric form: 4-amino-5-fluoropyrimidin-2(1H)-one), (15.120) propyl 3,4,5-trihydroxybenzoate, (15.121) 1,3-dimethyl-N-(1,1,3-trimethyl-2,3-dihydro-1H-inden-4-yl)-1H-pyrazole-4-carboxamide, (15.122) 1,3-dimethyl-N-[(3R)-1,1,3-trimethyl-2,3-dihydro-1H-inden-4-yl]-1H-pyrazole-4-carboxamide, (15.123) 1,3-dimethyl-N-[(3S)-1,1,3-trimethyl-2,3-dihydro-1H-inden-4-yl]-1H-pyrazole-4-carboxamide, (15.124) [3-(4-chloro-2-fluorophenyl)-5-(2,4-difluorophenyl)-1,2-oxazol-4-yl](pyridin-3-yl)methanol, (15.125) (S)-[3-(4-chloro-2-fluorophenyl)-5-(2,4-difluorophenyl)-1,2-oxazol-4-yl](pyridin-3-yl)methanol, (15.126) (R)-[3-(4-chloro-2-fluorophenyl)-5-(2,4-difluorophenyl)-1,2-oxazol-4-yl](pyridin-3-yl)methanol, (15.127) 2-{[3-(2-chlorophenyl)-2-(2,4-difluorophenyl)oxiran-2-yl]methyl}-2,4-dihydro-3H-1,2,4-triazole-3-thione, (15.128) 1-{[3-(2-chlorophenyl)-2-(2,4-difluorophenyl)oxiran-2-yl]methyl}-1H-1,2,4-triazol-5-yl thiocyanate, (15.129) 5-(allylsulfanyl)-1-{[3-(2-chlorophenyl)-2-(2,4-difluorophenyl)oxiran-2-yl]methyl}-1H-1,2,4-triazole, (15.130) 2-[1-(2,4-dichlorophenyl)-5-hydroxy-2,6,6-trimethylheptan-4-yl]-2,4-dihydro-3H-1,2,4-triazole-3-thione, (15.131) 2-{[rel(2R,3S)-3-(2-chlorophenyl)-2-(2,4-difluorophenyl)oxiran-2-yl]methyl}-2,4-dihydro-3H-1,2,4-triazole-3-thione, (15.132) 2-{[rel(2R,3R)-3-(2-chlorophenyl)-2-(2,4-difluorophenyl)oxiran-2-yl]methyl}-2,4-dihydro-3H-1,2,4-triazole-3-thione, (15.133) 1-{[rel(2R,3S)-3-(2-chlorophenyl)-2-(2,4-difluorophenyl)oxiran-2-yl]methyl}-1H-1,2,4-triazol-5-yl thiocyanate, (15.134) 1-{[rel(2R,3R)-3-(2-chlorophenyl)-2-(2,4-difluorophenyl)oxiran-2-yl]methyl}-1H-1,2,4-triazol-5-yl thiocyanate, (15.135) 5-(allylsulfanyl)-1-{[rel(2R,3S)-3-(2-chlorophenyl)-2-(2,4-difluorophenyl)oxiran-2-yl]methyl}-1H-1,2,4-triazole, (15.136) 5-(allylsulfanyl)-1-{[rel(2R,3R)-3-(2-chlorophenyl)-2-(2,4-difluorophenyl)oxiran-2-yl]methyl}-1H-1,2,4-triazole, (15.137) 2-[(2S,4S,5S)-1-(2,4-dichlorophenyl)-5-hydroxy-2,6,6-trimethylheptan-4-yl]-2,4-dihydro-3H-1,2,4-triazole-3-thione, (15.138) 2-[(2R,4S,5S)-1-(2,4-dichlorophenyl)-5-hydroxy-2,6,6-trimethylheptan-4-yl]-2,4-dihydro-3H-1,2,4-triazole-3-thione, (15.139) 2-[(2R,4R,5R)-1-(2,4-dichlorophenyl)-5-hydroxy-2,6,6-trimethylheptan-4-yl]-2,4-dihydro-3H-1,2,4-triazole-3-thione, (15.140) 2-[(2S,4R,5R)-1-(2,4-dichlorophenyl)-5-hydroxy-2,6,6-trimethylheptan-4-yl]-2,4-dihydro-3H-1,2,4-triazole-3-thione, (15.141) 2-[(2S,4S,5R)-1-(2,4-dichlorophenyl)-5-hydroxy-2,6,6-trimethylheptan-4-yl]-2,4-dihydro-3H-1,2,4-triazole-3-thione, (15.142) 2-[(2R,4S,5R)-1-(2,4-dichlorophenyl)-5-hydroxy-2,6,6-trimethylheptan-4-yl]-2,4-dihydro-3H-1,2,4-triazole-3-thione, (15.143) 2-[(2R,4R,5S)-1-(2,4-dichlorophenyl)-5-hydroxy-2,6,6-trimethylheptan-4-yl]-2,4-dihydro-3H-1,2,4-triazole-3-thione, (15.144) 2-[(2S,4R,5S)-1-(2,4-dichlorophenyl)-5-hydroxy-2,6,6-trimethylheptan-4-yl]-2,4-dihydro-3H-1,2,4-triazole-3-thione, (15.145) 2-fluoro-6-(trifluoromethyl)-N-(1,1,3-trimethyl-2,3-dihydro-1H-inden-4-yl)benzamide, (15.146) 2-(6-benzylpyridin-2-yl)quinazoline, (15.147) 2-[6-(3-fluoro-4-methoxyphenyl)-5-methylpyridin-2-yl]quinazoline, (15.148) 3-(4,4-difluoro-3,3-dimethyl-3,4-dihydroisoquinolin-1-yl)quinoline, (15.149) Abscisic acid, (15.150) 3-(difluoromethyl)-N-methoxy-1-methyl-N-[1-(2,4,6-trichlorophenyl)propan-2-yl]-1H-pyrazole-4-carboxamide, (15.151) N′-[5-bromo-6-(2,3-dihydro-1H-inden-2-yloxy)-2-methylpyridin-3-yl]-N-ethyl-N-methylimidoformamide, (15.152) N′-{5-bromo-6-[1-(3,5-difluorophenyl)ethoxy]-2-methylpyridin-3-yl}-N-ethyl-N-methylimidoformamide, (15.153) N′-{5-bromo-6-[(1R)-1-(3,5-difluorophenyl)ethoxy]-2-methylpyridin-3-yl}-N-ethyl-N-methylimidoformamide, (15.154) N′-{5-bromo-6-[(1S)-1-(3,5-difluorophenyl)ethoxy]-2-methylpyridin-3-yl}-N-ethyl-N-methylimidoformamide, (15.155) N′-{5-bromo-6-[(cis-4-isopropylcyclohexyl)oxy]-2-methylpyridin-3-yl}-N-ethyl-N-methylimidoformamide, (15.156) N′-{5-bromo-6-[(trans-4-isopropylcyclohexyl)oxy]-2-methylpyridin-3-yl}-N-ethyl-N-methylimidoformamide, (15.157)N-cyclopropyl-3-(difluoromethyl)-5-fluoro-N-(2-isopropylbenzyl)-1-methyl-1H-pyrazole-4-carboxamide, (15.158)N-cyclopropyl-N-(2-cyclopropylbenzyl)-3-(difluoromethyl)-5-fluoro-1-methyl-1H-pyrazole-4-carboxamide, (15.159)N-(2-tert-butylbenzyl)-N-cyclopropyl-3-(difluoromethy 1)-5-fluoro-1-methyl-1H-pyrazole-4-carboxamide, (15.160)N-(5-chloro-2-ethylbenzyl)-N-cyclopropyl-3-(difluoromethyl)-5-fluoro-1-methyl-1H-pyrazole-4-carboxamide, (15.161)N-(5-chloro-2-isopropylbenzyl)-N-cyclopropyl-3-(difluoromethyl)-5-fluoro-1-methyl-1H-pyrazole-4-carboxamide, (15.162)N-cyclopropyl-3-(difluoromethyl)-N-(2-ethyl-5-fluorobenzyl)-5-fluoro-1-methyl-1H-pyrazole-4-carboxamide, (15.163) N-cyclopropyl-3-(difluoromethyl)-5-fluoro-N-(5-fluoro-2-isopropylbenzyl)-1-methyl-1H-pyrazole-4-carboxamide, (15.164)N-cyclopropyl-N-(2-cyclopropyl-5-fluorobenzyl)-3-(difluoromethyl)-5-fluoro-1-methyl-1H-pyrazole-4-carboxamide, (15.165)N-(2-cyclopentyl-5-fluorobenzyl)-N-cyclopropyl-3-(difluoromethyl)-5-fluoro-1-methyl-1H-pyrazole-4-carboxamide, (15.166)N-cyclopropyl-3-(difluoromethyl)-5-fluoro-N-(2-fluoro-6-isopropylbenzyl)-1-methyl-1H-pyrazole-4-carboxamide, (15.167) N-cyclopropyl-3-(difluoromethyl)-N-(2-ethyl-5-methylbenzyl)-5-fluoro-1-methyl-1H-pyrazole-4-carboxamide, (15.168)N-cyclopropyl-3-(difluoromethyl)-5-fluoro-N-(2-isopropyl-5-methylbenzyl)-1-methyl-1H-pyrazole-4-carboxamide, (15.169)N-cyclopropyl-N-(2-cyclopropyl-5-methylbenzyl)-3-(difluoromethyl)-5-fluoro-1-methyl-1H-pyrazole-4-carboxamide, (15.170)N-(2-tert-butyl-5-methylbenzyl)-N-cyclopropyl-3-(difluoromethyl)-5-fluoro-1-methyl-1H-pyrazole-4-carboxamide, (15.171) N-[5-chloro-2-(trifluoromethyl)benzyl]-N-cyclopropyl-3-(difluoromethyl)-5-fluoro-1-methyl-1H-pyrazole-4-carboxamide, (15.172)N-cyclopropyl-3-(difluoromethyl)-5-fluoro-1-methyl-N-[5-methyl-2-(trifluoromethyl)benzyl]-1H-pyrazole-4-carboxamide, (15.173)N-[2-chloro-6-(trifluoromethypbenzyl]-N-cyclopropyl-3-(difluoromethyl)-5-fluoro-1-methyl-1H-pyrazole-4-carboxamide, (15.174)N-[3-chloro-2-fluoro-6-(trifluoromethyl)benzyl]-N-cyclopropyl-3-(difluoromethyl)-5-fluoro-1-methyl-1H-pyrazole-4-carboxamide, (15.175)N-cyclopropyl-3-(difluoromethyl)-N-(2-ethyl-4,5-dimethylbenzyl)-5-fluoro-1-methyl-1H-pyrazole-4-carboxamide, (15.176)N-cyclopropyl-3-(difluoromethyl)-5-fluoro-N-(2-isopropylbenzyl)-1-methyl-1H-pyrazole-4-carbothioamide, (15.177) 3-(difluoromethyl)-N-(7-fluoro-1,1,3-trimethyl-2,3-dihydro-1H-inden-4-yl)-1-methyl-1H-pyrazole-4-carboxamide, (15.178) 3-(difluoromethyl)-N-[(3R)-7-fluoro-1,1,3-trimethyl-2,3-dihydro-1H-inden-4-yl]-1-methyl-1H-pyrazole-4-carboxamide, (15.179) 3-(difluoromethyl)-N-[(3S)-7-fluoro-1,1,3-trimethyl-2,3-dihy dro-1H-inden-4-yl]-1-methy 1-1H-pyrazole-4-carboxamide, (15.180) N′-(2,5-dimethyl-4-phenoxyphenyl)-N-ethyl-N-methylimidoformamide, (15.181) N′-{4-[(4,5-dichloro-1,3-thiazol-2-yl)ox)]-2,5-dimethylphenyl}-N-ethyl-N-methylimidoformamide, (15.182)N-(4-chloro-2,6-difluorophenyl)-4-(2-chloro-4-fluorophenyl)-1,3-dimethyl-1H-pyrazol-5-amine.

Mixtures with insecticides are also particularly advantageous. Examples of suitable insecticide mixing partners can be selected from the list consisting of

(1) Acetylcholinesterase (AChE) inhibitors, for example carbamates, e.g. Alanycarb, Aldicarb, Bendiocarb, Benfuracarb, Butocarboxim, Butoxycarboxim, Carbaryl, Carbofuran, Carbosulfan, Ethiofencarb, Fenobucarb, Formetanate, Furathiocarb, Isoprocarb, Methiocarb, Methomyl, Metolcarb, Oxamyl, Pirimicarb, Propoxur, Thiodicarb, Thiofanox, Triazamate, Trimethacarb, XMC and Xylylcarb or organophosphates, e.g. Acephate, Azamethiphos, Azinphos-ethyl, Azinphos-methyl, Cadusafos, Chlorethoxyfos, Chlorfenvinphos, Chlormephos, Chlorpyrifos, Chlorpyrifos-methyl, Coumaphos, Cyanophos, Demeton-S-methyl, Diazinon, Dichlorvos/DDVP, Dicrotophos, Dimethoate, Dimethylvinphos, Disulfoton, EPN, Ethion, Ethoprophos, Famphur, Fenamiphos, Fenitrothion, Fenthion, Fosthiazate, Heptenophos, Imicyafos, Isofenphos, Isopropyl O-(methoxyaminothio-phosphoryl)salicylate, Isoxathion, Malathion, Mecarbam, Methamidophos, Methidathion, Mevinphos, Monocrotophos, Naled, Omethoate, Oxydemeton-methyl, Parathion, Parathion-methyl, Phenthoate, Phorate, Phosalone, Phosmet, Phosphamidon, Phoxim, Pirimiphos-methyl, Profenofos, Propetamphos, Prothiofos, Pyraclofos, Pyridaphenthion, Quinalphos, Sulfotep, Tebupirimfos, Temephos, Terbufos, Tetrachlorvinphos, Thiometon, Triazophos, Trichlorfon and Vamidothion.

(2) GABA-gated chloride channel antagonists, for example cyclodiene organochlorines, e.g. Chlordane and Endosulfan or phenylpyrazoles (fiproles), e.g. Ethiprole and Fipronil.

(3) Sodium channel modulators/voltage-dependent sodium channel blockers, for example pyrethroids, e.g. Acrinathrin, Allethrin, d-cis-trans Allethrin, d-trans Allethrin, Bifenthrin, Bioallethrin, Bioallethrin S-cyclopentenyl isomer, Bioresmethrin, Cycloprothrin, Cyfluthrin, beta-Cyfluthrin, Cyhalothrin, lambda-Cyhalothrin, gamma-Cyhalothrin, Cypermethrin, alpha-Cypermethrin, beta-Cypermethrin, theta-Cypermethrin, zeta-Cypermethrin, Cyphenothrin [(1R)-trans isomers], Deltamethrin, Empenthrin [(EZ)-(1R) isomers), Esfenvalerate, Etofenprox, Fenpropathrin, Fenvalerate, Flucythrinate, Flumethrin, tau-Fluvalinate, Halfenprox, Imiprothrin, Kadethrin, Permethrin, Phenothrin [(1R)-trans isomer), Prallethrin, Pyrethrine (pyrethrum), Resmethrin, Silafluofen, Tefluthrin, Tetramethrin, Tetramethrin [(1R) isomers)], Tralomethrin and Transfluthrin or DDT or Methoxychlor.

(4) Nicotinic acetylcholine receptor (nAChR) agonists, for example neonicotinoids, e.g. Acetamiprid, Clothianidin, Dinotefuran, Imidacloprid, Nitenpyram, Thiacloprid and Thiamethoxam or Nicotine or Sulfoxaflor.

(5) Nicotinic acetylcholine receptor (nAChR) allosteric activators, for example spinosyns, e.g. Spinetoram and Spinosad.

(6) Chloride channel activators, for example avermectins/milbemycins, e.g. Abamectin, Emamectin benzoate, Lepimectin and Milbemectin.

(7) Juvenile hormone mimics, for example juvenile hormon analogues, e.g. Hydroprene, Kinoprene and Methoprene or Fenoxycarb or Pyriproxyfen.

(8) Miscellaneous non-specific (multi-site) inhibitors, for example alkyl halides, e.g. Methyl bromide and other alkyl halides; or Chloropicrin or Sulfuryl fluoride or Borax or Tartar emetic.

(9) Selective homopteran feeding blockers, e.g. Pymetrozine or Flonicamid.

(10) Mite growth inhibitors, e.g. Clofentezine, Hexythiazox and Diflovidazin or Etoxazole.

(11) Microbial disruptors of insect midgut membranes, e.g. Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis and BT crop proteins: Cry1Ab, Cry1Ac, Cry1Fa, Cry2Ab, mCry3A, Cry3Ab, Cry3Bb, Cry34/35Ab 1.

(12) Inhibitors of mitochondrial ATP synthase, for example Diafenthiuron or organotin miticides, e.g. Azocyclotin, Cyhexatin and Fenbutatin oxide or Propargite or Tetradifon.

(13) Uncouplers of oxidative phoshorylation via disruption of the proton gradient, for example Chlorfenapyr, DNOC and Sulfluramid.

(14) Nicotinic acetylcholine receptor (nAChR) channel blockers, for example Bensultap, Cartap hydrochloride, Thiocyclam and Thiosultap-sodium.

(15) Inhibitors of chitin biosynthesis, type 0, for example Bistrifluron, Chlorfluazuron, Diflubenzuron, Flucycloxuron, Flufenoxuron, Hexaflumuron, Lufenuron, Novaluron, Noviflumuron, Teflubenzuron and Triflumuron.

(16) Inhibitors of chitin biosynthesis, type 1, for example Buprofezin. (17) Moulting disruptors, for example Cyromazine.

(18) Ecdysone receptor agonists, for example Chromafenozide, Halofenozide, Methoxyfenozide and Tebufenozide.

(19) Octopamine receptor agonists, for example Amitraz.

(20) Mitochondrial complex III electron transport inhibitors, for example Hydramethylnon or Acequinocyl or Fluacrypyrim.

(21) Mitochondrial complex I electron transport inhibitors, for example METI acaricides, e.g. Fenazaquin, Fenpyroximate, Pyrimidifen, Pyridaben, Tebufenpyrad and Tolfenpyrad or Rotenone (Derris).

(22) Voltage-dependent sodium channel blockers, e.g. Indoxacarb or Metaflumizone.

(23) Inhibitors of acetyl CoA carboxylase, for example tetronic and tetramic acid derivatives, e.g. Spirodiclofen, Spiromesifen and Spirotetramat.

(24) Mitochondrial complex IV electron transport inhibitors, for example phosphines, e.g. Aluminium phosphide, Calcium phosphide, Phosphine and Zinc phosphide or Cyanide.

(25) Mitochondrial complex II electron transport inhibitors, for example Cyenopyrafen and Cyflumetofen.

(28) Ryanodine receptor modulators, for example diamides, e.g. Chlorantraniliprole, Cyantraniliprole and Flubendiamide.

Further active ingredients with unknown or uncertain mode of action, for example Afidopyropen, Azadirachtin, Benclothiaz, Benzoximate, Bifenazate, Bromopropylate, Chinomethionat, Cryolite, Dicofol, Diflovidazin, Fluensulfone, Flometoquin, Flufenerim, Flufenoxystrobin, Flufiprole, Fluopyram, Flupyradifurone, Fufenozide, Heptafluthrin, Imidaclothiz, Iprodione, Meperfluthrin, Paichongding, Pyflubumide, Pyrifluquinazon, Pyriminostrobin, Tetramethylfluthrin and Iodomethane; furthermore products based on Bacillus firmus (including but not limited to strain CNCM 1-1582, such as, for example, VOTiVO™, BioNem) or one of the following known active compounds:3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) and 1-{2-fluoro-4-methyl-5-[(2,2,2-trifluorethyl)sulfinyl]phenyl}-3-(trifluoromethyl)-1H-1,2,4-triazol-5-amine (known from WO2006/043635), {1′-[(2E)-3-(4-chlorophenyl)prop-2-en-1-yl]-5-fluorospiro[indole-3,4′-piperidin]-1(2H)-yl}(2-chloropyridin-4-yl)methanone (known from WO2003/106457), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl ethyl carbonate (known from WO2009/049851), 4-(but-2-yn-1-yloxy)-6-(3,5-dimethylpiperidin-1-yl)-5-fluoropyrimidine (known from WO2004/099160), 4-(but-2-yn-1-yloxy)-6-(3-chlorophenyl)pyrimidine (known from WO2003/076415), PF1364 (CAS-Reg. No. 1204776-60-2), 4-[5-(3,5-dichlorophenyl)-5-(trifluoromethyl)-4,5-dihy dro-1,2-oxazol-3-yl]-2-methyl-N-{2-oxo-2-[(2,2,2-trifluoroethyl)amino]ethyl}benzamide (known from WO2005/085216), 4-{5-[3-chloro-5-(trifluoromethyl)phenyl]-5-(trifluoromethyl)-4,5-dihy dro-1,2-oxazol-3-yl}-N-{2-oxo-2-[(2,2,2-trifluoroethyl)amino]ethyl}-1-naphthamide (known from WO2009/002809), methyl 2-[2-({[3-bromo-1-(3-chloropyridin-2-yl)-1H-pyrazol-5-yl]carbonyl}amino)-5-chloro-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO2005/085216), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO2010/069502), N-[2-(5-amino-1,3,4-thiadiazol-2-yl)-4-chloro-6-methylphenyl]-3-bromo-1-(3-chloropyridin-2-yl)-1H-pyrazole-5-carboxamide (known from CN102057925), 3-chloro-N-(2-cyanopropan-2-yl)-N-[4-(1,1,1,2,3,3,3-heptafluoropropan-2-yl)-2-methylphenyl]phthalamide (known from WO2012/034472), 8-chloro-N-[(2-chloro-5-methoxyphenyl)sulfonyl]-6-(trifluoromethyl)imidazo[1,2-a]pyridine-2-carboxamide (known from WO2010/129500), 8-chloro-N-[(2-chloro-5-methoxyphenyl)sulfonyl]-6-(trifluoromethyl)imidazo[1,2-a]pyridine-2-carboxamide (known from WO2009/080250), N-[(2E)-1-[(6-chloropyridin-3-yl)methyl]pyridin-2(1H)-ylidene]-2,2,2-trifluoroacetamide (known from WO2012/029672), 1-[(2-chloro-1,3-thiazol-5-yl)methyl]-4-oxo-3-phenyl-4H-pyrido[1,2-a]pyrimidin-1-ium-2-olate (known from WO2009/099929), 1-[(6-chloropyridin-3-yl)methyl]-4-oxo-3-phenyl-4H-pyrido[1,2-a]pyrimidin-1-ium-2-olate (known from WO2009/099929), (5S,8R)-1-[(6-chloropyridin-3-yl)methyl]-9-nitro-2,3,5,6,7,8-hexahydro-1H-5,8-epoxy imidazo[1,2-a]azepine (known from WO2010/069266), (2E)-1-[(6-chloropyridin-3-yl)methyl]-N′-nitro-2-pentylidenehydrazinecarboximidamide (known from WO2010/060231), 4-(3-{2,6-dichloro-4-[(3,3-dichloroprop-2-en-1-yl)oxy]phenoxy}propoxy)-2-methoxy-6-(trifluoromethyl)pyrimidine (known from CN101337940), N-[2-(tert-butylcarbamoyl)-4-chloro-6-methylphenyl]-1-(3-chloropyridin-2-yl)-3-(fluoromethoxy)-1H-pyrazole-5-carboxamide (known from WO2008/134969).

Mixtures with nematicides are also particularly advantageous. Examples of suitable nematicide mixing partners can be selected from the list consisting of

Dichloropropene, Metam sodium, Metam potassium, Chloropicrin, Oxamyl, Carbofuran, Fosthiazate, Aldicarb, Fenamiphos, Cadusafos, Abamectin, Cyanamide, Dazomet, Methylbromide, Terbufos, Ethoprophos, Ethylen dibromide, Phorate, Methylisothiocyanate, Thiodicarb, Sodium tetrathiocarbonate, Iprodione, Fluensulfone, Imicyafos, Dimethyl disulfide, Spirotetramate, Fluopyram, 1-{2-fluoro-4-methyl-5-[(2,2,2-trifluoroethyl)sulfinyl]phenyl}-3-(trifluoromethyl)-1H-1,2,4-triazol-5-amine, 8-chloro-N-[(2-chloro-5-methoxyphenyl)sulfonyl]-6-(trifluoromethyl)imidazo[1,2-a]pyridine-2-carboxamide, 8-chloro-N-[(4-cyano-2,5-dimethylphenyl)sulfonyl]-6-(trifluoromethyl)imidazo[1,2-a]pyridine-2-carboxamide, 2-(4-chlorophenyl)-5-(2-thienyl)-1,3,4-oxadiazole, 5-phenyl-2-(2-thienyl)-1,3-oxazole, 5-(4-chlorophenyl)-2-(2-thienyl)-1,3-oxazole, 5-(4-bromophenyl)-2-(2-thienyl)-1,3-oxazole or 2-(4-chlorophenyl)-5-(2-thienyl)-2H-tetrazole.

All named mixing partners can, if their functional groups enable this, optionally form salts with suitable bases or acids.

The active ingredients specified herein by their “common name” are known and described, for example, in the Pesticide Manual (“The Pesticide Manual”, 14th Ed., British Crop Protection Council 2006) or can be searched in the internet (e.g. http://www.alanwood.net/oesticides).

The active substances, active substance combinations or compositions according to the invention can also be combined with microbials.

The microbials according to the invention, in combination with good plant tolerance and favourable toxicity to warm-blooded animals and being tolerated well by the environment, are suitable for protecting plants and plant organs, for increasing harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They can be preferably employed as plant protection agents. They are active against normally sensitive and resistant species and against all or some stages of development. The abovementioned microbials include:

Microbials from the domain Bacteria, microbials from the domain Fungi, insecticidal microbials from the domain Protozoa, insecticidal microbials from the domain Viruses, and microbials from the domain of entomopathogenic nematodes.

The various aspects of the invention will now be illustrated with reference to the following production and use examples in a non limiting manner.

### PREPARATION EXAMPLES

¹H-NMR data

¹H-NMR-data were determined with a Bruker Avance 400 equipped with a flow cell (60 μl volume) or with a Bruker AVIII 400 equipped with 1 7 mm cryo-CPTCI probe head or with a Bruker AVII 600 (600.13 MHz) equipped with a cyroTCI probe head or with a Bruker AVIII 600 (601.6 MHz) equipped with a cryo CPMNP probe head with tetramethylsilane as reference (0.0) and the solvents CD₃CN, CDCl₃, [D₆]-DMSO.

¹H-NMR-data of selected examples are listed in classic format (chemical shift 6, multiplicity, number of hydrogen atoms) or as NMR-peak-lists.

NMR-peak-lists:

If NMR-data of selected examples are provided in form of ¹H-NMR-peak lists, then for every peak first the chemical shift δ in ppm and then, separated by a blank, the intensity of the signal in round brackets is listed. Between the δ-value—signal intensity pairs are semicolons as delimiters.

The peak list of an example is therefore listed as: δ₁ (intensity₁); δ₂ (intensity₂); . . . ; δ (intensityᵢ); . . . ; (intensity.).

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for ¹H spectra, we use tetramethylsilane and/or the chemical shift of the solvent used, especially in the case of spectra measured in DMSO. Therefore in NMR peak lists, tetramethylsilane peak can occur but not necessarily.

The ¹H-NMR peak lists are similar to classical ¹H-NMR prints and contain therefore usually all peaks, which are listed at classical NMR-interpretation.

Additionally they can show like classical ¹H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D6 and the peak of water are shown in our ¹H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical ¹H-NMR interpretation.

Further details of NMR-data description with peak lists you find in the publication “Citation of NMR Peaklist Data within Patent Applications” of the Research Disclosure Database Number 564025.

The solvent, in which the NMR-spectrum was measured, is specified in squared brackets.

### Preparation Example 1

### Synthesis of 5-(((tert-Butyldimethylsilyboxy)methyl)-2-chlorobenzene-sulfonamide

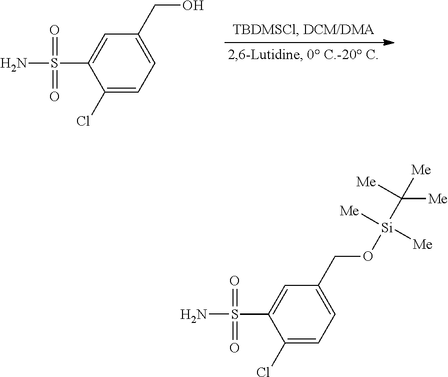 [SMILES: NS(=O)(=O)c1cc(CO)ccc1Cl ; CC(C)(C)[Si](C)(C)OCc1ccc(Cl)c(S(N)(=O)=O)c1]

500 mg (2.26 mmol) 2-Chloro-5-(hydroxymethyl)benzenesulfonamide (known from WO 2009/118292) was dissolved in 22 ml dichloromethane (DCM) and 485 mg (4.52 mmol) 2,6-dimethyllutidine was added at 0° C. The mixture was stirred at 0° C. for 30 min, then 511 mg (3.39 mmol)tert. butyl-dimethyl silylchloride (TBDMSCl), dissolved in 8 ml dimethylacetamide (DMA) was added while maintaining the cooling. The resulted mixture was stirred for 10 min at 0° C., and for additional 10-20 h at 20° C. The progress of the reaction was monitored by TLC analysis (CHCl₃:MeOH 10:1). When the reaction was completed, 5 ml of water was added, and the phases were separated. The aqueous layer was extracted with dichloromethane, the combined organic phase was washed with water, dried over MgSO₄, filtered and evaporated to dryness. The residue was re-dissolved in toluene (0.5 mL), and evaporated to dryness. This procedure was repeated four times in order to completely remove the residual dimethylacetamide. Yield: 720 mg (2.14 mmol, 95%) 5-(((tert-butyldimethylsilyl)oxy)methyl)-2-chlorobenzene-sulfonamide as a light yellow solid, ESI MS, m/z 353 [M+NH₄]+, HPLC-MS (220 nm) 96% (AUC).

1H-NMR, Solvent: <[D₆]-DMSO>, spectrometer: 399.95 MHz

0.09528 (0.7326); 0.91442 (1); 2.49561 (0.0856); 2.5 (0.1188); 2.50439 (0.0866); 3.29593 (0.1608); 4.76673 (0.1528); 7.48693 (0.0204); 7.49133 (0.0209); 7.50701 (0.0301); 7.51202 (0.0314); 7.55279 (0.0979); 7.5898 (0.0709); 7.60987 (0.0468); 7.97114 (0.0494); 7.97553 (0.0483).

### Preparation Example 2

### Synthesis of N-{[5-({[tert-butyl(dimethyl)silyl]oxy}methyl)-2-chlorophenyl]sulfonyl}-8-chloro-6-(trifluoromethyl)imidazol[1,2-a]pyridine-2-carb oxamide

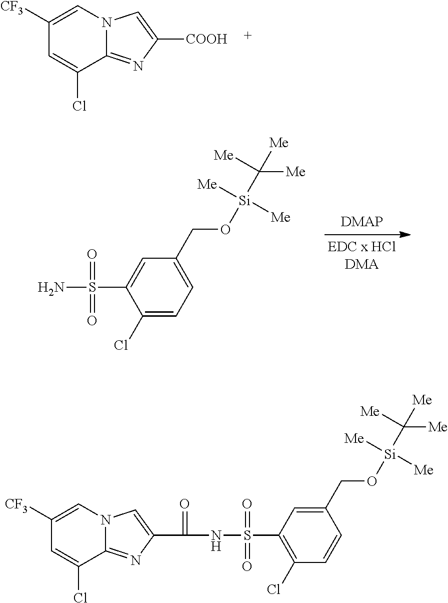 [SMILES: O=C(O)c1cn2cc(C(F)(F)F)cc(Cl)c2n1 ; CC(C)(C)[Si](C)(C)OCc1ccc(Cl)c(S(N)(=O)=O)c1 ; CC(C)(C)[Si](C)(C)OCc1ccc(Cl)c(S(=O)(=O)NC(=O)c2cn3cc(C(F)(F)F)cc(Cl)c3n2)c1]

10.39 g (85.0 mmol) Dimethylaminopyridine (DMAP) and 13.59 g (70.9 mmol) 1-ethyl-3-(3-dimethylaminopropyl)carbodiimide hydrochloride (EDC×HCl) were dissolved in 115 ml dimethylacetamide and 7.50 g (28.35 mmol) 8-chloro-6-(trifluoromethyl)imidazo[1,2-a]pyridine-2-carboxylic acid were added.

The mixture was stirred for 60 min at 20° C., then 10.0 g (29.8 mmol) 5-(((tert-butyldimethylsilyl)oxy)methyl)-2-chlorobenzene-sulfonamide was added. The mixture was stirred at 20° C. overnight. The progress of the reaction was monitored by TLC analysis (CHCl₃:MeOH 10:1). When the reaction was completed, the mixture was poured onto 115 ml water, the aqueous solution was extracted two times with 300 ml ethyl acetate, the combined organic phase was dried (MgSO₄), filtered and evaporated to dryness. The residue was re-dissolved in toluene (0.5 mL), and evaporated to dryness. This procedure was repeated four times in order to completely remove the residual dimethylacetamide. Yield: 12.28 g (21.08 mmol)N-{[5-({[tert-butyl(dimethyl)silyl]oxy}methyl)-2-chlorophenyl]sulfonyl}-8-chloro-6-(trifluoromethyl)imidazo[1,2-a]pyridine-2-carboxamide as colourless solid, ESI MS, m/z 582 [M]+, HPLC-MS (220 nm) 77% (AUC).

1H-NMR, Solvent: <CDCl₃>, spectrometer: 399.95 MHz

0.00246 (0.8652); 0.80528 (1); 0.82034 (0.2787); 0.85483 (0.0095); 0.868 (0.016); 0.87302 (0.0144); 0.88557 (0.0261); 0.90375 (0.0128); 1.01163 (0.0105); 1.86464 (0.0532); 2.41156 (0.1215); 2.43038 (0.0229); 2.58091 (0.0767); 2.69318 (0.0494); 2.79353 (0.0072); 2.81172 (0.0101); 2.83117 (0.0074); 2.85124 (0.0562); 2.89263 (0.007); 2.91019 (0.0093); 2.92525 (0.0099); 2.93026 (0.0109); 2.94657 (0.0104); 2.9842 (0.0086); 3.0187 (0.8655); 3.05445 (0.0069); 4.65634 (0.1661); 4.67516 (0.0469; 6.76753 (0.0959); 6.78509 (0.0977); 7.22602 (0.0185); 7.22978 (0.0184); 7.24671 (0.0469); 7.25048 (0.0481); 7.27055 (0.0834); 7.29062 (0.0311); 7.40163 (0.0068); 7.41857 (0.0111); 7.42233 (0.0112); 7.47251 (0.0127); 7.4976 (0.0192); 7.51829 (0.012); 7.65691 (0.0725); 7.88584 (0.0159); 7.8896 (0.0159); 7.91908 (0.0624); 8.15679 (0.0932); 8.17498 (0.0901); 8.29227 (0.1212); 8.4911 (0.0075); 9.15405 (0.0657).

### Preparation Example 3

### Synthesis of 8-chloro-N-{[2-chloro-5-(hydroxymethyl)phenyl]sulfonyl}-6-(trifluoromethyl)imidazo[1,2-a]pyridine-2-carboxamide

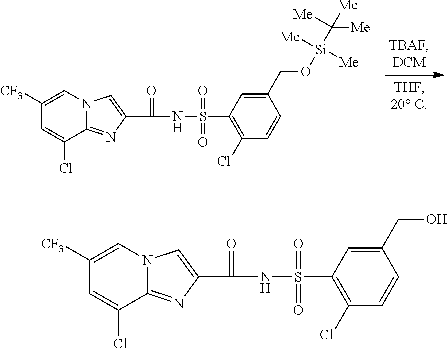 [SMILES: CC(C)(C)[Si](C)(C)OCc1ccc(Cl)c(S(=O)(=O)NC(=O)c2cn3cc(C(F)(F)F)cc(Cl)c3n2)c1 ; O=C(NS(=O)(=O)c1cc(CO)ccc1Cl)c1cn2cc(C(F)(F)F)cc(Cl)c2n1]

12.25 g (21.03 mmol)N-{[5-({[tert-butyl(dimethyl)silyl]oxy}methyl)-2-chlorophenyl]sulfonyl}-8-chloro-6-(trifluoromethyl)imidazo[1,2-a]pyridine-2-carboxamide was dissolved in 75 ml dichloromethane, excess (5-10 equiv) tetrabutylammoniumfluoride as a 1 M solution in tetrahydrofurane was added and the mixture was stirred for 10-24 h at 20° C. The progress of the reaction was monitored by TLC analysis (CHCl₃:MeOH 10:1). When the reaction was completed, 100 ml of water was added, and the phases were separated. The aqueous layer was washed two times with 100 ml dichloromethane and once with 20 ml n-butanol. The combined organic phase was evaporated to dryness, the residue was taken up in 60 ml chloroform and 1.5 ml 3% aqueous hydrochloric acid was added. The solid precipitate was filtered off, and triturated with 40 ml diisopropylether. Yield: 5.34 g (11.4 mmol) 8-chloro-N-{[2-chloro-5-(hydroxymethyl)phenyl]sulfonyl}-6-(trifluoromethyl)imidazo[1,2-a]pyridine-2-carboxamide as a colourless solid, ESI MS, m/z 467 [M]+, HPLC-MS (220 nm) 94% (AUC).

1H-NMR, Solvent: <[D₆]-DMSO>, spectrometer: 399.95 MHz

0.04647 (0.0876); 0.83602 (0.1271); 0.89686 (0.0272); 0.91442 (0.0995); 0.93261 (0.2203); 0.9508 (0.1122); 1.28322 (0.0312); 1.3014 (0.0582); 1.31959 (0.0587); 1.33778 (0.032); 1.55229 (0.0305); 1.5711 (0.0389); 1.58929 (0.0273); 2.5 (0.4248); 3.14226 (0.0436); 3.16296 (0.041); 3.18366 (0.0421); 4.61244 (1); 5.74329 (0.2575); 7.58854 (0.0424); 7.60799 (0.9262); 7.63245 (0.032); 7.93351 (0.4065); 7.93602 (0.4088); 8.16746 (0.4027); 8.82038 (0.6444); 9.32842 (0.3617).

### Preparation Example 4

### Synthesis of 8-chloro-N-{[2-chloro-5-(chloromethyl)phenyl]sulfonyl}-6-(trifluoromethyl)imidazo[1,2-a]pyridine-2-carboxamide

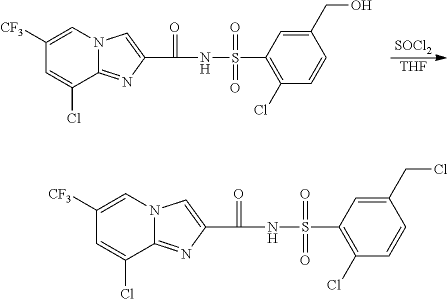

4.67 g (9.97 mmol) 8-chloro-N-{[2-chloro-5-(hydroxymethyl)phenyl]sulfonyl}-6-(trifluoromethyl)imidazo[1,2-a]pyridine-2-carboxamide were dissolved in 30 ml tetrahydrofurane (THF), and the solution was cooled to 0° C. Maintaining the temperature 1.43 g (12.0 mmol) thionyl chloride was added dropwise. The mixture was stirred at 20° C. for 3-6 h, meanwhile the progress of the reaction was monitored by TLC analysis (CHCl₃:MeOH 5:1). After completion of the reaction, the mixture was evaporated to dryness. The residue was taken up in 20 ml toluene and evaporated to dryness, then the procedure was repeated once more. Yield: 4.7 g (9.66 mmol) 8-chloro-N-{[2-chloro-5-(chloromethyl)phenyl]sulfonyl}-6-(trifluoromethyl)imidazo[1,2-a]pyridine-2-carboxamide as colourless solid, AP MS, m/z 488 [M+H]+, HPLC-MS (220 nm) 95% (AUC).

1H-NMR, Solvent: <[D₆]-DMSO>, spectrometer: 399.95 MHz: The spectroscopic data are provided in FIG. 1.

### Preparation Example 5

### Synthesis of 8-chloro-N-({2-chloro-5-[(4-cyano-1H-pyrazol-1-yl)methyl]phenyl}sulfonyl)-6-(trifluoromethyl)imidazo[1,2-a]pyridine-2-carboxamide, Ex. 1a-14

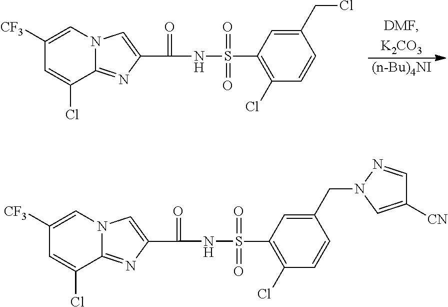

22.9 mg (0.246 mmol) 1H-pyrazole-4-carbonitrile were dissolved under argon in 2 ml dimethyl formamide and 170 mg (1.23 mmol) potassium carbonate were added at 10° C. After 20 min stirring 99.6 mg (0.205 mmol) 8-chloro-N-{[2-chloro-5-(chloromethyl)phenyl]sulfonyl}-6-(trifluoromethyl)imidazo[1,2-a]pyridine-2-carboxamide and 7.6 mg (0.02 mmol) tetrabutylammonium iodide were added and heated for 5 h to 80° C. The progress of the reaction was monitored by TLC analysis (CH₂Cl₂:isopropanol 10:1). After completion of the reaction, the mixture was poured onto 25 ml water and acidified with 6N hydrochloric acid. Yield: 75 mg (0.138 mmol) 8-chloro-N-({2-chloro-5-[(4-cyano-1H-pyrazol-1-yl)methyl]phenyl}sulfonyl)-6-(trifluoromethyl)imidazo[1,2-a]pyridine-2-carboxamide.

### Preparation Examples of Intermediates

Synthesis of intermediates of formula (IIIA) according to scheme 9 as previously described, where T=Y═H.

### Preparation Example 6

### Preparation of 3-(Hydroxymethyl)Benzenesulfonamide, (XI-1)

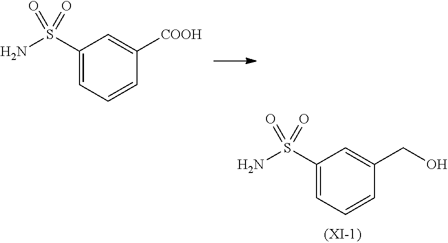 [SMILES: NS(=O)(=O)c1cccc(C(=O)O)c1 ; NS(=O)(=O)c1cccc(CO)c1]

4.36 g (21.6 mmol) 3-sulfamoylbenzoic acid were suspended in 65 ml tetrahydrofurane and cooled to 0° C. During 25 min 65 ml of a solution of 1 M borane tetrahydrofurane complex (BH₃.THF, 65 mmol) were added dropwise. After 2 h stirring at room temperature further 32 ml borane complex were added (32 mmol) and 2 h stirred After cooling to 0° C. 80 ml methanol were added carefully and after 15 min 180 ml aqueous 2 N hydrochloric acid.

After 16 h at room temperature the mixture was evaporated to dryness and the residue extracted three times with ethyl acetate. The combined organic phases were washed with brine, dried over magnesium sulfate and concentrated. Purification by liquid chromatography with cyclohexane/ethyl acetate gave 2.78 g (purity 97.4%, 66.7% of theory) 3-(hydroxymethyl)benzenesulfonamide, (XI-1).

Compounds of general formula (XI) (T=H, Y═H) are to some extent commercially available (“Comm av.”), described in the literature or can be prepared by the method described above, see table 2:

TABLE 2
Compounds of general formula (XI)

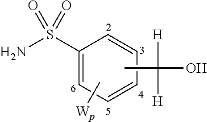

T = Y = H

| | | | | | Position | |
|---|---|---|---|---|---|---|
| | | Position- | Position- | Position- | of | |
| Ex. No | p | W | W | W | CH₂—OH | comment |
| XI-1 | 0 | — | — | — | 3 | Preparative |
| | | | | | | example, |
| | | | | | | NMR |
| XI-2 | 1 | 2-C₂H₅ | — | — | 5 | NMR |
| XI-3 | 1 | 2-Cl | — | — | 5 | WO |
| | | | | | | 2009/118292 |
| | | | | | | NMR |
| XI-4 | 1 | 3-Cl | — | — | 5 | Comm. av. |
| XI-5 | 1 | 3-F | — | — | 5 | Comm. av. |
| XI-6 | 1 | 3-Br | — | — | 5 | Comm. av. |
| XI-7 | 2 | 2-F | 4-Cl | — | 5 | WO |
| | | | | | | 2011/071565 |
| | | | | | | NMR |
| XI-8 | 1 | 4-F | — | — | 3 | WO |
| | | | | | | 2008/017932 |
| | | | | | | NMR |
| XI-9 | 1 | 3-CH₃ | — | — | 5 | WO |
| | | | | | | 2007/085833 |
| XI-10 | 2 | 2-Cl | 3-NO₂ | — | 5 | WO |
| | | | | | | 2007/085833 |
| XI-11 | 1 | 4-OCH₃ | — | — | 3 | WO |
| | | | | | | 2007/002313 |
| | | | | | | NMR |
| XI-12 | 2 | 2-Cl | 4-F | — | F | WO |
| | | | | | | 2005/082890 |
| XI-13 | 1 | 2-OCH₃ | — | — | 5 | Comm. av. |
| | | | | | | NMR |
| XI-14 | 1 | 4-Br | — | — | 3 | U.S. Pat. No. |
| | | | | | | 3,868,374 |
| | | | | | | NMR |
| XI-15 | 2 | 2-Cl | 4-Cl | — | 5 | Journal Med |
| | | | | | | Chem 17(9), |
| | | | | | | 956-65 (1974) |
| | | | | | | NMR |
| XI-16 | 1 | 3-F | — | — | 4 | WO |
| | | | | | | 2008/017932 |
| XI-17 | 1 | 2-Cl | — | — | 4 | |
| XI-18 | 1 | 2-F | — | — | 4 | |
| XI-19 | 1 | 2-Br | — | — | 4 | |
| XI-20 | 2 | 2-Cl | 5-Cl | — | 4 | |
| XI-21 | 1 | 2-Cl | — | — | 6 | |
| XI-22 | 1 | 2-OCF₃ | — | — | 5 | NMR |
| XI-23 | 2 | 3-OCH₃ | 4-OCH₄ | — | 5 | NMR |
| XI-24 | 1 | 4-Cl | — | — | 3 | NMR |
| XI-25 | 2 | 2-CH₃ | 3-CH₃ | — | 5 | NMR |
| XI-26 | 1 | 2-CF₃ | — | — | 5 | |
| XI-27 | 1 | 2-CH₃ | — | — | 5 | NMR |
| XI-28 | 1 | 2-F | — | — | 5 | |
| XI-29 | 1 | 2-Br | — | — | 5 | |
| XI-30 | 1 | 2-Br | — | — | 3 | |
| XI-31 | 1 | 3-Br | — | — | 4 | |
| XI-32 | 1 | 3-Cl | — | — | 4 | |
| XI-33 | 1 | 2-Br | — | — | 6 | |
| XI-34 | 1 | 2-OC₂H₅ | — | — | 6 | |

¹H-NMR-Data of Compounds of Table 2:

No. XI-1, Solvent: <[D₆]-DMSO>, spectrometer: 399.95 MHz

δ=7.816 (5.0); 7.701 (1.5); 7.696 (1.5); 7.690 (2.9); 7.686 (2.6); 7.679 (1.9); 7.674 (1.7); 7.532 (0.4); 7.513 (8.7); 7.501 (7.7); 7.327 (9.6); 5.158 (0.5); 4.572 (16.0); 4.055 (0.8); 4.037 (2.4); 4.020 (2.4); 4.002 (0.8); 3.449 (0.4); 3.431 (0.4); 3.414 (0.3); 3.346 (0.7); 2.510 (10.3); 2.506 (21.2); 2.501 (28.3); 2.497 (20.5); 2.492 (9.9); 2.093 (1.2); 1.988 (10.2); 1.908 (0.8); 1.192 (2.8); 1.175 (5.7); 1.157 (2.8); 1.055 (0.4); 0.008 (1.3); 0.000 (40.4); −0.009 (1.6)

No. XI-2, Solvent: <[D₆]-DMSO>, spectrometer: 399.95 MHz

δ=7.831 (5.2); 7.451 (2.0); 7.447 (2.0); 7.431 (3.1); 7.428 (3.1); 7.394 (1.8); 7.379 (8.3); 7.366 (5.9); 7.346 (3.4); 4.784 (1.2); 4.511 (14.1); 3.380 (0.4); 2.998 (1.7); 2.979 (5.8); 2.961 (6.0); 2.942 (2.1); 2.506 (19.8); 2.501 (27.4); 2.497 (21.1); 1.355 (0.9); 1.222 (7.4); 1.203 (16.0); 1.184 (7.7); 0.000 (11.1); −0.008 (0.8)

No. XI-3, Solvent: <[D₆]-DMSO>, spectrometer: 399.95 MHz

δ=8.506 (0.4); 8.501 (0.3); 7.968 (5.0); 7.963 (5.1); 7.784 (0.4); 7.600 (0.5); 7.587 (4.5); 7.567 (16.0); 7.516 (3.5); 7.511 (3.3); 7.496 (2.1); 7.491 (2.0); 7.386 (0.6); 6.529 (0.8); 5.482 (1.1); 5.469 (2.1); 5.456 (1.2); 4.819 (0.6); 4.551 (5.7); 4.540 (5.6); 4.038 (0.4); 4.020 (0.4); 3.328 (1.3); 2.524 (0.5); 2.506 (23.8); 2.502 (30.0); 2.497 (22.1); 1.989 (1.8); 1.337 (0.5); 1.192 (0.5); 1.175 (1.0); 1.157 (0.5); 0.008 (2.5); 0.000 (53.0)

No. XI-7, Solvent: <[D₆]-DMSO>, spectrometer: 399.95 MHz

δ=7.984 (5.1); 7.964 (5.0); 7.932 (0.4); 7.912 (0.4); 7.759 (12.8); 7.737 (0.6); 7.712 (0.5); 7.685 (6.4); 7.661 (6.3); 4.850 (1.0); 4.561 (16.0); 3.693 (0.3); 3.676 (0.5); 3.665 (0.5); 3.649 (0.4); 3.602 (0.4); 3.584 (0.4); 3.537 (0.4); 3.521 (0.5); 3.454 (0.5); 3.433 (0.5); 3.427 (0.5); 3.396 (1.0); 3.381 (1.6); 3.364 (1.0); 3.330 (0.5); 3.314 (0.5); 3.304 (0.4); 3.269 (0.3); 3.169 (0.5); 2.507 (49.7); 2.503 (64.4); 2.498 (48.1); 2.334 (0.4); 2.329 (0.5); 2.325 (0.4); 1.426 (0.3); 1.355 (1.1); 1.320 (0.3); 1.303 (0.4); 1.284 (0.3); 1.234 (0.5); 0.882 (0.7); 0.875 (0.3); 0.864 (1.4); 0.846 (0.6); 0.008 (1.0); 0.000 (44.9); −0.008 (2.9)

No. XI-8, Solvent: <[D₆]-DMSO>, spectrometer: 399.95 MHz

δ=7.995 (2.8); 7.989 (3.0); 7.978 (2.8); 7.972 (2.8); 7.772 (1.8); 7.766 (1.8); 7.760 (1.9); 7.751 (2.4); 7.744 (1.9); 7.738 (1.9); 7.732 (1.6); 7.412 (1.9); 7.393 (10.0); 7.374 (4.3); 7.352 (4.1); 7.349 (4.5); 7.327 (3.2); 4.880 (1.4); 4.604 (16.0); 3.677 (0.6); 3.661 (0.4); 3.396 (0.4); 3.380 (1.0); 3.364 (0.5); 2.506 (40.3); 2.502 (61.4); 2.497 (52.3); 2.493 (32.4); 2.329 (0.7); 2.183 (0.3); 1.355 (1.6); 1.303 (0.4); 1.286 (0.4); 0.882 (0.6); 0.864 (1.2); 0.846 (0.6); −0.001 (60.5); −0.150 (0.5)

No. XI-11, Solvent: <[D₆]-DMSO>, spectrometer: 399.95 MHz

δ=7.883 (2.7); 7.807 (0.3); 7.701 (1.5); 7.696 (1.4); 7.679 (1.5); 7.674 (1.3); 7.182 (5.2); 7.144 (0.3); 7.122 (0.4); 7.099 (2.8); 7.078 (2.5); 4.797 (0.9); 4.513 (8.3); 3.861 (1.9); 3.845 (16.0); 3.381 (0.6); 2.503 (23.2); 1.355 (0.8)

No. XI-13, Solvent: <[D₆]-DMSO>, spectrometer: 399.95 MHz

δ=8.304 (0.3); 8.299 (0.4); 7.715 (2.6); 7.709 (2.7); 7.485 (1.2); 7.480 (1.3); 7.464 (1.3); 7.459 (1.3); 7.320 (0.4); 7.235 (0.5); 7.178 (0.7); 7.158 (2.9); 7.136 (2.3); 7.061 (0.5); 7.014 (2.8); 6.870 (0.3); 4.733 (1.2); 4.500 (0.7); 4.460 (7.0); 3.978 (1.7); 3.898 (1.5); 3.889 (3.5); 3.882 (16.0); 3.842 (0.8); 3.617 (0.6); 3.607 (0.6); 3.601 (1.6); 3.585 (0.7); 3.397 (0.5); 3.381 (0.9); 3.370 (1.1); 3.365 (0.7); 3.268 (0.5); 3.169 (1.8); 2.506 (15.1); 2.501 (21.3); 2.497 (17.1); 2.183 (0.5); 1.776 (0.6); 1.768 (0.7); 1.759 (1.8); 1.751 (0.8); 1.743 (0.7); 1.355 (3.2); 1.303 (0.4); 1.284 (0.4); 0.882 (0.7); 0.875 (0.7); 0.864 (1.3); 0.857 (0.5); 0.846 (0.6); 0.000 (32.5)

No. XI-14, Solvent: <[D₆]-DMSO>, spectrometer: 399.95 MHz

δ=8.003 (8.9); 7.999 (9.3); 7.797 (1.2); 7.777 (1.5); 7.763 (8.3); 7.743 (9.2); 7.582 (1.1); 7.543 (16.0); 7.503 (0.3); 7.421 (5.0); 7.417 (4.9); 7.401 (4.3); 6.870 (0.4); 4.903 (0.7); 4.828 (0.9); 4.802 (2.0); 4.572 (0.4); 4.528 (14.6); 3.665 (0.4); 3.616 (2.3); 3.601 (5.9); 3.585 (2.6); 3.469 (0.7); 3.397 (1.1); 3.381 (1.2); 3.370 (1.7); 3.169 (1.8); 2.670 (0.3); 2.506 (43.2); 2.501 (56.7); 2.497 (44.6); 2.328 (0.3); 2.183 (0.6); 1.776 (2.3); 1.767 (2.9); 1.760 (6.8); 1.751 (3.1); 1.743 (2.6); 1.440 (0.4); 1.425 (0.4); 1.408 (0.5); 1.390 (0.6); 1.373 (0.5); 1.355 (4.5); 1.321 (0.6); 1.304 (0.8); 1.288 (0.7); 1.267 (0.4); 0.892 (0.7); 0.883 (1.4); 0.874 (1.3); 0.864 (2.3); 0.847 (1.1); 0.146 (0.4); 0.000 (78.5); −0.150 (0.4)

No. XI-15, Solvent: <[D₆]-DMSO>, spectrometer: 399.95 MHz

δ=8.358 (0.4); 8.169 (8.7); 8.108 (0.7); 7.919 (0.4); 7.845 (1.3); 7.796 (10.9); 7.697 (11.6); 7.075 (0.3); 6.947 (0.4); 4.865 (1.3); 4.577 (16.0); 4.470 (0.5); 3.665 (0.4); 3.601 (0.5); 3.397 (0.6); 3.381 (1.0); 3.371 (0.5); 3.365 (0.6); 3.169 (0.5); 2.508 (31.7); 2.503 (41.0); 2.499 (32.6); 2.330 (0.6); 2.183 (0.6); 1.760 (0.5); 1.751 (0.3); 1.355 (3.4); 1.320 (0.4); 1.303 (0.5); 1.282 (0.5); 1.235 (0.4); 0.882 (0.7); 0.875 (0.5); 0.864 (1.4); 0.846 (0.7); −0.001 (1.0)

No. XI-22, Solvent: <[D₆]-DMSO>, spectrometer: 399.95 MHz

δ=7.918 (6.2); 7.913 (6.5); 7.638 (3.1); 7.633 (3.1); 7.617 (5.5); 7.605 (13.1); 7.510 (3.6); 7.506 (3.8); 7.489 (2.6); 7.485 (2.7); 6.531 (0.7); 5.499 (0.4); 5.490 (0.4); 4.840 (0.9); 4.568 (16.0); 3.396 (0.9); 3.380 (1.4); 3.370 (1.4); 3.364 (1.6); 3.346 (1.7); 3.168 (0.7); 2.671 (0.4); 2.524 (1.1); 2.511 (22.7); 2.507 (46.5); 2.502 (61.7); 2.498 (44.9); 2.493 (22.0); 2.329 (0.4); 1.355 (1.9); 0.864 (0.4); 0.008 (1.0); 0.000 (28.0); −0.009 (1.1)

No. XI-23, Solvent: <[D₆]-DMSO>, spectrometer: 399.95 MHz

δ=7.536 (2.1); 7.531 (2.3); 7.467 (0.3); 7.419 (0.4); 7.377 (2.4); 7.371 (2.3); 7.272 (0.8); 7.257 (4.0); 4.824 (0.8); 4.545 (6.9); 3.863 (2.0); 3.855 (14.2); 3.779 (2.3); 3.758 (16.0); 3.380 (0.4); 2.506 (13.2); 2.502 (18.6); 2.497 (14.9); 1.355 (0.9); 0.864 (0.4); 0.000 (19.4); −0.008 (1.7)

No. XI-24, Solvent: <[D₆]-DMSO>, spectrometer: 399.95 MHz

δ=8.038 (5.5); 8.036 (5.4); 7.964 (0.4); 7.735 (0.4); 7.729 (0.4); 7.716 (2.5); 7.711 (2.3); 7.695 (3.8); 7.690 (3.5); 7.673 (0.5); 7.653 (0.4); 7.624 (6.7); 7.603 (4.2); 7.459 (11.1); 4.899 (1.0); 4.611 (16.0); 3.397 (0.5); 3.381 (0.6); 3.365 (0.5); 2.503 (29.4)

No. XI-25, Solvent: <[D₆]-DMSO>, spectrometer: 399.95 MHz

δ=7.716 (3.6); 7.340 (0.9); 7.322 (6.2); 7.311 (3.8); 4.745 (0.7); 4.472 (9.3); 3.168 (0.4); 2.510 (8.6); 2.506 (18.4); 2.502 (24.9); 2.497 (18.4); 2.493 (9.3); 2.458 (15.4); 2.295 (16.0); 1.355 (0.4); 0.008 (0.4); 0.000 (18.2); −0.009 (1.0)

No. XI-27, Solvent: <[D₆]-DMSO>, spectrometer: 399.95 MHz

δ=7.835 (6.6); 7.401 (2.5); 7.398 (2.5); 7.382 (3.8); 7.379 (3.8); 7.340 (2.1); 7.317 (10.8); 7.296 (3.9); 4.784 (1.7); 4.510 (16.0); 3.681 (0.5); 3.677 (0.7); 3.665 (1.0); 3.648 (0.5); 3.396 (2.1); 3.381 (4.6); 3.370 (0.6); 3.364 (2.5); 3.169 (0.3); 2.559 (26.6); 2.510 (14.4); 2.506 (34.0); 2.501 (49.0); 2.497 (39.4); 2.492 (22.7); 2.404 (0.6); 2.397 (0.6); 2.333 (0.4); 2.328 (0.5); 2.324 (0.5); 1.441 (0.4); 1.423 (0.6); 1.407 (1.0); 1.401 (0.5); 1.390 (1.4); 1.385 (1.0); 1.373 (1.5); 1.355 (2.2); 1.338 (0.4); 1.327 (0.6); 1.320 (1.2); 1.303 (1.8); 1.283 (1.7); 1.265 (1.0); 1.248 (0.3); 0.893 (0.7); 0.882 (3.6); 0.875 (2.0); 0.864 (6.9); 0.857 (1.6); 0.846 (3.2); 0.000 (64.1); −0.009 (5.6); −0.150 (0.4)

### Preparation Example 7

### Preparation of 3-(bromomethyl)benzenesulfonamide, (XII-1)

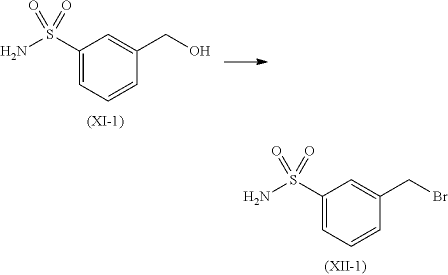 [SMILES: NS(=O)(=O)c1cccc(CO)c1 ; NS(=O)(=O)c1cccc(CBr)c1]

1.48 g (7.90 mmol)) 3-(hydroxymethyl)benzenesulfonamide were suspended in 25 ml dichloromethane and 2.35 g (8.69 mmol) phosphorus tribromide were added at 20° C. The mixture was stirred overnight. Water was added carefully and the organic phase separated. The aqueous phase was two times extracted with dichloromethane. The combined organic phases were washed with brine, dried over magnesium sulfate and concentrated in vacuo.

Yield: 1.9 g (94%) 3-(bromomethyl)benzenesulfonamide as raw product, which was pure enough for the next step.

Compounds of general formula (XII) (T=H, Y═H) are to some extent commercially available (“Comm av.”), described in the literature or can be prepared by the method described above, see table 3:

TABLE 3
Compounds of general formula (XII)

| | |
|---|---|
| | (XII) |
| 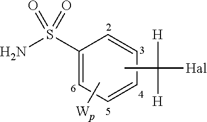 | |

T = Y = H

| | | | | | Position | |
|---|---|---|---|---|---|---|
| Ex. | | Position- | Position- | Position- | of | |
| No | p | W | W | W | CH₂-Hal | comment |
| XII-1 | 0 | — | — | — | 3-CH₂—Br | Preparative |
| | | | | | | example, |
| | | | | | | NMR |
| XII-2 | 1 | 2-C₂H₅ | — | — | 5-CH₂—Br | NMR |
| XII-3 | 1 | 2-Cl | — | — | 5-CH₂—Br | WO 2010/ |
| | | | | | | 070753 |
| | | | | | | NMR |
| XII-4 | 1 | 3-Cl | — | — | 5-CH₂—Br | Comm. av. |
| XII-5 | 1 | 3-F | — | — | 5-CH₂—Br | Comm. av. |
| XII-6 | 1 | 3-Br | — | — | 5-CH₂—Br | Comm. av. |
| XII-7 | 2 | 2-F | 4-Cl | — | 5-CH2—Br | WO 2011/ |
| | | | | | | 071565 |
| | | | | | | NMR |
| XII-8 | 1 | 4-F | — | — | 3-CH₂—Br | WO 2011/ |
| | | | | | | 071565) |
| | | | | | | NMR |
| XII-9 | 1 | 3-CH₃ | — | — | 5-CH₂—Br | |
| XII-10 | 2 | 2-Cl | 3-NO₂ | — | 5-CH₂—Br | |
| XII-11 | 1 | 4-OCH₃ | — | — | 3-CH₂—Br | |
| XII-12 | 2 | 2-Cl | 4-F | — | 5-CH₂—Br | |
| XII-13 | 1 | 2-OCH₃ | — | — | 5-CH₂—Br | NMR |
| XII-14 | 1 | 4-Br | — | — | 3-CH₂—Br | Comm. av. |
| | | | | | | NMR |
| XII-15 | 2 | 2-Cl | 4-Cl | — | 5-CH₂—Br | J. Medicinal |
| | | | | | | Chem 55(5), |
| | | | | | | 2089-2101 |
| | | | | | | (2012) |
| | | | | | | NMR |
| XII-16 | 1 | 3-F | — | — | 4-CH₂—Br | |
| XII-17 | 1 | 2-Cl | — | — | 4-CH₂—Br | |
| XII-18 | 1 | 2-F | — | — | 4-CH₂—Br | |
| XII-19 | 1 | 2-Br | — | — | 4-CH₂—Br | Comm. av. |
| XII-20 | 2 | 2-Cl | 5-Cl | — | 4-CH₂—Br | |
| XII-21 | 1 | 2-Cl | — | — | 6-CH₂—Br | |
| XII-22 | 1 | 2-OCF₃ | — | — | 5-CH₂—Br | NMR |
| XII-23 | 2 | 3-OCH₃ | 4-OCH₃ | — | 5-CH₂—Br | NMR |
| XII-24 | 1 | 4-Cl | — | — | 3-CH₂—Br | NMR |
| XII-25 | 2 | 2-CH₃ | 3-CH₃ | — | 3-CH₂—Br | NMR |
| XII-26 | 1 | 2-CF₃ | — | — | 5-CH₃—Br | |
| XII-27 | 1 | 2-CH₃ | — | — | 5-CH₂—Br | NMR |
| XII-28 | 1 | 2-F | — | — | 5-CH₂—Cl | Comm. av. |
| | | | | | | NMR |
| XII-29 | 1 | 2-Br | — | — | 5-CH₂—Br | Comm. av. |
| XII-30 | 1 | 2-Br | — | — | 3-CH₂—Br | Comm. av. |
| XII-31 | 1 | 3-Br | — | — | 4-CH₂—Br | Comm. av. |
| XII-32 | 1 | 3-Cl | — | — | 4-CH₂—Br | WO 2010/ |
| | | | | | | 093845 |
| XII-33 | 1 | 2-Br | — | — | 6-CH₂—Br | Comm. av. |
| XII-34 | 1 | 2-OC₂H₅ | — | — | 6-CH₂—Cl | EP 1344771 |

Example XII-35, cf. preparation example 9

¹H-NMR-Data of Compounds of Table 3:

No. XII-1, Solvent: <[D₆]-DMSO>, spectrometer: 399.95 MHz

δ=7.909 (2.7); 7.905 (4.5); 7.901 (2.7); 7.781 (1.6); 7.778 (2.1); 7.773 (1.6); 7.761 (1.9); 7.758 (2.5); 7.754 (1.8); 7.691 (0.4); 7.685 (1.6); 7.682 (2.0); 7.678 (1.4); 7.665 (2.3); 7.662 (2.8); 7.659 (1.8); 7.589 (3.1); 7.569 (4.4); 7.550 (1.8); 7.470 (0.6); 7.427 (7.4); 5.564 (0.3); 5.393 (0.9); 4.800 (16.0); 3.334 (12.9); 2.524 (0.5); 2.511 (10.3); 2.506 (20.4); 2.502 (26.3); 2.497 (18.6); 2.493 (8.7); 2.093 (0.4); 0.000 (1.3)

No. XII-2, Solvent: <[D₆]-DMSO>, spectrometer: 399.95 MHz

δ=7.958 (0.4); 7.954 (0.4); 7.929 (5.0); 7.925 (5.2); 7.609 (2.4); 7.604 (2.4); 7.589 (2.9); 7.585 (2.9); 7.525 (1.1); 7.493 (8.8); 7.427 (4.7); 7.407 (3.8); 5.343 (1.0); 4.757 (16.0); 3.336 (6.6); 3.026 (0.4); 3.011 (2.0); 2.993 (5.8); 2.974 (5.9); 2.955 (2.0); 2.506 (21.2); 2.502 (27.4); 2.498 (20.5); 1.240 (0.7); 1.229 (7.2); 1.222 (1.9); 1.210 (14.8); 1.192 (6.8); 0.000 (2.1)

No. XII-3, Solvent: <[D₆]-DMSO>, spectrometer: 399.95 MHz

δ=8.074 (4.8); 8.069 (5.1); 7.735 (0.4); 7.714 (0.4); 7.705 (0.8); 7.689 (2.0); 7.683 (2.3); 7.669 (10.9); 7.663 (6.3); 7.647 (7.4); 7.626 (2.5); 5.754 (0.8); 5.563 (0.3); 5.369 (0.8); 4.803 (16.0); 3.454 (0.5); 2.507 (18.1); 2.502 (23.7); 2.498 (18.6); 0.008 (0.6); 0.000 (13.6)

No. XII-7, Solvent: <[D₆]-DMSO>, spectrometer: 399.95 MHz

δ=8.098 (4.9); 8.078 (4.9); 7.956 (0.4); 7.887 (0.5); 7.877 (0.3); 7.841 (9.2); 7.816 (5.0); 7.792 (4.9); 5.659 (0.5); 5.440 (0.5); 4.827 (16.0); 3.361 (7.0); 2.525 (0.4); 2.512 (10.1); 2.507 (21.3); 2.503 (28.9); 2.498 (21.7); 2.494 (11.2)

No. XII-8, Solvent: <[D₆]-DMSO>, spectrometer: 399.95 MHz

δ=8.036 (2.8); 8.030 (3.1); 8.018 (2.9); 8.013 (3.1); 7.873 (1.5); 7.867 (1.6); 7.861 (1.7); 7.851 (2.1); 7.845 (1.8); 7.839 (1.9); 7.833 (1.7); 7.521 (0.6); 7.494 (3.3); 7.472 (14.5); 7.448 (3.0); 5.455 (0.6); 4.781 (16.0); 2.506 (17.9); 2.502 (24.4); 2.498 (19.4); 0.008 (0.6); 0.000 (15.6)

No. XII-11, Solvent: <[D₆]-DMSO>, spectrometer: 399.95 MHz

δ=7.865 (2.7); 7.859 (3.3); 7.803 (1.7); 7.797 (1.4); 7.782 (1.8); 7.776 (1.6); 7.316 (0.3); 7.275 (3.8); 7.221 (2.7); 7.199 (2.5); 5.345 (0.5); 4.687 (8.6); 3.949 (1.3); 3.942 (0.6); 3.932 (16.0); 3.427 (0.5); 2.511 (5.2); 2.506 (11.0); 2.502 (15.1); 2.497 (11.7); 2.493 (6.3); 0.000 (3.8)

No. XII-13, Solvent: <[D₆]-DMSO>, spectrometer: 399.95 MHz

δ=7.895 (0.5); 7.889 (0.4); 7.830 (3.0); 7.824 (3.1); 7.662 (1.6); 7.657 (1.5); 7.641 (1.8); 7.636 (1.6); 7.386 (0.3); 7.242 (0.8); 7.206 (2.9); 7.184 (2.7); 7.170 (0.5); 7.125 (5.4); 6.987 (0.4); 5.756 (0.8); 5.461 (0.9); 5.297 (0.6); 4.773 (9.5); 4.736 (0.4); 3.958 (1.8); 3.937 (1.2); 3.909 (16.0); 3.882 (0.4); 3.856 (0.8); 3.835 (0.9); 3.423 (0.4); 3.408 (0.4); 2.506 (21.3); 2.501 (26.3); 2.498 (20.1); 1.355 (1.3); 0.000 (6.0)

No. XII-14, Solvent: <[D₆]-DMSO>, spectrometer: 399.95 MHz

δ=8.149 (0.4); 8.144 (0.4); 8.099 (5.0); 8.093 (5.3); 8.020 (0.4); 7.999 (0.9); 7.829 (4.7); 7.809 (5.5); 7.763 (0.6); 7.743 (1.3); 7.674 (0.6); 7.644 (8.9); 7.618 (0.5); 7.612 (0.5); 7.597 (0.4); 7.591 (0.5); 7.582 (2.9); 7.576 (3.0); 7.562 (2.5); 7.556 (2.6); 7.543 (1.1); 5.755 (0.6); 5.555 (0.9); 5.359 (0.6); 4.786 (16.0); 4.528 (1.5); 3.566 (0.4); 2.506 (20.5); 2.502 (27.0); 2.498 (21.2); 1.356 (1.1); 0.008 (1.0); 0.000 (24.2)

No. XII-15, Solvent: <[D₆]-DMSO>, spectrometer: 399.95 MHz

δ=8.231 (10.0); 8.192 (0.4); 8.116 (0.4); 8.008 (0.4); 7.928 (8.5); 7.880 (0.5); 7.815 (0.5); 7.775 (10.2); 5.675 (0.5); 5.453 (0.6); 4.830 (16.0); 3.361 (6.5); 2.506 (33.0); 2.502 (41.4); 2.498 (32.6); 0.000 (7.7)

No. XII-22, Solvent: <[D₆]-DMSO>, spectrometer: 399.95 MHz

δ=8.032 (5.0); 8.026 (5.2); 7.813 (2.5); 7.807 (2.3); 7.791 (3.0); 7.785 (3.0); 7.722 (0.7); 7.697 (8.2); 7.572 (2.2); 7.568 (2.3); 7.564 (1.0); 7.551 (1.9); 7.547 (1.9); 5.387 (0.8); 4.821 (16.0); 3.381 (2.0); 2.525 (0.4); 2.512 (10.6); 2.507 (21.5); 2.503 (27.8); 2.498 (19.6); 2.494 (9.1); 1.356 (0.7); 0.008 (0.4); 0.000 (11.7); −0.009 (0.4)

No. XII-23, Solvent: <[D₆]-DMSO>, spectrometer: 399.95 MHz

δ=7.506 (2.2); 7.501 (3.0); 7.471 (2.7); 7.465 (2.2); 7.348 (4.2); 5.346 (0.4); 4.686 (7.3); 3.926 (1.1); 3.897 (16.0); 3.884 (13.4); 2.511 (4.7); 2.506 (9.7); 2.502 (13.2); 2.497 (10.0); 2.493 (5.2); 0.000 (10.0); −0.008 (0.5)

No. XII-24, Solvent: <[D₆]-DMSO>, spectrometer: 399.95 MHz

δ=8.066 (4.8); 8.060 (5.2); 7.806 (1.9); 7.801 (1.9); 7.793 (0.4); 7.785 (3.6); 7.780 (3.8); 7.743 (6.4); 7.722 (3.2); 7.580 (0.4); 7.532 (9.0); 5.755 (0.6); 5.481 (0.5); 4.828 (16.0); 3.364 (2.2); 2.507 (14.1); 2.502 (18.9); 2.498 (14.8); 0.000 (5.1)

No. XII-25, Solvent: <[D₆]-DMSO>, spectrometer: 399.95 MHz

δ=7.812 (3.1); 7.808 (3.2); 7.543 (0.6); 7.474 (3.2); 7.471 (3.3); 7.437 (6.0); 5.757 (0.5); 5.455 (0.7); 5.301 (0.6); 4.713 (11.3); 3.355 (1.4); 2.538 (1.3); 2.524 (0.5); 2.511 (11.1); 2.506 (20.7); 2.502 (26.7); 2.497 (19.0); 2.493 (9.0); 2.476 (16.0); 2.369 (1.2); 2.343 (0.9); 2.304 (15.2); 0.000 (5.6)

No. XII-27, Solvent: <[D₆]-DMSO>, spectrometer: 399.95 MHz

δ=7.931 (3.3); 7.926 (3.5); 7.585 (0.3); 7.562 (1.9); 7.558 (1.7); 7.543 (2.2); 7.538 (2.2); 7.465 (0.5); 7.458 (0.3); 7.435 (5.9); 7.375 (2.8); 7.356 (2.3); 5.510 (0.7); 5.343 (0.6); 4.760 (11.5); 3.345 (1.6); 2.644 (1.2); 2.614 (0.9); 2.576 (16.0); 2.510 (8.0); 2.506 (16.4); 2.502 (21.9); 2.497 (16.3); 2.493 (8.1); 0.000 (3.2)

No. XII-28, Solvent: <[D₆]-DMSO>, spectrometer: 399.95 MHz

δ=7.900 (2.1); 7.897 (2.2); 7.889 (2.2); 7.885 (2.2); 7.741 (9.6); 7.732 (1.9); 7.726 (1.5); 7.722 (1.5); 7.718 (1.2); 7.464 (2.2); 7.450 (2.4); 7.447 (2.7); 7.433 (2.1); 4.872 (0.4); 4.859 (16.0); 3.348 (8.3); 2.506 (15.6); 2.503 (21.3); 2.500 (15.7); 2.498 (7.4); 0.005 (1.0); 0.000 (29.9); −0.006 (1.1)

No. XII-35, Solvent: <[D₆]-DMSO>, spectrometer: 399.95 MHz

δ=8.092 (6.9); 8.086 (7.0); 8.064 (1.7); 8.060 (1.6); 7.773 (0.7); 7.761 (3.1); 7.754 (4.1); 7.740 (6.0); 7.735 (4.9); 7.725 (3.7); 7.688 (12.7); 7.661 (8.4); 7.640 (5.6); 7.630 (0.7); 7.626 (0.7); 5.788 (0.9); 5.772 (1.0); 5.757 (1.0); 5.642 (1.2); 5.625 (4.0); 5.608 (4.0); 5.590 (1.2); 3.344 (16.4); 2.672 (0.4); 2.507 (53.5); 2.502 (67.5); 2.498 (49.7); 2.329 (0.4); 1.991 (16.0); 1.974 (15.8); 1.681 (3.5); 1.665 (3.5); 1.313 (0.3); 1.235 (0.3); 0.008 (1.9); 0.000 (40.6)

### Preparation Example 8

### Preparation of 3-(1H-pyrazol-1-ylmethyl)benzenesulfonamide (IIIA-1)

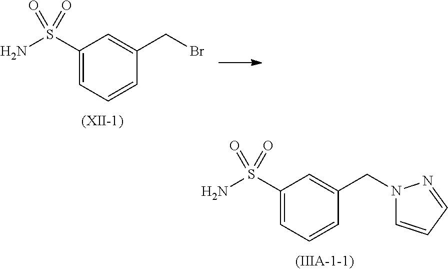 [SMILES: NS(=O)(=O)c1cccc(CBr)c1 ; NS(=O)(=O)c1cccc(Cn2cccn2)c1]

625 mg (2.50 mmol) 3-(bromomethyl)benzenesulfonamide and 851 mg (12.5 mmol) 1H-pyrazole were solved in 25 ml acetonitrile and heated under reflux for 16 h. The cooled solution was poured into water and saturated sodium hydrogencarbonate solution was added until neutral reaction. The aqueous phase was extracted three times with methylenchloride, dried over magnesium sulfate and concentrated in vacuo. To remove impurities of pyrazole the residue was taken up in methylenchloride and washed with about 10 ml 1 N aqueous hydrochloric acid. After drying and solvent evaporation 310 mg (purity 96.5%, 50.4% of theory) of 3-(1H-pyrazol-1-ylmethyl)benzenesulfonamide.

Compounds of general formula (IIIA) (T=H, Y═H) are to some extent commercially available (“Comm av.”), described in the literature or can be prepared by the method described above, see table 4:

TABLE 4
Compounds of general formula (IIIA)

| | |
|---|---|
| | (IIIA) |
| 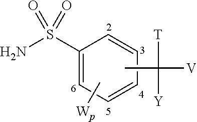 | |

T = Y = H

| Ex. | | Position- | Position- | Position- | Position | | |
|---|---|---|---|---|---|---|---|
| No | p | W | W | W | of CH₂—V | V | comment |
| IIIA-1-1 | 0 | — | — | — | 3 | 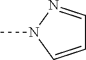 | Preparative example, NMR |
| IIIA-1-2 | 0 | — | — | — | 3 | 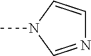 | Comm. av. |
| IIIA-2-1 | 1 | 2-C₂H₅ | — | — | 5 | 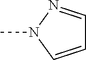 | NMR |
| IIIA-3-1 | 1 | 2-Cl | — | — | 5 | 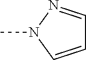 | NMR |
| IIIA-3-2 | 1 | 2-Cl | — | — | 5 | 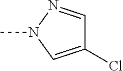 | |
| IIIA-4-1 | 1 | 3-Cl | — | — | 5 | 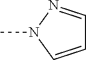 | |
| IIIA-5-1 | 1 | 3-F | — | — | 5 | 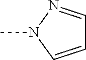 | |
| IIIA-6-1 | 1 | 3-Br | — | — | 5 | 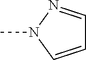 | |
| IIIA-7-1 | 2 | 2-F | 4-Cl | — | 5 | 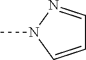 | NMR |
| IIIA-8-1 | 1 | 4-F | — | — | 3 | 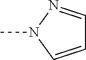 | NMR |
| IIIA-9-1 | 1 | 3-CH₃ | — | — | 5 | 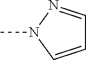 | |
| IIIA-10-1 | 2 | 2-Cl | 3-NO₂ | — | 5 | 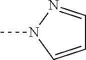 | |
| IIIA-11-1 | 1 | 4-OCH₃ | — | — | 3 | 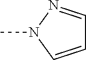 | NMR |
| IIIA-12-1 | 2 | 2-Cl | 4-F | — | 5 | 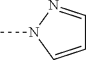 | |
| IIIA-13-1 | 1 | 2-OCH₃ | — | — | 5 | 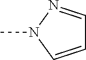 | NMR |
| IIIA-14-1 | 1 | 4-Br | — | — | 3 | 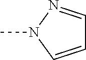 | NMR |
| IIIA-15-1 | 2 | 2-Cl | 4-Cl | — | 5 | 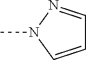 | NMR |
| IIIA-16-1 | 1 | 3-F | — | — | 4 | 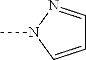 | |
| IIIA-17-1 | 1 | 2-Cl | — | — | 4 | 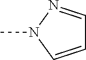 | |
| IIIA-18-1 | 1 | 2-F | — | — | 4 | 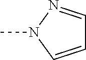 | |
| IIIA-19-1 | 1 | 2-Br | — | — | 5 | 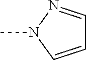 | |
| IIIA-20-1 | 2 | 2-Cl | 5-Cl | — | 4 | 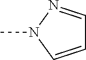 | |
| IIIA-21-1 | 1 | 2-Cl | — | — | 6 | 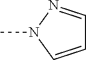 | |
| IIIA-22-1 | 1 | 2-OCF₃ | — | — | 5 | 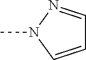 | NMR |
| IIIA-23-1 | 2 | 3-OCH₃ | 4-OCH₃ | — | 5 | 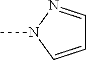 | NMR |
| IIIA-24-1 | 1 | 4-Cl | — | — | 3 | 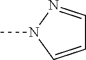 | NMR |
| IIIA-25-1 | 2 | 2-CH₃ | 3-CH₃ | — | 5 | 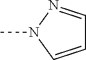 | NMR |
| IIIA-26-1 | 1 | 2-CF₃ | — | — | 5 | 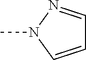 | |
| IIIA-27-1 | 1 | 2-CH₃ | — | — | 5 | 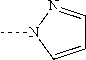 | NMR |
| IIIA-28-1 | 1 | 2-F | — | — | 5 | 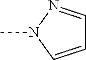 | NMR |
| IIIA-29-1 | 1 | 2-Br | — | — | 5 | 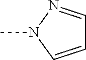 | NMR |
| IIIA-30-1 | 1 | 2-Br | — | — | 4 | 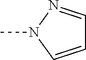 | |
| IIIA-31-1 | 1 | 3-Br | — | — | 5 | 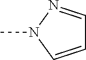 | |
| IIIA-32-1 | 1 | 3-Cl | — | — | 4 | 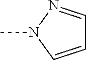 | |
| IIIA-33-1 | 1 | 2-Br | — | — | 6 | 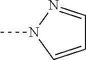 | |
| IIIA-34-1 | 1 | 2-OC₂H₅ | — | — | 6 | 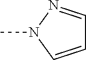 | |
| IIIA-36-1 | 1 | 5-Cl | — | — | 2 | 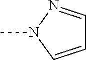 | |

Example IIIA-35-1, cf. preparation example 10.

Example IIIA-36-1, cf. preparation example 13.

¹H-NMR-Data of Compounds of Table 4:

No. IIIA-1-1, Solvent: <[D₆]-DMSO>, spectrometer: 399.95 MHz

δ=7.8753 (5.16); 7.8698 (5.12); 7.757 (2.44); 7.7374 (2.95); 7.676 (4.98); 7.5601 (2.46); 7.5408 (5.12); 7.5215 (2.81); 7.4883 (4.9); 7.4841 (4.97); 7.4313 (3.24); 7.4122 (2.47); 7.3762 (9.74); 6.2992 (3.23); 6.2944 (5.69); 6.2895 (3.14); 5.4309 (16); 3.3304 (21.02); 2.5052 (20.86); 2.5011 (26.77); 2.4971 (19.79); 2.0929 (0.47); 2.0471 (0.33); −0.0002 (6.4); −0.007 (0.32)

No. IIIA-2-1, Solvent: <[D₆]-DMSO>, spectrometer: 399.95 MHz

δ=7.8449 (5.01); 7.8394 (5.11); 7.7569 (5.31), 7.467 (4.82); 7.4627 (5.02); 7.4221 (9.24); 7.3989 (1.67); 7.3791 (8.09); 7.3728 (5.49); 7.369 (4.84); 7.3529 (0.89); 7.3489 (0.98); 6.2806 (3.54); 6.2757 (5.92); 6.2704 (3.46); 5.3596 (16); 3.3311 (34.97); 2.9856 (1.94); 2.967 (6.2); 2.9484 (6.3); 2.9298 (2.03); 2.5096 (10.34); 2.5054 (20.41); 2.501 (26.63); 2.4966 (19.39); 1.2069 (7.56); 1.1883 (15.84); 1.1697 (7.25); 0.0079 (1.06); −0.0002 (25.37); −0.0085 (1.03)

No. IIIA-3-1, Solvent: <[D₆]-DMSO>, spectrometer: 399.95 MHz

δ=7.877 (5.2); 7.872 (5.4); 7.842 (5.2); 7.838 (5.3); 7.626 (5.0); 7.605 (15.8); 7.490 (5.3); 7.487 (5.4); 7.440 (3.1); 7.436 (3.0); 7.420 (2.5); 7.415 (2.4); 6.297 (3.6); 6.292 (5.7); 6.287 (3.3); 5.421 (16.0); 3.391 (0.6); 3.374 (0.6); 3.356 (0.6); 3.335 (8.3); 2.501 (32.3); 1.108 (0.5); 1.091 (1.1); 1.073 (0.5); 0.000 (25.6)

No. IIIA-7-1, Solvent: <[D₆]-DMSO>, spectrometer: 399.95 MHz

δ=7.883 (5.8); 7.878 (5.8); 7.786 (6.6); 7.773 (11.0); 7.761 (6.7); 7.519 (5.7); 7.516 (5.6); 7.421 (4.8); 7.402 (4.8); 6.324 (4.4); 6.319 (7.1); 6.314 (4.3); 5.481 (16.0); 3.326 (36.4); 2.510 (15.0); 2.506 (29.7); 2.501 (38.9); 2.497 (28.4); 2.493 (13.9); 0.008 (0.8); 0.000 (21.9); −0.009 (0.8) No. IIIA-8-1, Solvent: <[D₆]-DMSO>, spectrometer: 399.95 MHz

δ=7.869 (6.1); 7.863 (6.4); 7.841 (1.6); 7.835 (1.9); 7.829 (1.9); 7.823 (2.1); 7.820 (2.2); 7.813 (2.2); 7.807 (2.0); 7.801 (2.0); 7.630 (2.9); 7.624 (3.0); 7.612 (3.1); 7.607 (3.0); 7.490 (6.0); 7.486 (6.3); 7.485 (6.2); 7.472 (3.9); 7.448 (4.9); 7.420 (12.3); 6.304 (4.9); 6.298 (8.0); 6.293 (5.3); 5.467 (16.0); 3.391 (0.4); 3.374 (0.4); 3.326 (20.2); 2.523 (0.5); 2.518 (0.8); 2.510 (11.2); 2.505 (23.7); 2.501 (32.4); 2.496 (24.7); 2.492 (13.0); 1.108 (0.4); 1.091 (0.9); 1.073 (0.4); 0.008 (1.1); 0.000 (34.9); −0.008 (2.0)

No. IIIA-11-1, Solvent: <[D₆]-DMSO>, spectrometer: 399.95 MHz

δ=7.779 (3.0); 7.773 (4.3); 7.768 (1.8); 7.752 (1.6); 7.746 (1.6); 7.472 (2.8); 7.468 (2.8); 7.318 (2.6); 7.313 (2.6); 7.211 (8.4); 7.190 (2.9); 6.286 (1.9); 6.281 (3.3); 6.276 (2.0); 5.327 (8.7); 3.901 (16.0); 3.325 (9.6); 2.505 (12.9); 2.501 (17.0); 2.496 (13.0); 0.008 (0.7); 0.000 (15.6); −0.008 (1.0)

No. IIIA-13-1, Solvent: <[D₆]-DMSO>, spectrometer: 399.95 MHz

δ=7.826 (3.0); 7.821 (3.2); 7.649 (2.8); 7.644 (3.2); 7.490 (1.4); 7.485 (1.5); 7.469 (1.7); 7.464 (1.7); 7.449 (2.9); 7.445 (3.1); 7.183 (3.0); 7.162 (2.6); 7.051 (5.9); 6.986 (0.4); 6.258 (2.2); 6.253 (3.9); 6.248 (2.6); 5.312 (9.0); 5.289 (0.5); 3.897 (0.5); 3.876 (16.0); 3.806 (1.1); 3.356 (0.4); 3.335 (0.4); 2.504 (12.6); 2.500 (16.8); 2.496 (13.6); 0.000 (9.9)

No. IIIA-14-1, Solvent: <[D₆]-DMSO>, spectrometer: 399.95 MHz

δ=7.881 (5.4); 7.874 (8.2); 7.868 (5.8); 7.801 (5.1); 7.781 (5.6); 7.575 (10.3); 7.543 (0.4); 7.489 (5.1); 7.485 (5.3); 7.332 (2.8); 7.327 (2.9); 7.312 (2.6); 7.307 (2.6); 6.297 (3.4); 6.292 (5.8); 6.287 (3.6); 5.403 (16.0); 3.391 (0.5); 3.374 (0.6); 3.327 (18.4); 2.505 (24.5); 2.501 (31.8); 2.497 (25.2); 1.108 (0.6); 1.091 (1.1); 1.073 (0.5); 0.000 (6.6)

No. IIIA-15-1, Solvent: <[D₆]-DMSO>, spectrometer: 399.95 MHz

δ=7.898 (11.0); 7.892 (5.6); 7.886 (5.2); 7.700 (9.4); 7.550 (9.1); 7.526 (5.0); 7.522 (5.0); 6.331 (3.7); 6.326 (6.1); 6.321 (3.6); 5.498 (16.0); 3.326 (46.0); 2.510 (16.8); 2.506 (32.2); 2.501 (41.6); 2.497 (30.4); 0.008 (0.9); 0.000 (21.2); −0.008 (0.9)

No. IIIA-22-1, Solvent: <[D₆]-DMSO>, spectrometer: 399.95 MHz

δ=7.892 (4.7); 7.886 (4.6); 7.796 (5.3); 7.620 (9.0); 7.540 (10.1); 7.498 (4.4); 7.494 (4.3); 6.305 (3.4); 6.300 (5.7); 6.295 (3.4); 5.445 (16.0); 3.392 (0.6); 3.374 (0.6); 3.326 (24.6); 2.524 (0.4); 2.510 (10.6); 2.506 (21.4); 2.501 (28.2); 2.497 (20.5); 2.493 (9.9); 2.057 (0.4); 1.109 (0.6); 1.091 (1.2); 1.074 (0.6); 0.008 (0.5); 0.000 (13.5); −0.009 (0.4)

No. IIIA-23-1, Solvent: <[D₆]-DMSO>, spectrometer: 399.95 MHz

δ=7.809 (2.2); 7.805 (2.3); 7.463 (2.2); 7.459 (2.3); 7.448 (2.4); 7.443 (2.6); 7.285 (4.7); 7.092 (2.3); 7.087 (2.4); 6.285 (1.6); 6.280 (2.7); 6.275 (1.7); 5.359 (7.5); 3.868 (13.3); 3.765 (16.0); 3.325 (11.8); 2.505 (10.8); 2.501 (14.5); 2.496 (11.0); 0.008 (0.7); 0.000 (18.7); −0.008 (1.1)

No. IIIA-24-1, Solvent: <[D₆]-DMSO>, spectrometer: 399.95 MHz

δ=7.891 (5.0); 7.886 (5.2); 7.781 (1.8); 7.775 (1.8); 7.760 (3.9); 7.754 (4.2); 7.726 (8.0); 7.705 (3.5); 7.529 (4.8); 7.525 (5.1); 7.474 (9.8); 7.384 (4.7); 7.378 (4.8); 6.336 (3.7); 6.331 (6.1); 6.326 (3.8); 5.517 (16.0); 3.325 (12.9); 2.523 (0.6); 2.510 (11.3); 2.506 (22.7); 2.501 (30.3); 2.497 (22.8); 2.492 (11.8); 0.008 (1.4); 0.000 (34.6); −0.008 (1.8)

No. IIIA-25-1, Solvent: <[D₆]-DMSO>, spectrometer: 399.95 MHz

δ=7.823 (3.7); 7.818 (3.7); 7.640 (3.3); 7.637 (3.4); 7.456 (3.5); 7.452 (3.4); 7.362 (6.8); 7.276 (3.3); 6.270 (2.7); 6.265 (4.5); 6.260 (2.7); 5.308 (10.6); 3.327 (22.5); 2.523 (0.5); 2.509 (9.0); 2.505 (18.0); 2.501 (23.7); 2.496 (17.4); 2.492 (8.6); 2.449 (15.9); 2.275 (16.0); 2.073 (0.4); 0.008 (0.7); 0.000 (18.9); −0.009 (0.8)

No. IIIA-27-1, Solvent: <[D₆]-DMSO>, spectrometer: 399.95 MHz

δ=7.838 (5.0); 7.837 (5.1); 7.833 (5.3); 7.747 (5.9); 7.465 (4.8); 7.462 (5.1); 7.363 (9.6); 7.332 (14.5); 7.309 (0.4); 6.278 (4.0); 6.273 (6.5); 6.268 (4.1); 5.361 (16.0); 3.325 (13.8); 2.551 (26.3); 2.523 (0.8); 2.518 (1.1); 2.510 (13.8); 2.505 (28.4); 2.501 (37.8); 2.496 (27.9); 2.492 (14.1); 0.008 (1.4); 0.000 (40.6); −0.009 (1.7)

No. IIIA-28-1, Solvent: <[D₆]-DMSO>, spectrometer: 399.95 MHz

δ=7.875 (6.2); 7.872 (6.4); 7.676 (13.2); 7.662 (3.7); 7.516 (2.4); 7.482 (6.6); 7.418 (2.7); 7.402 (3.5); 7.387 (2.3); 6.285 (6.7); 5.433 (0.4); 5.399 (16.0); 3.351 (10.3); 2.503 (43.4); 2.083 (1.4); 0.000 (31.1)

No. IIIA-29-1, Solvent: <[D₆]-DMSO>, spectrometer: 399.95 MHz

δ=7.881 (6.2); 7.875 (10.1); 7.870 (6.4); 7.802 (5.0); 7.782 (5.5); 7.578 (9.8); 7.489 (5.2); 7.486 (5.2); 7.333 (3.0); 7.328 (3.0); 7.312 (2.8); 7.307 (2.7); 6.297 (3.7); 6.292 (5.8); 6.287 (3.6); 5.404 (16.0); 5.381 (0.6); 3.391 (0.5); 3.374 (0.5); 3.356 (0.4); 3.330 (29.2); 2.671 (0.3); 2.506 (41.2); 2.502 (50.3); 2.498 (39.8); 2.328 (0.3); 1.108 (0.5); 1.091 (0.9); 1.073 (0.5); 0.000 (37.4)

No. IIIA-35-1, Solvent: <[D₆]-DMSO>, spectrometer: 399.95 MHz

δ=7.905 (1.4); 7.900 (1.4); 7.839 (1.3); 7.834 (1.4); 7.608 (14.8); 7.603 (16.0); 7.593 (2.6); 7.496 (1.3); 7.492 (1.4); 7.466 (0.8); 7.461 (0.8); 7.446 (0.6); 7.440 (0.6); 6.293 (0.8); 6.288 (1.4); 6.283 (0.9); 6.262 (4.9); 6.257 (9.2); 6.252 (5.2); 5.762 (0.7); 5.744 (0.7); 3.850 (0.4); 2.506 (21.5); 2.502 (29.5); 2.498 (23.5); 1.806 (3.1); 1.788 (3.2)

No. IIIA-36-1, Solvent: <[D₆]-DMSO>, spectrometer: 399.95 MHz

δ=8.149 (2.4); 7.928 (5.7); 7.923 (5.7); 7.899 (1.6); 7.889 (6.2); 7.884 (6.3); 7.851 (12.3); 7.687 (2.1); 7.663 (0.8); 7.657 (0.7); 7.642 (1.0); 7.636 (3.8); 7.630 (3.0); 7.615 (3.2); 7.609 (3.1); 7.579 (5.7); 7.575 (5.7); 6.699 (1.0); 6.678 (1.0); 6.617 (4.4); 6.596 (4.3); 6.370 (3.8); 6.365 (6.5); 6.360 (3.6); 5.764 (16.0); 5.750 (0.8); 5.729 (3.3); 5.717 (0.3); 4.038 (0.4); 4.020 (0.4); 3.327 (51.9); 2.675 (0.5); 2.671 (0.6); 2.666 (0.5); 2.506 (70.7); 2.502 (90.5); 2.498 (66.2); 2.333 (0.4); 2.329 (0.6); 1.989 (1.5); 1.259 (0.4); 1.235 (0.7); 1.193 (0.4); 1.175 (0.8); 1.157 (0.4); 0.008 (0.6); 0.000 (14.6); −0.008 (0.6)

### Preparation Example 9

### Preparation of 5-(1-Bromoethyl)-2-Chlorobenzenesulfonamide (XII-35)

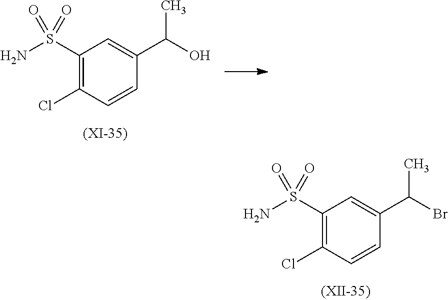

200 mg (0.85 mmol) 2-Chloro-5-(1-hydroxyethyl)benzenesulfonamide (known from JP 39019359, CAS 1965:58710) are suspended in 17 ml dichloromethane. 252.7 mg (0.93 mmol) phosphorous tribromide PBr₃ were added under stirring. After 16 h stirring at 20° C. water was added carefully and the organic phase separated. The aqueous phase was reextracted two times with dichloromethane. The combined organic phases were washed with saturated brine, dried over magnesium sulfate and concentrated in vacuo. Yield: 215.4 mg (purity by HPLC 92.6%, 78.7% of theory) 5-(1-bromoethyl)-2-chlorobenzenesulfonamide.

### Preparation Example 10

### Preparation of 2-chloro-5-[1-(1H-pyrazol-1-yl)ethyl]benzenesulfonamide (IIIA-35-1)

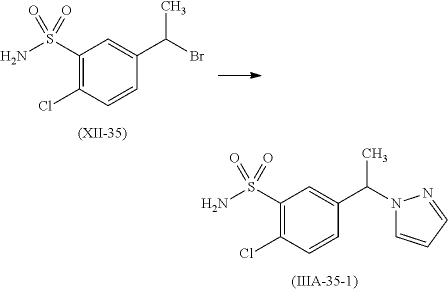 [SMILES: CC(Br)c1ccc(Cl)c(S(N)(=O)=O)c1 ; CC(c1ccc(Cl)c(S(N)(=O)=O)c1)n1cccn1]

207 mg (0.69 mmol) 5-(1-bromoethyl)-2-chlorobenzenesulfonamide (XII-35) and 236 mg (3.46 mmol) 1H-pyrazole were dissolved in 7 ml acetonitrile and heated under reflux for 2 days. 118 mg (1.73 mmol) 1H-pyrazole were added and heated further 16 h under reflux. Water was added to the cold reaction mixture and the mixture was extracted three times with dichloromethane After drying with magnesium sulfate and concentration in vacuo 198 mg (purity by HPLC 95%, 95.3% of theory) of 2-chloro-5-[1-(1H-pyrazol-1-yl)ethyl]benzenesulfonamide (IIIA-35-1).

### Preparation Example 11

### Preparation of 5-Chloro-2-(1H-Pyrazol-1-yl)methyl)benzenesulfonyl chloride/-sulfonic acid

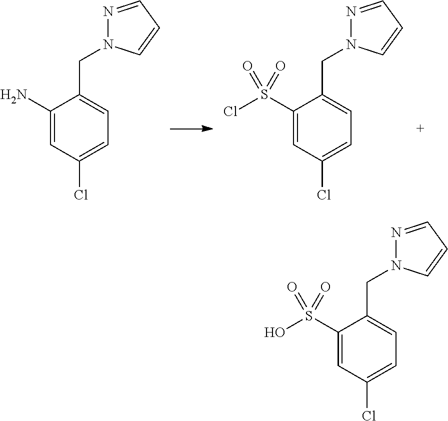 [SMILES: Nc1cc(Cl)ccc1Cn1cccn1 ; O=S(=O)(Cl)c1cc(Cl)ccc1Cn1cccn1 ; O=S(=O)(O)c1cc(Cl)ccc1Cn1cccn1]

2.0 g (9.61 mmol) 5-chloro-2-(1H-pyrazol-1-ylmethyl)aniline (known from WO 2007/014054) was taken up in 1 ml water and under cooling 2.4 g (24.35 mmol) 37% hydrochloric acid was added. A solution of 694 mg (10.06 mmol) sodium nitrite in 1.1 ml of water was added dropwise under stirring at 0° C.

This freshly prepared diazonium salt solution and 2.5 g (9.6 mmol, 40%) of a sodium hydrogen sulfite solution were simultaneously added at −10° C. to a mixture of 2 ml water, 7.83 g (37%) hydrochloric acid, 154 mg (0.963 mmol) copper sulfate pentahydrate and 2.5 g (9.6 mmol, 40%) sodium hydrogen sulfite solution.

After 3 h stirring at room temperature the reaction mixture was extracted several times with dichloromethane. The combined organic phases were washed with saturated sodium hydrogencarbonate solution, dried over magnesium sulfate and concentrated in vacuo. Yield 2.8 g raw material.

### Preparation Example 12

### Preparation of ammonium 5-chloro-2-(1H-pyrazol-1-ylmethyl)benzenesulfonate

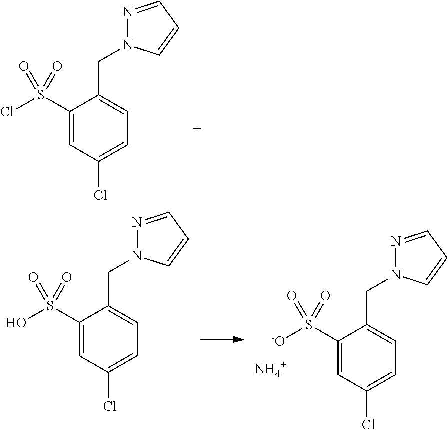

2.8 g Raw product 5-chloro-2-(1H-pyrazol-1-ylmethyl)benzenesulfonyl chloride was dissolved in 20 ml methyl tert. butyl ether (MTBE) and added at 0° C. to a mixture of 2.4 ml aqueous ammonia (28%) solution, 2.4 ml water and 10 ml MTBE. Some drops of acetone were added and the mixture stirred over night.

The organic phase was extracted several times with water and then concentrated in vacuo. The residue (7.42 g) was stirred with methanol. After filtration of insoluble salts the filtrate was evaporated, The residue (5.2 g) was stirred with 400 ml ethylacetate/methanol (1/1 by volume). Insoluble salts were removed by filtration, the filtrate evaporated to give 1.38 g of ammonium 5-chloro-2-(1H-pyrazol-1-ylmethyl)benzenesulfonate as raw material.

¹H-NMR, Solvent: <[D₆]-DMSO>, spectrometer: 399.95 MHz

δ=7.873 (4.9); 7.868 (5.0); 7.737 (5.2); 7.731 (5.3); 7.512 (6.3); 7.509 (6.4); 7.481 (1.6); 7.327 (3.0); 7.322 (2.9); 7.307 (3.0); 7.301 (2.9); 6.508 (4.5); 6.488 (4.2); 6.302 (3.5); 6.297 (5.8); 6.292 (3.4); 5.751 (16.0); 4.208 (0.6); 4.202 (1.3); 4.195 (1.5); 4.190 (2.2); 4.182 (1.5); 4.177 (1.7); 4.170 (0.6); 4.164 (0.5); 4.038 (0.6); 4.020 (0.6); 3.377 (74.4); 3.169 (16.0); 3.156 (15.6); 2.895 (0.7); 2.733 (0.7); 2.678 (0.6); 2.513 (75.3); 2.509 (94.8); 2.504 (70.9); 2.335 (0.6); 2.331 (0.5); 1.990 (2.4); 1.193 (0.6); 1.175 (1.3); 1.157 (0.7); 0.146 (0.6); 0.007 (5.3); 0.000 (112.4); −0.150 (0.6) ppm

### Preparation Example 13

### Preparation of 5-chloro-2-(1H-pyrazol-1-ylmethyl)benzenesulfonyl chloride

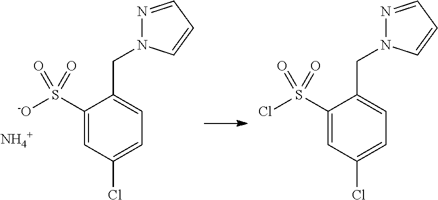

865 mg (2.98 mmol) ammonium 5-chloro-2-(1H-pyrazol-1-ylmethyl)benzenesulfonate was mixed with 6.86 g phosphoric trichloride POCl₃ and 932 mg (4.48 mmol) pentachlorophosphorane PCl₅.The mixture was heated to 60° C. and stirred for 5-6 h. The cooled reaction mixture was concentrated and two times evaporated with toluene, 869 mg 5-chloro-2-(1H-pyrazol-1-ylmethyl)benzenesulfonyl chloride as raw material.

### Preparation Example 14

### Preparation of 5-chloro-2-(1H-pyrazol-1-ylmethyl)benzenesulfonamide

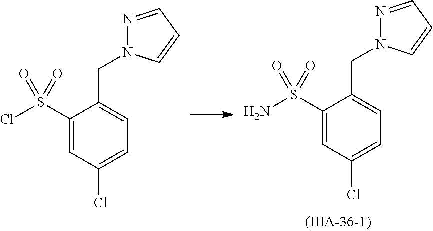 [SMILES: O=S(=O)(Cl)c1cc(Cl)ccc1Cn1cccn1 ; NS(=O)(=O)c1cc(Cl)ccc1Cn1cccn1]

869 mg 5-chloro-2-(1H-pyrazol-1-ylmethyl)benzenesulfonyl chloride raw material were taken up in 9 ml tetrahydrofurane and cooled to −10° C. 0.75 ml aqueous ammonia solution (25%) were added dropwise below 0° C. After stirring over night the mixture is concentrated in vacuo, taken up in ethyl acetate, washed with water and dried over magnesium sulfate. After evaporation 180 mg 5-chloro-2-(1H-pyrazol-1-ylmethyl)benzenesulfonamide of purity 82% (HPLC).

¹H-NMR, Solvent: <[D₆]-DMSO>, spectrometer: 399.95 MHz

δ=8.149 (2.4); 7.928 (5.7); 7.923 (5.7); 7.899 (1.6); 7.889 (6.2); 7.884 (6.3); 7.851 (12.3); 7.687 (2.1); 7.663 (0.8); 7.657 (0.7); 7.642 (1.0); 7.636 (3.8); 7.630 (3.0); 7.615 (3.2); 7.609 (3.1); 7.579 (5.7); 7.575 (5.7); 6.699 (1.0); 6.678 (1.0); 6.617 (4.4); 6.596 (4.3); 6.370 (3.8); 6.365 (6.5); 6.360 (3.6); 5.764 (16.0); 5.750 (0.8); 5.729 (3.3); 5.717 (0.3); 4.038 (0.4); 4.020 (0.4); 3.327 (51.9); 2.675 (0.5); 2.671 (0.6); 2.666 (0.5); 2.506 (70.7); 2.502 (90.5); 2.498 (66.2); 2.333 (0.4); 2.329 (0.6); 1.989 (1.5); 1.259 (0.4); 1.235 (0.7); 1.193 (0.4); 1.175 (0.8); 1.157 (0.4); 0.008 (0.6); 0.000 (14.6); −0.008 (0.6) ppm

According to the methods described above, the following compounds of general formula (I) have been prepared.

TABLE 5
Compounds of general formula (Ia), A¹ = C—R^{1a}, A² = C—R^{1b}; A³ = C—R^{1c}, A⁴ = C—R^{1d}:

| | |
|---|---|
| | (Ia) |
| 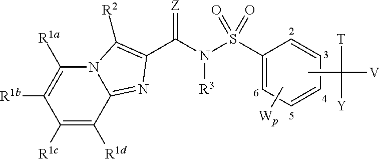 | |

| Ex. | | | | | | | | | | | Postion of | |
|---|---|---|---|---|---|---|---|---|---|---|---|---|
| No | R^{1a} | R^{1b} | R^{1c} | R^{1d} | R² | Z | R³ | W | T | Y | C(T)(Y)(V) | V |
| Ia-1 | H | CF₃ | H | Cl | H | O | H | 2-Cl | H | H | 5 | 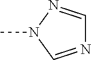 |
| Ia-2 | H | CF₃ | H | Cl | H | O | H | 2-Cl | H | H | 5 | 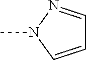 |
| Ia-3 | H | CF₃ | H | Cl | H | O | H | 2-Cl | H | H | 5 | 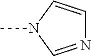 |
| Ia-4 | H | CF₃ | H | Cl | H | O | H | 2-Cl | H | H | 5 | 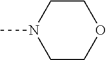 |
| Ia-5 | H | CF₃ | H | Cl | H | O | H | 2-Cl | H | H | 5 | 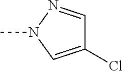 |
| Ia-6 | H | CF₃ | H | Cl | H | O | H | 2-Cl | H | H | 5 | 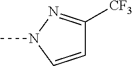 |
| Ia-7 | H | CF₃ | H | Cl | H | O | H | 2-Cl | H | H | 5 | 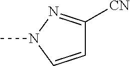 |
| Ia-8 | H | CF₃ | H | Cl | H | O | H | 2-Cl | H | H | 5 | 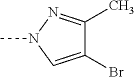 |
| Ia-9 | H | CF₃ | H | Cl | H | O | H | 2-Cl | H | H | 5 | 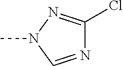 |
| Ia-10 | H | CF₃ | H | Cl | H | O | H | 2-Cl | H | H | 5 | 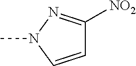 |
| Ia-11 | H | CF₃ | H | Cl | H | O | H | 2-Cl | H | H | 5 | 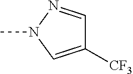 |
| Ia-12 | H | CF₃ | H | Cl | H | O | H | 2-Cl | H | H | 5 | 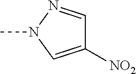 |
| Ia-13 | H | CF₃ | H | Cl | H | O | H | 2-Cl | H | H | 5 | 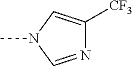 |
| Ia-14 | H | CF₃ | H | Cl | H | O | H | 2-Cl | H | H | 5 | 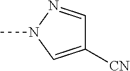 |
| Ia-15 | H | CF₃ | H | Cl | H | O | H | 2-Cl | H | H | 5 | 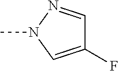 |
| Ia-16 | H | CF₃ | H | Cl | H | O | H | 2-Cl | H | H | 5 | 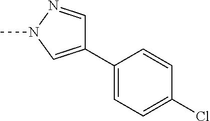 |
| Ia-17 | H | CF₃ | H | Cl | H | O | H | 2-Cl | H | H | 5 | 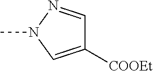 |
| Ia-18 | H | CF₃ | H | Cl | H | O | H | 2-Cl | H | H | 5 | 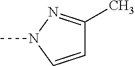 |
| Ia-19 | H | CF₃ | H | Cl | H | O | H | 2-Cl | H | H | 5 | 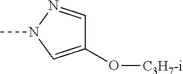 |
| Ia-20 | H | CF₃ | H | Cl | H | O | H | H | H | H | 3 | 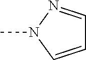 |
| Ia-21 | H | CF₃ | H | Cl | H | O | H | 2-C₂H₅ | H | H | 5 | 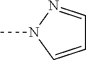 |
| Ia-22 | H | CF₃ | H | Cl | H | O | H | 2-Cl | H | H | 5 | 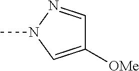 |
| Ia-23 | H | CF₃ | H | Cl | H | O | H | 2-OCF₃ | H | H | 5 | 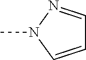 |
| Ia-24 | H | CF₃ | H | Cl | H | O | H | 2-CH₃, 3-CH₃ | H | H | 5 | 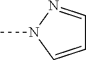 |
| Ia-25 | H | CF₃ | H | Cl | H | O | H | 2-Cl, 4-Cl | H | H | 5 | 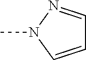 |
| Ia-26 | H | CF₃ | H | Cl | H | O | H | 2-F, 4-Cl | H | H | 5 | 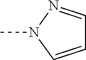 |
| Ia-27 | H | CF₃ | H | Cl | H | O | H | 2-Cl | H | H | 5 | 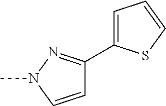 |
| Ia-28 | H | CF₃ | H | Cl | H | O | H | 2-F | H | H | 5 | 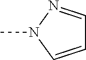 |
| Ia-29 | H | CF₃ | H | Cl | H | O | H | 4-Cl | H | H | 3 | 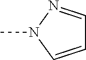 |
| Ia-30 | H | CF₃ | H | Cl | H | O | H | 4-OMe | H | H | 3 | 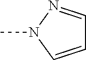 |
| Ia-31 | H | CF₃ | H | Cl | H | O | H | 2-Cl | H | H | 5 | 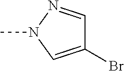 |
| Ia-32 | H | CF₃ | H | Cl | H | O | H | 2-Cl | H | H | 5 | 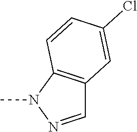 |
| Ia-33 | H | CF₃ | H | Cl | H | O | H | 2-Cl | H | H | 5 | 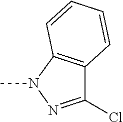 |
| Ia-34 | H | CF₃ | H | Cl | H | O | H | 2-Cl | H | H | 5 | 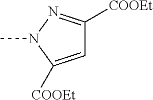 |
| Ia-35 | H | CF₃ | H | Cl | H | O | H | 2-Cl | H | H | 5 | 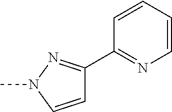 |
| Ia-36 | H | CF₃ | H | Cl | H | O | H | 2-Cl | H | H | 5 | 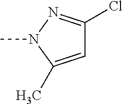 |
| Ia-37 | H | CF₃ | H | Cl | H | O | H | 2-Cl | H | H | 5 | 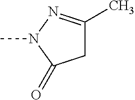 |
| Ia-38 | H | CF₃ | H | Cl | H | O | H | 2-Cl | H | H | 5 | 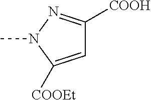 |
| Ia-39 | H | CF₃ | H | Cl | H | O | H | 3-OMe, 4-OMe | H | H | 5 | 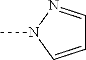 |
| Ia-40 | H | CF₃ | H | Cl | H | O | H | 4-F | H | H | 3 | 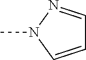 |
| Ia-41 | H | CF₃ | H | Cl | H | O | H | 4-Br | H | H | 3 | 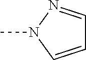 |
| Ia-42 | H | CF₃ | H | Cl | H | O | H | 2-OMe | H | H | 5 | 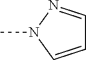 |
| Ia-43 | H | CF₃ | H | Cl | H | O | H | 2-CH₃ | H | H | 5 | 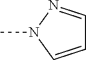 |
| Ia-44 | H | CF₃ | H | Cl | H | O | H | 2-Cl | H | H | 5 | 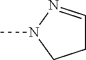 |
| Ia-45 | H | CF₃ | H | Cl | H | O | H | 2-Cl | H | H | 5 | 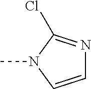 |
| Ia-46 | H | CF₃ | H | Cl | H | O | H | 2-Cl | H | H | 5 | 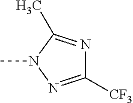 |
| Ia-47 | H | CF₃ | H | Cl | H | O | H | 2-Cl | H | H | 5 | 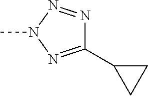 |
| | | | | | | | | | | | | 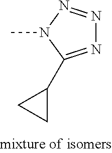 |
| Ia-48 | H | CF₃ | H | Cl | H | O | H | 2-Cl | H | H | 5 | 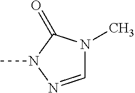 |
| Ia-49 | H | CF₃ | H | Cl | H | O | H | 2-Cl | H | H | 5 | 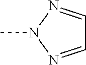 |
| | | | | | | | | | | | | 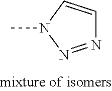 |
| Ia-50 | H | CF₃ | H | Cl | H | O | H | 2-Cl | H | H | 5 | 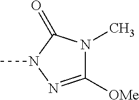 |
| Ia-51 | H | CF₃ | H | Cl | H | O | H | 2-Cl | H | H | 5 | 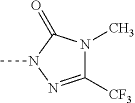 |
| Ia-52 | H | CF₃ | H | Cl | H | O | H | 2-Cl | H | H | 5 | 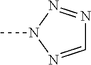 |
| | | | | | | | | | | | | 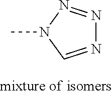 |
| Ia-53 | H | CF₃ | H | Cl | H | O | H | 2-Cl | H | H | 5 | 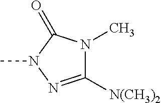 |
| Ia-54 | H | CF₃ | H | Cl | H | O | H | 2-Cl | H | H | 5 | 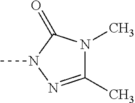 |
| Ia-55 | H | CF₃ | H | Cl | H | O | H | 2-Cl | H | H | 5 | 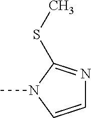 |
| Ia-56 | H | CF₃ | H | Cl | H | O | H | 2-Cl | H | H | 5 | 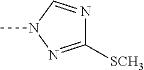 |
| Ia-57 | H | CF₃ | H | Cl | H | O | H | 2-Cl | H | H | 5 | 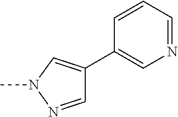 |
| Ia-58 | H | CF₃ | H | Cl | H | O | H | 2-Cl | H | H | 5 | 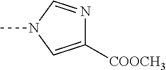 |
| | | | | | | | | | | | | 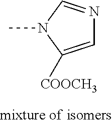 |
| Ia-59 | H | CF₃ | H | Cl | H | O | H | 2-Cl | H | H | 5 | 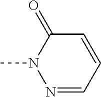 |
| Ia-60 | H | CF₃ | H | Cl | H | O | H | 5-Cl | H | H | 2 | 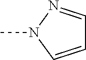 |
| Ia-61 | H | CF₃ | H | Cl | H | O | H | 2-Cl | CH₃ | H | 5 | 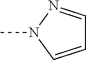 |
| Ia-62 | H | CF₃ | H | Cl | H | O | H | 2-Br | H | H | 5 | 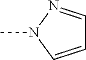 |

¹H-NMR Data of Compounds of Table 5:

No. Ia-1, Solvent: <[D₆]-DMSO>, spectrometer: 399.95 MHz

δ=2.53889 (0.1944); 3.1686 (0.3198); 3.29655 (0.0525); 5.35756 (0.0399); 5.48426 (0.3735); 7.10057 (0.0343); 7.26239 (0.0585); 7.26804 (0.0588); 7.28309 (0.0845); 7.28874 (0.0851); 7.37404 (0.1709); 7.39411 (0.1281); 7.72088 (0.1599); 7.72402 (0.1613); 7.99623 (0.333); 8.20634 (0.0407); 8.30732 (0.0536); 8.34245 (0.3243); 8.61089 (0.0635); 8.68051 (0.267); 9.21678 (0.1381);

No. Ia-2, Solvent: <[D₆]-DMSO>, spectrometer: 399.95 MHz

δ=9.3388 (4.59); 9.3358 (5.9); 8.8303 (1.2); 8.8171 (11.7); 8.166 (0.53); 8.0672 (5.42); 8.062 (5.58); 7.9614 (6.71); 7.958 (6.39); 7.9115 (5.7); 7.9062 (5.69); 7.6544 (5.19); 7.6339 (6.69); 7.6181 (1.28); 7.5185 (5.48); 7.5143 (5.55); 7.4912 (3.12); 7.4859 (3.13); 7.4705 (2.48); 7.4653 (2.49); 6.3197 (4.15); 6.3145 (6.99); 6.3095 (4.14); 5.5034 (16); 4.613 (1.29); 2.6758 (0.57); 2.6714 (0.79); 2.6669 (0.59); 2.5244 (2.72); 2.5112 (49.73); 2.5069 (97.73); 2.5024 (127.61); 2.4979 (94.18); 2.4936 (47.48); 2.3336 (0.7); 2.3292 (0.91); 2.3246 (0.69); 2.075 (2.82); 1.0452 (0.71); 1.03 (0.7); 0.0076 (0.72); −0.0002 (17.43); −0.0083 (0.77)

No. Ia-3, Solvent: <[D₆]-DMSO>, spectrometer: 399.95 MHz

δ=5.48739 (0.2736); 7.46812 (0.0832); 7.47314 (0.0974); 7.48066 (0.1617); 7.56973 (0.1126); 7.69203 (0.1218); 7.76102 (0.1092); 7.76416 (0.11); 8.1223 (0.0974); 8.12669 (0.0987); 8.42775 (0.1613); 8.98345 (0.0932); 9.23685 (0.1001).

No. Ia-4, Solvent: <[D₆]-DMSO>, spectrometer: 399.95 MHz

δ=2.53889 (0.1006); 2.97668 (0.047); 3.30534 (0.0214); 3.73936 (0.0751); 4.1897 (0.0227); 7.49948 (0.2841); 7.78486 (0.1282); 8.1317 (0.0239); 8.17875 (0.1164); 8.30732 (0.0349); 8.48043 (0.0794); 9.2519 (0.135).

No. Ia-5, Solvent: <[D₆]-DMSO>, spectrometer: 399.95 MHz

δ=9.3395 (4.52); 9.3364 (6); 8.83 (0.46); 8.8134 (11.72); 8.7677 (0.39); 8.3162 (0.39); 8.1744 (0.95); 8.1675 (10.89); 8.1662 (10.93); 8.1324 (0.84); 8.1207 (0.37); 8.0968 (5.82); 8.0916 (5.97); 8.0164 (0.34); 7.9617 (6.79); 7.9581 (6.95); 7.7673 (0.66); 7.6699 (5.53); 7.6494 (7.2); 7.6345 (0.5); 7.6203 (10.29); 7.619 (10.4); 7.5484 (0.51); 7.5337 (3.51); 7.5285 (3.46); 7.5132 (2.62); 7.5078 (2.61); 5.4689 (16); 4.6131 (0.34); 3.6763 (0.37); 3.5072 (0.57); 3.492 (0.63); 3.4749 (0.56); 3.4328 (0.48); 3.4046 (0.46); 3.3741 (0.44); 3.2895 (0.35); 3.1839 (0.35); 3.1635 (0.33); 3.1398 (0.32); 2.891 (5.11); 2.7314 (4.24); 2.6758 (0.87); 2.6713 (1.21); 2.6669 (0.89); 2.5244 (3.99); 2.5111 (74.61); 2.5068 (147.38); 2.5023 (192.49); 2.4978 (140.81); 2.4934 (70.14); 2.3334 (1); 2.329 (1.33); 2.3246 (1); 1.2399 (0.41); 0.9548 (0.37); 0.9366 (0.74); 0.858 (0.44); −0.0003 (3.59)

No. Ia-6, Solvent: <[D₆]-DMSO>, spectrometer: 399.95 MHz

δ=9.337 (7.03); 8.8184 (13.57); 8.7851 (0.68); 8.1766 (5.61); 8.1734 (5.78); 8.1644 (6.78); 8.1591 (6.67); 8.0542 (0.39); 7.9999 (0.51); 7.9946 (0.54); 7.9634 (7.71); 7.9601 (7.76); 7.7971 (0.49); 7.7141 (0.33); 7.699 (5.67); 7.6785 (8.32); 7.6579 (0.74); 7.5728 (3.73); 7.5675 (3.79); 7.5522 (2.78); 7.5469 (2.81); 7.2603 (0.34); 7.0143 (0.51); 6.8019 (6.02); 6.7964 (5.8); 5.666 (1.33); 5.6351 (1.09); 5.6206 (16); 5.5889 (0.41); 3.5067 (0.36); 3.1842 (0.37); 3.1618 (0.34); 3.1409 (0.36); 2.8913 (3.01); 2.8177 (0.55); 2.7319 (2.49); 2.6763 (0.64); 2.6718 (0.91); 2.6673 (0.65); 2.525 (2.43); 2.5114 (57.1); 2.5072 (113.41); 2.5028 (148.83); 2.4983 (109.34); 2.4941 (54.58); 2.3339 (0.81); 2.3296 (1.06); 2.325 (0.8); 1.3195 (0.42); 1.3008 (0.43); 1.2346 (0.55); 0.9552 (0.84); 0.937 (1.71); 0.9186 (0.71); 0.1458 (0.39); 0.0077 (3.39); −0.0003 (92.17); −0.0084 (3.92); −0.1498 (0.44)

No. Ia-7, Solvent: <[D₆]-DMSO>, spectrometer: 399.95 MHz

δ=9.3393 (5.53); 9.3362 (6.75); 8.8215 (14.08); 8.2493 (0.45); 8.2374 (8.3); 8.2313 (8.04); 8.1477 (6.16); 8.1426 (5.97); 8.0571 (0.7); 8.0522 (0.76); 7.9642 (7.8); 7.9608 (7.45); 7.8453 (0.93); 7.84 (0.92); 7.7141 (0.77); 7.697 (5.41); 7.6765 (7.74); 7.5982 (3.85); 7.5929 (3.58); 7.5775 (2.56); 7.5722 (2.43); 7.5471 (0.36); 7.5418 (0.34); 7.2794 (1.02); 7.2741 (0.94); 7.0495 (8.52); 7.0435 (8.13); 5.7425 (1.85); 5.638 (16); 3.5023 (0.33); 2.8921 (1.85); 2.7331 (1.6); 2.6769 (0.56); 2.6728 (0.71); 2.6681 (0.55); 2.5123 (42.1); 2.5081 (79.51), 2.5036 (100.7); 2.4991 (71.85); 2.495 (34.54); 2.3348 (0.47); 2.3304 (0.62); 2.3258 (0.42); 1.2339 (0.43); 0.9376 (0.49); 0.0079 (2.84); −0.0002 (61.98); −0.0085 (2.4)

No. Ia-8, Solvent: <[D₆]-DMSO>, spectrometer: 399.95 MHz

δ=9.3411 (3.47); 8.8241 (6.99); 8.1129 (2.46); 8.1078 (2.51); 8.0783 (5.02); 8.0676 (0.34); 8.0035 (1.48); 7.9984 (1.5); 7.9654 (4.66); 7.9622 (4.67); 7.9533 (0.99); 7.6696 (2.38); 7.663 (1.67); 7.6491 (3.19); 7.6426 (2.01); 7.6221 (0.33); 7.5875 (3.04); 7.5276 (1.49); 7.5223 (1.45); 7.507 (1.19); 7.5017 (1.13); 7.4243 (0.89); 7.419 (0.95); 7.4037 (0.75); 7.3983 (0.76); 5.5249 (3.89); 5.3973 (6.45); 4.6137 (0.32); 3.6678 (0.36); 3.6526 (0.35); 3.6189 (0.39); 3.6134 (0.4); 3.53 (0.44); 3.5134 (0.45); 3.5073 (0.47); 3.4907 (0.48); 3.4751 (0.44); 3.4488 (0.4); 3.4289 (0.39); 3.3998 (0.37); 3.3592 (0.33); 2.8913 (4.61); 2.7319 (3.9); 2.6758 (0.67); 2.6714 (0.9); 2.6669 (0.67); 2.5415 (0.54); 2.5244 (3.06); 2.5111 (59.42); 2.5069 (115.2); 2.5024 (148.52); 2.4979 (108.07); 2.4938 (53.59); 2.334 (0.87); 2.3292 (1); 2.3249 (0.77); 2.3029 (0.4); 2.2755 (0.34); 2.2351 (9.56); 2.1603 (0.52); 2.1245 (1.22); 2.115 (16); 2.1015 (0.81); 0.1462 (0.39); 0.0079 (3.87), −0.0001 (92.27); −0.0084 (3.79); −0.1496 (0.43)

No. Ia-9, Solvent: <[D₆]-DMSO>, spectrometer: 399.95 MHz

δ=9.3398 (4.89); 9.3367 (6.73); 8.8854 (0.59); 8.8174 (13.37); 8.8061 (0.97); 8.7973 (14.01); 8.3166 (0.33); 8.2089 (5.95); 8.2037 (6.02); 8.1595 (1.97); 8.1104 (0.79); 8.1051 (0.8); 7.963 (7.94); 7.9595 (8.06); 7.7142 (0.46); 7.7064 (4.5); 7.6859 (8.6); 7.6461 (4.41); 7.6409 (4.08); 7.6254 (2.21); 7.6201 (2.32); 7.5666 (0.51); 7.5612 (0.47); 7.5459 (0.39); 7.5404 (0.35); 5.6121 (2.32); 5.5746 (16); 5.4485 (0.61); 3.5114 (0.37); 2.8913 (2.41); 2.7319 (1.97); 2.6761 (0.82); 2.6717 (1.1); 2.6671 (0.81); 2.5418 (0.61); 2.5247 (3.77); 2.5115 (67.1); 2.5071 (132.2); 2.5026 (172.75); 2.4981 (124.9); 2.4937 (60.83); 2.3381 (0.46); 2.3339 (0.86); 2.3293 (1.14); 2.3248 (0.85); 1.2345 (0.36); 0.8581 (0.32); 0.1459 (0.5); 0.0079 (4.96); −0.0002 (115.25), −0.0085 (4.5); −0.1496 (0.51)

No. Ia-10, Solvent: <[D₆]-DMSO>, spectrometer: 399.95 MHz

δ=10.1428 (0.71); 9.3379 (4.99); 9.3346 (6.46); 8.83 (0.42); 8.8121 (13.75); 8.7959 (0.46); 8.7802 (0.54); 8.6647 (0.59); 8.6599 (0.38); 8.3161 (0.65); 8.2474 (8.86); 8.241 (8.81); 8.1982 (6.31); 8.1931 (6.33); 7.9636 (7.32); 7.96 (7.32); 7.9532 (1.87); 7.9147 (0.35); 7.7118 (4.79); 7.6913 (8.68); 7.6473 (4.43); 7.642 (4.16); 7.6266 (2.35); 7.6213 (2.4); 7.1184 (9.24); 7.1121 (8.99); 5.8829 (0.42); 5.6569 (16); 4.5861 (0.34); 3.8671 (0.42); 3.8135 (0.5); 3.6749 (0.75); 3.5338 (0.98); 3.5171 (1.09); 3.5075 (1); 3.5022 (1); 3.4815 (0.91); 3.4143 (0.8); 3.3976 (0.77); 3.2584 (0.49); 3.1834 (0.64); 3.1632 (0.59); 3.1417 (0.59); 2.8916 (8.15); 2.7324 (6.73); 2.6807 (0.63); 2.6763 (1.17); 2.6717 (1.57); 2.6672 (1.14); 2.6626 (0.57); 2.5418 (0.98); 2.525 (5.17); 2.5116 (92.29); 2.5072 (180.04); 2.5027 (231.17), 2.4981 (163.78); 2.4937 (77.03); 2.3384 (0.58); 2.334 (1.13); 2.3294 (1.51); 2.3248 (1.09); 2.3203 (0.53); 1.3188 (0.4); 1.3009 (0.45); 1.2356 (0.52); 0.9552 (0.89); 0.9369 (1.85); 0.9185 (0.73); 0.8584 (0.36); 0.1461 (0.65); 0.0079 (6.68); −0.0002 (164.86); −0.0085 (5.83); −0.1495 (0.69)

No. Ia-11, Solvent: <[D₆]-DMSO>, spectrometer: 399.95 MHz

δ=10.1568 (0.51), 9.3494 (0.65); 9.334 (6.12); 8.8121 (1.12); 8.8 (9.8); 8.6027 (8.16); 8.4592 (0.86); 8.3163 (0.71); 8.2373 (0.33); 8.1576 (6.18); 8.1525 (6.06); 8.1358 (0.44); 8.0967 (0.65); 7.9728 (9.35); 7.9571 (7.25); 7.9541 (7.37); 7.699 (0.43); 7.6804 (5.09); 7.66 (6.88); 7.6372 (0.34); 7.5711 (3.66); 7.5661 (3.61); 7.5504 (2.52); 7.5454 (2.43); 5.5608 (16); 3.7493 (0.44); 3.5186 (1.27); 3.503 (1.45); 3.4864 (1.39); 3.4526 (1.41); 3.4326 (1.41); 3.3912 (1.33); 3.2488 (0.73); 3.232 (0.72); 3.1863 (0.94); 3.1637 (0.76); 3.1404 (0.75); 3.0898 (0.37); 3.0223 (0.32); 2.891 (5.42); 2.8307 (0.53); 2.7315 (4.6); 2.6754 (1.77); 2.6711 (2.33); 2.6667 (1.73); 2.5413 (1.32); 2.5064 (273.83); 2.502 (349.36); 2.4976 (252.56); 2.3331 (1.71); 2.3288 (2.28); 2.3243 (1.65); 1.3191 (0.49); 1.3011 (0.51), 1.2829 (0.33); 1.2348 (0.53); 0.9549 (1); 0.9366 (2); 0.9182 (0.83); 0.146 (0.89); 0.0079 (7.36); −0.0001 (196.7); −0.0083 (7.35); −0.1495 (0.93)

No. Ia-12, Solvent: <[D₆]-DMSO>, spectrometer: 399.95 MHz

δ=9.3334 (6.17); 9.1075 (11.37); 8.8227 (0.37); 8.8058 (11.13); 8.3212 (10.56); 8.1945 (6.03); 8.1897 (6.17); 7.9603 (6.95); 7.957 (7.16); 7.6937 (3.71); 7.6732 (8.36); 7.6457 (4.37); 7.6407 (4.47); 7.625 (1.93); 7.62 (2.09); 5.5756 (16); 4.5824 (0.32); 3.5654 (0.54); 3.5261 (0.63); 3.5103 (0.73); 3.4946 (0.65); 3.2343 (0.37); 3.1825 (0.52); 3.1623 (0.51); 3.1411 (0.49); 2.8912 (4.09); 2.7316 (3.51); 2.6759 (0.98); 2.6715 (1.28); 2.667 (0.99); 2.5418 (0.83); 2.5068 (147.99); 2.5024 (191.91); 2.498 (141.95); 2.3335 (0.93); 2.3292 (1.24); 2.3248 (0.94); 1.3187 (0.37); 1.3001 (0.37); 1.24 (0.58); 0.9549 (0.69); 0.9368 (1.39); 0.9184 (0.58); 0.8582 (0.53); 0.146 (0.54); 0.0077 (5.02); −0.0002 (116.2); −0.0083 (5.24); −0.1497 (0.54)

No. Ia-13, Solvent: <[D₆]-DMSO>, spectrometer: 399.95 MHz

δ=9.3363 (7.74); 8.8333 (0.5); 8.8212 (3.88); 8.8121 (13.25); 8.3163 (0.43); 8.2416 (6.84); 8.2366 (8.42); 8.0579 (7.7); 8.0242 (1.45); 8.0189 (1.43); 7.9634 (15.74); 7.9606 (13.28); 7.7071 (4.77); 7.6928 (2.13); 7.6865 (8.83); 7.6724 (1.76); 7.6438 (5.73); 7.6387 (4.47); 7.623 (2.59); 7.618 (2.48); 7.4353 (0.77); 7.4301 (0.75); 7.4147 (0.71); 7.4092 (0.64); 5.539 (3.59); 5.4348 (16); 4.6135 (0.32); 4.5869 (0.37); 3.5116 (0.35); 2.8915 (2.21); 2.7318 (1.85); 2.6763 (0.89); 2.6718 (1.19); 2.6675 (0.88); 2.5114 (77.11); 2.5073 (146.33); 2.5028 (187.63); 2.4983 (136.74); 2.4942 (67.87); 2.3339 (0.97); 2.3296 (1.27); 2.3251 (0.95); 1.2357 (0.36); −0.0002 (3.68)

No. Ia-14, Solvent: <[D₆]-DMSO>, spectrometer: 399.95 MHz

δ=9.3346 (5.79); 8.8187 (10.58); 8.7568 (10.85); 8.1375 (9.58); 8.131 (5.98); 8.1277 (5.79); 7.9613 (6.57); 7.9594 (6.09); 7.6833 (4.83); 7.6696 (6.28); 7.578 (3.22); 7.5747 (3.18); 7.5643 (2.58); 7.561 (2.53); 5.587 (16); 3.5007 (0.64); 2.892 (0.43); 2.6151 (0.45); 2.5242 (0.34); 2.5213 (0.45); 2.5183 (0.35); 2.5061 (53.55); 2.5033 (71.79); 2.5005 (53.95); 2.3877 (0.55); −0.0002 (10.07)

No. Ia-15, Solvent: <[D₆]-DMSO>, spectrometer: 399.95 MHz

δ=9.2359 (5.12); 8.3857 (1.58); 8.3157 (0.64); 8.0251 (0.42); 8.0135 (0.5); 8.0001 (4.69); 7.9887 (4.7); 7.9529 (4.53); 7.948 (4.57); 7.7514 (3.88); 7.7132 (0.42); 7.7029 (0.38); 7.5129 (4.21); 7.5025 (4.45); 7.4373 (0.46); 7.4036 (1.74); 7.3835 (2.19); 7.3603 (0.34); 7.2478 (1.78); 7.2293 (1.46); 5.3086 (13.3); 4.3357 (0.64); 4.0022 (0.49); 3.8525 (0.85); 3.7703 (0.38); 3.3237 (7.29); 2.6753 (1.14); 2.6708 (1.6); 2.6664 (1.18); 2.5411 (0.84); 2.5241 (3.79); 2.5106 (89.11); 2.5062 (181.54); 2.5017 (241.93); 2.4972 (177.67); 2.4929 (87.45); 2.333 (1.21); 2.3285 (1.64); 2.324 (1.21); 1.2354 (4.05); 1.0448 (16); 1.0295 (15.92); 0.8538 (0.46); 0.0079 (0.66); −0.0002 (21.48); −0.0085 (0.8) No. Ia-16, Solvent: <[D₆]-DMSO>, spectrometer: 399.95 MHz

δ=9.3172 (1.56); 9.3141 (2.01); 8.783 (4.16); 8.1119 (2.04); 8.1067 (2.03); 7.985 (2.62); 7.9792 (2.59); 7.9513 (2.23); 7.9478 (2.16); 7.8104 (0.51); 7.8042 (3.43); 7.7995 (1.15); 7.7875 (1.3); 7.7828 (3.8); 7.7765 (0.49); 7.6785 (1.73); 7.658 (2.44); 7.5628 (1.19); 7.5575 (1.14); 7.5421 (0.86); 7.5369 (0.84); 7.4249 (0.57); 7.4188 (3.82); 7.4141 (1.19); 7.4019 (1.18); 7.3974 (3.34); 7.3912 (0.41); 6.8144 (2.72); 6.8086 (2.66); 5.5464 (4.64); 2.5249 (0.77); 2.5115 (15.81); 2.5072 (30.59); 2.5027 (39.39); 2.4982 (28.42); 2.4939 (13.82); 2.0864 (16); 0.0078 (0.41); −0.0002 (9.58); −0.0084 (0.36)

No. Ia-17, Solvent: <[D₆]-DMSO>, spectrometer: 399.95 MHz

δ=9.2227 (4.47); 8.5116 (8.4); 8.3392 (10.2); 8.3159 (0.33); 7.9634 (4.35); 7.9581 (4.55); 7.8762 (7.9); 7.7315 (4.86); 7.7278 (5.18); 7.386 (4.01); 7.3658 (5.77); 7.2817 (2.81); 7.2762 (2.84); 7.2613 (1.95); 7.2558 (2); 5.4347 (11.17); 4.2324 (2.36); 4.2147 (7.3); 4.197 (7.35); 4.1792 (2.36); 3.3235 (120.53); 2.6754 (0.8); 2.6711 (1.08); 2.6669 (0.82); 2.5409 (1.37); 2.5107 (62.89); 2.5064 (123.03); 2.502 (162.92); 2.4976 (123.15); 2.4936 (64.29); 2.3375 (0.42); 2.3331 (0.79); 2.3288 (1.06); 2.3243 (0.81); 2.0859 (14.97); 1.7541 (1.46); 1.2808 (7.82); 1.263 (16); 1.2453 (7.56); 0.0077 (1.66); −0.0002 (37.09); −0.0077 (1.97)

No. Ia-18, Solvent: <[D₆]-DMSO>, spectrometer: 399.95 MHz

δ=9.3287 (5.23); 8.8093 (0.36); 8.784 (5.12); 8.0596 (3.14); 7.9434 (6.52); 7.7653 (3.34); 7.6293 (2.54); 7.6089 (3.42); 7.4568 (1.77); 7.4359 (1.46); 7.3957 (2.4); 7.3518 (1.04); 7.3315 (0.93); 6.1046 (2.32); 6.0707 (3.2); 5.4482 (5.39); 5.3789 (8.28); 4.6093 (0.4); 3.3731 (2.28); 2.9739 (0.52); 2.6705 (1.66); 2.5017 (268.16); 2.3977 (0.69); 2.3566 (0.77); 2.329 (2.02); 2.2434 (10.19); 2.1874 (0.36); 2.1494 (16); 2.1173 (0.44); 2.1099 (0.48); 2.0859 (12.79); 2.0352 (0.97); 1.7545 (0.95); 1.1406 (0.39); −0.0002 (42.84)

No. Ia-19, Solvent: <[D₆]-DMSO>, spectrometer: 399.95 MHz

δ=9.226 (2.6); 8.3408 (5.02); 7.9259 (2.45); 7.9208 (2.45); 7.7313 (2.91); 7.5536 (4.22); 7.3624 (2.16); 7.3421 (2.64); 7.2017 (3.95); 7.17 (1.4); 7.1649 (1.41); 7.1496 (1.16); 7.1444 (1.13); 5.2396 (6.45); 4.1854 (0.48); 4.1702 (1.15); 4.155 (1.56); 4.1399 (1.17); 4.1247 (0.47); 3.6539 (0.52); 3.3232 (73.17); 2.6706 (0.74); 2.5408 (0.65); 2.5059 (89.27); 2.5019 (112.79); 2.4977 (87.37); 2.3287 (0.75); 2.086 (1.72); 1.754 (1.23); 1.2159 (16); 1.2007 (15.92); −0.0002 (20.11)

No. Ia-20, Solvent: <[D₆]-DMSO>, spectrometer: 399.95 MHz

δ=9.218 (5.4); 8.322 (10.1); 7.841 (5.3); 7.835 (5.2); 7.777 (3.0); 7.758 (3.3); 7.735 (6.2); 7.714 (5.5); 7.457 (5.4); 7.454 (5.1); 7.371 (2.3); 7.352 (4.8); 7.333 (2.7); 7.222 (3.3); 7.203 (2.5); 6.268 (3.4); 6.264 (5.7); 6.259 (3.2); 5.372 (16.0); 5.102 (0.3); 4.134 (0.5); 4.121 (1.5); 4.107 (1.5); 4.094 (0.5); 3.345 (215.1); 3.175 (6.2); 3.162 (6.0); 2.673 (0.5); 2.542 (0.4); 2.504 (85.7); 2.331 (0.5); 2.086 (1.2); 2.073 (0.6); 1.763 (5.6); 1.235 (0.6); 0.146 (0.7); 0.000 (139.8); −0.150 (0.7)

No. Ia-21, Solvent: <[D₆]-DMSO>, spectrometer: 399.95 MHz

δ=9.326 (5.6); 8.771 (1.6); 8.758 (10.2); 8.227 (1.0); 8.209 (0.9); 7.963 (13.1); 7.878 (5.9); 7.873 (5.6); 7.487 (6.4); 7.484 (6.4); 7.446 (3.0); 7.433 (14.7); 6.993 (1.4); 6.974 (1.2); 6.295 (4.3); 6.290 (6.4); 6.285 (3.8); 5.451 (3.0); 5.438 (16.0); 5.361 (0.5); 3.392 (2.3); 3.374 (2.3); 3.358 (2.2); 3.200 (2.1); 3.187 (12.3); 3.058 (3.2); 3.040 (6.9); 3.021 (6.4); 3.003 (2.3); 2.967 (0.4); 2.949 (0.3); 2.758 (0.5); 2.748 (0.4); 2.673 (0.7); 2.508 (94.2); 2.503 (110.5); 2.499 (82.9); 2.330 (0.7); 1.990 (0.8); 1.206 (0.5); 1.189 (1.2); 1.175 (1.7); 1.150 (8.6); 1.132 (15.2); 1.113 (6.8); 1.091 (0.6); 0.986 (0.3); 0.013 (2.1); 0.000 (17.0)

No. Ia-22, Solvent: <[D₆]-DMSO>, spectrometer: 399.95 MHz

δ=9.334 (2.6); 8.798 (3.5); 8.261 (0.4); 8.047 (2.5); 8.042 (2.6); 8.031 (0.7); 7.952 (2.9); 7.923 (1.0); 7.644 (1.9); 7.621 (6.7); 7.610 (1.9); 7.523 (1.9); 7.464 (1.7); 7.443 (1.5); 7.433 (1.2); 7.415 (1.9); 7.273 (4.6); 7.265 (1.3); 5.357 (7.0); 3.748 (3.3); 3.661 (16.0); 3.653 (10.6); 3.608 (0.4); 3.480 (0.9); 3.470 (0.9); 3.442 (1.0); 3.418 (1.1); 3.395 (1.1); 3.213 (0.5); 2.671 (0.9); 2.502 (129.3); 2.329 (0.9); 2.086 (13.9); 0.000 (20.3)

No. Ia-23, Solvent: <[D₆]-DMSO>, spectrometer: 399.95 MHz

δ=9.257 (5.2); 8.338 (12.1); 8.328 (0.5); 7.918 (4.9); 7.914 (5.1); 7.882 (5.2); 7.877 (5.2); 7.742 (5.5); 7.739 (5.6); 7.490 (5.0); 7.486 (5.1); 7.327 (0.9); 7.322 (0.7); 7.305 (4.7); 7.300 (6.3); 7.273 (0.8); 6.296 (3.7); 6.291 (6.1); 6.286 (3.6); 5.422 (16.0); 4.363 (0.4); 3.461 (0.3); 3.449 (0.3); 3.444 (0.4); 3.431 (0.4); 3.340 (109.7); 2.683 (0.4); 2.678 (0.6); 2.673 (0.4); 2.513 (60.7); 2.509 (79.0); 2.504 (58.1); 2.340 (0.4); 2.336 (0.5); 2.331 (0.4); 1.241 (0.9); 1.079 (0.6); 1.061 (1.3); 1.044 (0.6)

No. Ia-24, Solvent: <[D₆]-DMSO>, spectrometer: 399.95 MHz

δ=9.321 (4.4); 8.754 (6.8); 7.968 (5.1); 7.856 (9.0); 7.480 (4.5); 7.352 (4.5); 6.284 (4.4); 5.389 (10.7); 5.310 (0.4); 3.467 (0.9); 3.449 (1.2); 3.432 (1.3); 3.414 (1.2); 2.672 (0.4); 2.502 (71.1); 2.450 (1.2); 2.329 (0.6); 2.258 (16.0); 1.056 (0.6); 0.146 (0.3); 0.000 (63.8); −0.150 (0.4)

No. Ia-25, Solvent: <[D₆]-DMSO>, spectrometer: 399.95 MHz

δ=9.333 (5.6); 8.794 (10.5); 7.962 (6.3); 7.960 (6.2); 7.944 (9.7); 7.909 (5.8); 7.904 (5.7); 7.760 (9.9); 7.545 (5.8); 7.541 (5.8); 6.343 (4.0); 6.339 (6.4); 6.333 (3.7); 5.565 (16.0); 2.672 (0.5); 2.507 (62.4); 2.503 (78.1); 2.499 (58.4); 2.334 (0.4); 2.330 (0.5); 0.146 (0.4); 0.000 (80.5); −0.150 (0.4)

No. Ia-26, Solvent: <[D₆]-DMSO>, spectrometer: 399.95MH

δ=9.328 (6.3); 8.764 (11.4); 7.966 (6.9); 7.964 (6.9); 7.898 (6.3); 7.893 (6.2); 7.828 (4.7); 7.803 (4.8); 7.648 (4.5); 7.629 (4.6); 7.538 (6.4); 7.535 (6.5); 6.338 (4.4); 6.333 (6.9); 6.328 (4.5); 5.538 (16.0); 2.672 (0.5); 2.507 (63.0); 2.503 (79.1); 2.499 (62.6); 2.330 (0.6); 1.989 (0.6); 0.146 (0.3); 0.000 (77.6); −0.150 (0.4)

No δ=. Ia-27, Solvent: <[D₆]-DMSO>, spectrometer: 399.95 MHz

δ=9.319 (6.6); 8.782 (8.0); 8.316 (0.7); 8.113 (6.3); 8.108 (6.5); 7.954 (8.2); 7.948 (12.4); 7.907 (1.1); 7.902 (1.1); 7.690 (0.8); 7.677 (0.9); 7.663 (4.5); 7.642 (6.0); 7.633 (1.9); 7.622 (0.9); 7.602 (0.9); 7.515 (3.3); 7.511 (3.4); 7.494 (2.6); 7.490 (2.6); 7.429 (5.1); 7.417 (5.4); 7.395 (5.0); 7.393 (5.2); 7.386 (5.6); 7.384 (5.2); 7.274 (0.5); 7.258 (0.5); 7.226 (0.9); 7.220 (2.5); 7.175 (0.8); 7.166 (0.7); 7.163 (0.8); 7.154 (0.6); 7.092 (1.8); 7.071 (4.4); 7.062 (4.6); 7.058 (4.9); 7.049 (4.0); 6.964 (1.6); 6.677 (7.8); 6.671 (7.9); 6.593 (1.2); 6.588 (1.3); 5.756 (0.8); 5.622 (2.4); 5.513 (16.0); 4.038 (0.5); 4.020 (0.6); 3.863 (0.4); 3.853 (0.4); 3.836 (0.4); 3.753 (0.6); 3.725 (0.7); 3.667 (0.9); 3.395 (3.2); 2.671 (1.6); 2.507 (179.9); 2.502 (232.3); 2.498 (177.5); 2.329 (1.7); 2.086 (13.3); 2.075 (2.9); 1.989 (1.6); 1.417 (0.7); 1.193 (0.5); 1.175 (0.9); 1.158 (0.5); 0.146 (0.8); 0.000 (175.0); −0.150 (0.9)

No. Ia-28, Solvent: <[D₆]-DMSO>, spectrometer: 399.95 MHz

δ=9.332 (5.9); 8.788 (12.1); 7.966 (6.4); 7.963 (6.8); 7.906 (8.2); 7.900 (6.9); 7.890 (2.9); 7.597 (1.1); 7.592 (1.3); 7.586 (1.4); 7.576 (1.8); 7.570 (1.8); 7.564 (1.8); 7.559 (1.5); 7.509 (5.7); 7.506 (6.0); 7.458 (2.8); 7.436 (2.7); 7.433 (3.4); 7.411 (2.2); 6.311 (4.3); 6.306 (7.1); 6.301 (4.5); 5.471 (16.0); 3.770 (0.3); 3.757 (0.3); 3.590 (0.4); 3.567 (0.4); 3.554 (0.4); 3.538 (0.4); 3.525 (0.4); 3.373 (0.3); 3.359 (0.3); 3.187 (2.4); 2.677 (0.4); 2.673 (0.5); 2.668 (0.4); 2.508 (51.3); 2.504 (68.7); 2.499 (52.0); 2.330 (0.5); 2.326 (0.4); 0.000 (0.5)

No. Ia-29, Solvent: <[D₆]-DMSO>, spectrometer: 399.95 MHz

δ=9.301 (4.8); 8.713 (10.9); 7.998 (2.3); 7.992 (2.4); 7.976 (7.3); 7.972 (8.2); 7.902 (4.9); 7.896 (5.1); 7.803 (5.8); 7.782 (4.8); 7.532 (9.2); 6.343 (3.4); 6.338 (5.9); 6.333 (3.7); 5.549 (13.7); 3.187 (1.2); 3.170 (16.0); 2.672 (0.3); 2.526 (0.8); 2.512 (18.3); 2.508 (37.8); 2.503 (51.0); 2.499 (38.9); 2.330 (0.3); 0.008 (1.4); 0.000 (42.1); −0.008 (2.3)

No. Ia-30, Solvent: <[D₆]-DMSO>, spectrometer: 399.95 MHz

δ=9.296 (3.0); 8.701 (5.8); 8.020 (1.4); 8.015 (1.5); 7.999 (1.6); 7.993 (1.8); 7.965 (3.0); 7.962 (3.3); 7.797 (2.9); 7.792 (3.2); 7.480 (2.9); 7.476 (3.2); 7.459 (2.8); 7.453 (3.0); 7.286 (2.7); 7.264 (2.6); 6.293 (2.1); 6.288 (3.7); 6.283 (2.5); 5.348 (8.4); 3.930 (16.0); 3.903 (0.4); 3.340 (1.4); 3.187 (1.4); 3.169 (13.9); 2.525 (0.6); 2.511 (16.8); 2.507 (34.8); 2.503 (47.5); 2.498 (37.2); 0.008 (1.2); 0.000 (34.4); −0.008 (2.4)

No. Ia-31, Solvent: <[D₆]-DMSO>, spectrometer: 399.95 MHz

δ=9.334 (6.0); 8.804 (9.6); 8.316 (0.6); 8.169 (11.0); 8.119 (0.4); 8.101 (5.7); 8.096 (6.1); 8.043 (0.4); 7.955 (6.4); 7.952 (6.7); 7.663 (4.9); 7.642 (6.7); 7.626 (10.1); 7.528 (3.2); 7.523 (3.3); 7.507 (2.5); 7.502 (2.5); 5.756 (13.1); 5.486 (16.0); 4.547 (0.8); 4.442 (0.5); 3.638 (0.4); 3.621 (0.4); 3.596 (0.4); 3.581 (0.4); 3.571 (0.4); 3.555 (0.4); 3.497 (0.7); 3.481 (0.9); 3.465 (0.7); 3.416 (0.5); 3.395 (0.5); 3.355 (0.5); 3.316 (0.4); 3.239 (0.3); 2.676 (1.0); 2.671 (1.4); 2.667 (1.1); 2.524 (3.9); 2.511 (73.2); 2.507 (147.4); 2.502 (197.9); 2.498 (151.8); 2.333 (0.9); 2.329 (1.3); 2.324 (1.0); 1.234 (0.5); 0.146 (1.5); 0.008 (13.1); 0.000 (310.2); −0.008 (19.8); −0.150 (1.5)

No. Ia-32, Solvent: <[D₆]-DMSO>, spectrometer: 399.95 MHz

δ=9.222 (5.2); 9.219 (6.4); 8.530 (9.8); 8.528 (9.9); 8.371 (0.3); 8.355 (0.4); 8.328 (15.8); 8.316 (0.8); 8.118 (1.2); 8.116 (1.3); 8.023 (6.1); 8.017 (6.4); 7.967 (0.9); 7.962 (0.9); 7.851 (0.9); 7.847 (0.9); 7.812 (6.1); 7.808 (6.1); 7.778 (0.7); 7.735 (7.8); 7.732 (7.6); 7.657 (5.1); 7.634 (5.5); 7.398 (0.8); 7.391 (5.0); 7.371 (9.4); 7.356 (0.4); 7.335 (1.3); 7.329 (4.5); 7.324 (4.5); 7.315 (1.4); 7.309 (2.5); 7.303 (2.5); 7.204 (4.6); 7.199 (4.6); 7.181 (4.1); 7.176 (4.3); 7.166 (0.6); 7.151 (0.4); 7.146 (0.4); 5.722 (16.0); 3.326 (87.9); 2.676 (0.7); 2.671 (1.0); 2.667 (0.7); 2.565 (0.5); 2.525 (2.4); 2.511 (53.2); 2.507 (109.1); 2.502 (146.9); 2.498 (110.9); 2.494 (57.8); 2.334 (0.7); 2.329 (0.9); 2.325 (0.7); 1.755 (0.5); 0.146 (0.9); 0.008 (7.4); 0.000 (198.9); −0.008 (10.9); −0.150 (0.9)

No. Ia-33, Solvent: <[D₆]-DMSO>, spectrometer: 399.95 MHz

δ=9.223 (6.3); 8.326 (11.6); 8.316 (1.6); 7.991 (5.9); 7.986 (6.3); 7.959 (0.5); 7.954 (0.4); 7.841 (4.6); 7.819 (5.3); 7.734 (7.4); 7.731 (7.6); 7.681 (4.4); 7.660 (5.1); 7.636 (0.4); 7.599 (0.3); 7.578 (0.5); 7.525 (2.4); 7.507 (3.8); 7.488 (2.4); 7.393 (0.4); 7.373 (0.6); 7.357 (5.1); 7.337 (6.8); 7.316 (0.4); 7.272 (3.2); 7.253 (4.3); 7.234 (2.8); 7.223 (3.8); 7.217 (3.7); 7.202 (2.9); 7.197 (2.9); 5.756 (3.7); 5.706 (16.0); 3.324 (61.3); 2.675 (1.4); 2.671 (1.7); 2.563 (1.4); 2.506 (190.0); 2.502 (246.4); 2.498 (192.6); 2.333 (1.1); 2.329 (1.5); 2.325 (1.2); 1.755 (1.3); 1.234 (1.3); 0.146 (1.3); 0.043 (0.5); 0.000 (256.1); −0.150 (1.2)

No. Ia-34, Solvent: <[D₆]-DMSO>, spectrometer: 399.95 MHz

δ=9.335 (3.7); 8.799 (0.4); 8.774 (4.1); 8.315 (0.5); 7.990 (3.6); 7.948 (4.4); 7.649 (2.4); 7.628 (3.3); 7.464 (1.9); 7.442 (1.5); 7.344 (0.9); 7.338 (4.3); 7.335 (6.9); 5.912 (7.9); 5.758 (1.4); 5.755 (2.3); 4.574 (0.5); 4.555 (0.6); 4.423 (0.4); 4.327 (1.9); 4.319 (2.4); 4.309 (5.9); 4.301 (5.8); 4.291 (6.1); 4.283 (5.4); 4.274 (2.3); 4.266 (1.8); 3.665 (0.3); 3.656 (0.3); 3.637 (0.4); 3.622 (0.4); 3.611 (0.4); 3.582 (0.5); 3.549 (0.5); 3.513 (0.8); 3.497 (1.0); 3.484 (0.9); 3.459 (0.7); 3.441 (0.7); 3.409 (0.8); 3.396 (0.8); 3.394 (0.8); 3.384 (0.8); 3.374 (0.8); 3.371 (0.8); 3.273 (0.5); 3.254 (0.5); 3.238 (0.5); 3.173 (0.3); 2.671 (1.3); 2.505 (171.1); 2.502 (184.4); 2.332 (1.0); 2.329 (1.1); 1.754 (0.8); 1.316 (5.5); 1.298 (16.0); 1.280 (15.7); 1.263 (5.1); 1.234 (0.4); 1.169 (0.8); 0.146 (0.9); 0.008 (25.1); 0.002 (111.0); 0.000 (186.3); −0.008 (9.4); −0.150 (0.9)

No. Ia-35, Solvent: <[D₆]-DMSO>, spectrometer: 399.95 MHz

δ=9.314 (6.8); 8.759 (8.4); 8.585 (3.5); 8.574 (3.7); 8.316 (1.7); 8.137 (6.5); 8.132 (7.0); 8.022 (7.3); 8.016 (7.6); 7.950 (3.7); 7.934 (9.4); 7.856 (1.8); 7.837 (2.9); 7.818 (1.4); 7.658 (4.6); 7.637 (6.5); 7.609 (0.6); 7.541 (3.4); 7.537 (3.6); 7.521 (2.6); 7.516 (2.7); 7.331 (2.2); 7.318 (2.7); 7.301 (2.0); 6.906 (6.7); 6.901 (7.0); 5.756 (8.0); 5.584 (16.0); 4.609 (0.5); 4.589 (0.4); 3.842 (0.4); 3.813 (0.3); 3.758 (0.5); 3.744 (0.5); 3.726 (0.5); 3.700 (0.6); 3.683 (0.7); 3.667 (0.9); 3.649 (0.9); 3.528 (2.0); 3.522 (1.9); 3.389 (4.0); 3.201 (1.2); 3.169 (2.2); 3.115 (0.7); 3.059 (0.5); 3.037 (0.5); 2.994 (0.4); 2.970 (0.4); 2.675 (2.8); 2.671 (3.8); 2.666 (3.0); 2.541 (37.3); 2.524 (9.0); 2.510 (208.7); 2.506 (431.1); 2.502 (584.0); 2.497 (447.7); 2.402 (0.4); 2.333 (2.8); 2.329 (3.8); 2.324 (2.9); 2.074 (4.7); 1.754 (1.5); 1.297 (0.7); 1.280 (0.6); 1.233 (0.5); 0.146 (3.3); 0.008 (26.4); 0.000 (760.0); −0.008 (46.7); −0.037 (1.1); −0.055 (0.5); −0.150 (3.5)

No. Ia-36, Solvent: <[D₆]-DMSO>, spectrometer: 399.95 MHz

δ=9.336 (3.5); 8.820 (4.6); 8.017 (1.2); 7.992 (3.0); 7.988 (3.1); 7.960 (4.9); 7.672 (3.6); 7.651 (4.2); 7.424 (2.3); 7.404 (2.0); 6.294 (2.3); 6.195 (5.0); 5.756 (9.2); 5.432 (9.3); 2.672 (0.7); 2.507 (81.7); 2.503 (105.8); 2.498 (82.0); 2.435 (0.5); 2.329 (0.7); 2.314 (0.3); 2.252 (16.0); 2.196 (0.3); 2.156 (7.3); 1.577 (0.7); 0.146 (0.4); 0.000 (85.7); −0.150 (0.4)

No. Ia-37, Solvent: <[D₆]-DMSO>, spectrometer: 399.95 MHz

δ=9.337 (2.8); 8.813 (4.1); 8.315 (0.4); 8.222 (2.7); 8.217 (2.9); 8.129 (0.6); 7.953 (4.2); 7.730 (1.0); 7.726 (1.0); 7.710 (1.8); 7.705 (2.0); 7.668 (3.5); 7.647 (2.0); 7.637 (1.6); 5.756 (7.6); 5.527 (4.6); 5.404 (0.6); 5.227 (8.1); 5.186 (0.3); 4.592 (1.3); 4.018 (0.4); 4.003 (0.8); 3.987 (0.4); 3.548 (0.8); 3.533 (1.2); 3.517 (1.0); 3.502 (0.8); 3.484 (0.8); 3.426 (0.8); 3.410 (0.9); 3.394 (0.9); 3.161 (0.3); 2.676 (0.7); 2.671 (1.0); 2.667 (0.8); 2.507 (112.3); 2.502 (150.6); 2.498 (115.5); 2.333 (0.8); 2.329 (1.0); 2.324 (0.8); 2.151 (16.0); 2.119 (2.6); 1.754 (0.7); 1.718 (0.3); 1.705 (0.3); 1.692 (0.3); 1.234 (0.4); 0.146 (0.7); 0.008 (5.5); 0.000 (140.2); −0.008 (8.7); −0.150 (0.7)

No. Ia-39, Solvent: <[D₆]-DMSO>, spectrometer: 399.95 MHz

δ=9.298 (2.8); 8.720 (5.0); 7.974 (3.1); 7.819 (2.8); 7.814 (2.8); 7.658 (2.8); 7.654 (2.9); 7.468 (2.8); 7.465 (2.9); 7.275 (2.8); 7.270 (2.8); 6.289 (1.9); 6.284 (3.1); 6.279 (1.9); 5.391 (8.0); 3.887 (13.8); 3.795 (16.0); 3.467 (0.9); 3.449 (2.1); 3.432 (2.2); 3.414 (1.2); 3.394 (0.8); 3.368 (0.8); 2.671 (0.4); 2.502 (62.7); 2.329 (0.4); 1.073 (1.8); 1.056 (3.4); 1.039 (1.7); 0.000 (11.8)

No. Ia-40, Solvent: <[D₆]-DMSO>, spectrometer: 399.95 MHz

δ=9.294 (6.3); 8.711 (12.6); 8.073 (1.4); 8.067 (1.7); 8.062 (1.7); 8.055 (1.9); 8.045 (1.9); 8.040 (1.8); 8.034 (1.6); 7.973 (6.8); 7.970 (7.0); 7.879 (6.2); 7.874 (6.4); 7.807 (2.7); 7.801 (2.8); 7.790 (2.9); 7.784 (2.7); 7.547 (2.8); 7.524 (4.5); 7.501 (3.1); 7.494 (6.5); 7.490 (6.5); 6.308 (4.3); 6.303 (7.3); 6.298 (4.5); 5.504 (16.0); 3.467 (0.7); 3.450 (1.1); 3.432 (1.1); 3.415 (0.8); 3.388 (0.7); 3.343 (0.7); 3.197 (0.3); 3.187 (0.8); 2.676 (0.5); 2.671 (0.7); 2.667 (0.6); 2.507 (84.2); 2.502 (110.7); 2.498 (84.8); 2.333 (0.6); 2.329 (0.7); 2.325 (0.6); 1.073 (0.5); 1.056 (0.9); 1.039 (0.5); 0.000 (23.1)

No. Ia-41, Solvent: <[D₆]-DMSO>, spectrometer: 399.95 MHz

δ=9.343 (5.3); 8.844 (10.6); 8.098 (5.3); 8.093 (5.5); 7.965 (6.2); 7.962 (6.3); 7.909 (5.8); 7.903 (5.9); 7.876 (0.4); 7.834 (5.6); 7.813 (6.2); 7.803 (0.4); 7.576 (0.5); 7.516 (5.8); 7.512 (5.9); 7.390 (2.9); 7.385 (2.9); 7.370 (2.7); 7.365 (2.7); 6.317 (4.1); 6.312 (6.9); 6.307 (4.1); 6.298 (0.3); 6.293 (0.4); 5.756 (1.4); 5.489 (16.0); 5.405 (0.8); 3.568 (0.6); 3.187 (3.5); 3.170 (0.5); 2.677 (0.5); 2.672 (0.6); 2.667 (0.5); 2.525 (1.9); 2.512 (36.5); 2.508 (71.6); 2.503 (93.4); 2.499 (68.9); 2.334 (0.4); 2.330 (0.6); 2.325 (0.4); 0.146 (0.5); 0.008 (3.9); 0.000 (95.8); −0.008 (4.1); −0.150 (0.5)

No. Ia-42, Solvent: <[D₆]-DMSO>, spectrometer: 399.95 MHz

δ=9.433 (0.5); 9.347 (1.4); 9.337 (3.2); 8.806 (5.9); 8.752 (0.5); 8.745 (0.5); 8.078 (0.5); 7.992 (0.5); 7.962 (3.6); 7.873 (3.2); 7.866 (5.5); 7.860 (3.6); 7.557 (1.4); 7.551 (1.5); 7.535 (1.6); 7.530 (1.7); 7.483 (3.0); 7.479 (3.2); 7.224 (2.8); 7.203 (2.5); 6.761 (0.3); 6.757 (0.4); 6.754 (0.4); 6.750 (0.4); 6.288 (2.1); 6.283 (3.6); 6.279 (2.2); 5.387 (8.8); 3.827 (16.0); 3.766 (0.3); 3.450 (0.5); 3.433 (0.5); 3.337 (1.2); 2.507 (37.5); 2.503 (48.5); 2.499 (38.1); 1.074 (0.5); 1.057 (0.9); 1.039 (0.5); 0.000 (44.9)

No. Ia-43, Solvent: <[D₆]-DMSO>, spectrometer: 399.95 MHz

δ=9.321 (2.6); 9.317 (3.7); 9.314 (2.7); 8.756 (9.1); 7.966 (7.7); 7.869 (3.6); 7.864 (3.7); 7.488 (3.3); 7.487 (3.5); 7.484 (3.7); 7.407 (0.6); 7.403 (0.5); 7.387 (3.5); 7.383 (4.8); 7.380 (5.7); 7.360 (0.8); 6.295 (2.9); 6.290 (4.7); 6.285 (3.0); 5.436 (10.2); 3.376 (0.3); 3.366 (0.3); 3.362 (0.3); 3.187 (0.7); 3.170 (0.6); 2.672 (0.4); 2.592 (16.0); 2.525 (0.9); 2.521 (1.4); 2.512 (21.2); 2.507 (44.2); 2.503 (59.2); 2.498 (43.8); 2.494 (21.8); 2.330 (0.4); 0.008 (2.3); 0.000 (73.4); −0.009 (2.8); −0.150 (0.3)

No. Ia-44, Solvent: <[D₆]-DMSO>, spectrometer: 399.95 MHz

δ=9.338 (0.7); 8.832 (1.0); 8.179 (0.6); 8.174 (0.6); 7.953 (2.4); 7.658 (0.4); 7.653 (0.5); 7.635 (0.9); 7.615 (0.4); 6.893 (0.7); 4.202 (1.7); 2.959 (0.4); 2.935 (0.9); 2.912 (0.6); 2.891 (16.0); 2.732 (13.9); 2.641 (0.4); 2.616 (0.7); 2.525 (0.4); 2.512 (8.2); 2.507 (16.3); 2.503 (21.5); 2.498 (15.7); 2.494 (7.8); 1.910 (7.5); 0.000 (2.3)

No. Ia-45, Solvent: <[D₆]-DMSO>, spectrometer: 399.95 MHz

δ=9.336 (1.9); 8.822 (3.8); 8.076 (1.8); 8.070 (1.9); 7.964 (2.2); 7.961 (2.5); 7.954 (2.4); 7.701 (1.8); 7.680 (2.1); 7.488 (2.6); 7.485 (2.8); 7.480 (1.2); 7.474 (1.1); 7.459 (0.8); 7.454 (0.9); 6.977 (2.5); 6.973 (2.6); 5.379 (5.1); 2.892 (16.0); 2.733 (13.7); 2.526 (0.5); 2.513 (11.7); 2.508 (23.9); 2.504 (31.6); 2.499 (23.4); 2.495 (11.7); 2.087 (10.8); 1.911 (5.8); 1.234 (0.4); 0.937 (0.6); 0.008 (0.8); 0.000 (23.8); −0.008 (0.9)

No. Ia-46, Solvent: <[D₆]-DMSO>, spectrometer: 399.95 MHz

δ=9.334 (7.2); 8.826 (0.8); 8.812 (12.2); 8.155 (6.1); 8.150 (6.5); 8.108 (0.7); 8.103 (0.7); 7.965 (7.9); 7.962 (8.3); 7.701 (5.8); 7.691 (0.5); 7.680 (7.7); 7.645 (0.8); 7.565 (3.4); 7.560 (3.5); 7.544 (2.7); 7.539 (3.0); 7.515 (0.3); 5.705 (1.5); 5.675 (16.0); 4.591 (0.8); 4.240 (0.3); 4.222 (0.5); 4.204 (0.4); 3.869 (0.4); 3.587 (0.6); 3.542 (0.8); 3.527 (1.1); 3.511 (0.9); 3.494 (0.7); 3.396 (0.6); 3.381 (0.6); 3.328 (0.9); 3.305 (0.7); 3.243 (0.5); 3.203 (0.4); 3.190 (0.4); 2.892 (3.8); 2.732 (3.1); 2.710 (0.4); 2.677 (1.0); 2.672 (1.3); 2.668 (1.0); 2.549 (46.5); 2.525 (3.5); 2.512 (70.1); 2.508 (142.3); 2.503 (187.7); 2.499 (136.9); 2.494 (67.1); 2.475 (2.9); 2.435 (0.8); 2.425 (0.4); 2.348 (4.9); 2.334 (1.0); 2.330 (1.3); 2.325 (0.9); 2.321 (0.9); 2.113 (0.5); 1.502 (0.5); 1.235 (0.4); 0.146 (0.6); 0.008 (5.0); 0.000 (141.3); −0.009 (4.8); −0.150 (0.6)

No. Ia-47, Solvent: <[D₆]-DMSO>, spectrometer: 399.95 MHz

δ=9.338 (7.6); 9.336 (7.7); 8.836 (0.5); 8.826 (1.3); 8.808 (14.7); 8.317 (0.4); 8.209 (5.7); 8.204 (6.0); 8.192 (4.2); 8.187 (4.4); 8.128 (0.4); 7.963 (11.5); 7.960 (11.6); 7.710 (5.9); 7.690 (9.9); 7.644 (4.9); 7.639 (4.3); 7.623 (2.4); 7.618 (2.4); 7.595 (2.4); 7.589 (2.4); 7.574 (1.7); 7.569 (1.8); 7.210 (0.5); 7.082 (0.5); 6.955 (0.5); 6.044 (16.0); 5.946 (11.2); 4.624 (0.4); 4.614 (0.4); 4.607 (0.7); 4.589 (0.8); 4.577 (1.2); 4.122 (0.4); 4.104 (0.4); 4.090 (0.3); 3.990 (0.3); 3.967 (0.4); 3.929 (0.4); 3.918 (0.4); 3.912 (0.4); 3.898 (0.4); 3.870 (0.5); 3.840 (0.5); 3.827 (0.5); 3.806 (0.5); 3.780 (0.5); 3.768 (0.5); 3.760 (0.5); 3.730 (0.5); 3.700 (0.5); 3.685 (0.6); 3.669 (0.6); 3.652 (0.6); 3.624 (0.6); 3.621 (0.6); 3.594 (0.6); 3.552 (0.7); 3.537 (0.8); 3.526 (0.9); 3.511 (1.1); 3.495 (0.8); 3.475 (0.6); 3.469 (0.5); 3.458 (0.5); 3.431 (0.5); 3.385 (0.5); 3.377 (0.5); 3.328 (1.3); 3.299 (0.4); 3.285 (0.4); 3.254 (0.4); 3.227 (0.4); 3.183 (0.5); 3.162 (0.5); 3.155 (0.5); 3.142 (0.5); 2.891 (3.5); 2.732 (2.8); 2.681 (0.5); 2.677 (1.0); 2.672 (1.4); 2.668 (1.0); 2.525 (2.6); 2.521 (4.2); 2.512 (75.1); 2.508 (158.3); 2.503 (214.4); 2.499 (160.2); 2.494 (81.1); 2.428 (0.4); 2.366 (0.7); 2.354 (1.3); 2.345 (1.4); 2.342 (1.2); 2.333 (3.3); 2.325 (1.9); 2.321 (2.0); 2.312 (1.4); 2.300 (0.8); 2.245 (1.0); 2.233 (1.9); 2.224 (2.0); 2.221 (1.5); 2.212 (3.8); 2.203 (1.4); 2.199 (2.3); 2.191 (2.1); 2.178 (1.3); 2.113 (0.7); 1.577 (0.4); 1.557 (0.4); 1.537 (0.3); 1.503 (0.9); 1.336 (0.4); 1.321 (0.5); 1.300 (0.5); 1.282 (0.4); 1.271 (0.3); 1.234 (0.6); 1.174 (1.2); 1.169 (1.1); 1.163 (3.3); 1.156 (4.0); 1.146 (2.2); 1.142 (3.5); 1.135 (3.9); 1.126 (1.7); 1.112 (0.4); 1.099 (1.8); 1.088 (4.9); 1.081 (6.1); 1.072 (3.0); 1.067 (5.3); 1.060 (6.1); 1.051 (2.7); 1.039 (0.6); 1.035 (0.6); 1.030 (0.4); 1.014 (0.4); 0.998 (1.6); 0.989 (4.1); 0.982 (4.0); 0.977 (4.3); 0.970 (4.3); 0.959 (1.4); 0.955 (1.2); 0.942 (2.6); 0.933 (6.4); 0.926 (5.8); 0.921 (6.4); 0.915 (6.2); 0.903 (2.2); 0.896 (0.8); 0.890 (0.7); 0.884 (0.6); 0.874 (0.4); 0.869 (0.3); 0.008 (1.1); 0.000 (38.3); −0.009 (1.7)

No. Ia-48, Solvent: <[D₆]-DMSO>, spectrometer: 399.95 MHz

δ=9.342 (2.8); 8.827 (5.1); 8.088 (2.6); 8.083 (2.6); 7.991 (5.2); 7.966 (3.3); 7.666 (2.0); 7.646 (3.4); 7.574 (1.6); 7.569 (1.5); 7.554 (1.0); 7.549 (1.0); 5.020 (7.0); 4.576 (0.4); 3.685 (0.4); 3.668 (0.5); 3.652 (0.4); 3.612 (0.3); 3.587 (0.3); 3.552 (0.3); 3.546 (0.4); 3.529 (0.4); 3.513 (0.5); 3.497 (0.6); 3.481 (0.4); 3.461 (0.4); 3.438 (0.3); 3.387 (0.4); 3.350 (0.3); 3.329 (0.6); 3.255 (0.8); 3.244 (0.4); 3.213 (16.0); 3.164 (1.1); 3.158 (0.5); 3.123 (0.6); 2.891 (1.2); 2.732 (1.0); 2.676 (0.4); 2.672 (0.5); 2.507 (56.7); 2.503 (71.3); 2.499 (53.0); 2.330 (0.4); 2.326 (0.3); 0.000 (65.5); −0.008 (3.2)

No. Ia-49, Solvent: <[D₆]-DMSO>, spectrometer: 399.95 MHz

δ=9.335 (7.9); 8.827 (1.0); 8.814 (14.4); 8.284 (0.3); 8.274 (8.6); 8.272 (9.1); 8.164 (5.2); 8.158 (5.4); 8.135 (0.5); 8.098 (2.9); 8.092 (3.0); 7.963 (9.4); 7.960 (9.6); 7.886 (16.0); 7.804 (7.9); 7.802 (8.3); 7.758 (1.0); 7.710 (0.5); 7.696 (4.8); 7.680 (3.5); 7.676 (7.4); 7.659 (4.0); 7.644 (0.9); 7.593 (3.2); 7.588 (3.2); 7.573 (2.3); 7.567 (2.4); 7.560 (2.0); 7.555 (1.9); 7.540 (1.3); 7.534 (1.4); 5.853 (8.9); 5.809 (15.1); 4.581 (0.6); 4.570 (0.7); 4.449 (0.5); 4.432 (0.4); 4.415 (0.4); 3.713 (0.4); 3.671 (0.4); 3.599 (0.4); 3.530 (0.5); 3.525 (0.5); 3.510 (0.8); 3.495 (0.8); 3.479 (0.6); 3.453 (0.4); 3.419 (0.4); 3.389 (0.4); 3.329 (0.9); 3.203 (0.4); 2.891 (2.7); 2.732 (2.2); 2.677 (0.7); 2.672 (1.0); 2.668 (0.8); 2.525 (2.4); 2.512 (54.8); 2.508 (112.8); 2.503 (150.7); 2.498 (110.8); 2.494 (54.9); 2.334 (0.7); 2.330 (1.0); 2.325 (0.8); 2.113 (0.5); 1.510 (0.5); 1.503 (0.7); 1.234 (0.5); 0.008 (0.9); 0.000 (26.5); −0.009 (1.0)

No. Ia-50, Solvent: <[D₆]-DMSO>, spectrometer: 399.95 MHz

δ=9.354 (2.7); 8.843 (5.7); 8.105 (2.6); 8.100 (2.6); 7.975 (3.2); 7.972 (3.3); 7.954 (0.8); 7.671 (2.1); 7.650 (3.4); 7.588 (1.7); 7.583 (1.7); 7.567 (1.1); 7.562 (1.1); 4.957 (6.5); 4.597 (0.3); 3.892 (16.0); 3.669 (0.4); 3.531 (0.3); 3.048 (15.1); 2.917 (0.6); 2.892 (4.0); 2.733 (3.6); 2.539 (0.4); 2.509 (31.0); 2.505 (38.6); 2.500 (29.3); 0.000 (17.4)

No. Ia-51, Solvent: <[D₆]-DMSO>, spectrometer: 399.95 MHz

δ=9.344 (6.7); 8.831 (1.2); 8.823 (14.5); 8.167 (6.3); 8.162 (6.7); 8.125 (0.4); 7.968 (7.7); 7.965 (7.9); 7.954 (1.3); 7.691 (4.9); 7.670 (9.5); 7.647 (0.9); 7.645 (0.9); 7.633 (4.6); 7.627 (4.5); 7.612 (2.3); 7.607 (2.4); 5.176 (16.0); 4.585 (0.9); 3.935 (0.3); 3.922 (0.3); 3.871 (0.4); 3.863 (0.4); 3.852 (0.4); 3.842 (0.4); 3.823 (0.6); 3.805 (0.8); 3.788 (0.6); 3.769 (0.5); 3.758 (0.5); 3.754 (0.5); 3.721 (0.5); 3.699 (0.5); 3.610 (0.8); 3.591 (0.6); 3.566 (0.6); 3.530 (0.8); 3.514 (1.0); 3.507 (0.8); 3.499 (0.7); 3.481 (0.6); 3.464 (0.5); 3.441 (0.5); 3.416 (0.5); 3.409 (0.5); 3.330 (28.9); 3.287 (1.8); 3.247 (1.2); 3.185 (0.3); 3.149 (0.4); 2.892 (7.6); 2.732 (6.3); 2.677 (0.6); 2.673 (0.8); 2.669 (0.6); 2.526 (2.0); 2.513 (41.2); 2.508 (83.9); 2.504 (111.0); 2.499 (82.5); 2.495 (41.5); 2.335 (0.5); 2.331 (0.8); 2.326 (0.6); 2.114 (0.4); 1.503 (0.5); 1.234 (0.4); 1.170 (0.4); 0.937 (0.4); 0.146 (0.5); 0.008 (3.8); 0.000 (108.4); −0.008 (4.5); −0.150 (0.5)

No. Ia-52, Solvent: <[D₆]-DMSO>, spectrometer: 399.95 MHz

δ=9.574 (9.8); 9.336 (7.5); 9.065 (5.6); 8.843 (0.6); 8.835 (1.4); 8.832 (1.2); 8.822 (16.0); 8.258 (4.5); 8.254 (4.6); 8.241 (2.9); 8.236 (2.9); 7.967 (8.8); 7.964 (8.7); 7.727 (4.5); 7.707 (11.6); 7.697 (0.9); 7.683 (5.5); 7.679 (3.8); 7.662 (2.2); 7.658 (1.6); 7.650 (0.9); 6.187 (8.5); 5.906 (12.9); 5.206 (0.7); 4.746 (0.4); 4.590 (0.6); 4.583 (0.6); 4.505 (0.3); 3.669 (0.4); 3.537 (0.5); 3.531 (0.4); 3.521 (0.7); 3.506 (0.5); 3.330 (0.8); 2.892 (2.4); 2.732 (2.1); 2.678 (0.6); 2.673 (0.7); 2.669 (0.6); 2.526 (2.1); 2.513 (39.6); 2.508 (79.3); 2.504 (104.6); 2.499 (77.0); 2.495 (38.3); 2.335 (0.5); 2.331 (0.7); 2.326 (0.5); 2.114 (1.6); 1.566 (0.3); 1.504 (0.6); 1.322 (0.4); 1.234 (0.4); 1.170 (0.3); 0.955 (0.5); 0.937 (1.0); 0.918 (0.4); 0.000 (6.5)

No. Ia-53, Solvent: <[D₆]-DMSO>, spectrometer: 399.95 MHz

δ=9.342 (1.0); 8.812 (1.7); 8.133 (0.5); 8.089 (0.9); 8.084 (0.9); 7.963 (1.3); 7.960 (1.5); 7.953 (2.4); 7.653 (0.7); 7.633 (1.1); 7.559 (0.5); 7.554 (0.6); 7.539 (0.4); 7.534 (0.4); 4.937 (2.4); 3.544 (0.4); 3.529 (0.5); 3.513 (0.5); 3.402 (0.8); 3.331 (0.6); 3.305 (0.5); 3.243 (0.4); 3.151 (6.4); 3.056 (0.5); 2.891 (16.0); 2.731 (13.7); 2.717 (0.4); 2.705 (13.7); 2.671 (1.2); 2.525 (1.0); 2.507 (38.9); 2.503 (51.1); 2.498 (38.1); 2.329 (0.3); 2.086 (0.5); 1.909 (0.7); 0.008 (0.8); 0.000 (25.7); −0.008 (1.0)

No. Ia-54, Solvent: <[D₆]-DMSO>, spectrometer: 399.95 MHz

δ=9.343 (3.1); 8.824 (5.1); 8.093 (2.7); 8.088 (2.7); 7.966 (3.6); 7.659 (2.1); 7.638 (3.2); 7.565 (1.7); 7.560 (1.7); 7.544 (1.1); 7.540 (1.1); 4.971 (7.1); 4.594 (0.5); 4.574 (0.3); 3.776 (0.3); 3.685 (0.6); 3.669 (0.8); 3.652 (0.7); 3.645 (0.6); 3.627 (0.7); 3.609 (0.7); 3.545 (0.9); 3.529 (1.1); 3.514 (1.0); 3.494 (1.0); 3.476 (0.9); 3.464 (0.9); 3.455 (0.9); 3.431 (0.9); 3.427 (0.9); 3.357 (0.8); 3.331 (1.1); 3.270 (0.6); 3.181 (0.6); 3.151 (16.0); 3.100 (0.8); 3.062 (0.4); 3.038 (0.3); 2.891 (1.1); 2.732 (1.0); 2.672 (0.7); 2.507 (73.9); 2.503 (92.3); 2.499 (72.4); 2.330 (0.7); 2.196 (0.3); 2.165 (15.3); 2.146 (0.7); 2.113 (0.3); 1.504 (0.5); 1.233 (0.4); 0.000 (63.5); −0.150 (0.4)

No. Ia-55, Solvent: <[D₆]-DMSO>, spectrometer: 399.95 MHz

δ=9.366 (2.5); 8.863 (0.4); 8.849 (5.0); 8.162 (2.4); 8.157 (2.5); 8.142 (0.5); 7.972 (3.2); 7.969 (3.2); 7.955 (2.3); 7.905 (2.4); 7.901 (2.5); 7.772 (2.2); 7.768 (2.2); 7.740 (2.0); 7.719 (2.9); 7.684 (0.5); 7.635 (1.5); 7.630 (1.5); 7.614 (1.0); 7.609 (1.0); 5.547 (5.4); 3.331 (0.4); 3.189 (2.0); 3.168 (2.0); 3.147 (2.1); 2.893 (13.3); 2.776 (0.8); 2.750 (0.8); 2.732 (12.2); 2.717 (16.0); 2.674 (0.4); 2.509 (42.8); 2.505 (54.7); 2.500 (42.0); 2.332 (0.3); 1.609 (0.5); 1.590 (1.3); 1.570 (1.8); 1.551 (1.4); 1.531 (0.7); 1.505 (0.4); 1.356 (0.5); 1.339 (1.5); 1.320 (2.8); 1.302 (2.8); 1.284 (1.6); 1.266 (0.4); 1.233 (0.4); 0.956 (5.6); 0.938 (11.1); 0.920 (4.7); 0.008 (1.1); 0.000 (26.5)

No. Ia-56, Solvent: <[D₆]-DMSO>, spectrometer: 399.95 MHz

δ=9.342 (6.8); 8.829 (16.0); 8.819 (0.5); 8.807 (0.8); 8.705 (11.9); 8.488 (0.4); 8.189 (5.2); 8.184 (5.4); 8.115 (0.3); 8.110 (0.4); 8.064 (1.2); 8.058 (1.3); 8.053 (2.8); 7.971 (7.7); 7.968 (7.9); 7.953 (1.0); 7.711 (0.6); 7.701 (4.7); 7.695 (1.5); 7.681 (7.2); 7.674 (1.8); 7.650 (0.8); 7.610 (3.3); 7.605 (3.4); 7.590 (2.2); 7.584 (2.3); 7.512 (0.8); 7.506 (0.8); 7.491 (0.5); 7.486 (0.6); 5.537 (13.2); 5.471 (3.0); 5.366 (0.9); 4.586 (0.6); 4.578 (0.4); 4.153 (0.4); 4.051 (0.4); 3.869 (0.3); 3.865 (0.3); 3.836 (0.4); 3.812 (0.4); 3.793 (0.4); 3.779 (0.4); 3.764 (0.4); 3.738 (0.4); 3.721 (0.4); 3.693 (0.4); 3.669 (0.4); 3.656 (0.4); 3.641 (0.4); 3.635 (0.4); 3.623 (0.4); 3.608 (0.4); 3.586 (0.4); 3.555 (0.4); 3.527 (0.5); 3.512 (0.7); 3.501 (0.6); 3.485 (0.5); 3.328 (0.7); 2.891 (5.6); 2.732 (4.6); 2.677 (0.8); 2.672 (1.0); 2.668 (0.7); 2.646 (9.1); 2.621 (0.7); 2.584 (2.5); 2.525 (1.7); 2.520 (3.3); 2.512 (66.4); 2.507 (139.4); 2.503 (218.2); 2.498 (141.1); 2.494 (71.5); 2.380 (0.5); 2.334 (1.1); 2.330 (1.4); 2.325 (1.3); 2.113 (0.5); 1.503 (0.6); 1.235 (0.5); 0.008 (1.6); 0.000 (59.5); −0.009 (2.7)

No. Ia-57, Solvent: <[D₆]-DMSO>, spectrometer: 399.95 MHz

δ=10.134 (0.4); 9.319 (7.1); 8.939 (5.8); 8.934 (5.8); 8.919 (0.5); 8.824 (0.9); 8.817 (0.9); 8.805 (0.8); 8.798 (0.5); 8.781 (13.8); 8.724 (0.3); 8.520 (11.7); 8.483 (3.9); 8.481 (4.3); 8.471 (4.2); 8.468 (4.3); 8.378 (0.4); 8.318 (0.6); 8.249 (0.5); 8.198 (2.5); 8.178 (2.8); 8.140 (12.8); 8.117 (6.9); 8.112 (7.1); 8.040 (0.4); 7.958 (0.9); 7.947 (8.0); 7.943 (7.9); 7.925 (0.5); 7.692 (0.3); 7.675 (6.0); 7.654 (8.7); 7.642 (0.9); 7.635 (0.7); 7.624 (1.1); 7.614 (0.8); 7.597 (0.4); 7.572 (4.8); 7.566 (6.5); 7.551 (5.4); 7.546 (5.4); 7.533 (2.4); 5.553 (16.0); 4.652 (0.4); 4.612 (0.6); 4.592 (0.9); 4.581 (0.8); 4.203 (0.4); 4.185 (0.5); 4.167 (0.4); 4.140 (0.3); 4.117 (0.4); 4.105 (0.4); 4.091 (0.4); 4.080 (0.4); 3.981 (0.4); 3.947 (0.5); 3.869 (0.6); 3.857 (0.7); 3.808 (0.7); 3.784 (0.8); 3.768 (0.8); 3.732 (0.9); 3.684 (1.4); 3.668 (1.7); 3.651 (1.5); 3.605 (1.5); 3.556 (1.7); 3.544 (2.0); 3.539 (2.0); 3.528 (2.4); 3.522 (2.2); 3.513 (2.2); 3.506 (2.1); 3.467 (2.0); 3.329 (2.3); 3.284 (1.4); 3.140 (0.8); 3.023 (0.5); 3.008 (0.5); 2.995 (0.5); 2.928 (0.4); 2.891 (1.6); 2.857 (0.4); 2.840 (0.3); 2.731 (1.4); 2.719 (0.3); 2.693 (0.4); 2.676 (1.9); 2.672 (2.6); 2.667 (2.0); 2.630 (0.4); 2.617 (0.4); 2.525 (7.1); 2.511 (135.1); 2.507 (273.1); 2.503 (361.7); 2.498 (266.5); 2.494 (131.9); 2.334 (1.7); 2.329 (2.3); 2.325 (1.7); 2.112 (0.5); 1.502 (0.8); 1.354 (0.3); 1.235 (0.7); 1.169 (0.6); 0.008 (1.2); 0.000 (39.2); −0.009 (1.4)

No. Ia-58, Solvent: <[D₆]-DMSO>, spectrometer: 399.95 MHz

δ=9.348 (1.5); 8.822 (2.8); 8.398 (0.7); 8.213 (0.7); 8.208 (0.7); 8.139 (0.4); 8.085 (1.0); 8.047 (0.8); 8.032 (0.9); 8.027 (0.9); 7.962 (2.4); 7.955 (2.8); 7.840 (0.7); 7.701 (0.5); 7.680 (1.0); 7.665 (0.8); 7.652 (0.7); 7.644 (1.4); 7.476 (0.5); 7.471 (0.5); 7.456 (0.4); 7.450 (0.4); 5.676 (1.9); 5.435 (1.7); 3.818 (0.4); 3.767 (5.2); 3.753 (0.7); 3.743 (4.5); 3.188 (1.9); 3.167 (1.9); 3.146 (2.0); 2.893 (16.0); 2.733 (14.4); 2.509 (28.5); 2.505 (36.4); 2.500 (27.7); 1.609 (0.5); 1.590 (1.2); 1.570 (1.8); 1.551 (1.4); 1.531 (0.7); 1.357 (0.4); 1.339 (1.4); 1.320 (2.6); 1.302 (2.7); 1.284 (1.6); 1.266 (0.5); 1.234 (0.4); 0.956 (5.3); 0.938 (10.7); 0.919 (4.5); 0.000 (4.5)

No. Ia-59, Solvent: <[D₆]-DMSO>, spectrometer: 399.95 MHz

δ=11.056 (0.5); 10.143 (0.6); 9.338 (6.0); 8.893 (0.9); 8.829 (1.5); 8.819 (12.4); 8.797 (0.6); 8.781 (0.5); 8.670 (0.4); 8.665 (0.6); 8.660 (0.4); 8.317 (0.6); 8.192 (0.3); 8.171 (0.4); 8.167 (0.3); 8.116 (5.6); 8.111 (5.6); 7.998 (4.1); 7.994 (4.1); 7.988 (4.3); 7.984 (4.6); 7.966 (6.8); 7.963 (6.8); 7.954 (1.8); 7.915 (0.4); 7.910 (0.3); 7.904 (0.3); 7.900 (0.4); 7.896 (0.4); 7.816 (0.4); 7.796 (0.4); 7.664 (3.6); 7.653 (0.7); 7.644 (9.2); 7.620 (4.5); 7.615 (4.3); 7.606 (0.7); 7.599 (1.8); 7.594 (1.9); 7.540 (0.5); 7.521 (0.7); 7.484 (3.9); 7.475 (3.7); 7.460 (4.3); 7.451 (4.1); 7.377 (0.4); 7.042 (4.0); 7.038 (4.1); 7.019 (3.6); 7.015 (3.6); 5.373 (16.0); 4.579 (0.9); 4.092 (0.5); 4.074 (0.7); 4.057 (0.5); 3.938 (0.3); 3.929 (0.3); 3.918 (0.3); 3.913 (0.4); 3.868 (0.4); 3.851 (0.4); 3.828 (0.5); 3.796 (0.5); 3.780 (0.7); 3.751 (0.6); 3.737 (0.8); 3.727 (0.6); 3.708 (0.7); 3.639 (0.8); 3.527 (1.2); 3.511 (1.5); 3.496 (1.2); 3.481 (1.0); 3.458 (1.0); 3.454 (1.0); 3.445 (1.0); 3.405 (0.9); 3.392 (1.1); 3.369 (0.8); 3.341 (1.0); 3.328 (1.3); 3.312 (1.0); 3.243 (0.6); 3.182 (0.5); 3.160 (0.4); 3.130 (0.3); 3.111 (0.3); 2.891 (5.6); 2.732 (4.7); 2.676 (1.2); 2.672 (1.6); 2.667 (1.2); 2.563 (0.4); 2.525 (4.1); 2.520 (6.1); 2.512 (83.8); 2.507 (171.6); 2.503 (227.9); 2.498 (167.0); 2.494 (81.7); 2.334 (1.1); 2.329 (1.5); 2.325 (1.1); 2.113 (0.4); 1.502 (0.6); 1.234 (0.6); 0.146 (1.5); 0.022 (0.4); 0.020 (0.4); 0.008 (10.9); 0.000 (323.9); −0.009 (12.2); −0.026 (0.3); −0.150 (1.5)

No. Ia-60, Solvent: <[D₆]-DMSO>, spectrometer: 399.95 MHz

δ=9.218 (6.2); 8.410 (12.6); 8.387 (1.7); 8.317 (0.4); 8.012 (1.3); 7.916 (0.9); 7.910 (1.0); 7.903 (6.0); 7.897 (6.1); 7.855 (0.3); 7.808 (6.0); 7.803 (6.1); 7.760 (6.9); 7.758 (6.6); 7.544 (0.3); 7.539 (0.4); 7.509 (1.2); 7.469 (6.1); 7.465 (6.2); 7.448 (0.5); 7.441 (0.4); 7.419 (3.0); 7.413 (3.0); 7.398 (3.1); 7.393 (3.2); 6.852 (0.6); 6.831 (0.6); 6.583 (4.9); 6.562 (4.7); 6.342 (0.4); 6.233 (3.7); 6.228 (6.5); 6.223 (3.7); 5.822 (16.0); 5.780 (1.8); 5.698 (1.0); 3.332 (48.5); 2.676 (1.0); 2.672 (1.3); 2.667 (0.9); 2.525 (3.4); 2.507 (141.4); 2.503 (186.0); 2.498 (137.9); 2.334 (0.9); 2.330 (1.2); 2.325 (0.9); 1.352 (1.3); 1.337 (0.8); 1.299 (2.1); 1.259 (2.9); 1.249 (1.3); 1.234 (4.2); 1.198 (0.3); 1.073 (0.5); 1.056 (1.0); 1.038 (0.5); 0.863 (0.4); 0.854 (0.7); 0.835 (0.4); 0.146 (1.2); 0.017 (0.5); 0.008 (8.4); 0.000 (230.6); −0.008 (9.0); −0.150 (1.2)

No. Ia-61, Solvent: <[D₆]-DMSO>, spectrometer: 399.95 MHz

δ=9.337 (7.3); 8.819 (10.8); 8.045 (7.2); 8.040 (7.4); 7.961 (7.8); 7.958 (8.0); 7.939 (7.4); 7.934 (7.4); 7.644 (5.1); 7.623 (7.6); 7.606 (0.9); 7.601 (0.9); 7.545 (4.1); 7.540 (4.3); 7.522 (9.6); 7.519 (10.0); 6.313 (4.9); 6.308 (7.8); 6.303 (5.0); 6.256 (0.5); 5.872 (1.1); 5.855 (3.8); 5.838 (3.9); 5.820 (1.2); 3.853 (0.4); 3.817 (0.4); 3.789 (0.4); 3.763 (0.5); 3.736 (0.5); 3.678 (0.6); 3.675 (0.6); 3.622 (0.7); 3.604 (0.7); 3.588 (0.7); 3.569 (0.7); 3.530 (0.8); 3.493 (0.8); 3.467 (2.1); 3.450 (5.0); 3.432 (5.0); 3.415 (2.2); 3.291 (0.6); 3.250 (0.5); 3.208 (0.5); 3.187 (0.5); 3.172 (0.4); 3.166 (0.4); 2.676 (0.7); 2.672 (0.9); 2.668 (0.8); 2.507 (105.4); 2.503 (134.0); 2.499 (103.7); 2.330 (1.0); 1.842 (15.9); 1.824 (16.0); 1.074 (4.1); 1.056 (8.0); 1.039 (4.0); 0.146 (0.5); 0.000 (110.7); −0.150 (0.6)

No. Ia-62, Solvent: <[D₆]-DMSO>, spectrometer: 399.95 MHz

δ=5.40 (S, 2H); 6.31 (t, 1H); 7.35 (d, 1H); 7.51 (d, 1H); 7.80 (d, 1H); 7.90 (br. s, 1H); 7.95 (br. s, 1H); 8.08 (br. s, 1H); 8.80 (br. s, 1H); 9.33 (br. s, 1H) ppm

TABLE 6
Compounds of general formula (Ib), A¹ = N; A² = C—R^{1b};
A³ = C—R^{1c}; A⁴ = C—R^{1d}:

| | |
|---|---|
| | (Ib) |
| 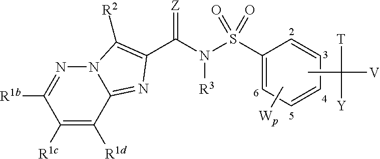 | |

| | | | | | | | | | | Posi- | |
|---|---|---|---|---|---|---|---|---|---|---|---|
| | | | | | | | | | | tion | |
| Ex. | | | | | | | | | | of | |
| No | R^{1b} | R^{1c} | R^{1d} | R² | Z | R³ | W | T | Y | C(T)(Y)(V) | V |
| Ib-1 | CF₃ | H | Cl | H | O | H | 2-Cl | H | H | 5 | 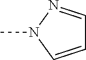 |
| Ib-2 | CF₃ | H | Cl | H | O | H | 2-Cl | H | H | 5 | 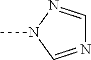 |
| Ib-3 | CF₃ | H | Cl | H | O | H | 2-Cl | H | H | 5 | 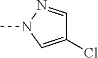 |
| Ib-4 | CF₃ | H | Cl | H | O | H | 2-Cl | H | H | 5 | 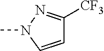 |
| Ib-5 | CF₃ | H | Cl | H | O | H | 2-Cl | H | H | 5 | 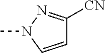 |
| Ib-6 | CF₃ | H | Cl | H | O | H | 2-Cl | H | H | 5 | 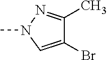 |
| Ib-7 | CF₃ | H | Cl | H | O | H | 2-Cl | H | H | 5 | 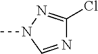 |
| Ib-8 | CF₃ | H | Cl | H | O | H | 2-Cl | H | H | 5 | 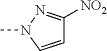 |
| Ib-9 | Cl | H | Cl | H | O | H | 2-Cl | H | H | 5 | 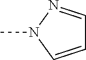 |
| Ib- 10 | CF₃ | H | Cl | H | O | H | 2-Cl | H | H | 5 | 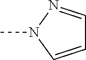 |
| Ib- 11 | Cl | H | Cl | H | O | H | 2-Cl | H | H | 5 | 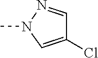 |

¹H-NMR Data of Compounds of Table 6:

No. Ib-9, Solvent: <[D₆]-DMSO>, spectrometer: 399.95 MHz

δ=8.367 (0.6); 8.340 (8.2); 8.317 (0.3); 8.155 (4.3); 8.131 (4.5); 7.948 (5.8); 7.944 (5.5); 7.853 (5.7); 7.848 (5.6); 7.478 (5.8); 7.364 (4.6); 7.342 (9.3); 7.317 (4.9); 7.189 (3.3); 7.185 (3.2); 7.169 (2.6); 7.165 (2.5); 6.283 (4.1); 6.279 (6.0); 5.830 (0.6); 5.388 (16.0); 3.328 (112.7); 3.081 (1.2); 2.671 (1.2); 2.502 (173.4); 2.329 (1.1); 2.086 (2.1); 1.235 (3.1); 1.177 (0.4); 1.140 (0.3); 0.854 (0.4); 0.000 (31.7)

No. Ib-10, Solvent: <[D₆]-DMSO>, spectrometer: 399.95 MHz

δ=8.555 (0.6); 8.526 (8.6); 8.388 (3.7); 8.364 (3.9); 8.177 (0.7); 8.164 (0.7); 7.972 (0.5); 7.960 (5.1); 7.955 (5.4); 7.859 (5.4); 7.854 (5.7); 7.638 (5.4); 7.614 (5.1); 7.481 (5.2); 7.477 (5.7); 7.370 (4.6); 7.350 (5.8); 7.331 (0.8); 7.198 (3.0); 7.193 (3.1); 7.178 (2.4); 7.173 (2.5); 6.796 (0.9); 6.780 (0.9); 6.287 (3.6); 6.282 (6.1); 6.277 (3.8); 5.844 (0.5); 5.757 (0.9); 5.421 (0.4); 5.395 (15.8); 3.428 (0.3); 3.328 (74.8); 3.284 (2.1); 3.254 (0.4); 3.242 (0.5); 3.067 (12.6); 3.048 (0.6); 3.032 (0.7); 3.017 (0.7); 2.999 (0.6); 2.981 (0.4); 2.803 (0.3); 2.672 (1.1); 2.602 (2.6); 2.541 (1.0); 2.507 (115.7); 2.503 (151.3); 2.499 (119.4); 2.329 (1.0); 2.086 (16.0); 1.647 (0.4); 1.235 (1.4); 1.169 (0.4); 1.151 (0.5); 1.116 (0.4); 1.099 (0.8); 1.081 (0.7); 0.997 (0.8); 0.979 (1.5); 0.961 (0.7); 0.000 (32.9)

No. Ib-11, Solvent: <[D₆]-DMSO>, spectrometer: 399.95 MHz

δ=8.626 (0.5); 8.353 (1.0); 8.326 (0.8); 8.317 (1.4); 8.301 (0.6); 8.275 (0.4); 8.238 (1.8); 8.219 (1.9); 8.153 (3.6); 8.128 (8.4); 8.021 (0.3); 7.961 (5.3); 7.925 (0.4); 7.583 (7.4); 7.516 (0.7); 7.492 (0.7); 7.480 (0.7); 7.469 (0.9); 7.445 (1.2); 7.390 (2.4); 7.343 (1.9); 7.255 (2.5); 6.980 (1.4); 6.962 (1.4); 6.529 (0.4); 5.757 (6.2); 5.443 (0.5); 5.372 (11.3); 5.328 (0.3); 3.712 (0.5); 3.468 (0.4); 3.328 (94.0); 3.204 (0.4); 3.199 (0.4); 3.178 (16.0); 3.021 (0.6); 2.995 (4.4); 2.929 (0.6); 2.711 (1.6); 2.675 (3.0); 2.671 (4.1); 2.667 (3.1); 2.604 (0.5); 2.585 (0.6); 2.541 (374.7); 2.524 (10.0); 2.511 (220.8); 2.506 (452.9); 2.502 (608.1); 2.497 (458.0); 2.493 (232.0); 2.367 (1.5); 2.333 (2.8); 2.329 (3.9); 2.324 (3.0); 2.296 (1.6); 1.378 (0.4); 1.302 (0.4); 1.287 (0.4); 1.234 (0.4); 0.146 (3.1); 0.029 (0.4); 0.008 (23.8); 0.000 (682.1); −0.008 (28.8); −0.033 (0.5); −0.039 (0.4); −0.150 (3.2)

TABLE 7
Compounds of general formula (Ic), A¹ = C—R^{1a}; A² = C—R^{1b};
A³ = N, A⁴ = C—R^{1d}:

| | |
|---|---|
| | (Ic) |
| 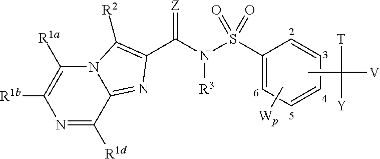 | |

| Ex. | | | | | | | | | | Position of | |
|---|---|---|---|---|---|---|---|---|---|---|---|
| No | R^{1a} | R^{1b} | R^{1d} | R² | Z | R³ | W | T | Y | C(T)(Y)(V) | V |
| Ic-1 | H | CF₃ | Cl | H | O | H | 2-Cl | H | H | 5 | 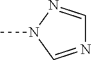 |
| Ic-2 | H | I | OEt | H | O | H | 2-Cl | H | H | 5 | 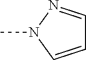 |
| Ic-3 | H | I | Cl | H | O | H | 2-Cl | H | H | 5 | 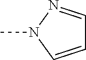 |

¹H-NMR Data of Compounds of Table 7:

No. Ic-2, Solvent: <[D₆]-DMSO>, spectrometer: 399.95 MHz

δ=8.545 (4.9); 8.209 (1.4); 8.191 (1.4); 8.103 (4.7); 7.935 (2.0); 7.930 (2.1); 7.845 (2.2); 7.840 (2.2); 7.474 (2.1); 7.470 (2.1); 7.354 (2.0); 7.334 (2.5); 7.184 (1.2); 7.179 (1.2); 7.164 (1.0); 7.159 (0.9); 6.834 (1.4); 6.816 (1.3); 6.281 (1.5); 6.276 (2.5); 6.271 (1.4); 5.383 (6.2); 4.465 (0.9); 4.448 (2.8); 4.430 (2.8); 4.412 (0.9); 3.327 (75.6); 3.284 (1.1); 3.091 (16.0); 3.033 (0.4); 3.017 (0.4); 2.676 (0.7); 2.671 (1.0); 2.667 (0.7); 2.642 (1.7); 2.541 (0.7); 2.510 (57.1); 2.507 (111.7); 2.502 (145.0); 2.498 (104.5); 2.333 (0.7); 2.329 (0.9); 2.325 (0.7); 2.086 (5.4); 1.411 (3.0); 1.393 (6.3); 1.376 (2.9); 1.235 (0.8); 1.140 (0.4); 1.100 (0.4); 1.083 (0.4); 0.997 (0.5); 0.980 (0.9); 0.962 (0.4); 0.008 (1.5); 0.000 (36.0); −0.008 (1.4)

No. Ic-3, Solvent: <[D₆]-DMSO>, spectrometer: 399.95 MHz

δ=9.109 (1.4); 9.100 (1.9); 8.725 (1.1); 8.705 (1.5); 8.054 (1.4); 7.907 (1.6); 7.902 (1.7); 7.644 (1.3); 7.624 (1.6); 7.515 (1.6); 7.511 (1.8); 7.482 (0.9); 7.477 (0.9); 7.461 (0.8); 7.456 (0.7); 6.317 (1.1); 6.312 (1.9); 6.307 (1.2); 5.757 (0.4); 5.498 (4.7); 5.484 (0.4); 3.386 (0.4); 3.187 (2.0); 2.766 (0.6); 2.753 (0.7); 2.507 (24.7); 2.503 (31.9); 2.499 (23.7); 2.087 (16.0); 1.910 (0.9); 1.234 (1.4); 0.987 (0.4); 0.008 (0.4); 0.000 (8.1)

### Application Examples

1. Meloidogyne incognita—test (MELGIN)

| | |
|---|---|
| Solvent: | 125.0 parts by weight of acetone |

To produce a suitable preparation of active compound, 1 part by weight of active compound is mixed with the stated amount of solvent, and the concentrate is diluted with water to the desired concentration.

Vessels are filled with sand, a solution of the active ingredient, a suspension containing eggs and larvae of the southern root-knot nematode (Meloidogyne incognita) and salad seeds. The salad seeds germinate and the seedlings grow. Galls develop in the roots.

After 14 days the nematicidal activity is determined on the basis of the percentage of gall formation. 100% means no galls were found; 0% means the number of galls found on the roots of the treated plants was equal to that in untreated control plants.

In this test, for example, the following compounds from the preparation examples showed good activity of 100% at an application rate of 20 ppm: Ia-2, Ia-5, Ia-6, Ia-7, Ia-8, Ia-9, Ia-10, Ia-11, Ia-12, Ia-14, Ia-15, Ia-16, Ia-18, Ia-22, Ia-24, Ia-29, Ia-31, Ia-41, Ia-43, Ia-55

In this test, for example, the following compounds from the preparation examples showed good activity of 90% at an application rate of 20 ppm: Ia-17, Ia-20, Ia-21, Ia-27, Ia-28, Ia-36, Ia-44, Ia-45, Ia-46, Ia-47, Ia-49, Ia-52, Ic-2

2. Meloidogyne incognita—test (MELGIN)

| | | |
|---|---|---|
| Solvent: | 7 | parts by weight of dimethylformamide |
| Emulsifier: | 2.5 | parts by weight of alkylarylpolyglycolether |

To produce a suitable preparation of active compound, 1 part by weight of active compound is mixed with the stated amount of solvent, and the concentrate is diluted with emulsifier-containing water to the desired concentration.

Vessels are filled with BI-soil, salad seeds, a solution of the active ingredient and a suspension containing eggs and larvae of the southern root-knot nematode (Meloidogyne incognita) and are covered with quartz sand. The salad seeds germinate and the seedlings grow. Galls develop on the roots.

After 21 days the nematicidal activity is determined on the basis of the percentage of gall formation. 100 means no galls were found; 0% means the number of galls found on the roots of the treated plants was equal to that in untreated control plants.

In this test, for example, the following compounds from the preparation examples showed good activity of 95% at an application rate of 2 ppm: Ia-2

In this test, for example, the following compounds from the preparation examples showed good activity of 97% at an application rate of 2 ppm: Ia-15

In this test, for example, the following compounds from the preparation examples showed good activity of 80% at an application rate of 2 ppm: Ia-24

In this test, for example, the following compounds from the preparation examples showed good activity of 95% at an application rate of 4 ppm: Ia-18

In this test, for example, the following compounds from the preparation examples showed good activity of 90% at an application rate of 4 ppm: Ia-5

In this test, for example, the following compounds from the preparation examples showed good activity of 80% at an application rate of 4 ppm: Ia-31, Ia-49

In this test, for example, the following compounds from the preparation examples showed good activity of 99% at an application rate of 8 ppm: Ia-41

In this test, for example, the following compounds from the preparation examples showed good activity of 85% at an application rate of 8 ppm: Ia-22, Ia-47

### DESCRIPTION OF THE FIGURE

FIG. 1: Spectroscopic data of 8-chloro-N-{[2-chloro-5-(chloromethyl)phenyl]sulfonyl}-6-(trifluoromethyl)imidazo[1,2-a]pyridine-2-carboxamide (see preparation example 4)

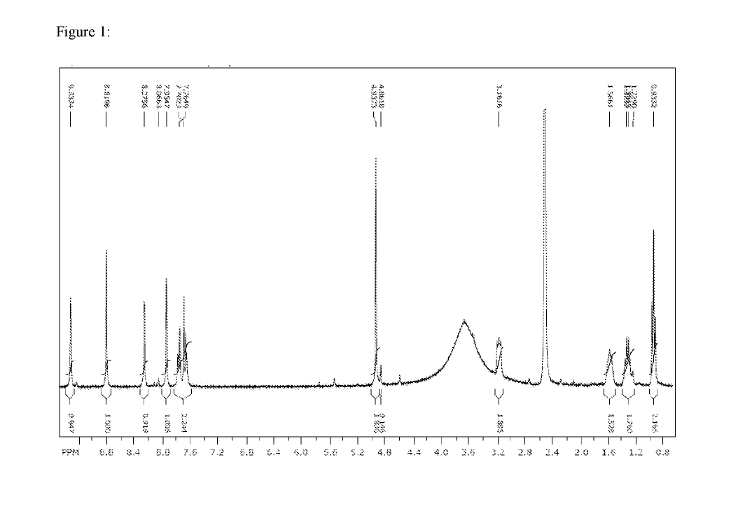

1. Compound of formula (I) 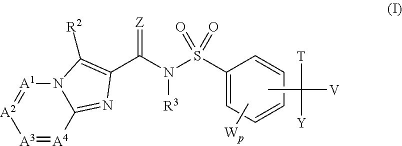
wherein
Z is O or S,
A¹, A², A³ and A⁴ are independently N or C—R¹;
each W is independently hydrogen, halogen, cyano, nitro, SF₅, OCN, SCN, Si(R¹⁴)₃, OR⁴, NR⁵R⁶, C₁-C₆-alkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹²; OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹, N(R¹⁰)S(O)₂NR¹¹R¹², C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
each R¹ is independently H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷, N(R¹⁰)C(O)NR¹¹R¹², N(R¹⁰)S(O)₂R⁹ or N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R² is H, halogen, cyano, nitro, SF₅, OCN, SCN, Si(R¹⁴)₃, OR⁴, NR⁵R⁶, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R⁷, C(O)OR⁸, C(O)NR¹¹R¹², S(O)ₘR⁹, S(O)₂NR¹¹R¹², OC(O)R⁷, OC(O)OR⁸, OC(O)NR¹¹R¹², OS(O)₂R⁹, OS(O)₂NR¹¹R¹², N(R¹⁰)C(O)R⁷,) N(R¹⁰)C(O)NR¹¹R¹², N(R¹⁰)S(O)₂R⁹ or N(R¹⁰)S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or phenyl, naphthalenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C(X)R⁷, C(O)OR⁸, C(O)NR¹¹R¹², S(O)ₘR⁹ and S(O)₂NR¹¹R¹²;
R³ is H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, OR^{4a}, and S(O)ₘR^{9a};
or C₁-C₆-alkyl substituted with 1 to 4 substituents independently selected from the group consisting of cyano, nitro, OR⁴, NR⁵R⁶, C(X)R⁷, C(O)OR⁸, C(O)NR¹¹R¹², S(O)ₘR⁹ and S(O)₂NR¹¹R¹²;
or C₁-C₆-alkyl substituted with 1 to 2 substituents independently selected from the group consisting of phenyl or a 5- or 6-membered heteroaromatic ring, each optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R⁷, C(O)OR⁸, C(O)NR¹¹R¹², S(O)ₘR⁹, S(O)₂NR¹¹R¹², OC(O)R^{7a} and N(R¹⁰)C(O)R^{7a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R¹, C(O)OR⁸, C(O)NR¹¹R¹², S(O)ₘR⁹, S(O)₂NR¹¹R¹², OC(O)R^{7a} and N(R¹⁰)C(O)R^{7a};
each R⁴ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)ₘR^{9a} and S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{4a} is independently H, C₁-C₆-alkyl or C₁-C₆-haloalkyl;
each R⁵ is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR¹¹R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR⁸a, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{5a} is independently H or C₁-C₆-alkyl;
each R⁶ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R^{6a} is independently H, C₁-C₆-alkyl, C(O)R¹³ or C(O)OR¹³;
each R⁷ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{7a} is independently C₁-C₆-alkyl or C₁-C₆-haloalkyl;
each R⁸ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)R^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{8a} is independently C₁-C₆-alkyl or C₁-C₆-haloalkyl;
each R⁹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl; or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR¹¹R¹², S(O)R^{9a} and S(O)₂NR¹¹R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R^{9a} is independently C₁-C₆-alkyl or C₁-C₆-haloalkyl;
each R¹⁰ is independently H, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₇-cycloalkyl, C(X)R^{7a}, C(O)OR^{8a}, C(O)NR^{H}R¹², S(O)ₘR^{9a} or S(O)₂NR¹¹R¹²;
each R¹¹ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or C₁-C₆-alkyl, C₂-C₆-alkenyl or C₂-C₆-alkynyl, each substituted with 1 to 4 substituents independently selected from the group consisting of, cyano, nitro, OR^{4a}, NR^{5a}R^{6a}, C(X)R^{7a}, C(O)OR^{8a}, C(O)NR^{11a}R¹², S(O)ₘR^{9a} and S(O)₂NR^{11a}R¹²;
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR^{11a}R¹², OR^{4a}, C₂-C₆-alkoxyalkyl, S(O)ₘR^{9a}, S(O)₂NR^{11a}R¹², NR^{5a}R^{6a}, OC(O)R^{7a} and N(R¹⁰)C(O)R^{7a};
each R^{11a} is independently H, C₁-C₆-alkyl, C₂-C₆-alkenyl or C₂-C₆-alkynyl;
each R¹² is independently H, NR^{5a}R^{6a}, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C(X)R⁷, C(O)OR⁸, C(O)NR^{H}R¹², S(O)ₘR⁹ or S(O)₂NR¹¹R¹²;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 3 substituents independently selected from the group consisting of halogen, cyano, nitro, C₁-C₄-alkyl, C₂-C₄-alkenyl, C₂-C₄-alkynyl, C₁-C₄-haloalkyl, C₂-C₄-haloalkenyl, C(X)R^{7a}, C(O)OR^{8a}, C(O)NR¹¹R¹², OR^{4a}, C₂-C₆-alkoxyalkyl, S(O)ₘR^{9a}, S(O)₂NR¹¹R¹², NR^{5a}R^{6a}, OC(O)R^{7a} and N(R¹⁰)C(O)R^{7a};
each R¹³ is independently H, C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl, C₆-C₁₄-cycloalkylcycloalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
each R¹⁴ is independently C₁-C₆-alkyl, C₁-C₆-haloalkyl, C₂-C₆-alkenyl, C₂-C₆-haloalkenyl, C₂-C₆-alkynyl or C₂-C₆-haloalkynyl;
or C₃-C₇-cycloalkyl, C₄-C₈-cycloalkylalkyl or C₅-C₇-cycloalkenyl, each optionally substituted with 1 to 4 substituents independently selected from the group consisting of halogen, cyano, nitro, C₁-C₄-alkyl, C₁-C₄-haloalkyl, OR^{4a} and S(O)ₘR^{9a};
or phenyl optionally substituted with 1 to 5 substituents independently selected from the group consisting of halogen, cyano, nitro, OR⁴, NR⁵R⁶, C₁-C₄-alkyl, C₂-C₄-alkenyl, C₂-C₄-alkynyl, C₁-C₄-haloalkyl, C₂-C₄-haloalkenyl, C₂-C₆-alkoxyalkyl, C(X)R¹, C(O)OR⁸, C(O)NR¹¹R¹², S(O)ₘR⁹, S(O)₂NR¹¹R¹², OC(O)R^{7a} and N(R¹⁰)C(O)R^{7a};
X is O or S;
T is hydrogen, halogen, C₁-C₄-alkyl or C₁-C₄-haloalkyl;
Y is hydrogen, halogen, C₁-C₄-alkyl or C₁-C₄-haloalkyl;
V is a saturated, partially unsaturated or fully unsaturated via nitrogen bonded heterocyclic group containing 3 to 7 ring atoms;
each m is independently 0, 1 or 2;
p is 0, 1, 2, 3 or 4.

2. Compound according to claim 1,
wherein;
V is selected from the group consisting of 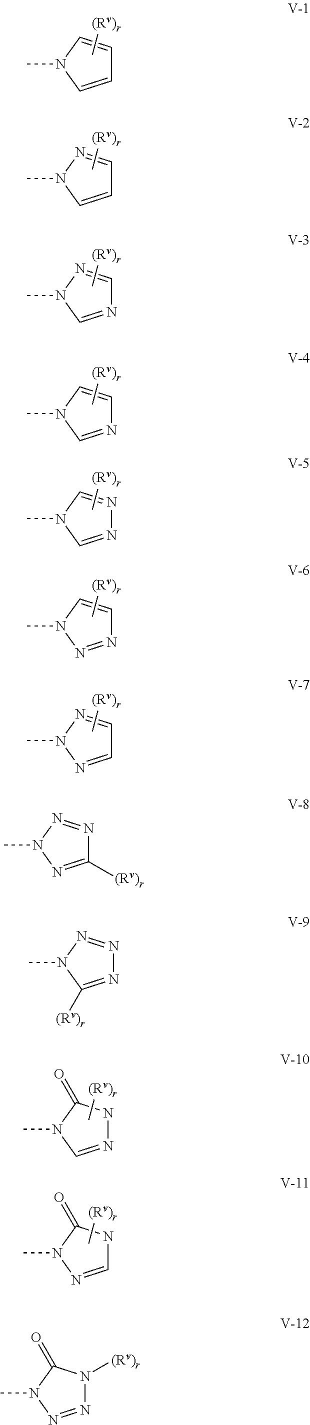 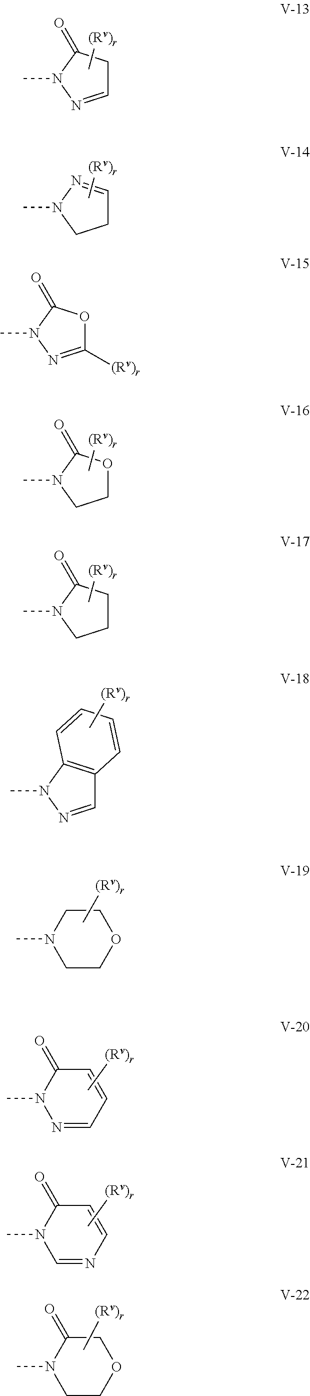
R^{v} is any substituent as defined for R¹, R², R³;
r is an integer from 0 to 4, limited by the number of available positions on each V group.

3. Compounds according to claim 1, wherein
A¹, A², A³ and A⁴ are C—R¹;
T is hydrogen, C₁-C₄-alkyl or C₁-C₄-haloalkyl;
Y is hydrogen, C₁-C₄-alkyl or C₁-C₄-haloalkyl.

4. Compound according to claim 3, wherein T is hydrogen or methyl and Y is hydrogen.

5. Compound according to claim 4, wherein T is hydrogen and Y is hydrogen.

6. Compound according to claim 1, wherein
one of A¹, A², A³ and A⁴ is N and the remaining are C—R¹;
T is hydrogen, C₁-C₄-alkyl or C₁-C₄-haloalkyl;
Y is hydrogen, C₁-C₄-alkyl or C₁-C₄-haloalkyl.

7. Compound according to claim 6, wherein T is hydrogen or methyl and Y is hydrogen.

8. Compound according to claim 7, wherein T is hydrogen and Y is hydrogen.

9. Compound of formula (Ia) 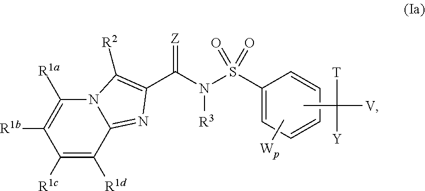
wherein
Z is O;
R^{1a}, R^{1b}, R^{1c} and R^{1d} are independently hydrogen, fluorine, chlorine, bromine, iodine, ethoxy or trifluoromethyl;
T is hydrogen or methyl;
Y is hydrogen;
V is selected from the group consisting of V-1 to V-22 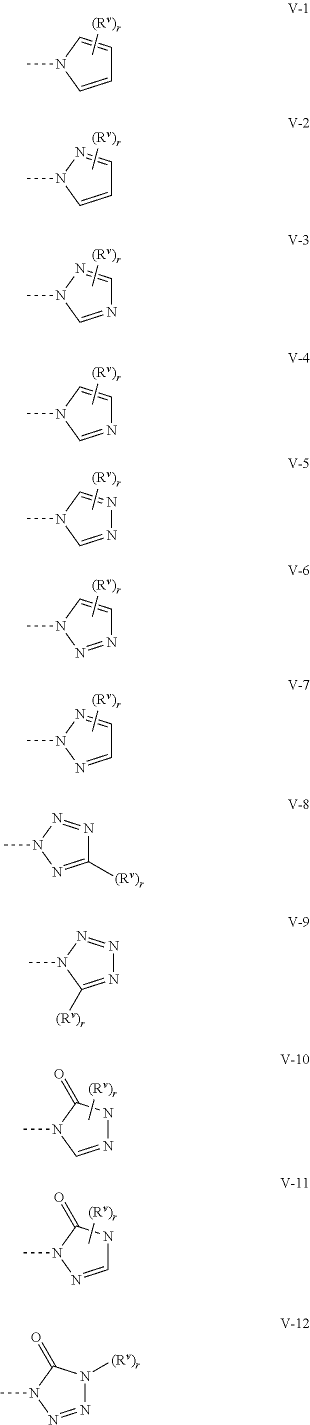 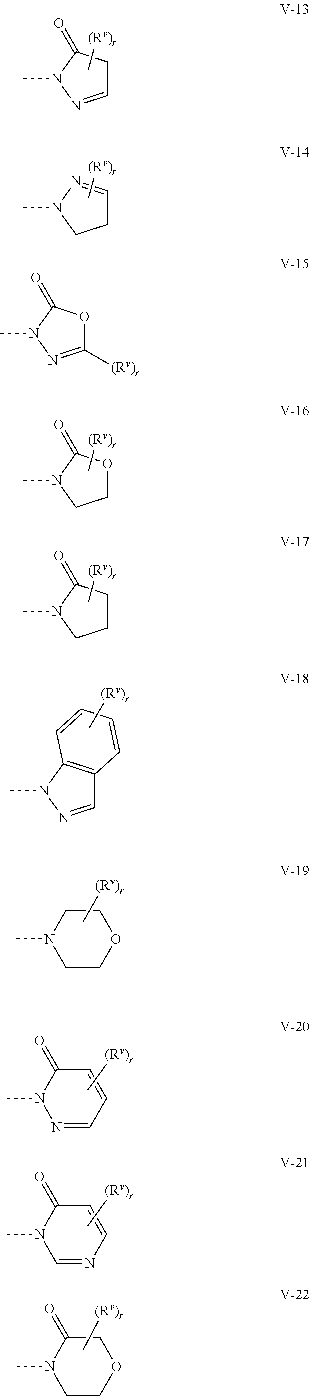
p is 0, 1 or 2;
each W is independently hydrogen, fluorine, chlorine, bromine, methoxy, trifluormethoxy, methyl or ethyl;
R² is hydrogen;
R³ is hydrogen;
each R^{v} is independently H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl;
r is 0, 1 or 2.

10. Compound of formula (Ib) 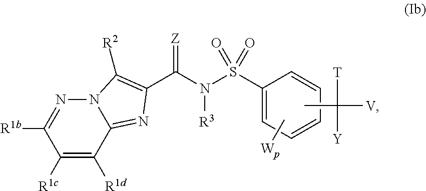
wherein
Z is O;
R^{1b}, R^{1c} and R^{1d} are independently hydrogen, fluorine, chlorine, bromine, iodine, ethoxy or trifluoromethyl;
T is hydrogen or methyl;
Y is hydrogen;
V is selected from the group consisting of V-1 to V-22; 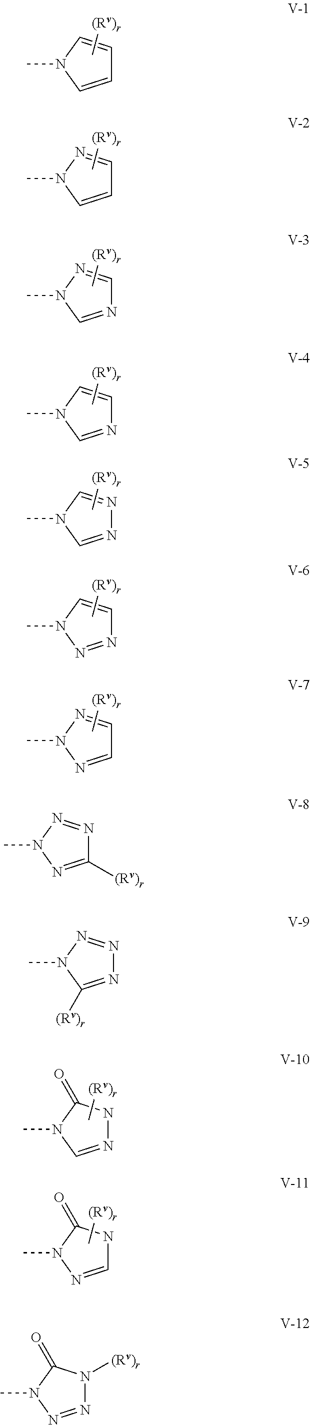 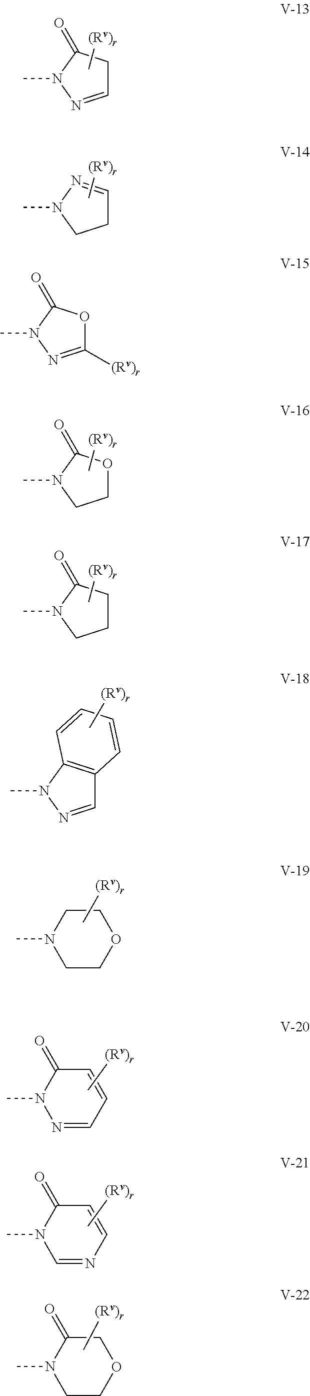
p is 0, 1 or 2;
each W is independently hydrogen, fluorine, chlorine, bromine, methoxy, trifluormethoxy, methyl or ethyl;
R² is hydrogen;
R³ is hydrogen;
each R^{v} is independently H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl;
r is 0, 1 or 2.

11. Compound of formula (Ic) 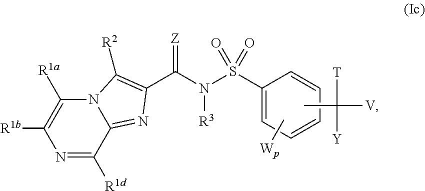
wherein
Z is O;
R^{1a}, R^{1b} and R^{1d} are independently hydrogen, fluorine, chlorine, bromine, iodine, ethoxy or trifluoromethyl;
T is hydrogen or methyl;
Y is hydrogen;
V is selected from the group consisting of 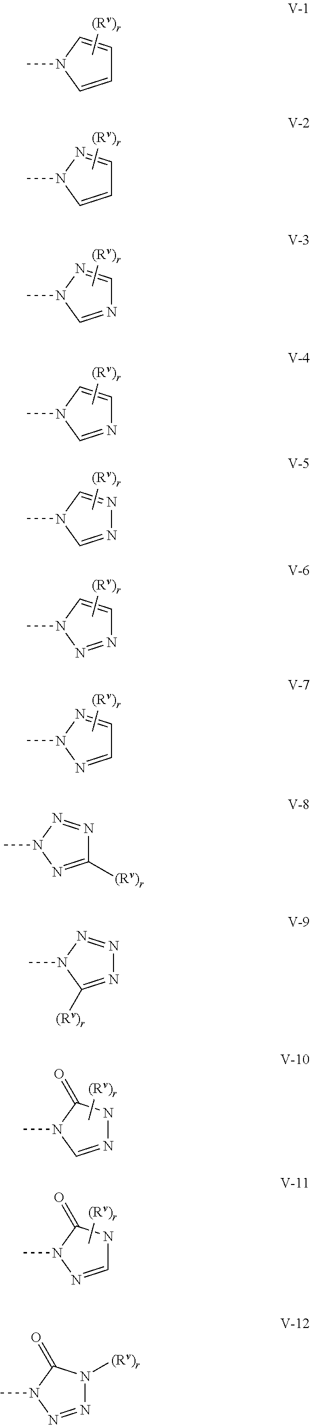 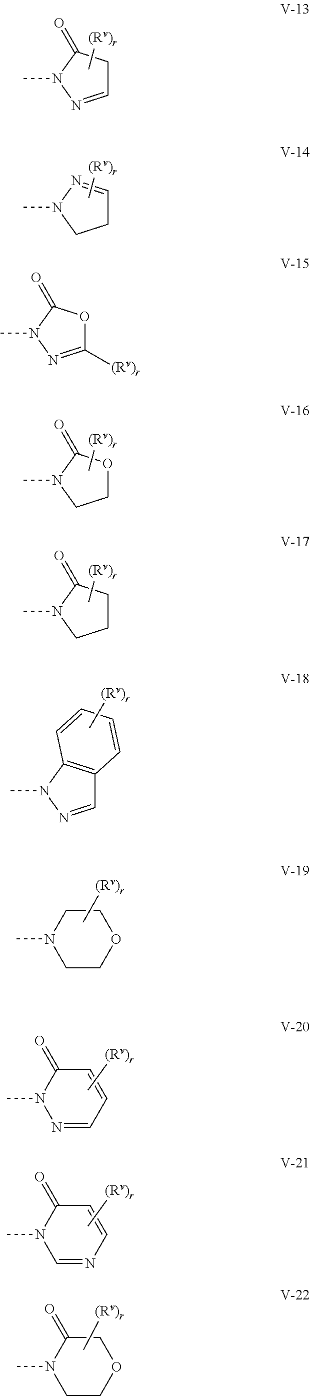
V-1 to V-22 as defined in claim 2;
p is 0, 1 or 2;
each W is independently hydrogen, fluorine, chlorine, bromine, methoxy, trifluormethoxy, methyl or ethyl;
R² is hydrogen;
R³ is hydrogen;
each R^{v} is independently H, fluorine, chlorine, bromine, cyano, nitro, methyl, trifluoromethyl, COOCH₃, COOC₂H₅, O—C₃H₇-iso, methoxy, thien-2-yl, pyridine-2-yl, pyridine-3-yl, carboxyl, cyclopropyl, N(CH₃)₂, SCH₃ or phenyl optionally substituted with one substituent selected from the group of halogen, especially 4-chlorophenyl;
r is 0, 1 or 2.

12. Compound of formula (IIIA) 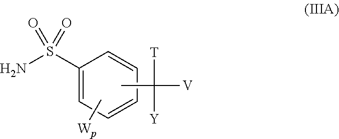
wherein
W, Y, T and p have the meanings as defined in claim 1;
V is a saturated, partially unsaturated or fully unsaturated via nitrogen bonded heterocyclic group containing 3 to 7 ring atoms.

13. Compositions comprising at least one compound of formula (I) according to claim 1.

14. Composition comprising an effective amount of at least one compound of formula (I) according to claim 1 and at least one surfactant, solid or liquid diluent.

15. Composition according to claim 13 which further comprises at least one additional biologically active compound or agent.

16. A compound of the formula (I) according to claim 1 capable of being used for controlling one or more animal pests, optionally parasitic nematodes, in crop protection, in the protection of materials and/or in the veterinary sector.

17. A method for controlling a parasitic nematode comprising contacting the parasitic nematode and/or an environment thereof with a biologically effective amount of a compound of formula (I) according to claim 1.

18. A method for controlling a parasitic nematode comprising contacting the parasitic nematode and/or an environment thereof with a composition according to claim 13.

19. A method for protecting a seed from a parasitic nematode comprising contacting the seed with a biologically effective amount of a compound of formula (I) according to claim 1.

20. A seed obtained by a method according to claim 19.
